(19) United States
(12) Patent Application Publication
(10) Pub. No.: US 2025/0375534 A1
(43) Pub. Date: Dec. 11, 2025

(54) COMPLEXES FOR DELIVERY OF NUCLEIC ACIDS

(71) Applicant: BioNTech SE, Mainz (DE)

(72) Inventors: Jorge Moreno Herrero, Mainz (DE); Irena Vlatkovic, Mainz (DE); Heinrich Haas, Mainz (DE); Stephanie Erbar, Chiclana de la Frontera (ES); Egon Jack Jacobus Ambuludi, Mainz (DE); Louisa Zimmermann, Ginsheim-Gustavsburg (DE); Katalin Karikó, Rydal, PA (US); Jason Matthew Keller, Gorinchem (NL); Maximiliano Luis Cacicedo, Mainz (DE); Hossam Hefesha, Wiesbaden (DE)

(21) Appl. No.: 18/869,559

(22) PCT Filed: May 29, 2023
(86) PCT No.: PCT/EP23/64341

§ 371 Date: Nov. 26, 2024

(30) Foreign Application Priority Data
May 30, 2022 (WO) PCT/EP22/64542

(51) Int. Cl. A61K 48/00

(52) U.S. Cl. CPC A61K 48/0041; A61K 48/0058

(57) ABSTRACT

The present disclosure provides nucleic acid particles comprising an immunomodulator, RNA, and a cationic lipid or a cationic polymer, wherein nucleic acid particles described herein reduce inflammatory response and/or increase protein or antigen expression associated with previous formulations.

### CROSS-REFERENCE TO RELATED APPLICATIONS

The present application claims priority to PCT Application No. PCT/EP22/64542, filed May 30, 2022, the entirety of which is incorporated by reference.

### BACKGROUND

Nucleic acid particles are complexes that are useful in the delivery of nucleic acid therapies to patients. Generally, nucleic acid particles comprise lipids (e.g., lipid nanoparticles (LNPs), liposomes, and lipoplexes) and/or cationic polymers (e.g., polyplexes), and a nucleic acid, e.g., ribonucleic acid (RNA).

### SUMMARY

Nucleic acid therapies face particular challenges when administered to patients. To be effective, the nucleic acid molecule needs to reach the target tissue and produce particular proteins of interest. Nucleic acids, however, can be unstable, are susceptible to degradation after administration, and in isolation have a limited ability to enter the target tissue. As such, nucleic acid particle delivery technologies are needed that encapsulate the nucleic acid and facilitate delivery to the patient and the target of interest within the patient. In particular, nucleic acid particles comprising lipids (e.g., lipid nanoparticles (LNPs), liposomes, and lipoplexes) as well as those comprising polymers (e.g., polyplexes) have emerged as vehicles to facilitate delivery of nucleic acids to a target of interest.

Particles comprising cationic lipids and/or cationic polymers are carefully constructed vehicles that can rely on an interplay of different components to provide a safe means of passage for nucleic acid cargo. Lipid nanoparticles (LNPs), for example, include cationic lipids and particular helper compounds that stabilize the particle, to ensure that the LNP can release the cargo at the right time and place. Certain particles, such as those comprising lipids, including LNPs, while widely considered to be safe and effective for the delivery of nucleic acid therapies, and indeed used in commercial products globally, can still cause an inflammatory reaction by the immune system after administration. For example, it has been noted that intradermal and intramuscular administration of LNPs triggers “inflammation characterized by leukocytic infiltration, activation of different inflammatory pathways, and secretion of a diverse pool of inflammatory cytokines and chemokines.” Ndeupen, et al., iScience, 24, 103479 (Dec. 17, 2021). These responses can be characterized as pain, swelling, fever, and the like.

The present disclosure encompasses an insight that inclusion of particular agents as part of the nucleic acid particle, such as lipid-based particles (e.g., LNPs, lipoplexes, and liposomes) as well as polymer-based particles (e.g., polyplexes) reduce inflammatory response upon administration, and further, in some embodiments, can increase translation. Without wishing to be bound by theory, it is hypothesized that provided nucleic acid particles not only retain stability for delivering nucleic acid to a target, but further, reduce inflammatory response in the patient, thereby avoiding particular side effects such as pain, fever, and the like. Nucleic acid particles described herein incorporate these agents into the structure of the particle itself. Moreover, nucleic acid particles described herein surprisingly also demonstrate improved expression of proteins or antigens that are encoded by nucleic acids that are delivered by said particles.

Other efforts to reduce said side effects have focused on modification of certain components of the nanoparticle, including, for example, modification of the cationic lipid to incorporate certain steroidal structural features. The present disclosure, in contrast, incorporates immunomodulatory compounds, e.g., TLR inhibitors and/or inflammasome inhibitors directly into the structure of the particle as discrete agents.

Further, others have attempted to use dexamethasone to reduce inflammation upon administration offormulations comprising nucleic acids (e.g., RNA). See, e.g., Chen, et al., J. of Controlled Release, 286:46-54 (2018); Zhang, et al., J. Biomed. Mater. Res. 2022; 1-8; I. Vlatkovic, Biomedicines, 9:520 (2021)). Applicant has discovered alternative agents that solve the problem of inflammation response upon administration of nucleic acid particles.

In some embodiments, the present disclosure provides a nucleic acid particle comprising RNA, an immunomodulator, and a cationic lipid or a cationic polymer. In some embodiments, an immunomodulator is not dexamethasone.

In some embodiments, a nucleic acid particle comprises an immunomodulator, a cationic lipid, and RNA. In some embodiments, a nucleic acid particle is in the form of a lipid nanoparticle. In some embodiments, a lipid nanoparticle further comprises one or more of a helper lipid and a polymer-conjugated lipid.

In some embodiments, a nucleic acid particle comprises an immunomodulator, a cationic polymer, and RNA.

In some embodiments, the present disclosure provides a method of increasing or causing increased expression of RNA in a target in a subject, the method comprising administering to the subject a nucleic acid particle as described herein.

In some embodiments, the present disclosure provides a method of treating a disease, disorder, or condition in a subject comprising administering to the subject a nucleic acid particle described herein.

In some embodiments, the present disclosure provides a nucleic acid particle as described herein for use as a medicament.

In some embodiments, the present disclosure provides a nucleic acid particle as described herein for use in the treatment and/or prevention of a disease or disorder, wherein the disease or disorder is an infectious disease, cancer, a genetic disorder, an autoimmune disease, or a rare disease.

### BRIEF DESCRIPTION OF THE DRAWINGS

FIG. 1 is a heatmap showing cytokine profile concentration in human PBMC (hPBMC) for Formulations 1-16 described herein after transfection of 3 μg or 0.3 μg of formulated modRNA per 5×10⁵ viable cells.

FIG. 2 is a heatmap illustrating luciferase activity in hepatocytes after transfection of 0.1 μg of formulated modRNA per 2.5×10⁴ cells.

FIG. 3 is a series of plots providing the cytokine profile across a variety of concentrations (0.01 to 3 μg/well) for Formulations 1-4, and the results from an XTT assay of Formulations 1-4.

FIG. 4 is a series of plots illustrating luciferase activity (measured as RLUs) on Days 1, 2, and 7 for provided Formulations 1-4.

FIG. 5 is a series of plots providing the cytokine profile across a variety of concentrations (0.01 to 3 μg/well) for Formulations 5-8, and the results from an XTT assay of Formulations 5-8.

FIG. 6 is a series of plots illustrating luciferase activity (measured as RLUs) on Days 1, 2, and 7 for provided Formulations 5-8.

FIG. 7 is a series of plots providing the cytokine profile across a variety of concentrations (0.01 to 3 μg/well) for Formulations 13-16, and the results from an XTT assay of Formulations 13-16.

FIG. 8 is a series of plots illustrating luciferase activity (measured as RLUs) on Days 1, 2, and 7 for provided Formulations 13-16.

FIG. 9 is a series of plots providing the cytokine profile across a variety of concentrations (0.01 to 3 μg/well) for Formulations 9-12, and the results from an XTT assay of Formulations 9-12.

FIG. 10 is a series of plots providing the cytokine profile across a variety of concentrations (0.01 to 3 μg/well) for Formulations 13-16, and the results from an XTT assay of Formulations 17-20.

FIG. 11 is a series of plots illustrating luciferase activity (measured as RLUs) on Days 1, 3, and 7 for provided Formulations 17-20.

### DETAILED DESCRIPTION OF CERTAIN EMBODIMENTS

The present disclosure provides, among other things, nucleic acid particles (e.g., cationic lipid-based particles such as lipid nanoparticles (LNPs), liposomes, lipoplexes, and cationic polymer-based particles such as polyplexes) useful for delivery of a nucleic acid, e.g., RNA, and uses thereof. The present disclosure also provides, among other things, complexes that avoid problems associated with previously known nucleic acid particles, including an inflammatory response, or increased levels of cytokines or interleukins that can cause pain, fever, and other adverse reactions after administration. As described herein, the present disclosure provides a nucleic acid particle (and compositions, e.g., pharmaceutical compositions that comprise said nucleic acid particle) comprising RNA, an immunomodulator, and a cationic lipid or cationic polymer. In some embodiments, nucleic acid particles described herein are useful for the treatment of a variety of diseases. In some embodiments, such nucleic acid particles can be administered via systemic, intravenous, or intranasal means.

### Definitions

Compounds of this disclosure include those described generally above and are further illustrated by the classes, subclasses, and species disclosed herein. As used herein, the following definitions shall apply unless otherwise indicated. For purposes of this disclosure, the chemical elements are identified in accordance with the Periodic Table of Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

About or approximately: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within (i.e., ±) 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to the administration of a composition (e.g., a pharmaceutical composition) to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be parenteral. In some embodiments, administration may be oral. In some particular embodiments, administration may be intravenous. In some particular embodiments, administration may be subcutaneous. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may comprise a prime-and-boost protocol. A prime-and-boost protocol can include administration of a first dose of a pharmaceutical composition (e.g., an immunogenic composition, e.g., a vaccine) followed by, after an interval of time, administration of a second or subsequent dose of a pharmaceutical composition (e.g., an immunogenic composition, e.g., a vaccine). In the case of an immunogenic composition, a prime-and-boost protocol can result in an increased immune response in a patient.

Agonist: As used herein, the term “agonist” generally refers to an agent whose presence or level correlates with elevated level or activity of a target, as compared with that observed absent the agent (or with the agent at a different level). In some embodiments, an agonist is one whose presence or level correlates with a target level or activity that is comparable to or greater than a particular reference level or activity (e.g., that observed under appropriate reference conditions, such as presence of a known agonist, e.g., a positive control). In some embodiments, an agonist may be a direct agonist in that it exerts its influence directly on (e.g., interacts directly with) the target; in some embodiments, an agonist may be an indirect agonist in that it exerts its influence indirectly (e.g., by acting on, such as interacting with, a regulator of the target, or with some other component or entity.

Aliphatic: The term “aliphatic” refers to a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point or more than one points of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, or alkynyl groups and hybrids thereof. A preferred aliphatic group is C₁₋₆ alkyl.

Alkyl: The term “alkyl”, used alone or as part of a larger moiety, refers to a saturated, optionally substituted straight or branched chain hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkylene: The term “alkylene” is refers to a bivalent alkyl group. In some embodiments, “alkylene” is a bivalent straight or branched alkyl group. In some embodiments, an “alkylene chain1” is a polymethylene group, i.e., —(CH₂)ₙ—, wherein n is a positive integer, e.g., from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In certain embodiments, two substituents can be taken together to form a 3- to 7-membered ring. The substituents can be on the same or different atoms. The suffix “-ene” or “-enyl” when appended to certain groups herein are intended to refer to a bifunctional moiety of said group. For example, “-ene” or “-enyl”, when appended to “cyclopropyl” becomes “cyclopropylene” or “cyclopropylenyl” and is intended to refer to a bifunctional cyclopropyl group, e.g.,

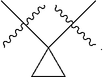 [SMILES: *C1(*)CC1]

Alkenyl: The term “alkenyl”, used alone or as part of a larger moiety, refers to an optionally substituted straight or branched chain or cyclic hydrocarbon group having at least one double bond and having (unless otherwise specified) 2-12, 2-10, 2-8, 2-6, 2-4, or 2-3 carbon atoms(e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Antagonist: As will be understood by those skilled in the art, the term “antagonist” generally refers to an agent whose presence or level correlates with decreased level or activity of a target, as compared with that observed absent the agent (or with the agent at a different level). In some embodiments, an antagonist is one whose presence or level correlates with a target level or activity that is comparable to or less than a particular reference level or activity (e.g., that observed under appropriate reference conditions, such as presence of a known antagonist, e.g., a positive control). In some embodiments, an antagonist may be a direct antagonist in that it exerts its influence directly on (e.g., interacts directly with) the target; in some embodiments, an antagonist may be an indirect antagonist in that it exerts its influence indirectly (e.g., by acting on, such as interacting with, a regulator of the target, or with some other component or entity.

Aryl: The term “aryl” refers to monocyclic and bicyclic ring systems having a total of six to fourteen ring members (e.g., C₆-C₁₄), wherein at least one ring in the system is aromatic and wherein each ring in the system contains three to seven ring members. In some embodiments, an “aryl” group contains between six and twelve total ring members (e.g., C₆-C₁₂). The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Unless otherwise specified, “aryl” groups are hydrocarbons. In some embodiments, an “aryl” ring system is an aromatic ring (e.g., phenyl) that is fused to a non-aromatic ring (e.g., cycloalkyl). Examples of aryl rings include that are fused include

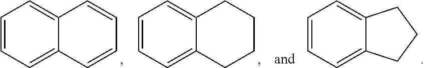

Biological sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Combination therapy: As used herein, the term “combination therapy” refers to those situations in which a subject is simultaneously exposed to two or more therapeutic regimens (e.g., two or more therapeutic agents or modality(ies)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form —e.g., gas, gel, liquid, solid, etc.

Cycloaliphatic: As used herein, the term “cycloaliphatic” refers to a monocyclic C₃₋₈ hydrocarbon or a bicyclic C₆₋₁₀ hydrocarbon that is completely saturated or that contains one or more units of unsaturation, but which is not aromatic, that has a single point or more than one points of attachment to the rest of the molecule.

Cycloalkyl: As used herein, the term “cycloalkyl” refers to an optionally substituted saturated ring monocyclic or polycyclic system of about 3 to about 10 ring carbon atoms.

Exemplary monocyclic cycloalkyl rings include cyclopropyl, cyclobutyl, cyclopentyl, cyclohexyl, and cycloheptyl.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Heteroaliphatic: The term “heteroaliphatic” or “heteroaliphatic group”, as used herein, denotes an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. The term “heteroatom” refers to nitrogen, oxygen, or sulfur, and includes any oxidized form of nitrogen or sulfur, and any quaternized form of a basic nitrogen. The term “nitrogen” also includes a substituted nitrogen. Unless otherwise specified, heteroaliphatic groups contain 1-10 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups. For example, a 1- to 10 atom heteroaliphatic group includes the following exemplary groups: —O—CH₃, —CH₂—O—CH₃, —O—CH₂—CH₂—O—CH₂—CH₂—O—CH₃, and the like.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 rr-electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridyl, imidazo[4,5-b]pyridyl, imidazo[4,5-c]pyridyl, pyrrolopyridyl, pyrrolopyrazinyl, thienopyrimidinyl, triazolopyridyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzotriazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, 4H-thieno[3,2-b]pyrrole, and benzoisoxazolyl. The term “heteroaryl” may be used interchangeably with the terms “heteroaryl ring”, “heteroaryl group”, or “heteroaromatic”, any of which terms include rings that are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, “heterocyclic radical”, and “heterocyclic ring” are used interchangeably and refer to a stable 3- to 8-membered monocyclic, a 6- to 10-membered bicyclic, or a 10- to 16-membered polycyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, azetidinyl, oxetanyl, tetrahydrofuranyl, tetrahydrothienyl, pyrrolidinyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 7- to 11-membered bridged heterocyclic ring having one, two, or three bridging atoms.

Lipid: The term “lipid,” as used herein, refers to molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). In some embodiments, hydrophilic groups may comprise polar and/or charged groups and include carbohydrates, phosphate, carboxylic, sulfate, amino, sulfhydryl, nitro, hydroxyl, and other like groups.

Modulator: The term “modulator,” as used herein, refers to a compound (e.g., a small molecule) that can alter the activity of another molecule (e.g., a protein). For example, in some embodiments, a modulator can cause an increase or decrease in the magnitude of a certain activity of a type of molecule as compared to the magnitude of the activity in the absence of the modulator. For example, a modulator can be an agonist or an antagonist of a particular target, as those terms are defined herein. For example, in some embodiments, a modulator is an agonist. In some embodiments, a modulator is an antagonist.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated: As used herein, the term “partially unsaturated” refers to a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amount appropriate for administration in a therapeutic or dosing regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977).

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics).

Polymer: As used herein, the term “polymer” refers to a composition comprising one or more molecules that comprise repeating units of one or more monomers. As used herein, “polymer” and “polymer composition” are used interchangeably, and unless otherwise specified, refer to a composition of polymer molecules. A person of skill in the art will appreciate that a polymer composition comprises polymer molecules having molecules of different lengths (e.g., comprising varying amounts of monomers). Polymer compositions described herein are characterized by one or more of a number average molecular weight (Mₙ), a weight average molecular weight (M_{w}), and/or a polydispersity index (PDI). Polymers described herein can also be characterized by the degree of polymerization (DP), which refers to the number of monomer units in the polymer. A polymer described herein can be a homopolymer, a heteropolymer, or a block-co-polymer. As used herein, a “homopolymer” refers to a polymer having a single type of monomer repeating throughout a polymer chain, e.g., -A-A-A-A-. As used herein, a “heteropolymer” refers to a polymer having more than one type (e.g., two or more) types of monomers present throughout a polymer chain, e.g., -A-B-A-B-A-. As used herein, a “block-co-polymer” refers to a polymer having an arrangement of blocks of polymerized monomers, e.g., -A-A-A-A-B-B-B-B- (a di block polymer) or -A-A-A-A-B-B-B-B-A-A-A- (a tri block polymer). Polymers described herein can be linear or branched.

Prevent or prevention: As used herein, the terms “prevent” or “prevention”, when used in connection with the occurrence of a disease, disorder, and/or condition, refer to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g; replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is a mRNA. In some embodiments where an RNA is a mRNA, a RNA typically comprises at its 3′ end a poly(A) region. In some embodiments where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods). As used herein, “monomeric RNA”, also referred to as “monomolecular RNA”, refers to an individual RNA molecule that is not an aggregate, a dimer, trimer, or oligomer of RNA.

Sample: As used herein, the term “sample” typically refers to an aliquot of material obtained or derived from a source of interest. In some embodiments, a source of interest is a biological or environmental source. In some embodiments, a source of interest may be or comprise a cell, tissue, or organism, such as a microbe, a plant, or an animal (e.g., a human). In some embodiments, a source of interest is or comprises biological tissue or fluid. In some embodiments, a source of interest may be or comprise a preparation generated in a production run. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample.

Substituted or optionally substituted: As described herein, compounds of the invention may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure (e.g.,

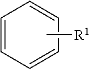

refers to at least

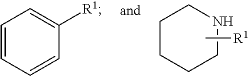

and refers to at least

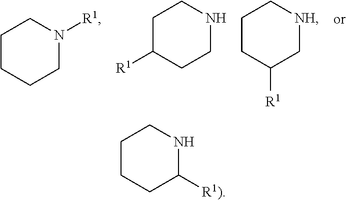

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; (CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; N(R^{∘})C(S)NR^{∘}₂; —(CH₂)₀₋₄N(R^{∘})C(O)OR^{∘}; —N(R^{∘})N(R^{∘})C(O)R^{∘}; N(R^{∘})N(R^{∘})C(O)NR^{∘}₂; N(R^{∘})N(R^{∘})C(O)OR^{∘}; —(CH₂)₀₋₄C(O)R^{∘}; C(S)R^{∘}; —(CH₂)₀₋₄C(O)OR^{∘}; —(CH₂)₀₋₄C(O)SR^{∘}; (CH₂)₀₋₄C(O)OSiR^{∘}₃; —(CH₂)₀₋₄OC(O)R^{◯}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, (CH₂)₀₋₄OC(O)NR^{∘}₂; C(O)N(OR^{∘})R^{∘}; —C(O)C(O)R^{∘}; —C(O)CH₂C(O)R^{∘}; —C(NOR^{∘})R^{∘}; (CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; (CH₂)₀₋₄S(O)R^{∘}; N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; P(O)R^{∘}₂; OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁ Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR●, —(CH₂)₀₋₂NR^{●}₂, —NO₂, —SiR^{●}₃, —OSiR^{●}₃, C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁ Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, (haloR^{●}), OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, S(O)₂R^{†}, S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, (haloR^{●}), —OH, —OR^{●}, —O(haloR^{●}), —CN, —C(O)OH, —C(O)OR^{●}, —NH₂, —NHR^{●}, —NR^{●}₂, or NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer.

In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid.

In some embodiments, a small molecule is a modulating agent (e.g., is an inhibiting agent or an activating agent). In some embodiments, a small molecule is biologically active.

In some embodiments, a small molecule is detectable (e.g., comprises at least one detectable moiety). In some embodiments, a small molecule is a therapeutic agent.

Those of ordinary skill in the art, reading the present disclosure, will appreciate that certain small molecule compounds described herein may be provided and/or utilized in any of a variety of forms such as, for example, crystal forms (e.g., polymorphs, solvates, etc), salt forms, protected forms, pro-drug forms, ester forms, isomeric forms (e.g., optical and/or structural isomers), isotopic forms, etc.

Those of ordinary skill in the art will appreciate that certain small molecule compounds have structures that can exist in one or more steroisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form.

Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms.

Those of skill in the art will appreciate that certain small molecule compounds have structures that permit isotopic substitution (e.g., ²H or ³H for H; ¹¹C, ¹³C or ¹⁴C for ¹²C; ¹³N or ¹⁵N for ¹⁴N; ¹⁷O or ¹⁸O for ¹⁶Q; ³⁶Cl for ³⁵Cl or ³⁷Cl; ¹⁸F for ¹⁹F; ¹³¹I for ¹²⁷I; etc.). In some embodiments, such a small molecule may be utilized in accordance with the present disclosure in one or more isotopically modified forms, or mixtures thereof.

In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form.

In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Those skilled in the art will further appreciate that, in small molecule structures, the symbol [symbol] , as used herein, refers to a point of attachment between two atoms. Additionally or alternatively, the symbol [symbol] refers to a point of attachment ring in a spirocyclic manner.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

### Complexes for Nucleic Acid Delivery

The present disclosure provides nucleic acid particles (including compositions comprising said nucleic acid particles), wherein the nucleic acid particle comprises a nucleic acid (e.g., RNA), an immunomodualtor, and a cationic lipid or cationic polymer. The present disclosure encompasses, among other things, the surprising insight that particular agents, such as immunomodulators, can be added to nucleic acid particles described herein, and cause the reduction in inflammatory response that is seen with previous particles. Moreover, nucleic acid particles described herein surprisingly also demonstrate improved expression of proteins or antigens that are encoded by nucleic acids that are delivered by said particles.

A “nucleic acid particle” (e.g., a ribonucleic acid particle) refers to a particle that encompasses or contains a nucleic acid, and, as part of a composition (e.g., a pharmaceutical composition) comprising multiple nucleic acid particles, is used to deliver nucleic acid to a target site of interest (e.g., cell, tissue, organ, and the like). As described herein, a nucleic acid particle (e.g., a ribonucleic acid particle) may be formed from i) at least one cationic or cationically ionizable lipid or lipid-like material; ii) at least one cationic polymer such as polyethyleneimine, protamine, or a mixture thereof (i.e., a mixture of i) and ii)), and iii) nucleic acid. Nucleic acid particles (e.g., a ribonucleic acid particle) include lipid nanoparticles (LNP), lipoplexes, liposomes, and polyplexes. In some embodiments, a nucleic acid particle described herein does not comprise an oligosaccharide.

In some embodiments, a particle for nucleic acid delivery as described herein is a lipid nanoparticle that comprises i) a nucleic acid, (ii) at least one cationic or cationically ionizable lipid, and (iii) an immunomodulator. In some embodiments, a particle for nucleic acid delivery comprises (i) a nucleic acid (such as DNA or RNA, for example, mRNA); (ii) at least one cationic or cationically ionizable lipid as disclosed herein; (iii) an immunomodualtor, and (iv) at least one additional lipid (e.g., a steroid, a helper lipid (also referred to as a “neutral lipid”), a polymer-conjugated lipid, or combinations thereof) as described herein.

Electrostatic interactions between positively charged molecules such as cationic polymers and cationic lipids and negatively charged nucleic acid are involved in particle formation. This results in complexation and spontaneous formation of nucleic acid particles.

Without intending to be bound by any theory, it is believed that the cationic or cationically ionizable lipid or lipid-like material and/or the cationic polymer combine together with the nucleic acid to form aggregates, and this aggregation results in colloidally stable particles. In some embodiments, particles comprise an amphiphilic lipid, in particular cationic or cationically ionizable amphiphilic lipid, and nucleic acid (such as DNA or RNA, e.g., mRNA) as described herein. In some embodiments, particles comprise or consist of a cationic/cationically ionizable lipid (e.g., a cationically ionizable lipid of formulae described herein); and at least one additional lipid such as a neutral lipid (such as a phospholipid), an immunomodulator, a polymer-conjugated lipid, optionally, a steroid (such as cholesterol), and combinations thereof. In some embodiments, particles comprise a cationic polymer and a nucleic acid.

In some embodiments, nucleic acid particles (e.g., ribonucleic acid particles) comprise more than one type of nucleic acid molecules, where the molecular parameters of the nucleic acid molecules may be similar or different from each other, like with respect to molar mass or fundamental structural elements such as molecular architecture, capping, coding regions or other features.

In some embodiments, a nucleic acid particle described herein is a nanoparticle. As used in the present disclosure, “nanoparticle” refers to a particle having an average diameter suitable for parenteral administration and is less than 1000 nm in diameter. In some embodiments, a composition comprising nanoparticles can have an average nanoparticle size (e.g., mean diameter) of about 30 nm to about 150 nm, about 40 nm to about 150 nm, about 50 nm to about 150 nm, about 60 nm to about 130 nm, about 70 nm to about 110 nm, about 70 nm to about 100 nm, about 70 to about 90 nm, or about 70 nm to about 80 nm. In some embodiments, a composition comprising nanoparticles can have an average nanoparticle size (e.g., mean diameter) of about 50 nm to about 100 nm. In some embodiments, a composition comprising nanoparticles can have an average nanoparticle size (e.g., mean diameter) of about 50 nm to about 150 nm. In some embodiments, a composition comprising nanoparticles can have an average nanoparticle size (e.g., mean diameter) of about 60 nm to about 120 nm. In some embodiments, a composition comprising nanoparticles can have an average nanoparticle size (e.g., mean diameter) of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

A composition comprising nucleic acid particles (e.g., ribonucleic acid particles) described herein may exhibit a polydispersity index less than about 0.5, less than about 0.4, less than about 0.3, or about 0.2 or less of said nanoparticles. By way of example, a composition comprising nucleic acid particles (e.g., ribonucleic acid particles) described herein can exhibit a polydispersity index in a range of about 0.1 to about 0.3 or about 0.2 to about 0.3.

Nucleic acid particles (e.g., ribonucleic acid particles) described herein can be characterized by an “N/P ratio,” which is the molar ratio of cationic (nitrogen) groups (the “N” in N/P) in the cationic polymer to the anionic (phosphate) groups (the “P” in N/P) in RNA. It is understood that a cationic group is one that is either in cationic form (e.g., N⁺), or one that is ionizable to become cationic. Use of a single number in an N/P ratio (e.g., an N/P ratio of about 5) is intended to refer to that number over 1, e.g., an N/P ratio of about 4 is intended to mean about 4:1. In some embodiments, a nucleic acid particle (e.g., a ribonucleic acid particle) described herein has an N/P ratio greater than or equal to 4. In some embodiments, a nucleic acid particle (e.g., a ribonucleic acid particle) described herein has an N/P ratio that is about 4 to about 12. In some embodiments, a nucleic acid particle (e.g., a ribonucleic acid particle) described herein has an N/P ratio that is about 4, 5, 6, 7, 8, 9, 10, 11, or 12. In some embodiments, an N/P ratio for a nucleic acid particle (e.g., a ribonucleic acid particle) described herein is from about 6.

Nucleic acid particles (e.g., ribonucleic acid particles) described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles. As used herein, an “ionizable” lipid, e.g., a “cationically ionizable” lipid or “ionizable” polymer, e.g., a “cationically ionizable” polymer is a lipid or polymer that may be, in some embodiment, neutral at physiological pH, but is capable of becoming cationic (i.e., becoming positively charged) at neutral pH.

The term “average diameter” or “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PDI), which is dimensionless (Koppel, D., J. Chem. Phys. 57, 1972, pp 4814-4820, ISO 13321). Here “average diameter,” “mean diameter,” “diameter,” or “size” for particles is used synonymously with this value of the Z-average.

The “polydispersity index” is preferably calculated based on dynamic light scattering measurements by the so-called cumulant analysis as mentioned in the definition of the “average diameter.” Under certain prerequisites, it can be taken as a measure of the size distribution of an ensemble of ribonucleic acid nanoparticles (e.g., ribonucleic acid nanoparticles).

Different types of nucleic acid particles have been described previously to be suitable for delivery of nucleic acid in particulate form (e.g. Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with the nucleic acid to form nucleic acid particles (e.g., ribonucleic acid particles, e.g., ribonucleic acid nanoparticles) and compositions comprising such particles. The nucleic acid particles (e.g., ribonucleic acid particles, e.g., ribonucleic acid nanoparticles) may comprise nucleic acid which is complexed in different forms by non-covalent interactions to the particle. In some embodiments, the particles described herein are not viral particles, in particular, they are not infectious viral particles, i.e., they are not able to virally infect cells.

Some embodiments described herein relate to compositions, methods and uses involving more than one, e.g., 2, 3, 4, 5, 6 or even more nucleic acid species.

In a nucleic acid particle (e.g., ribonucleic acid particle, e.g., ribonucleic acid nanoparticle) composition, it is possible that each nucleic acid species is separately formulated as an individual nucleic acid particle (e.g., ribonucleic acid particle, e.g., ribonucleic acid nanoparticle) formulation. In that case, each individual nucleic acid particle (e.g., ribonucleic acid particle, e.g., ribonucleic acid nanoparticle) formulation will comprise one nucleic acid species. The individual nucleic acid particle (e.g., ribonucleic acid particle, e.g., ribonucleic acid nanoparticle) formulations may be present as separate entities, e.g., in separate containers. Such formulations are obtainable by providing each nucleic acid species separately (typically each in the form of a nucleic acid-containing solution) together with a particle-forming agent, thereby allowing the formation of particles. Respective particles will contain exclusively the specific nucleic acid species that is being provided when the particles are formed (individual particulate formulations).

In some embodiments, a composition such as a pharmaceutical composition comprises more than one individual nucleic acid particle (e.g., ribonucleic acid particle, e.g., ribonucleic acid nanoparticle) formulation. Respective pharmaceutical compositions are referred to as “mixed particulate formulations.” Mixed particulate formulations according to the invention are obtainable by forming, separately, individual nucleic acid particle (e.g., ribonucleic acid particle, e.g., ribonucleic acid nanoparticle) formulations, as described above, followed by a step of mixing of the individual nucleic acid particle (e.g., ribonucleic acid particle, e.g., ribonucleic acid nanoparticle) formulations. By the step of mixing, a formulation comprising a mixed population of nucleic acid-containing particles is obtainable. Individual nucleic acid particle (e.g., ribonucleic acid particle, e.g., ribonucleic acid nanoparticle) populations may be together in one container, comprising a mixed population of individual nucleic acid particle (e.g., ribonucleic acid particle, e.g., ribonucleic acid nanoparticle) formulations.

Alternatively, it is possible that different nucleic acid species are formulated together as a “combined particulate formulation.” Such formulations are obtainable by providing a combined formulation (typically combined solution) of different nucleic acid species species together with a particle-forming agent, thereby allowing the formation of particles. As opposed to a “mixed particulate formulation,” a “combined particulate formulation” will typically comprise particles that comprise more than one nucleic acid species. In a combined particulate composition different nucleic acid species are typically present together in a single particle.

In certain embodiments, nucleic acids, when present in provided nucleic acid particles (e.g., ribonucleic acid particles, e.g., lipid nanoparticles, liposomes, lipoplexes, polyplexes) are resistant in aqueous solution to degradation with a nuclease.

### Lipid Nanoparticles

In some embodiments, nucleic acid particles (e.g., ribonucleic acid particles) are lipid nanoparticles. In some embodiments, lipid nanoparticles are cationic lipid nanoparticles comprising one or more cationic lipids (e.g., ones described herein), a nucleic acid (e.g., RNA) and an immunomodulator. In some embodiments, cationic lipid nanoparticles may comprise at least one cationic lipid, an immunomodulator, at least one polymer-conjugated lipid, and at least one helper lipid. Lipid nanoparticles (LNPs) have proven useful for the delivery of nucleic acid cargo to tissue of interest. LNPs are used, for example, in certain commercial vaccines for treatment of COVID-19. Some LNP formulations, however, cause an inflammatory response in the body, such as an increase of cytokines and interleukins. This inflammatory response is associated with pain, swelling, fever, and the like. LNPs of the present disclosure, however, do not suffer from the same deficiencies associated with previous formulations.

LNPs of the present disclosure comprise i) a cationic lipid; ii) a helper lipid; iii) a polymer-conjugated lipid (e.g., a polyethylene glycol bound lipid “a PEG lipid”); and iv) an immunomodulator. In some embodiments, LNPs described herein can further comprise additional additives, as described herein. LNPs of the present disclosure can be useful in a variety of contexts. For example, LNPs comprising a nucleic acid (e.g., an RNA) described herein are useful for delivery of said nucleic acid into the cell of a subject. In some embodiments, LNPs comprising a nucleic acid (e.g., an RNA) described herein are useful for causing increased expression of a protein in a subject. In some embodiments, LNPs comprising a nucleic acid (e.g., an RNA) described herein are useful for causing a pharmacological effect induced by expression of a protein in a subject. Lipid nanoparticles described herein are characterized by molar percentage (mol %) of components in the lipid nanoparticle. A mol % used in reference to a lipid component of a lipid nanoparticle is relative to the total other lipid components in the lipid nanoparticle.

### Cationic Lipids

As described herein, LNPs of the present disclosure comprise a cationic lipid. A cationic lipid, as described herein, is a lipid that is positively charged or is ionizable, such that the cationic lipid will become positively charged when subjected to particular physiological conditions, e.g., a pH of about 7.4 or less, and can promote lipid aggregation. In some embodiments, a cationic lipid is a lipid comprising one or more amine groups which bear or are capable of bearing (i.e., are ionizable) a positive charge.

In some embodiments, a cationic lipid is selected from 1,2-dimyristoyl-sn-glycero-3-ethylphosphocholine (DMEPC); 2-dimyristoyl-3-trimethylammonium propane (DMTAP); dioleyl ether phosphatidylcholine (DOEPC); N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N-(N′,N′dimethylaminoethane)-carbamoyl)cholesterol (DC-Chol), N-(1-(2,3-dioleoyloxy)propyl)N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE).

In some embodiments, a cationic lipid is one provided in WO2012/016184, which is incorporated herein by reference in its entirety. For example, in some embodiments, a cationic lipid is selected from 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In some embodiments, a cationic lipid is selected from N,N-dimethyl-2,3-dioleyloxypropylamine (DODMA), 1,2-di-O-octadecenyl-3-trimethylammonium propane (DOTMA), 3-(N-(N′,N′-dimethylaminoethane)-carbamoyl)cholesterol (DC-Chol), dimethyldioctadecylammonium (DDAB); 1,2-dioleoyl-3-trimethylammonium propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), I,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-I-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-Dilinoleylcarbamyl-3-dimethylaminopropane (DLincarbDAP), 1,2-dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-aminoethyl)-N,N-dimethyl-2,3-bis(tetradecyloxy)-1-propanaminium bromide (pAE-DMRIE), N-(4-carboxybenzyl)-N,N-dimethyl-2,3-bis(oleoyloxy)propan-1-aminium (DOBAQ), 2-({8-[(3p)-cholest-5-en-3-yloxy]octyl}oxy)-N,N-dimethyl-3-[(9Z,12Z)-octadeca-9,12-dien-1-yloxy]propan-1-amine (octyl-CLinDMA), 1,2-dimyristoyl-3-dimethylammonium-propane (DMDAP), 1,2-dipalmitoyl-3-dimethylammonium-propane (DPDAP), N1-[2-((1 S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)-dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)-oxy)heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethyl-amino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200), and the following structures (XV-1) to (XV-6):

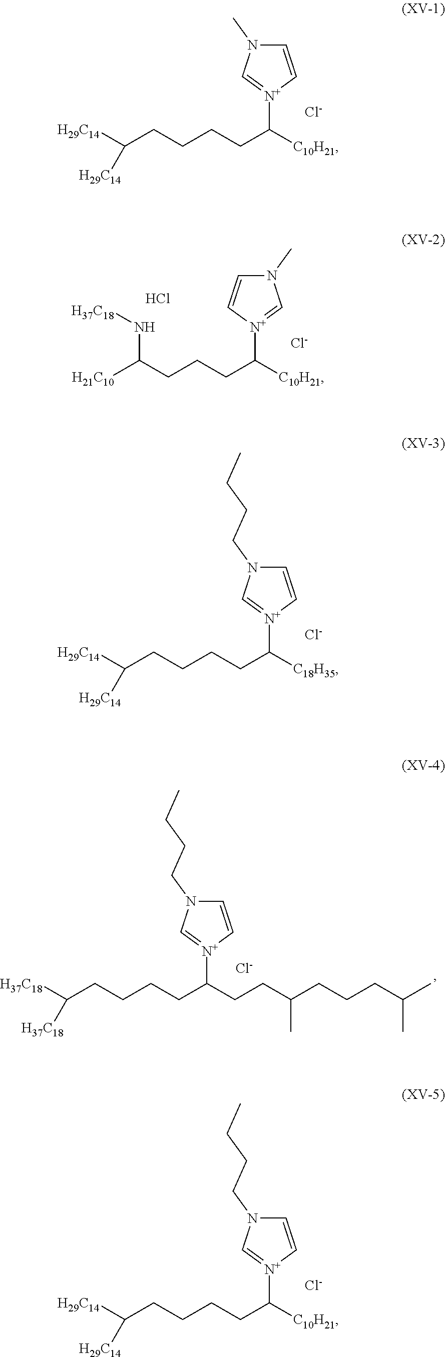

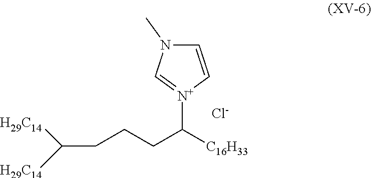

In some embodiments, a cationic lipid is one provided in WO2021/026358, WO2020/219941, WO2017/075531, WO2016/118725, WO2016/118724, WO2016/176330, WO2017/049245, U.S. Pat. No. 9,670,152, each of which is incorporated herein by reference in its entirety.

In some embodiments, a cationic lipid is a compound of Formula I:

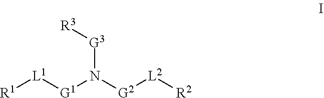

or a pharmaceutically acceptable salt thereof, wherein:
one of L¹ or L² is —OC(O)—, —C(O)O—, —C(O)—, —O—, —S(O)ₓ—, —S—S—, —C(O)S—, SC(O)—, —NR^{a}C(O)—, —C(O)NR^{a}—, —NR^{a}C(O)NR^{a}—, —OC(O)NR^{a}— or —NR^{a}C(O)O—, and the other of L¹ or L² is —OC(O)—, —C(O)O—, —C(O)—, —O—, —S(O)ₓ—, —S—S—, —C(O)S—, SC(O)—, —NR^{a}C(O)—, —C(O)NR^{a}—, —NR^{a}C(O)NR^{a}—, —OC(O)NR^{a}—, —NR^{a}C(O)O—, or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃—C cycloalkylene, C₃—C cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(O)OR⁴, —OC(O)R⁴ or —R⁵C(O)R⁴;
R⁴ is C₁-C₁₂ alkyl;
R⁵ is H or C₁-C₆ alkyl; and
x is 0, 1 or 2.

In some embodiments, one of L¹ or L² is —OC(O)— or —C(O)O—. In some embodiments, each of L¹ and L² is —OC(O)— or —C(O)O—.

In some embodiments, G¹ is C₁-C₁₂ alkylene. In some embodiments, G² is C₁-C₁₂ alkylene. In some embodiments G¹ and G² are each independently C₁-C₁₂ alkylene. In some embodiments G¹ and G² are each independently C₅-C₁₂ alkylene.

In some embodiments, G³ is C₁-C₂₄ alkylene. In some embodiments, G³ is C₁-C₆ alkylene.

In some embodiments, R¹ and R² are each independently selected from:

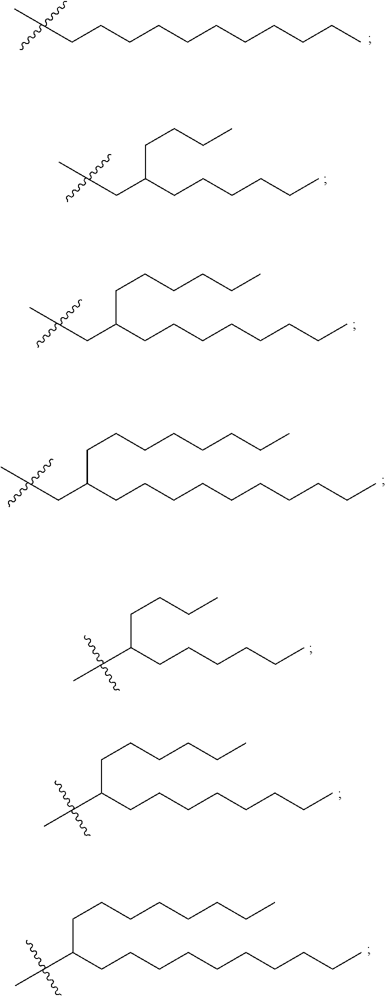

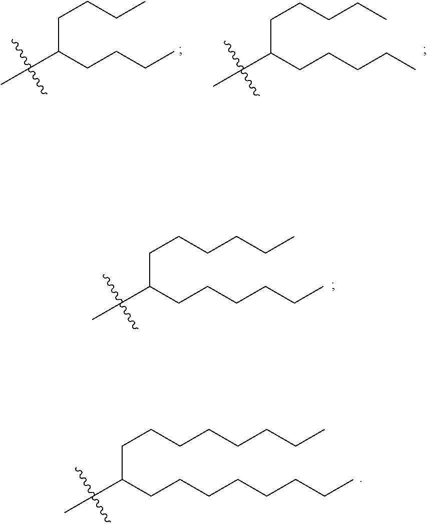

In some embodiments, R³ is OH.

In some embodiments, each of L¹ and L² is —OC(O)—, G¹ and G² are each independently C₅-C₁₂ alkylene, G³ is C₁-C₆ alkylene, R³ is OH, and R¹ and R² are each independently selected from:

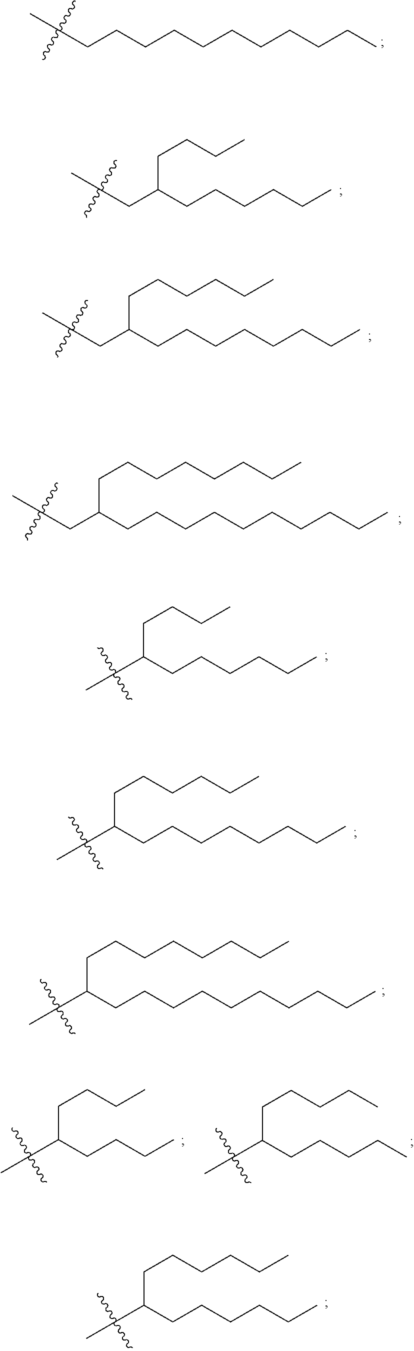

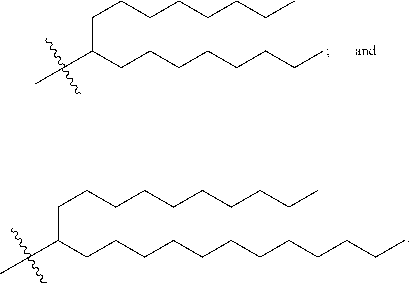 [SMILES: *C(CCCCCCCC)CCCCCCCC ; *C(CCCCCCCCCC)CCCCCCCCCCCC]

In some embodiments, a cationic lipid is a compound of Formula Ia or Ib

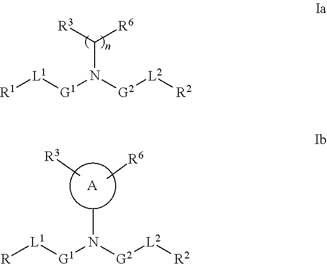

or a pharmaceutically acceptable salt thereof, where n is an integer from 1 to 15, A is C₃-C₈cycloaliphatic, each R⁶ is independently selected from H, OH, and C₁-C₂₄ aliphatic, and wherein R¹, R², R³, L¹, L², G¹, and G² are as described in classes and subclasses herein, both singly and in combination.

In some embodiments, a cationic lipid that may be useful in accordance with the present disclosure is an amino lipid comprising a titratable tertiary amino head group linked via ester bonds to at least two saturated alkyl chains, which ester bonds can be hydrolyzed easily to facilitate fast degradation and/or excretion via renal pathways. In some embodiments, such an amino lipid has an apparent pKₐ of about 6.0-6.5 (e.g., in one embodiment with an apparent pKₐ of approximately 6.25), resulting in an essentially fully positively charged molecule at an acidic pH (e.g., pH 5). In some embodiments, such an amino lipid, when incorporated in LNP, can confer distinct physicochemical properties that regulate particle formation, cellular uptake, fusogenicity and/or endosomal release of RNA(s). In some embodiments, introduction of an aqueous RNA solution to a lipid mixture comprising such an amino lipid at pH 4.0 can lead to an electrostatic interaction between the negatively charged RNA backbone and the positively charged cationic lipid. Without wishing to be bound by any particular theory, such electrostatic interaction leads to particle formation coincident with efficient encapsulation of RNA drug substance. After RNA encapsulation, adjustment of the pH of the medium surrounding the resulting LNP to a more neutral pH (e.g., pH 7.4) results in neutralization of the surface charge of the LNP. When all other variables are held constant, such charge-neutral particles display longer in vivo circulation lifetimes and better delivery to hepatocytes compared to charged particles, which are rapidly cleared by the reticuloendothelial system. Upon endosomal uptake, the low pH of the endosome renders LNP comprising such an amino lipid fusogenic and allows the release of the RNA into the cytosol of the target cell.

As described herein, a LNP comprises at least one cationic lipid. In some embodiments, a cationic lipid is selected from Table 1:

TABLE 1

| No. | Structure |
|---|---|
| I-1 | 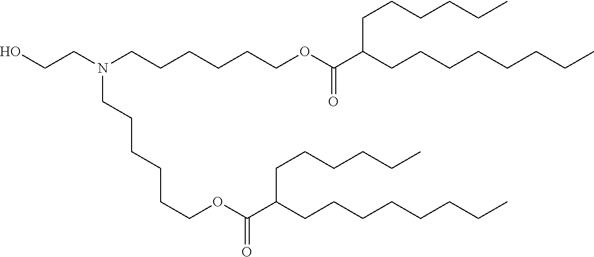 [SMILES: CCCCCCCCC(CCCCCC)C(=O)OCCCCCCN(CCO)CCCCCCOC(=O)C(CCCCCC)CCCCCCCC] |
| I-2 | 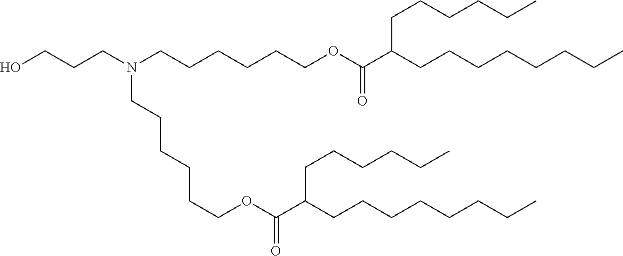 [SMILES: CCCCCCCCC(CCCCCC)C(=O)OCCCCCCN(CCCO)CCCCCCOC(=O)C(CCCCCC)CCCCCCCC] |
| I-3 | 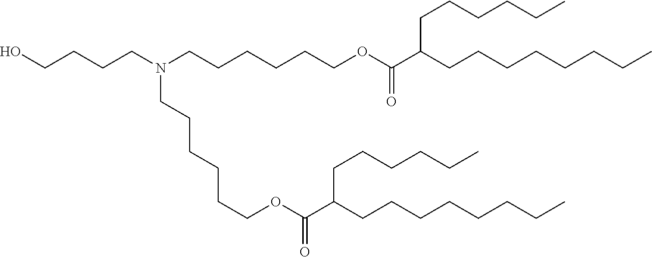 [SMILES: CCCCCCCCC(CCCCCC)C(=O)OCCCCCCN(CCCCO)CCCCCCOC(=O)C(CCCCCC)CCCCCCCC] |
| I-4 | 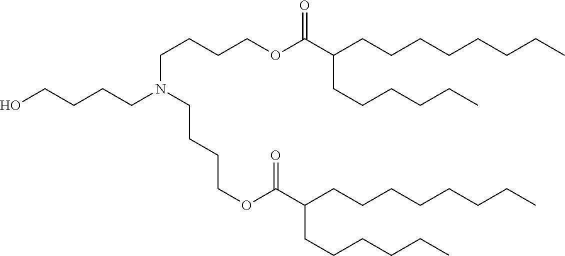 [SMILES: CCCCCCCCC(CCCCCC)C(=O)OCCCCN(CCCCO)CCCCOC(=O)C(CCCCCC)CCCCCCCC] |
| I-5 | 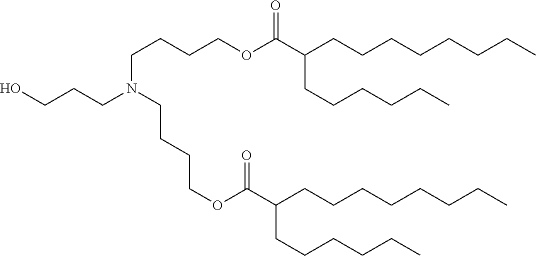 [SMILES: CCCCCCCCC(CCCCCC)C(=O)OCCCCN(CCCO)CCCCOC(=O)C(CCCCCC)CCCCCCCC] |
| I-6 | 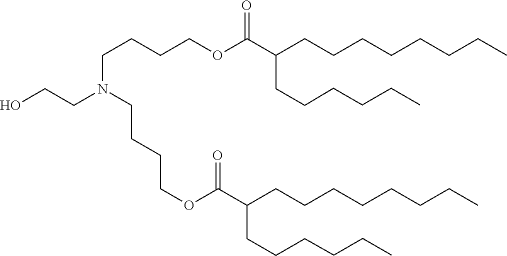 [SMILES: CCCCCCCCC(CCCCCC)C(=O)OCCCCN(CCO)CCCCOC(=O)C(CCCCCC)CCCCCCCC] |
| I-7 | 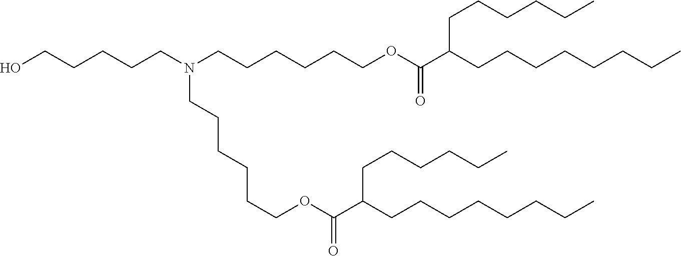 [SMILES: CCCCCCCCC(CCCCCC)C(=O)OCCCCCCN(CCCCCO)CCCCCCOC(=O)C(CCCCCC)CCCCCCCC] |
| I-8 | 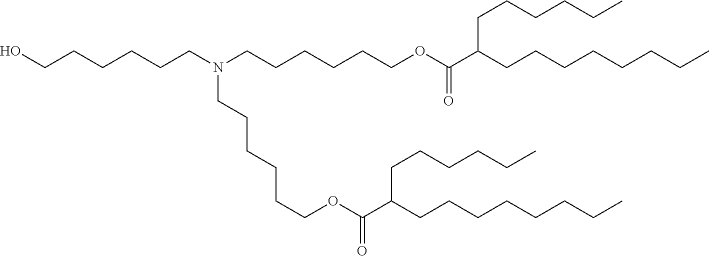 [SMILES: CCCCCCCCC(CCCCCC)C(=O)OCCCCCCN(CCCCCCO)CCCCCCOC(=O)C(CCCCCC)CCCCCCCC] |
| I-9 | 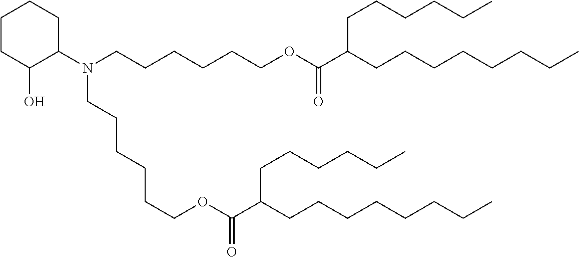 [SMILES: CCCCCCCCC(CCCCCC)C(=O)OCCCCCCN(CCCCCCOC(=O)C(CCCCCC)CCCCCCCC)C1CCCCC1O] |
| I-10 | 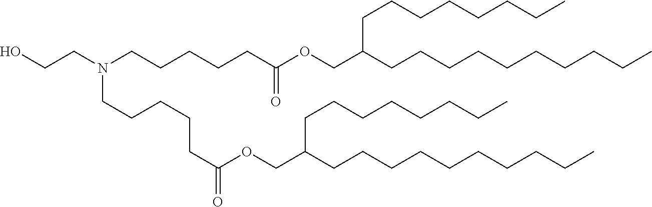 [SMILES: CCCCCCCCCCC(CCCCCCCC)COC(=O)CCCCCN(CCO)CCCCCC(=O)OCC(CCCCCCCC)CCCCCCCCCC] |
| I-11 | 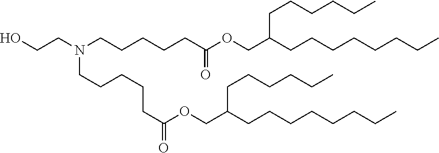 [SMILES: CCCCCCCCC(CCCCCC)COC(=O)CCCCCN(CCO)CCCCCC(=O)OCC(CCCCCC)CCCCCCCC] |
| I-12 | 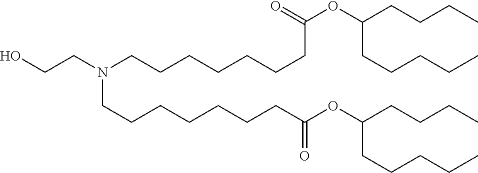 [SMILES: CCCCCC(CCCC)OC(=O)CCCCCCCN(CCO)CCCCCCCC(=O)OC(CCCC)CCCCC] |
| I-13 | 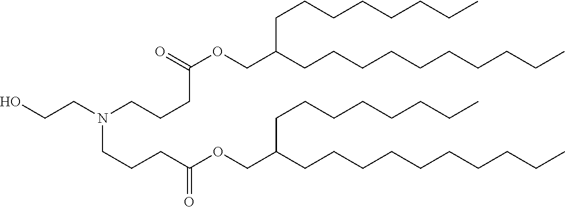 [SMILES: CCCCCCCCCCC(CCCCCCCC)COC(=O)CCCN(CCO)CCCC(=O)OCC(CCCCCCCC)CCCCCCCCCC] |
| I-14 | 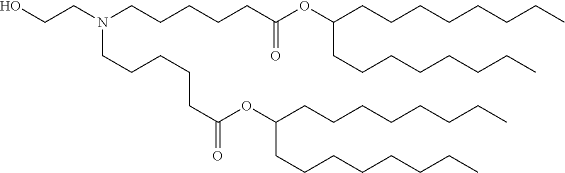 [SMILES: CCCCCCCCC(CCCCCCCC)OC(=O)CCCCCN(CCO)CCCCCC(=O)OC(CCCCCCCC)CCCCCCCC] |
| I-15 | 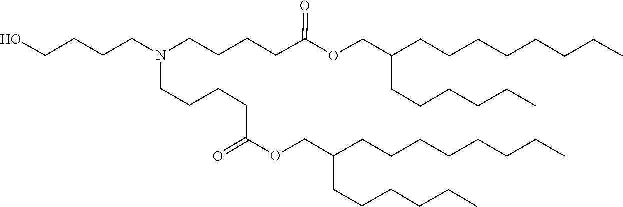 [SMILES: CCCCCCCCC(CCCCCC)COC(=O)CCCCN(CCCCO)CCCCC(=O)OCC(CCCCCC)CCCCCCCC] |
| I-16 | 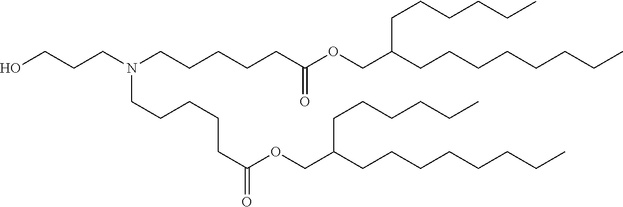 [SMILES: CCCCCCCCC(CCCCCC)COC(=O)CCCCCN(CCCO)CCCCCC(=O)OCC(CCCCCC)CCCCCCCC] |
| I-17 | 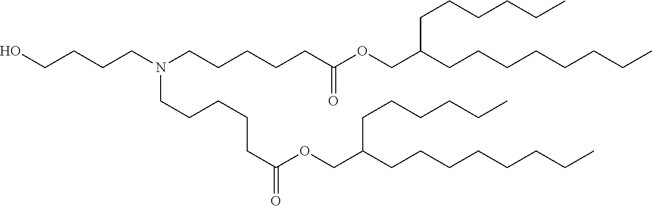 [SMILES: CCCCCCCCC(CCCCCC)COC(=O)CCCCCN(CCCCO)CCCCCC(=O)OCC(CCCCCC)CCCCCCCC] |
| I-18 | 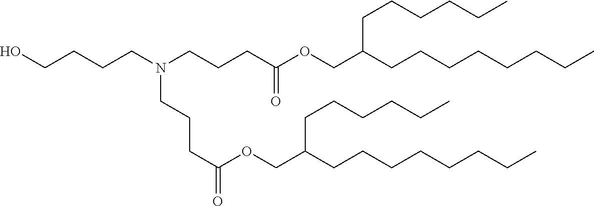 [SMILES: CCCCCCCCC(CCCCCC)COC(=O)CCCN(CCCCO)CCCC(=O)OCC(CCCCCC)CCCCCCCC] |
| I-19 | 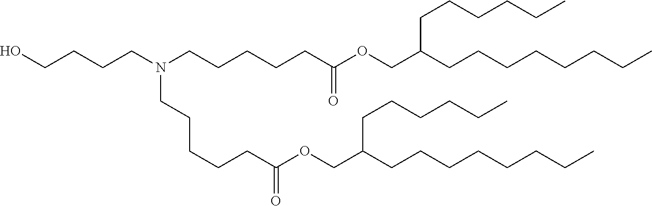 [SMILES: CCCCCCCCC(CCCCCC)COC(=O)CCCCCN(CCCCO)CCCCCC(=O)OCC(CCCCCC)CCCCCCCC] |
| I-20 | 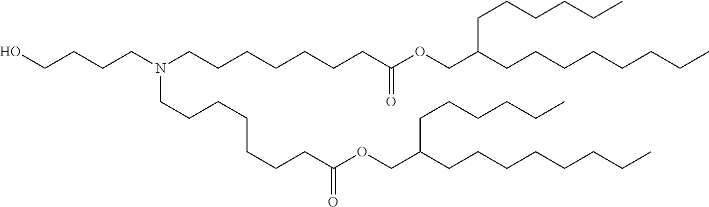 [SMILES: CCCCCCCCC(CCCCCC)COC(=O)CCCCCCCN(CCCCO)CCCCCCCC(=O)OCC(CCCCCC)CCCCCCCC] |
| I-21 | 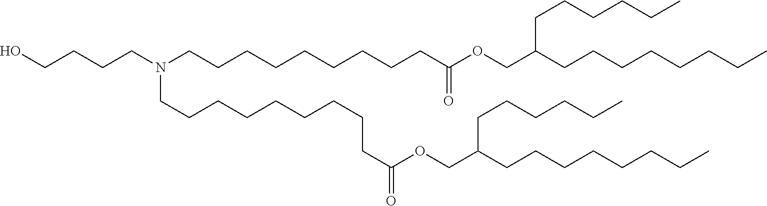 [SMILES: CCCCCCCCC(CCCCCC)COC(=O)CCCCCCCCCN(CCCCO)CCCCCCCCCC(=O)OCC(CCCCCC)CCCCCCCC] |
| I-22 | 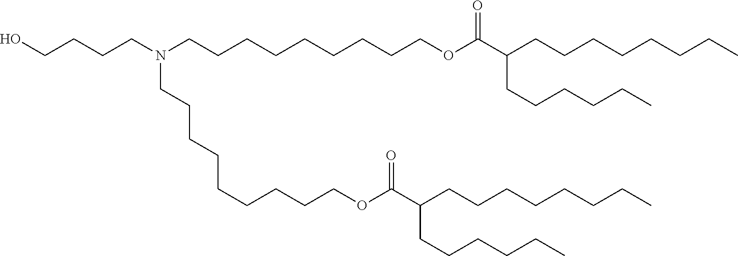 [SMILES: CCCCCCCCC(CCCCCC)C(=O)OCCCCCCCCCN(CCCCO)CCCCCCCCCOC(=O)C(CCCCCC)CCCCCCCC] |
| I-23 | 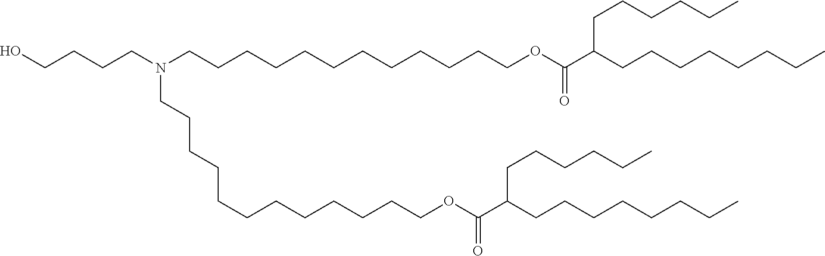 [SMILES: CCCCCCCCC(CCCCCC)C(=O)OCCCCCCCCCCCCN(CCCCO)CCCCCCCCCCCCOC(=O)C(CCCCCC)CCCCCCCC] |
| I-24 | 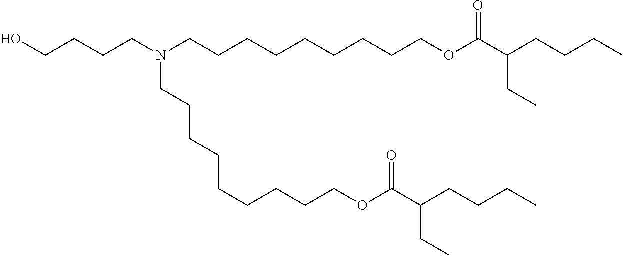 [SMILES: CCCCC(CC)C(=O)OCCCCCCCCCN(CCCCO)CCCCCCCCCOC(=O)C(CC)CCCC] |
| I-25 | 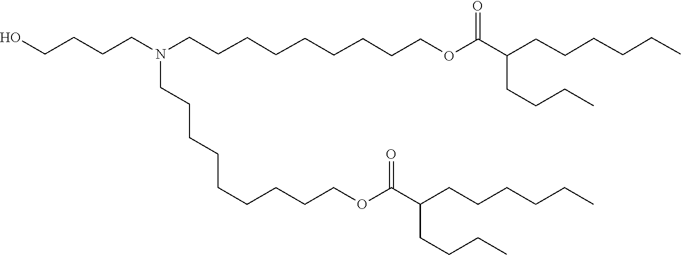 [SMILES: CCCCCCC(CCCC)C(=O)OCCCCCCCCCN(CCCCO)CCCCCCCCCOC(=O)C(CCCC)CCCCCC] |
| I-26 | 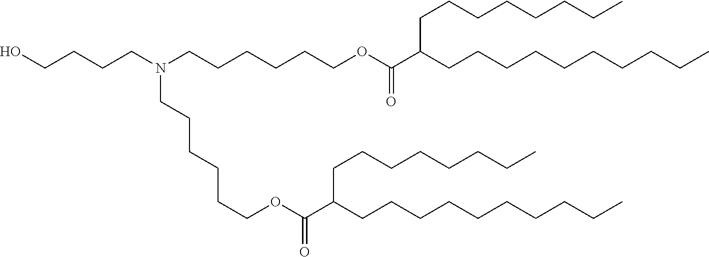 [SMILES: CCCCCCCCCCC(CCCCCCCC)C(=O)OCCCCCCN(CCCCO)CCCCCCOC(=O)C(CCCCCCCC)CCCCCCCCCC] |
| I-27 | 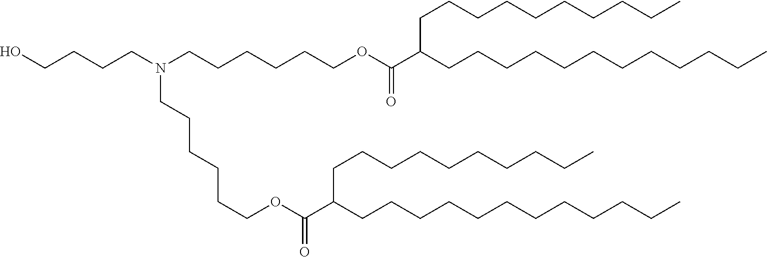 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| I-28 | 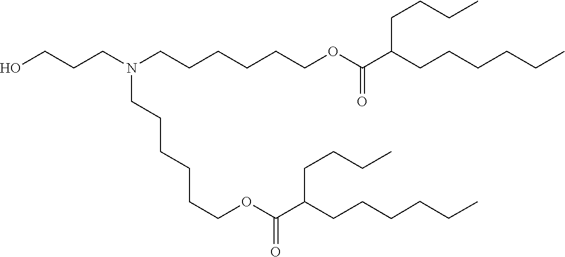 [SMILES: CCCCCCC(CCCC)C(=O)OCCCCCCN(CCCO)CCCCCCOC(=O)C(CCCC)CCCCCC] |
| I-29 | 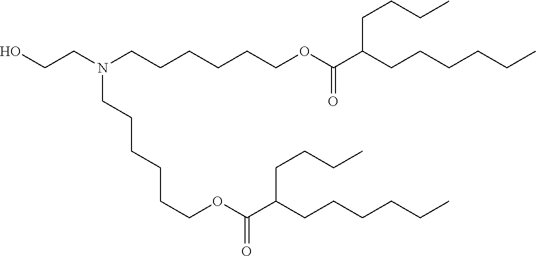 [SMILES: CCCCCCC(CCCC)C(=O)OCCCCCCN(CCO)CCCCCCOC(=O)C(CCCC)CCCCCC] |
| I-30 | 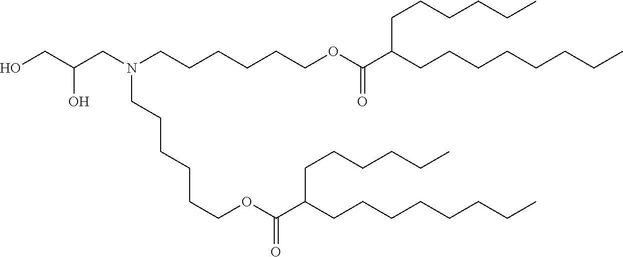 [SMILES: CCCCCCCCC(CCCCCC)C(=O)OCCCCCCN(CCCCCCOC(=O)C(CCCCCC)CCCCCCCC)CC(O)CO] |
| I-31 | 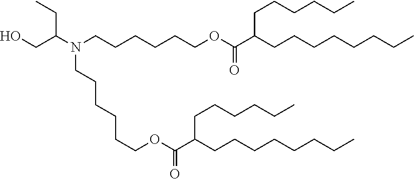 [SMILES: CCCCCCCCC(CCCCCC)C(=O)OCCCCCCN(CCCCCCOC(=O)C(CCCCCC)CCCCCCCC)C(CC)CO] |
| I-32 | 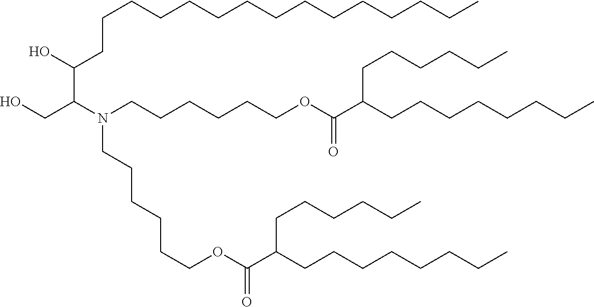 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| I-33 | 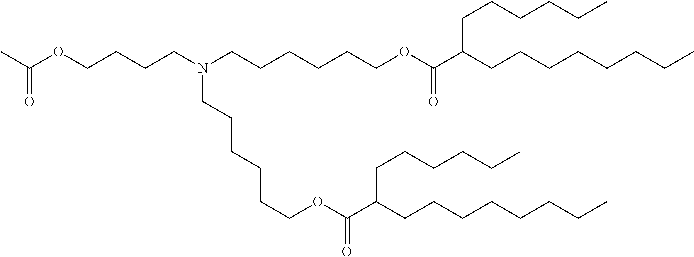 [SMILES: CCCCCCCCC(CCCCCC)C(=O)OCCCCCCN(CCCCCCOC(=O)C(CCCCCC)CCCCCCCC)CCCCOC(C)=O] |
| I-34 | 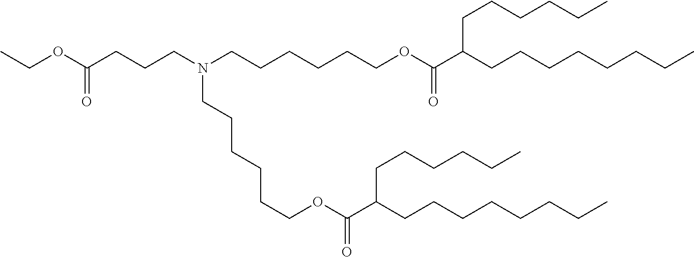 [SMILES: CCCCCCCCC(CCCCCC)C(=O)OCCCCCCN(CCCCCCOC(=O)C(CCCCCC)CCCCCCCC)CCCC(=O)OCC] |
| I-35 | 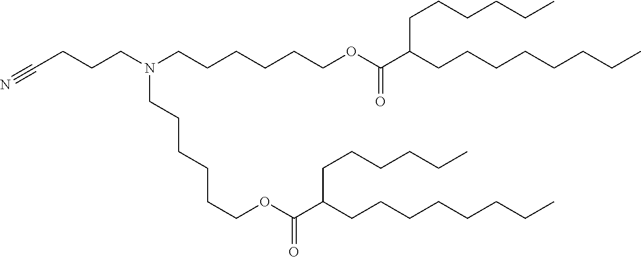 [SMILES: CCCCCCCCC(CCCCCC)C(=O)OCCCCCCN(CCCC#N)CCCCCCOC(=O)C(CCCCCC)CCCCCCCC] |
| I-36 | 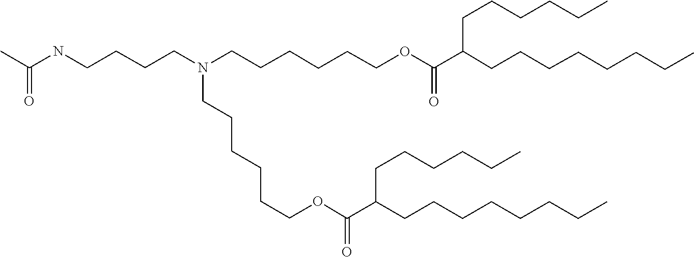 |
| I-37 | 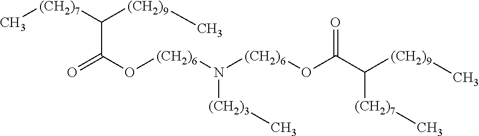 |
| I-38 | 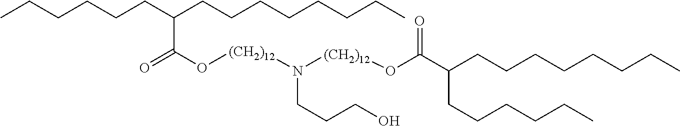 |
| I-39 | 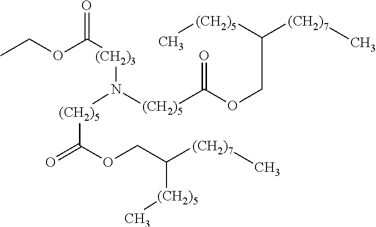 |
| I-40 | 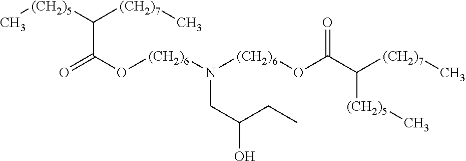 |
| I-41 | 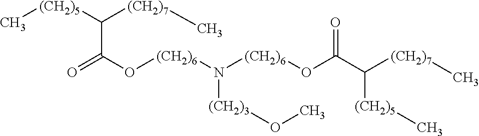 |
| I-42 | 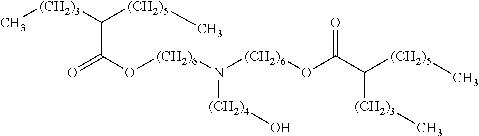 |
| I-43 | 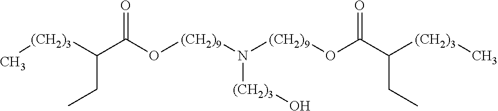 |
| I-44 | 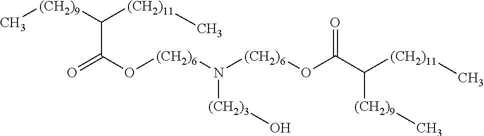 |
| I-45 | 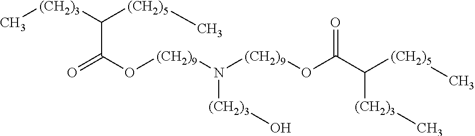 |
| I-46 | 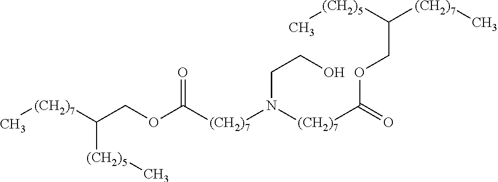 |
| I-47 | 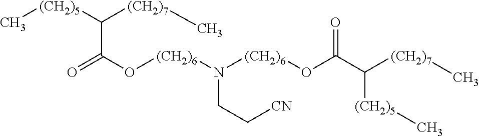 |
| I-48 | 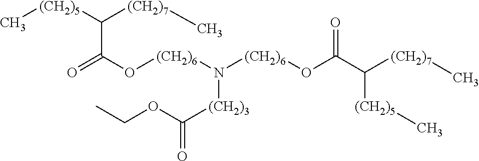 |
| I-49 | 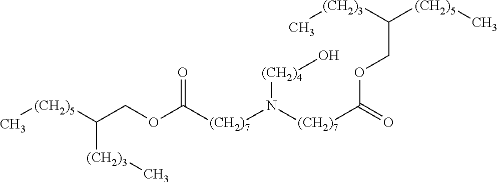 |
| I-50 | 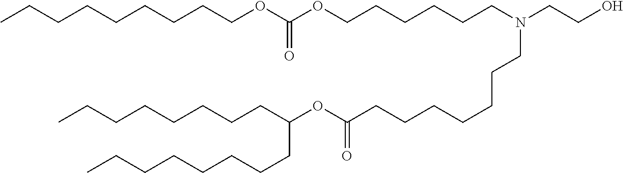 [SMILES: CCCCCCCCCOC(=O)OCCCCCCN(CCO)CCCCCCCC(=O)OC(CCCCCCCC)CCCCCCCC] |
| I-51 | 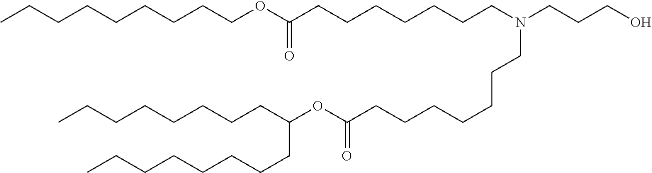 [SMILES: CCCCCCCCCOC(=O)CCCCCCCN(CCCO)CCCCCCCC(=O)OC(CCCCCCCC)CCCCCCCC] |
| I-52 | 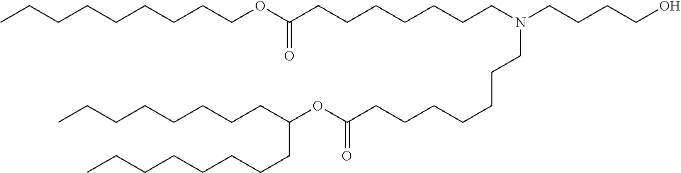 [SMILES: CCCCCCCCCOC(=O)CCCCCCCN(CCCCO)CCCCCCCC(=O)OC(CCCCCCCC)CCCCCCCC] |
| I-53 | 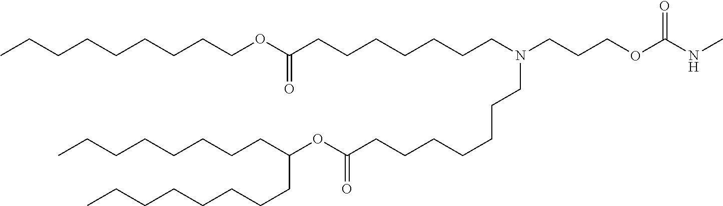 [SMILES: CCCCCCCCCOC(=O)CCCCCCCN(CCCCCCCC(=O)OC(CCCCCCCC)CCCCCCCC)CCCOC(=O)NC] |
| I-54 | 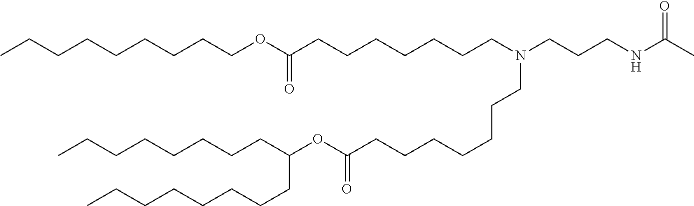 [SMILES: CCCCCCCCCOC(=O)CCCCCCCN(CCCCCCCC(=O)OC(CCCCCCCC)CCCCCCCC)CCCNC(C)=O] |
| I-55 | 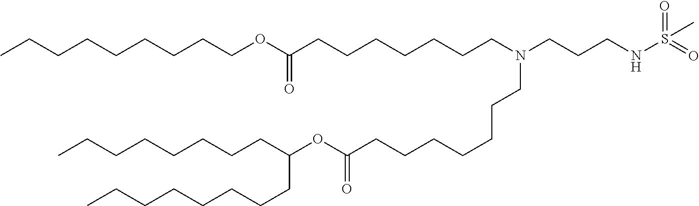 [SMILES: CCCCCCCCCOC(=O)CCCCCCCN(CCCCCCCC(=O)OC(CCCCCCCC)CCCCCCCC)CCCNS(C)(=O)=O] |
| I-56 | 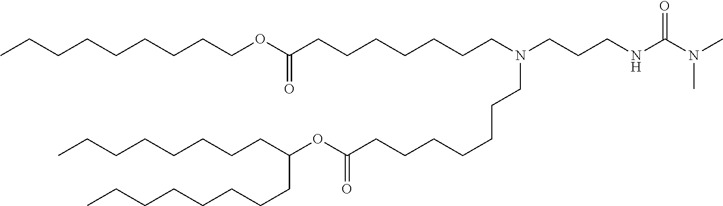 [SMILES: CCCCCCCCCOC(=O)CCCCCCCN(CCCCCCCC(=O)OC(CCCCCCCC)CCCCCCCC)CCCNC(=O)N(C)C] |
| I-57 | 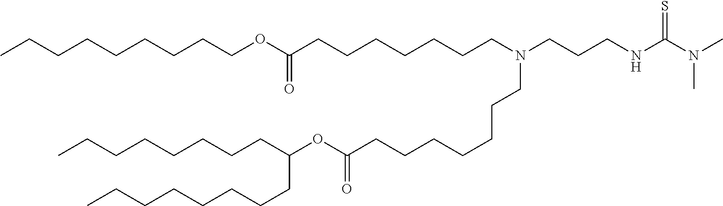 [SMILES: CCCCCCCCCOC(=O)CCCCCCCN(CCCCCCCC(=O)OC(CCCCCCCC)CCCCCCCC)CCCNC(=S)N(C)C] |
| I-58 | 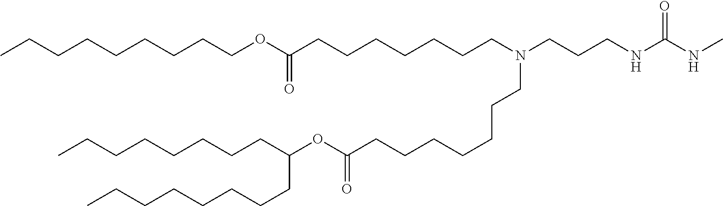 [SMILES: CCCCCCCCCOC(=O)CCCCCCCN(CCCCCCCC(=O)OC(CCCCCCCC)CCCCCCCC)CCCNC(=O)NC] |
| I-59 | 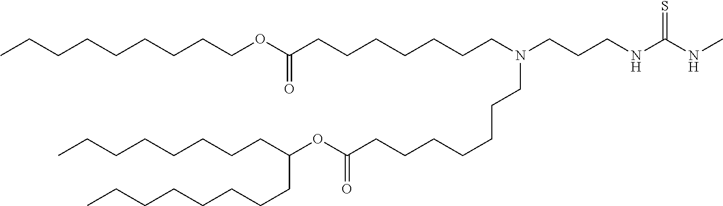 [SMILES: CCCCCCCCCOC(=O)CCCCCCCN(CCCCCCCC(=O)OC(CCCCCCCC)CCCCCCCC)CCCNC(=S)NC] |
| I-60 | 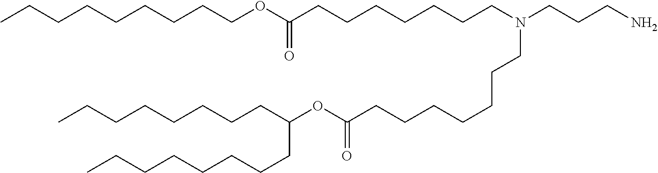 [SMILES: CCCCCCCCCOC(=O)CCCCCCCN(CCCN)CCCCCCCC(=O)OC(CCCCCCCC)CCCCCCCC] |
| I-61 | 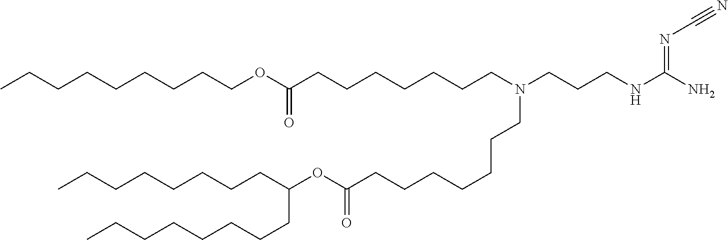 [SMILES: CCCCCCCCCOC(=O)CCCCCCCN(CCCCCCCC(=O)OC(CCCCCCCC)CCCCCCCC)CCCN/C(N)=N/C#N] |
| I-62 | 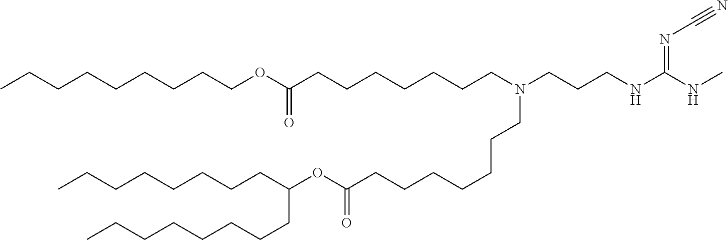 [SMILES: CCCCCCCCCOC(=O)CCCCCCCN(CCCCCCCC(=O)OC(CCCCCCCC)CCCCCCCC)CCCN/C(=N/C#N)NC] |
| I-63 | 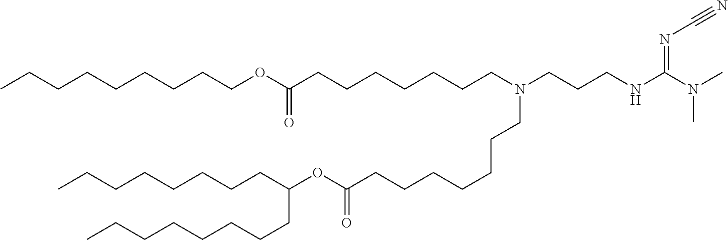 [SMILES: CCCCCCCCCOC(=O)CCCCCCCN(CCCCCCCC(=O)OC(CCCCCCCC)CCCCCCCC)CCCN/C(=N/C#N)N(C)C] |
| I-64 | 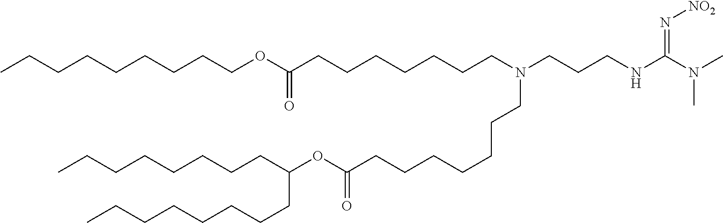 [SMILES: CCCCCCCCCOC(=O)CCCCCCCN(CCCCCCCC(=O)OC(CCCCCCCC)CCCCCCCC)CCCN/C(=N/[N+](=O)[O-])N(C)C] |
| I-65 | 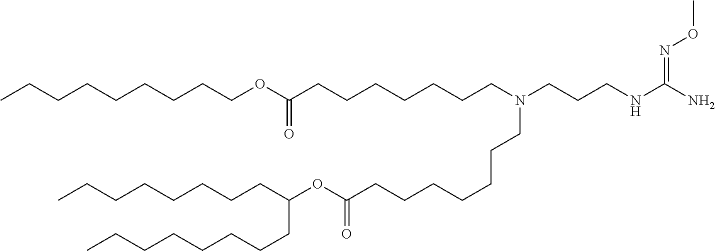 [SMILES: CCCCCCCCCOC(=O)CCCCCCCN(CCCCCCCC(=O)OC(CCCCCCCC)CCCCCCCC)CCCN/C(N)=N/OC] |
| I-66 | 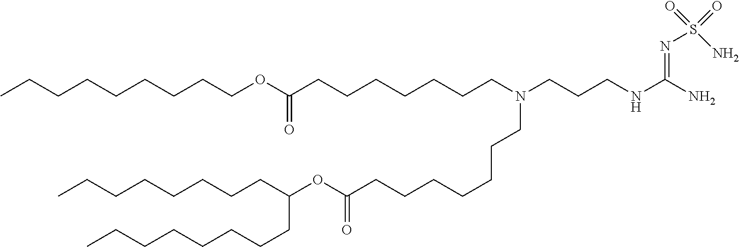 [SMILES: CCCCCCCCCOC(=O)CCCCCCCN(CCCCCCCC(=O)OC(CCCCCCCC)CCCCCCCC)CCCN/C(N)=N/S(N)(=O)=O] |
| I-67 | 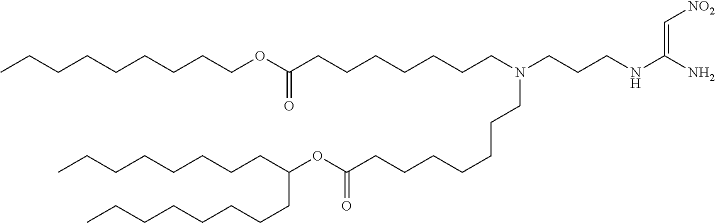 [SMILES: CCCCCCCCCOC(=O)CCCCCCCN(CCCCCCCC(=O)OC(CCCCCCCC)CCCCCCCC)CCCN/C(N)=C/[N+](=O)[O-]] |
| I-68 | 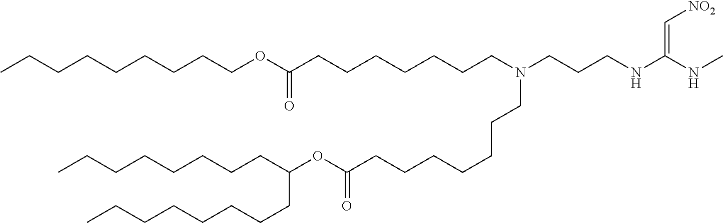 [SMILES: CCCCCCCCCOC(=O)CCCCCCCN(CCCCCCCC(=O)OC(CCCCCCCC)CCCCCCCC)CCCN/C(=C/[N+](=O)[O-])NC] |
| I-69 | 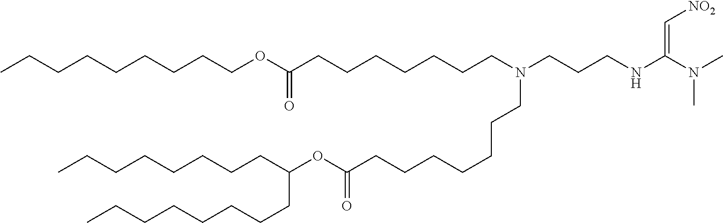 [SMILES: CCCCCCCCCOC(=O)CCCCCCCN(CCCCCCCC(=O)OC(CCCCCCCC)CCCCCCCC)CCCN/C(=C/[N+](=O)[O-])N(C)C] |
| I-70 | 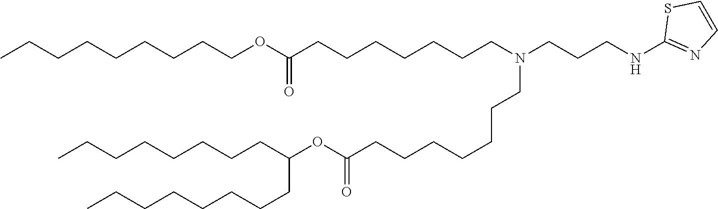 [SMILES: CCCCCCCCCOC(=O)CCCCCCCN(CCCCCCCC(=O)OC(CCCCCCCC)CCCCCCCC)CCCNc1nccs1] |
| I-71 | 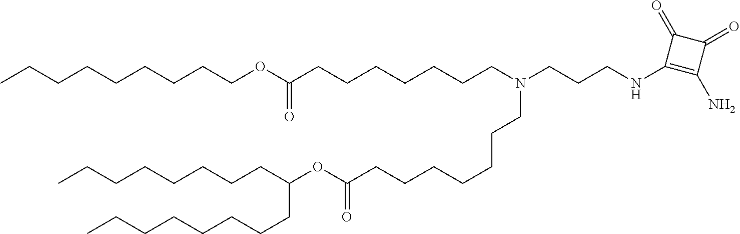 [SMILES: CCCCCCCCCOC(=O)CCCCCCCN(CCCCCCCC(=O)OC(CCCCCCCC)CCCCCCCC)CCCNc1c(N)c(=O)c1=O] |
| I-72 | 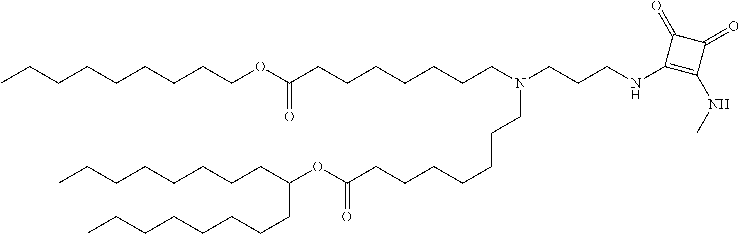 [SMILES: CCCCCCCCCOC(=O)CCCCCCCN(CCCCCCCC(=O)OC(CCCCCCCC)CCCCCCCC)CCCNc1c(NC)c(=O)c1=O] |
| I-73 | 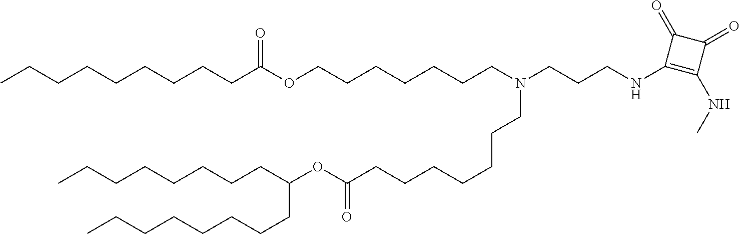 [SMILES: CCCCCCCCCC(=O)OCCCCCCCN(CCCCCCCC(=O)OC(CCCCCCCC)CCCCCCCC)CCCNc1c(NC)c(=O)c1=O] |
| I-74 | 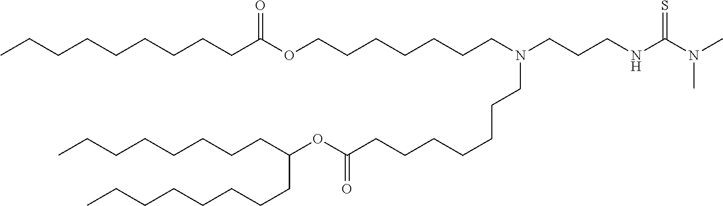 [SMILES: CCCCCCCCCC(=O)OCCCCCCCN(CCCCCCCC(=O)OC(CCCCCCCC)CCCCCCCC)CCCNC(=S)N(C)C] |
| I-75 | 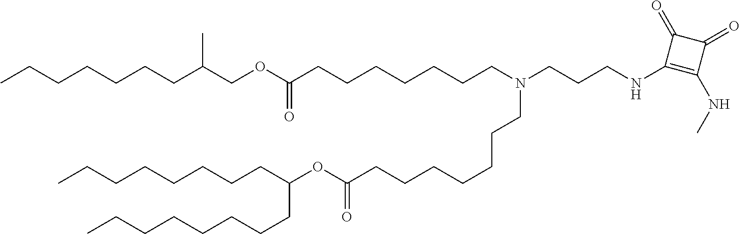 [SMILES: CCCCCCCCC(CCCCCCCC)OC(=O)CCCCCCCN(CCCCCCCC(=O)OCC(C)CCCCCCC)CCCNc1c(NC)c(=O)c1=O] |
| I-76 | 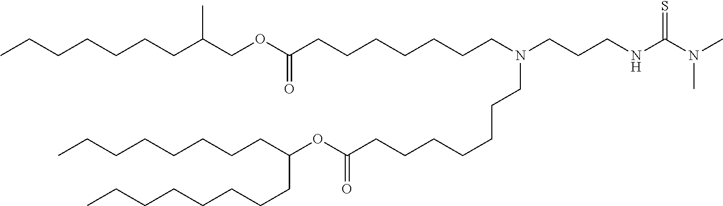 [SMILES: CCCCCCCCC(CCCCCCCC)OC(=O)CCCCCCCN(CCCCCCCC(=O)OCC(C)CCCCCCC)CCCNC(=S)N(C)C] |
| I-77 | 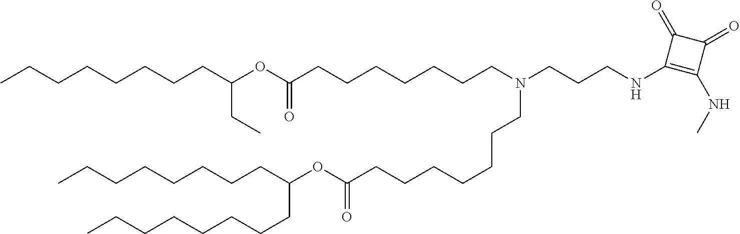 [SMILES: CCCCCCCCC(CC)OC(=O)CCCCCCCN(CCCCCCCC(=O)OC(CCCCCCCC)CCCCCCCC)CCCNc1c(NC)c(=O)c1=O] |
| I-78 | 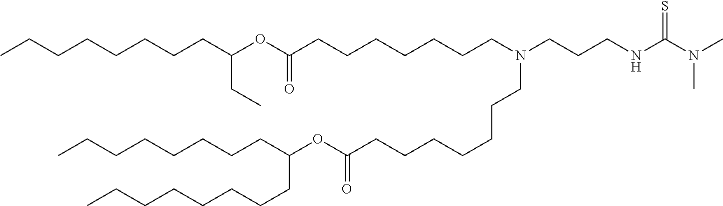 [SMILES: CCCCCCCCC(CC)OC(=O)CCCCCCCN(CCCCCCCC(=O)OC(CCCCCCCC)CCCCCCCC)CCCNC(=S)N(C)C] |
| I-79 | 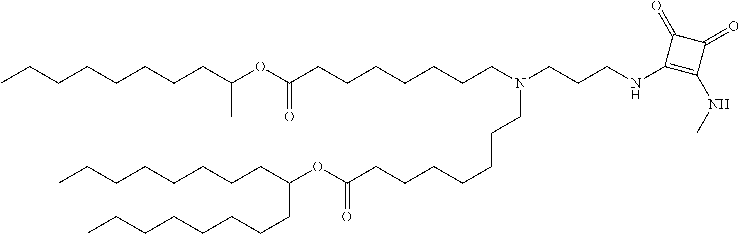 [SMILES: CCCCCCCCC(C)OC(=O)CCCCCCCN(CCCCCCCC(=O)OC(CCCCCCCC)CCCCCCCC)CCCNc1c(NC)c(=O)c1=O] |
| I-80 | 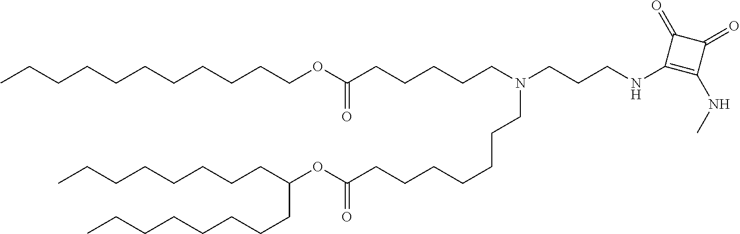 [SMILES: CCCCCCCCCCCOC(=O)CCCCCN(CCCCCCCC(=O)OC(CCCCCCCC)CCCCCCCC)CCCNc1c(NC)c(=O)c1=O] |
| I-81 | 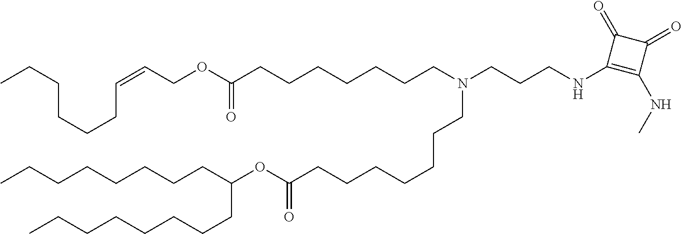 [SMILES: CCCCCC/C=C\COC(=O)CCCCCCCN(CCCCCCCC(=O)OC(CCCCCCCC)CCCCCCCC)CCCNc1c(NC)c(=O)c1=O] |
| I-82 | 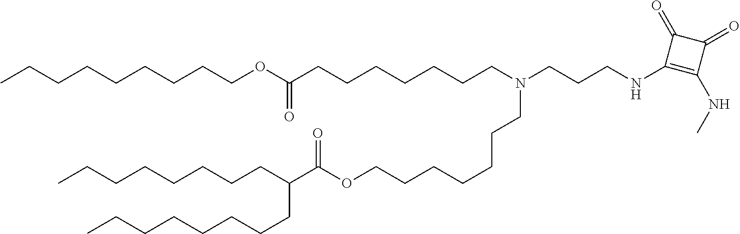 [SMILES: CCCCCCCCCOC(=O)CCCCCCCN(CCCCCCCOC(=O)C(CCCCCCCC)CCCCCCCC)CCCNc1c(NC)c(=O)c1=O] |
| I-83 | 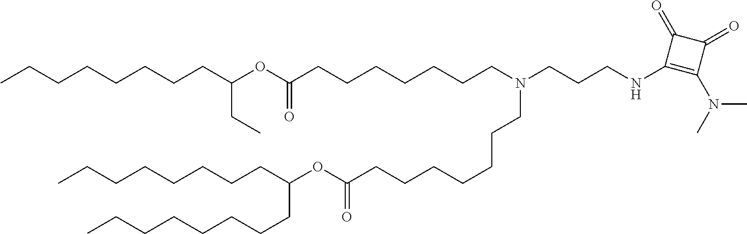 [SMILES: CCCCCCCCC(CC)OC(=O)CCCCCCCN(CCCCCCCC(=O)OC(CCCCCCCC)CCCCCCCC)CCCNc1c(N(C)C)c(=O)c1=O] |
| I-84 | 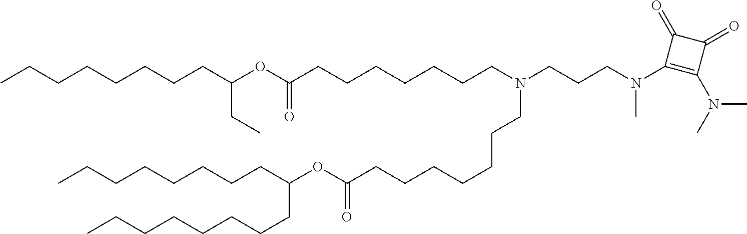 [SMILES: CCCCCCCCC(CC)OC(=O)CCCCCCCN(CCCCCCCC(=O)OC(CCCCCCCC)CCCCCCCC)CCCN(C)c1c(N(C)C)c(=O)c1=O] |
| I-85 | 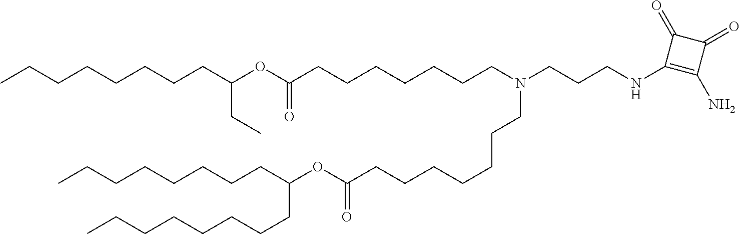 [SMILES: CCCCCCCCC(CC)OC(=O)CCCCCCCN(CCCCCCCC(=O)OC(CCCCCCCC)CCCCCCCC)CCCNc1c(N)c(=O)c1=O] |
| I-86 | 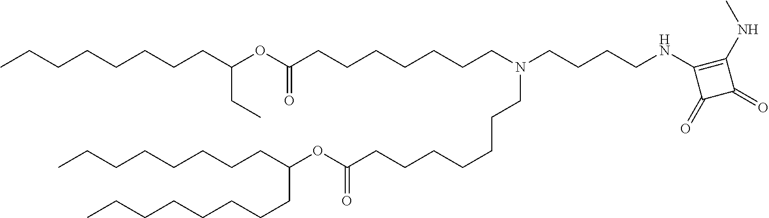 [SMILES: CCCCCCCCC(CC)OC(=O)CCCCCCCN(CCCCCCCC(=O)OC(CCCCCCCC)CCCCCCCC)CCCCNc1c(NC)c(=O)c1=O] |
| I-87 | 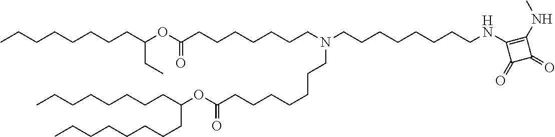 [SMILES: CCCCCCCCC(CC)OC(=O)CCCCCCCN(CCCCCCCCNc1c(NC)c(=O)c1=O)CCCCCCCC(=O)OC(CCCCCCCC)CCCCCCCC] |

or a pharmaceutically acceptable salt thereof. In some embodiments, provided compounds are provided and/or utilized in a salt form (e.g., a pharmaceutically acceptable salt form). Reference to a compound provided herein is understood to include reference to salts thereof, unless otherwise indicated.

In some embodiments, a cationic lipid is selected from Table 2:

TABLE 2

| Name | Structure |
|---|---|
| ALC- 0315 | 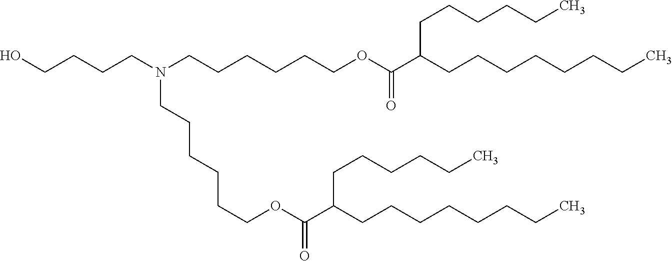 [SMILES: CCCCCCCCC(CCCCCC)C(=O)OCCCCCCN(CCCCO)CCCCCCOC(=O)C(CCCCCC)CCCCCCCC] |
| ALC366 | 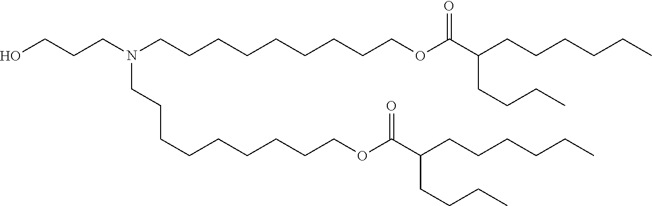 [SMILES: CCCCCCC(CCCC)C(=O)OCCCCCCCCCN(CCCO)CCCCCCCCCOC(=O)C(CCCC)CCCCCC] |
| SM-102 | 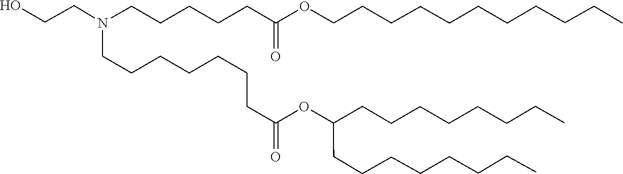 [SMILES: CCCCCCCCCCCOC(=O)CCCCCN(CCO)CCCCCCCC(=O)OC(CCCCCCCC)CCCCCCCC] |
| MC3 | 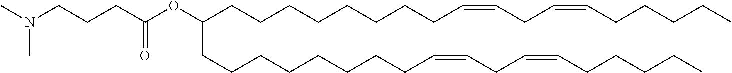 [SMILES: CCCCC/C=C\C/C=C\CCCCCCCCC(CCCCCCCC/C=C\C/C=C\CCCCC)OC(=O)CCCN(C)C] |
| Lipid 2,2 (8,8) 4C CH3 | 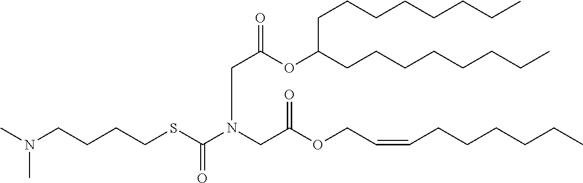 [SMILES: CCCCCC/C=C\COC(=O)CN(CC(=O)OC(CCCCCCCC)CCCCCCCC)C(=O)SCCCCN(C)C] |
| CL1 | 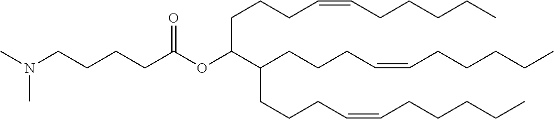 [SMILES: CCCCC/C=C\CCCC(CCC/C=C\CCCCC)C(CCC/C=C\CCCCC)OC(=O)CCCCN(C)C] |
| A9 | 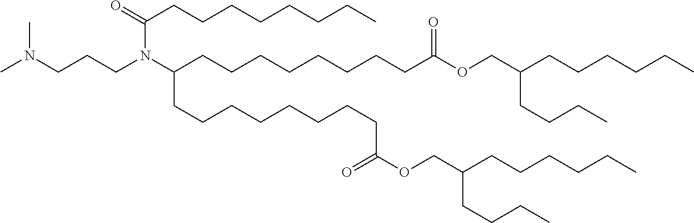 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] |
| Lipid 319 | 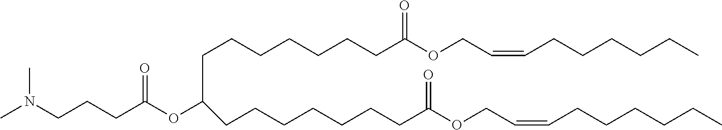 [SMILES: CCCCCC/C=C\COC(=O)CCCCCCCC(CCCCCCCC(=O)OC/C=C\CCCCCC)OC(=O)CCCN(C)C] |
| DLin- MC3- DMA | 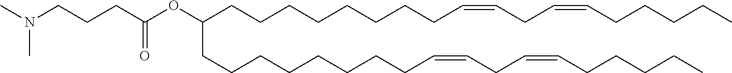 [SMILES: CCCCC/C=C\C/C=C\CCCCCCCCC(CCCCCCCC/C=C\C/C=C\CCCCC)OC(=O)CCCN(C)C] |
| OF-02 | 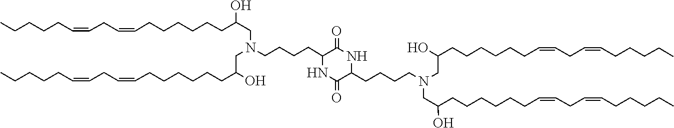 [SMILES: CCCCC/C=C\C/C=C\CCCCCCC(O)CN(CCCCC1NC(=O)C(CCCCN(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC] |
| A18-Iso5- 2DC18 | 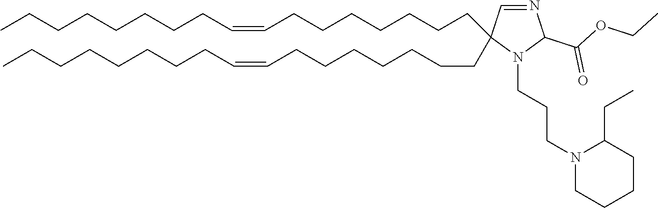 [SMILES: CCCCCCCC/C=C\CCCCCCCC1(CCCCCCC/C=C\CCCCCCCC)C=NC(C(=O)OCC)N1CCCN1CCCCC1CC] |
| 98N₁-5 | 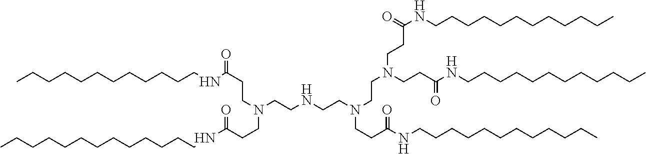 [SMILES: CCCCCCCCCCCCCNC(=O)CCN(CCNCCN(CCC(=O)NCCCCCCCCCCCC)CCN(CCC(=O)NCCCCCCCCCCCC)CCC(=O)NCCCCCCCCCCCC)CCC(=O)NCCCCCCCCCCCC] |
| 9A1P9 | 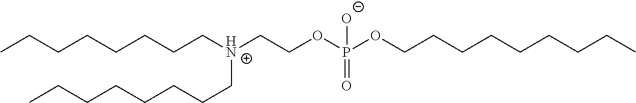 |
| C12-200 | 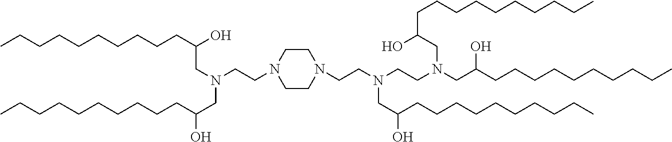 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC] |
| cKK-E12 | 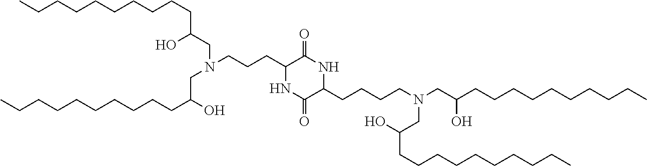 [SMILES: CCCCCCCCCCC(O)CN(CCCCC1NC(=O)C(CCCN(CC(O)CCCCCCCCCC)CC(O)CCCCCCCCCC)NC1=O)CC(O)CCCCCCCCCC] |
| 7C1 | 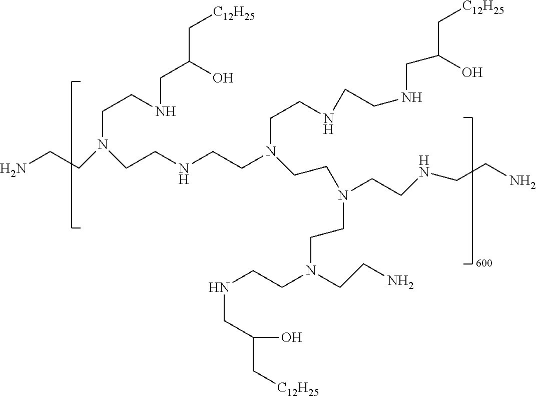 |
| G0-C14 | 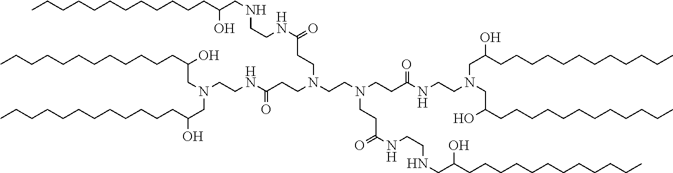 [SMILES: CCCCCCCCCCCCC(O)CNCCNC(=O)CCN(CCC(=O)NCCN(CC(O)CCCCCCCCCCCC)CC(O)CCCCCCCCCCCC)CCN(CCC(=O)NCCNCC(O)CCCCCCCCCCCC)CCC(=O)NCCN(CC(O)CCCCCCCCCCCC)CC(O)CCCCCCCCCCCC] |
| L319 | 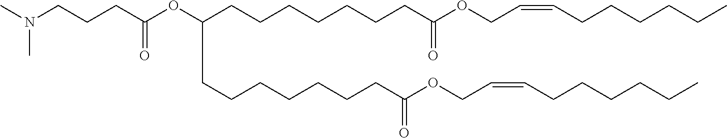 [SMILES: CCCCCC/C=C\COC(=O)CCCCCCCC(CCCCCCCC(=O)OC/C=C\CCCCCC)OC(=O)CCCN(C)C] |
| 304O₁₃ | 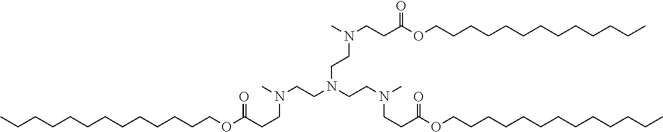 [SMILES: CCCCCCCCCCCCCOC(=O)CCN(C)CCN(CCN(C)CCC(=O)OCCCCCCCCCCCCC)CCN(C)CCC(=O)OCCCCCCCCCCCCC] |
| OF-Deg- Lin | 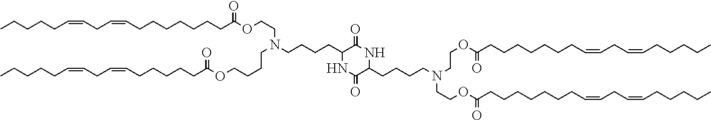 [SMILES: CCCCC/C=C\C/C=C\CCCCCCCC(=O)OCCN(CCCCOC(=O)CCCCC/C=C\C/C=C\CCCCC)CCCCC1NC(=O)C(CCCCN(CCOC(=O)CCCCCCC/C=C\C/C=C\CCCCC)CCOC(=O)CCCCCCC/C=C\C/C=C\CCCCC)NC1=O] |
| 306O12B | 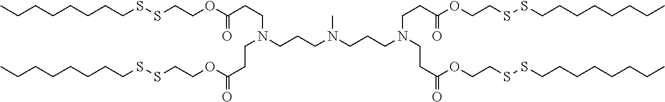 [SMILES: CCCCCCCCSSCCOC(=O)CCN(CCCN(C)CCCN(CCC(=O)OCCSSCCCCCCCC)CCC(=O)OCCSSCCCCCCCC)CCC(=O)OCCSSCCCCCCCC] |
| 306Oᵢ₁₀ | 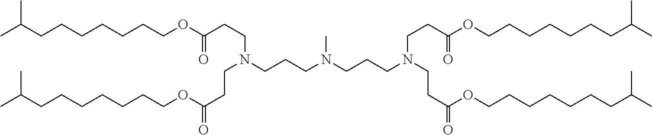 [SMILES: CC(C)CCCCCCCOC(=O)CCN(CCCN(C)CCCN(CCC(=O)OCCCCCCCC(C)C)CCC(=O)OCCCCCCCC(C)C)CCC(=O)OCCCCCCCC(C)C] |
| FTT5 | 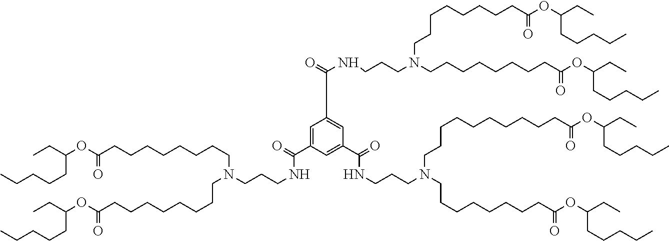 [SMILES: CCCCCC(CC)OC(=O)CCCCCCCCCCN(CCCCCCCCC(=O)OC(CC)CCCCC)CCCNC(=O)c1cc(C(=O)NCCCN(CCCCCCCCC(=O)OC(CC)CCCCC)CCCCCCCCC(=O)OC(CC)CCCCC)cc(C(=O)NCCCN(CCCCCCCCC(=O)OC(CC)CCCCC)CCCCCCCCC(=O)OC(CC)CCCCC)c1] |
| 10q | 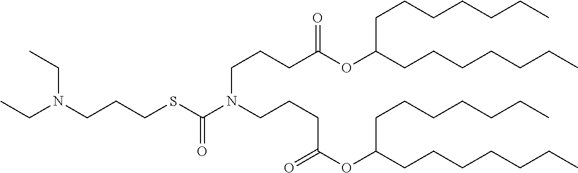 [SMILES: CCCCCCCC(CCCCCCC)OC(=O)CCCN(CCCC(=O)OC(CCCCCCC)CCCCCCC)C(=O)SCCCN(CC)CC] |
| ssPalmO- Phe | 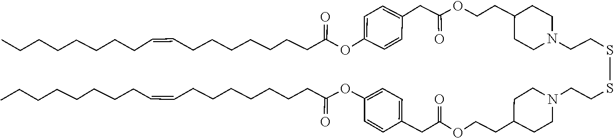 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1] |
| CL4H6 | 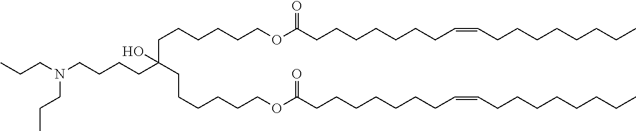 [SMILES: CCCCCCCC/C=C\CCCCCCCC(=O)OCCCCCCC(O)(CCCCCCOC(=O)CCCCCCC/C=C\CCCCCCCC)CCCCN(CCC)CCC] |
| YSK12- C4 | 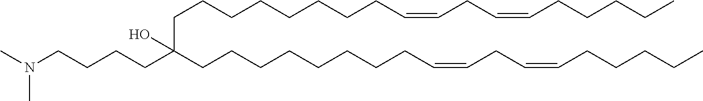 |
| EA2 | 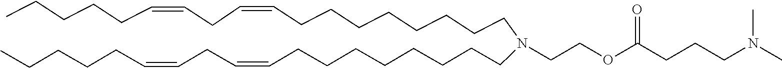 [SMILES: CCCCC/C=C\C/C=C\CCCCCCCCN(CCCCCCCC/C=C\C/C=C\CCCCC)CCOC(=O)CCCN(C)C] |
| HY-501 | 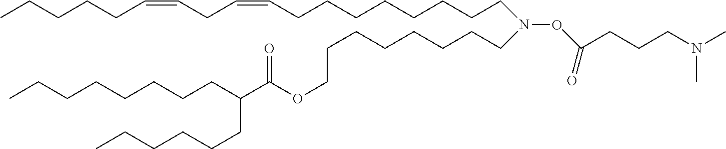 [SMILES: CCCCC/C=C\C/C=C\CCCCCCCCN(CCCCCCCCOC(=O)C(CCCCCC)CCCCCCCC)OC(=O)CCCN(C)C] |

or a pharmaceutically acceptable salt thereof. In some embodiments, provided compounds are provided and/or utilized in a salt form (e.g., a pharmaceutically acceptable salt form). Reference to a compound provided herein is understood to include reference to salts thereof, unless otherwise indicated.

In some embodiments, a cationic lipid is selected from Tables 1 and/or 2.

In some embodiments, a cationic lipid is selected from DODMA, HY-501, ALC-0315, ALC366, and SM-102. In some embodiments, a cationic lipid is selected from ALC-0315 and ALC366. In some embodiments, a cationic lipid is ALC-0315. In some embodiments, a cationic lipid is ALC366. In some embodiments, a cationic lipid is SM-102. In some embodiments, a cationic lipid is DODMA. In some embodiments, a cationic lipid is HY-501.

In some embodiments, LNPs of the present disclosure comprise about 30 to about 70 mol % of a cationic lipid relative to the total lipids in the LNP. In some embodiments, an LNP comprises about 35 to about 65 mol % of a cationic lipid. In some embodiments, an LNP comprises about 40 to about 60 mol % of a cationic lipid. In some embodiments, an LNP comprises about 41 to about 49 mol % of a cationic lipid. In some embodiments, an LNP comprises about 48 mol % of a cationic lipid. In some embodiments, an LNP comprises about 50 mol % of a cationic lipid.

In some embodiments, the cationically ionizable lipid has the structure of Formula (X)

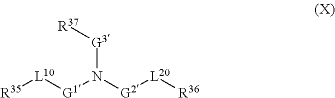

or a pharmaceutically acceptable salt, tautomer, prodrug or stereoisomer thereof,
wherein:
one of L¹⁰ and L²⁰ is —O(C═O)—, —(C═O)O—, —C(═O)—, —O—, —S(O)ₓ—, —S—S—, —C(═O)S—, SC(═O)—,
—NR^{a}C(═O)—, —C(═O)NR^{a}—, NR^{a}C(═O)NR^{a}—, —OC(═O)NR^{a}— or —NR^{a}C(═O)O—, and the other of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G^{1′} and G^{2′} are each independently unsubstituted C₁-C₁₂ alkylene or C₂₋₁₂ alkenylene;
G^{3′} is C₁₋₂₄ alkylene, C₂₋₂₄ alkenylene, C₃₋₈ cycloalkylene, or C₃₋₈ cycloalkenylene;
R^{a′} is H or C₁₋₁₂ alkyl;
R³⁵ and R³⁶ are each independently C₆₋₂₄ alkyl or C₆₋₂₄ alkenyl;
R³⁷ is H, OR⁵⁰, CN, —C(═O)OR⁴⁰, —OC(═O)R⁴⁰ or —NR⁵⁰C(═O)R⁴⁰;
R⁴⁰ is C₁₋₁₂ alkyl;
R⁵⁰ is H or C₁₋₆ alkyl; and
x′ is 0, 1 or 2.

In some of the foregoing embodiments of Formula (X), the lipid has one of the following structures (XA) or (XB):

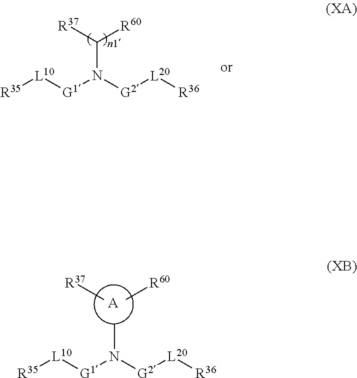

wherein R³⁵, L¹⁰, G^{1′}, G^{2′}, L²⁰, R³⁶, R³⁷, and R⁶⁰ are as described in classes and subclasses herein, both singly and in combination;
A is a 3 to 8-membered cycloalkyl or cycloalkylene group;
R⁶⁰ is, at each occurrence, independently H, OH or C₁-C₂₄ alkyl; and
n^{1′} is an integer ranging from 1 to 15.

In some of the foregoing embodiments of Formula (X), the lipid has structure (XA), and in other embodiments, the lipid has structure (XB).

In other embodiments of Formula (X), the lipid has one of the following structures (XC) or (XD):

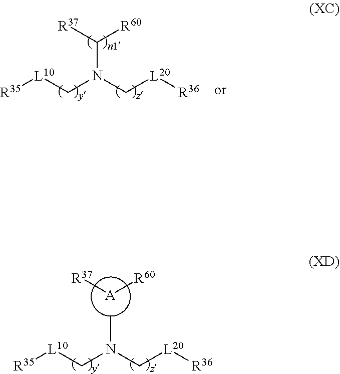

wherein R³⁵, L¹⁰, G^{1′}, G^{2′}, L²⁰, R³⁶, R³⁷, and R⁶⁰ are as described in classes and subclasses herein, both singly and in combination; and y′ and z′ are each independently integers ranging from 1 to 12.

In any of the foregoing embodiments of Formula (X), one of L¹⁰ and L²⁰ is —O(C═O)—. For example, in some embodiments each of L¹⁰ and L²⁰ are —O(C═O)—. In some different embodiments of any of the foregoing, L¹⁰ and L²⁰ are each independently —(C═O)O— or —O(C═O)—. For example, in some embodiments each of L¹⁰ and L²⁰ is —(C═O)O—.

In some embodiments of Formula (X), the lipid has one of the following structures (XE) or (XF):

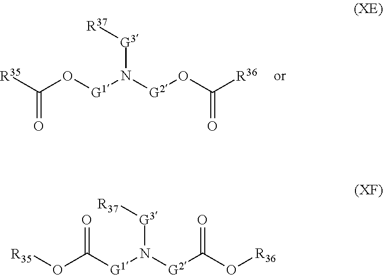

wherein R³⁵, R³⁶, R³⁷, G^{1′}, G^{2′}, and G^{3′} are as defined in classes and subclasses herein, both singly and in combination.

In some of the foregoing embodiments of Formula (X), the lipid has one of the following structures (XG), (XH), (XJ), or (XK):

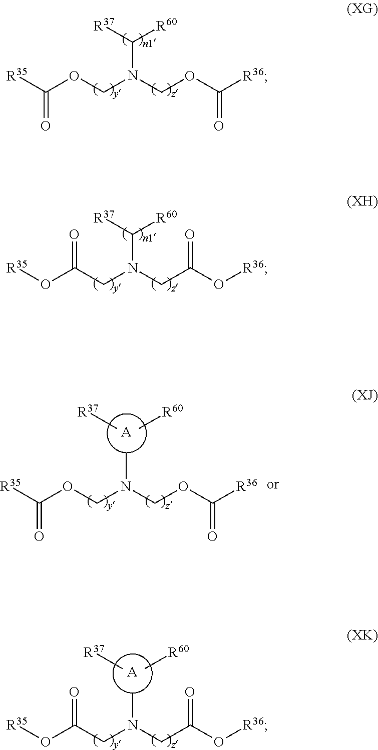

wherein R³⁵, R³⁶, R³⁷, R⁶⁰, y′, z′, n^{1′}, and A are as defined in classes and subclasses herein, both singly and in combination.

In some of the foregoing embodiments of Formula (X), n^{1′} is an integer ranging from 2 to 12, for example from 2 to 8 or from 2 to 4. For example, in some embodiments, n^{1′} is 3, 4, 5 or 6. In some embodiments, n^{1′} is 3. In some embodiments, n^{1′} is 4. In some embodiments, n^{1′} is 5. In some embodiments, n^{1′} is 6.

In some other of the foregoing embodiments of Formula (X), y′ and z′ are each independently an integer ranging from 2 to 10. For example, in some embodiments, y′ and z′ are each independently an integer ranging from 4 to 9 or from 4 to 6.

In some of the foregoing embodiments of Formula (X), R⁶⁰ is H. In other of the foregoing embodiments, R⁶⁰ is C₁-C₂₄ alkyl. In other embodiments, R⁶⁰ is OH. In some embodiments of Formula (X), G³ is unsubstituted. In other embodiments, G³ is substituted. In various different embodiments, G³ is linear C₁-C₂₄ alkylene or linear C₂-C₂₄alkenylene.

In some other foregoing embodiments of Formula (X), R³⁵ or R³⁶, or both, is C₆-C₂₄ alkenyl. For example, in some embodiments, R³⁵ and R³⁶ each, independently have the following structure:

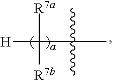

wherein:
R^{7a} and R^{7b} are, at each occurrence, independently H or C₁-C₁₂ alkyl; and
a is an integer from 2 to 12,
wherein R^{7a}, R^{7b} and a are each selected such that R³⁵ and R³⁶ each independently comprise from 6 to 20 carbon atoms. For example, in some embodiments a is an integer ranging from 5 to 9 or from 8 to 12.

In some of the foregoing embodiments of Formula (X), at least one occurrence of R^{7a} is H. For example, in some embodiments, R^{7a} is H at each occurrence. In other different embodiments of the foregoing, at least one occurrence of R^{7b} is C₁-C₂₄ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (X), R³⁵ or R³⁶, or both, has one of the following structures:

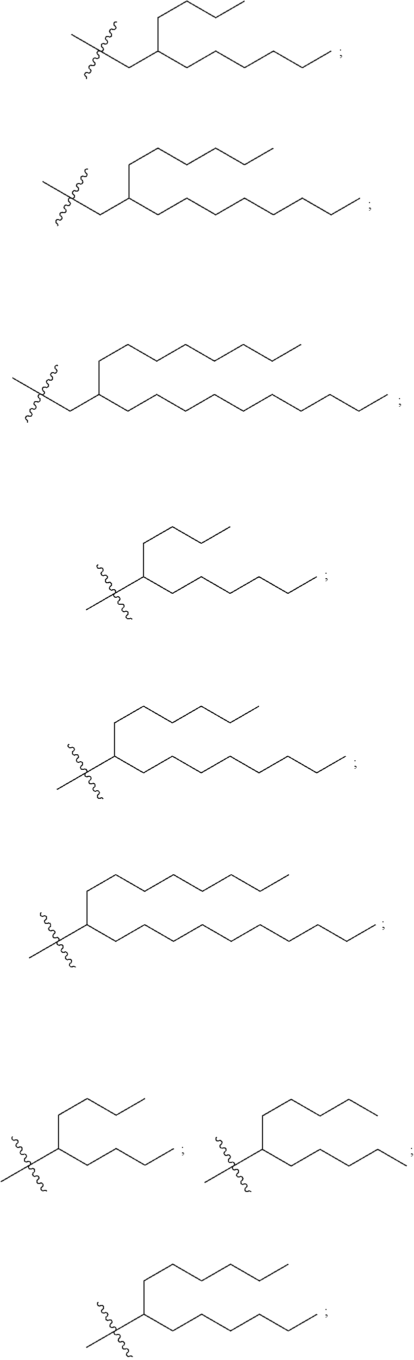

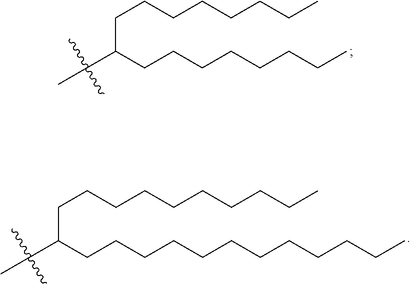 [SMILES: *C(CCCCCCCC)CCCCCCCC ; *C(CCCCCCCCCC)CCCCCCCCCCCC]

In some of the foregoing embodiments of Formula (X), R³⁷ is OH, CN, —C(═O)OR⁴⁰, —OC(═O)R⁴⁰ or —NHC(═O)R⁴⁰. In some embodiments, R⁴⁰ is methyl or ethyl.

In various different embodiments, a cationic lipid of Formula (X) has one of the structures set forth below.

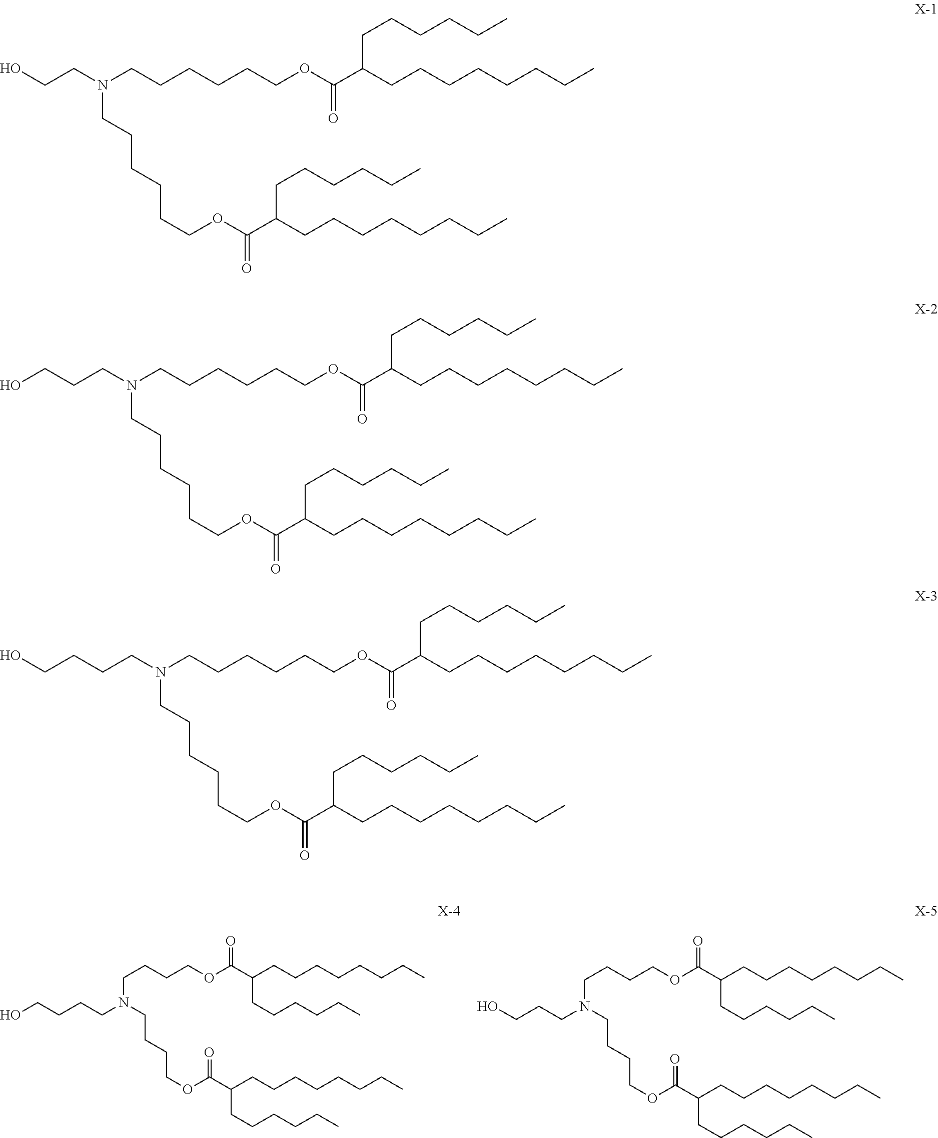 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)C(=O)OCCCCN(CCCCO)CCCCOC(=O)C(CCCCCC)CCCCCCCC ; CCCCCCCCC(CCCCCC)C(=O)OCCCCN(CCCO)CCCCOC(=O)C(CCCCCC)CCCCCCCC]

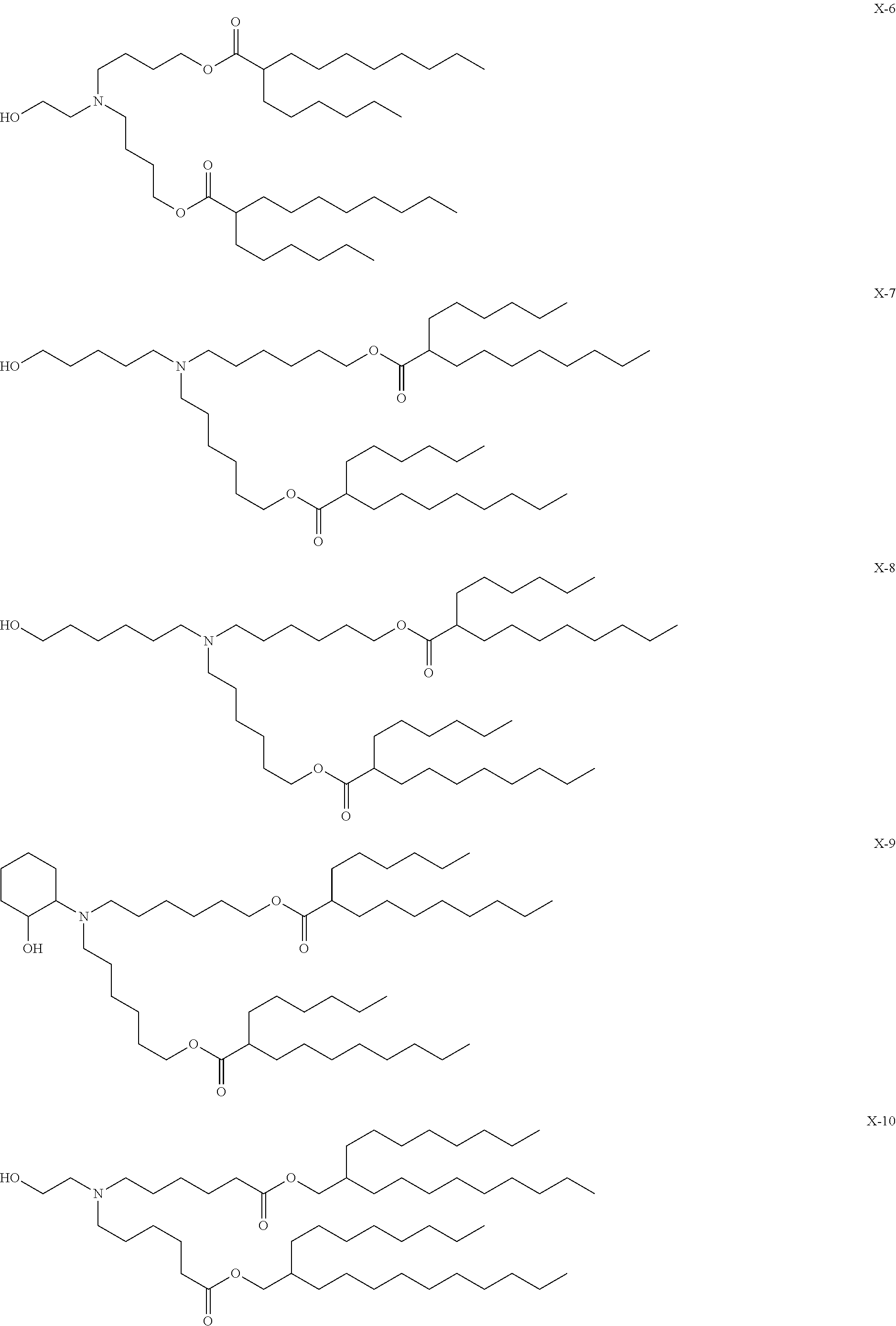

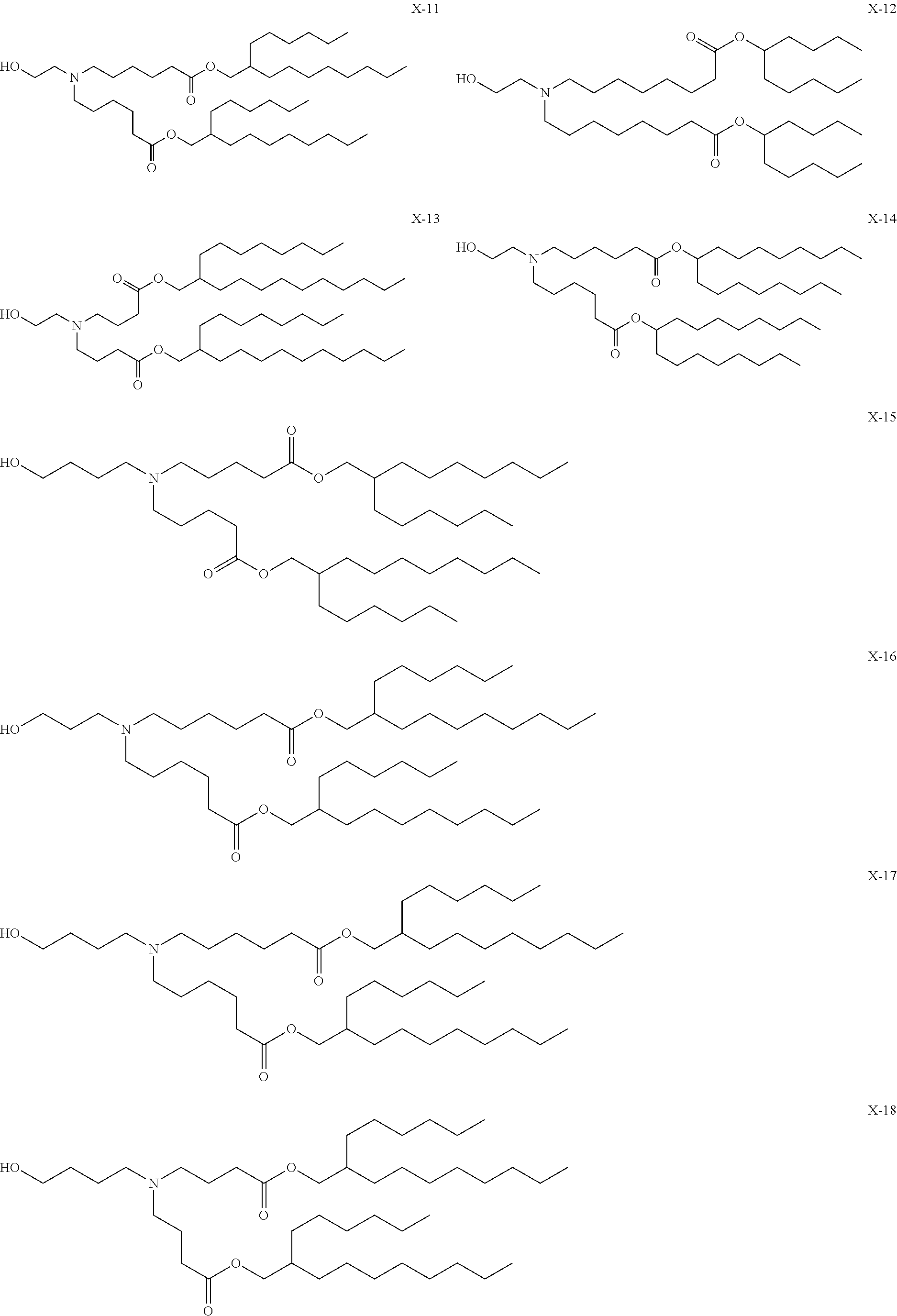

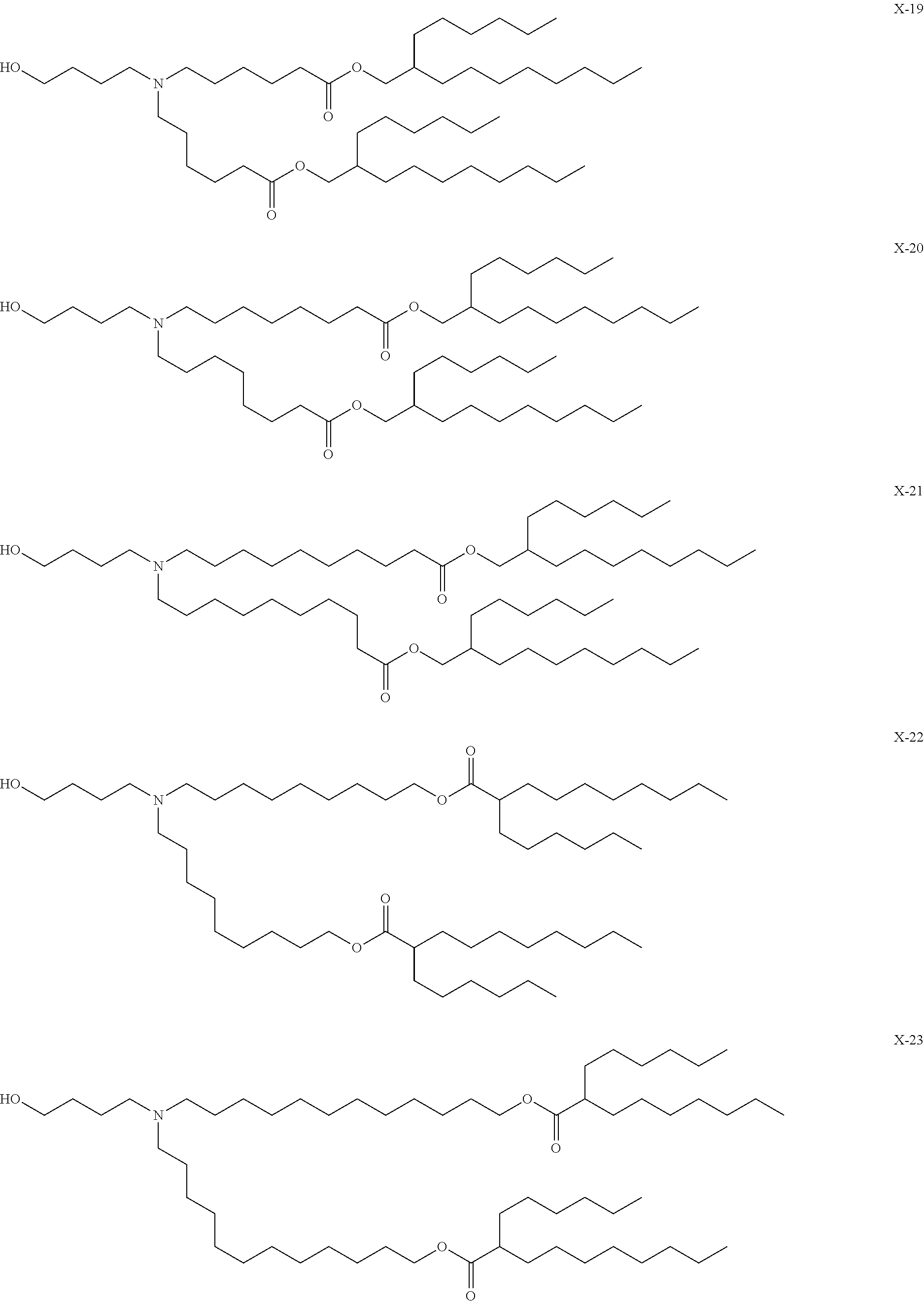 [SMILES: CCCCCCCCC(CCCCCC)COC(=O)CCCCCN(CCCCO)CCCCCC(=O)OCC(CCCCCC)CCCCCCCC ; CCCCCCCCC(CCCCCC)COC(=O)CCCCCCCN(CCCCO)CCCCCCCC(=O)OCC(CCCCCC)CCCCCCCC ; CCCCCCCCC(CCCCCC)COC(=O)CCCCCCCCCN(CCCCO)CCCCCCCCCC(=O)OCC(CCCCCC)CCCCCCCC ; CCCCCCCCC(CCCCCC)C(=O)OCCCCCCCCCN(CCCCO)CCCCCCCCCOC(=O)C(CCCCCC)CCCCCCCC ; CCCCCCCCC(CCCCCC)C(=O)OCCCCCCCCCCCCN(CCCCO)CCCCCCCCCCCCOC(=O)C(CCCCCC)CCCCCCC]

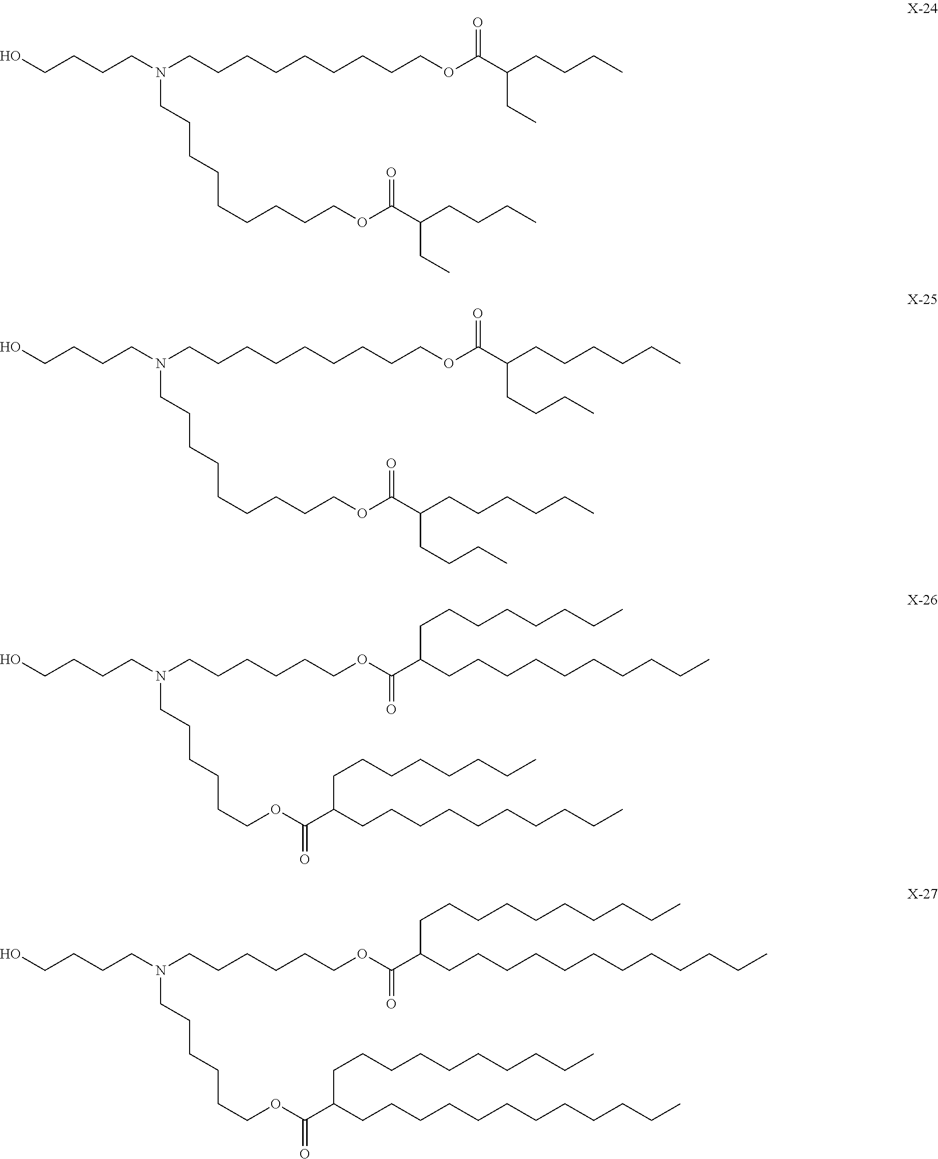

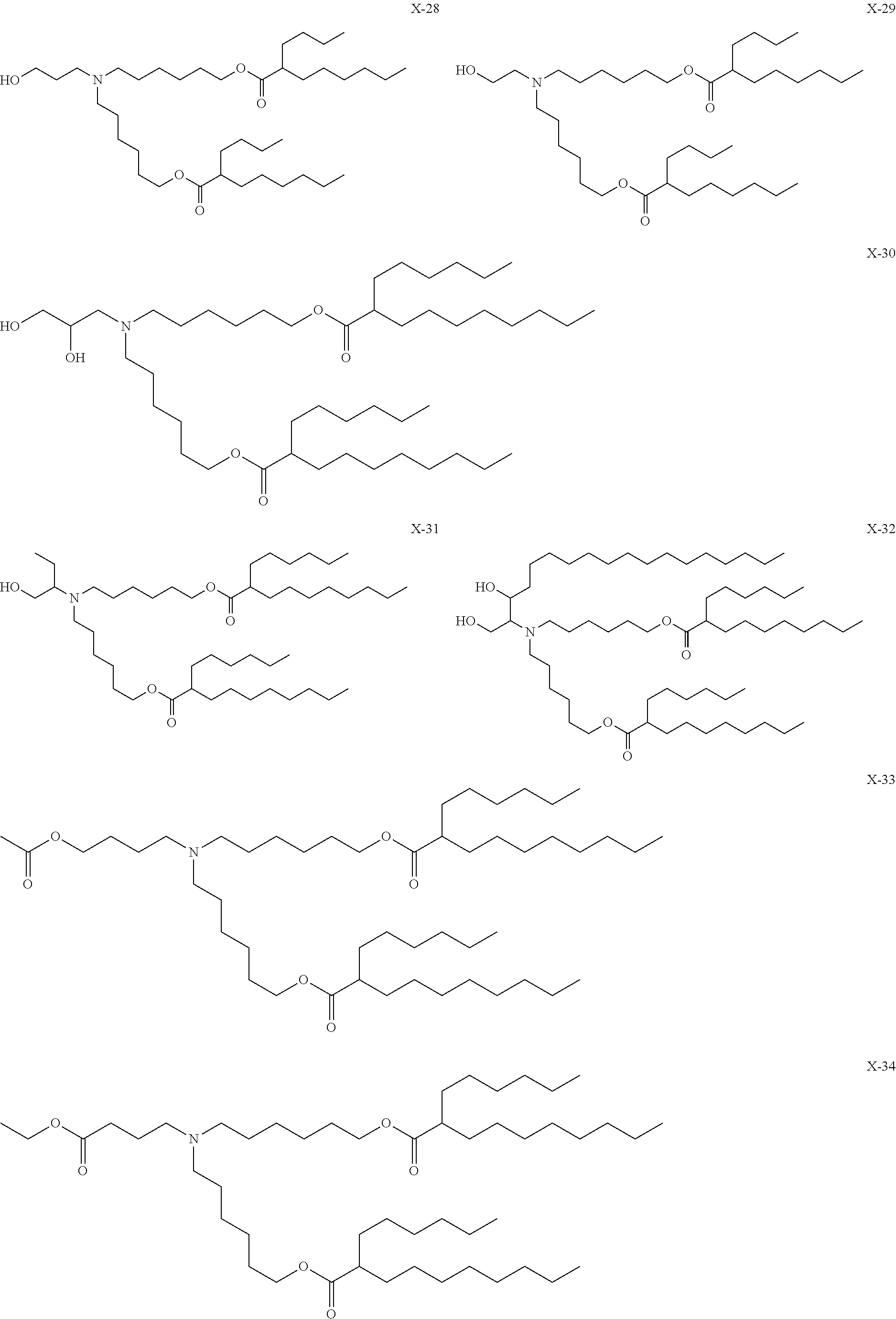

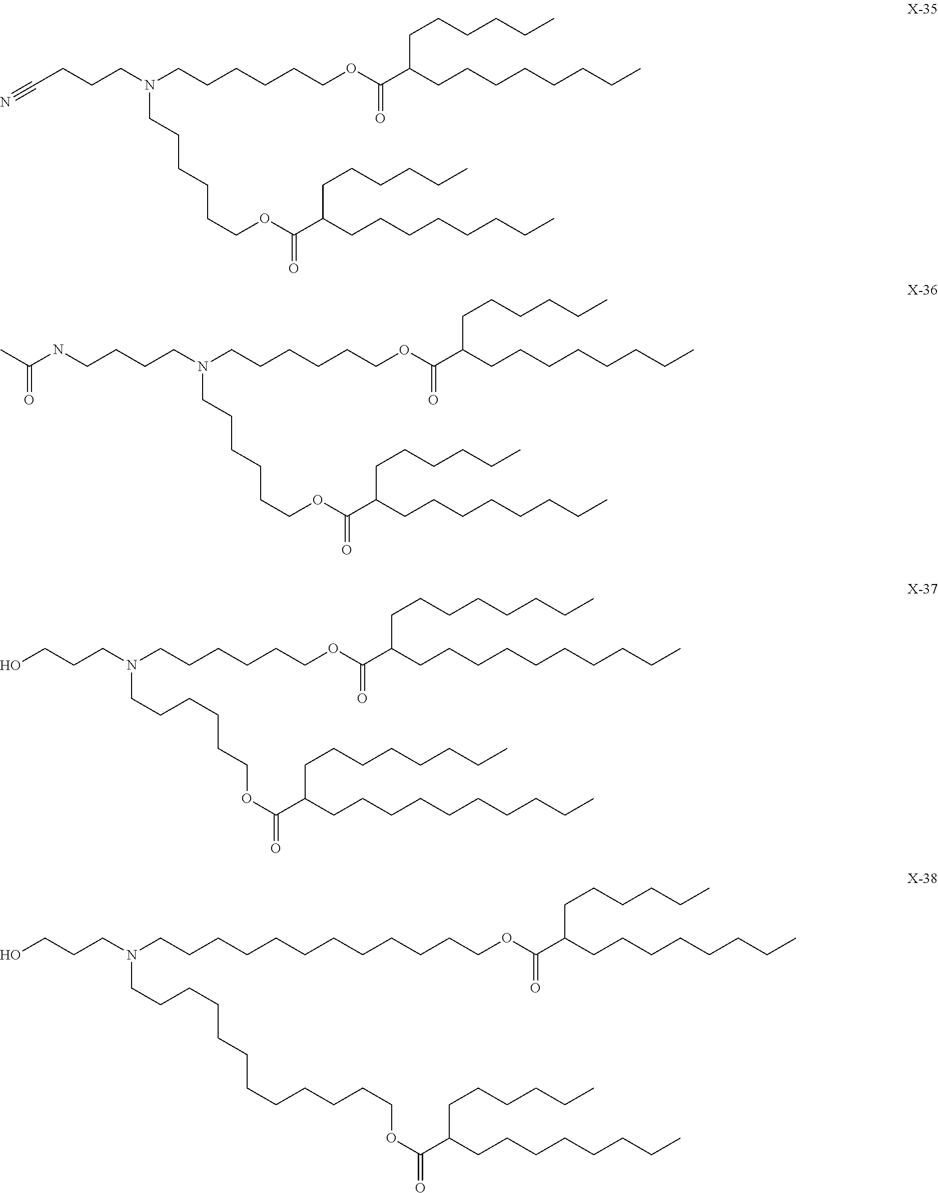

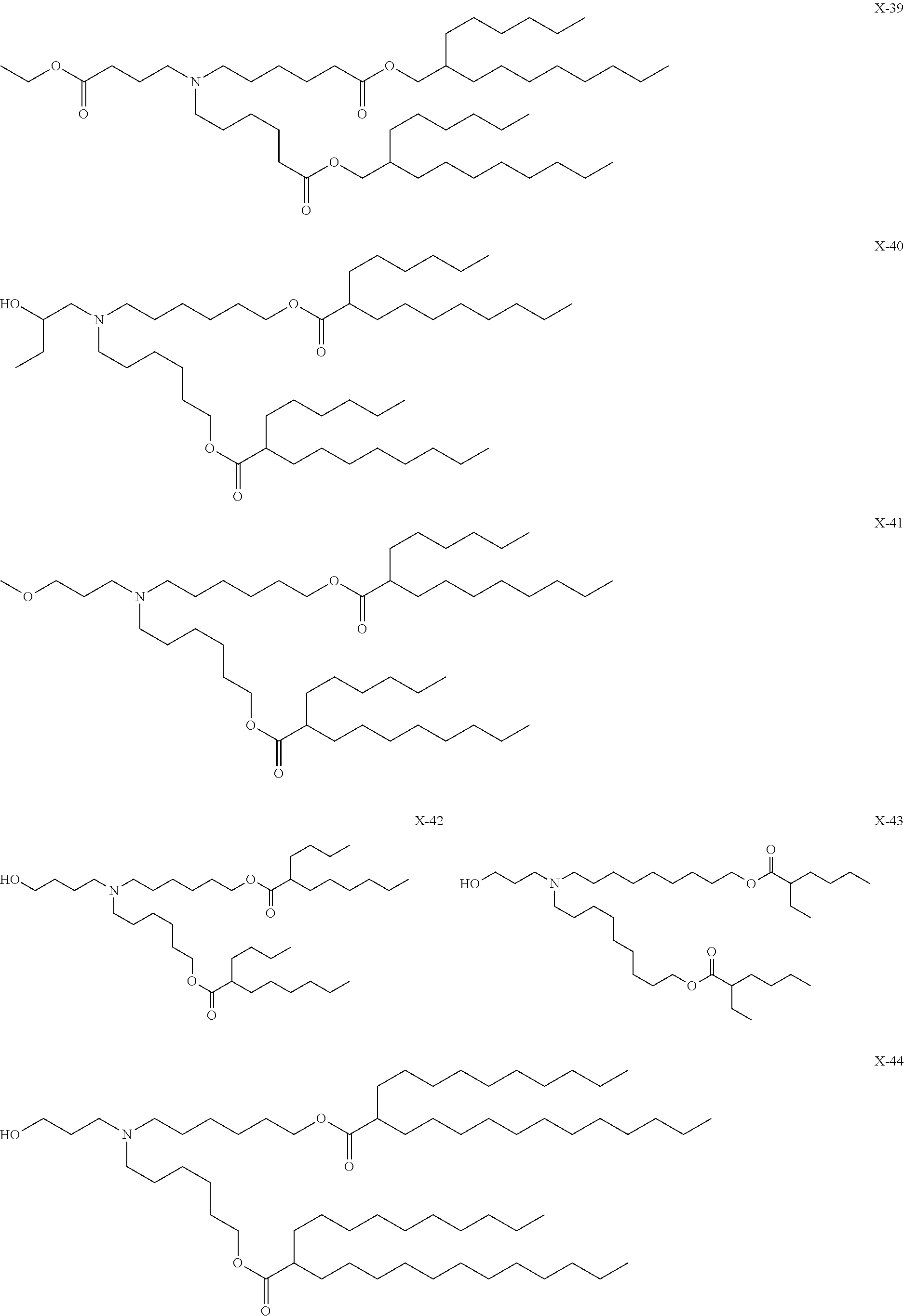

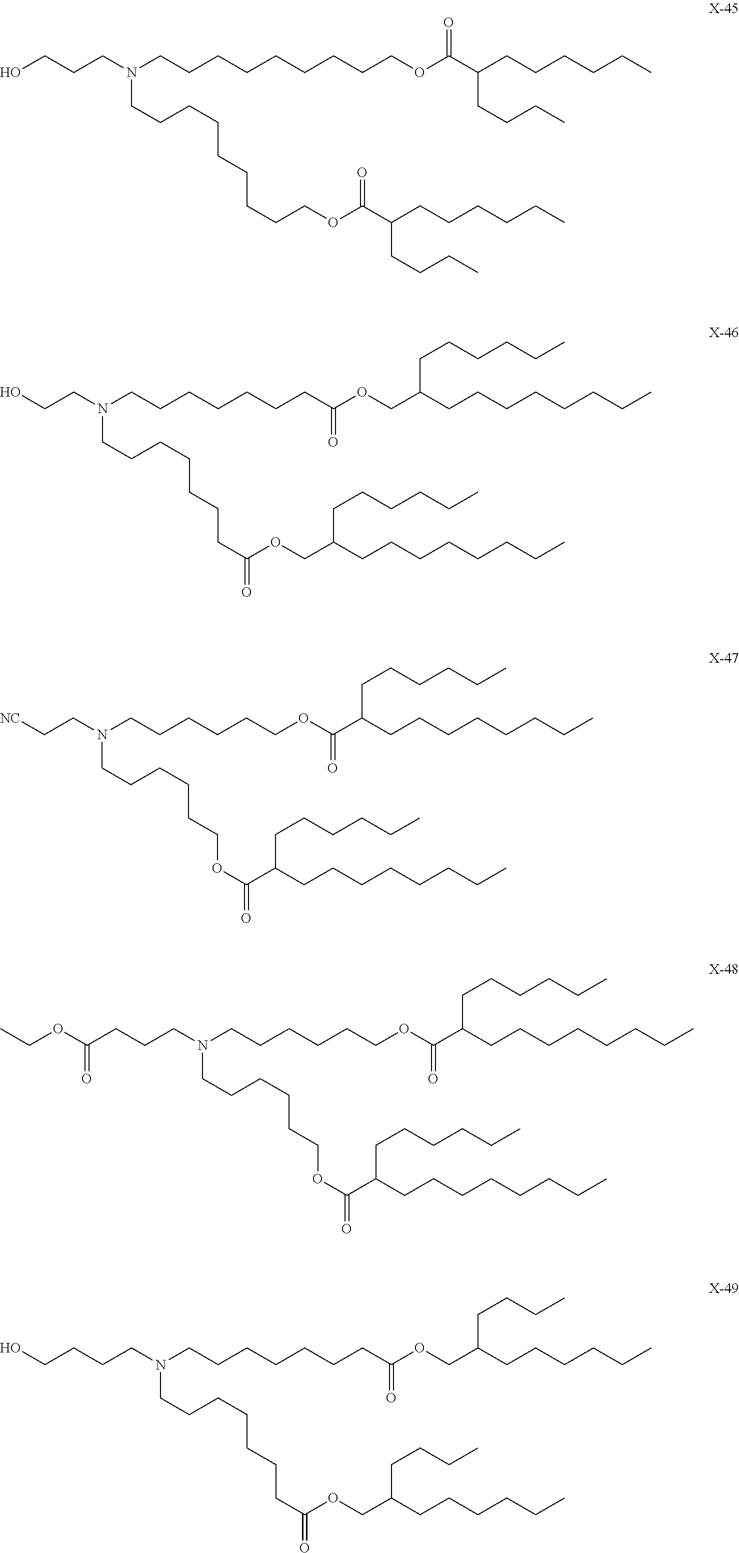

In various different embodiments, the cationically ionizable lipid has one of the structures set forth in the table below.

| No. | Structure |
|---|---|
| A | 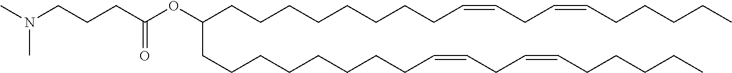 [SMILES: CCCCC/C=C\C/C=C\CCCCCCCCC(CCCCCCCC/C=C\C/C=C\CCCCC)OC(=O)CCCN(C)C] |
| B | 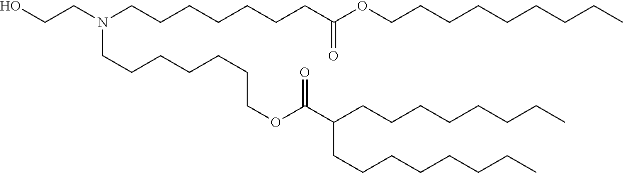 [SMILES: CCCCCCCCCOC(=O)CCCCCCCN(CCO)CCCCCCCOC(=O)C(CCCCCCCC)CCCCCCCC] |
| C | 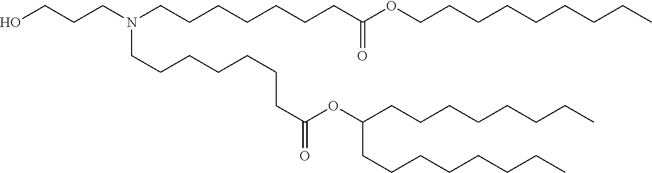 [SMILES: CCCCCCCCCOC(=O)CCCCCCCN(CCCO)CCCCCCCC(=O)OC(CCCCCCCC)CCCCCCCC] |
| D | 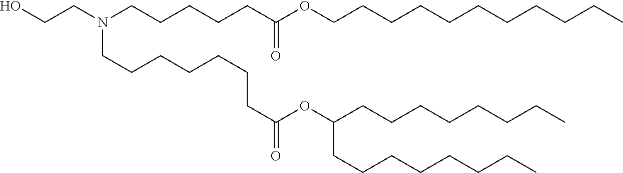 [SMILES: CCCCCCCCCCCOC(=O)CCCCCN(CCO)CCCCCCCC(=O)OC(CCCCCCCC)CCCCCCCC] |
| E | 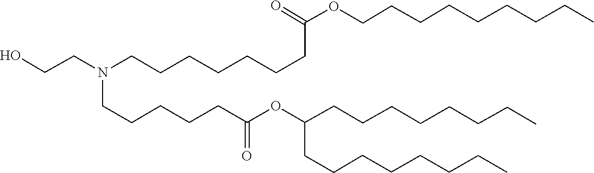 [SMILES: CCCCCCCCCOC(=O)CCCCCCCN(CCO)CCCCCC(=O)OC(CCCCCCCC)CCCCCCCC] |
| F | 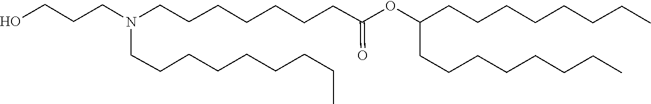 [SMILES: CCCCCCCCCN(CCCO)CCCCCCCC(=O)OC(CCCCCCCC)CCCCCCCC] |
| G | 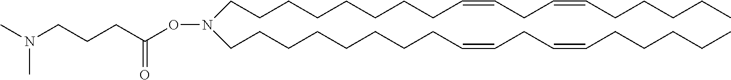 [SMILES: CCCCC/C=C\C/C=C\CCCCCCCCN(CCCCCCCC/C=C\C/C=C\CCCCC)OC(=O)CCCN(C)C] |

In some embodiments, the cationically ionizable lipid has the structure of Formula (XI):

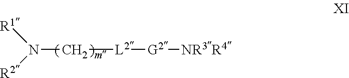

wherein
each of R^{1″} and R^{2″} is independently R^{5″} or -G^{1″}-L^{1″}-R^{6″}, wherein at least one of R^{1″} and R^{2″} is -G^{1″}-L^{1″} R^{6″};
each of R³ and R⁴ is independently selected from the group consisting of C₁₋₆ alkyl, C₂₋₆ alkenyl, aryl, and C₃₋₁₀ cycloalkyl;
each of R^{5″} and R^{6″} is independently a non-cyclic hydrocarbyl group having at least 10 carbon atoms;
each of G^{1″} and G^{2″} is independently unsubstituted C₁₋₁₂ alkylene or C₂₋₁₂ alkenylene;
each of L^{1″} and L^{2″} is independently selected from the group consisting of —O(C═O)—, —(C═O)O—, —C(═O)—, —O—, —S(O)_{x″}—, —S—S—, —C(═O)S—, —SC(═O)—, —NR^{a″}C(═O)—, —C(═O)NR^{a″}—, —NR^{a″}C(═O)NR^{a″}—, —OC(═O)NR^{a″}— and —NR^{a″}C(═O)O—;
R^{a″} is H or C₁₋₁₂ alkyl;
m″ is 0, 1, 2, 3, or 4; and
x″ is 0, 1 or 2.

In some of the foregoing embodiments of Formula (XI), G^{1″} is independently unsubstituted C₁-C₁₂ alkylene or unsubstituted C₂₋₁₂ alkenylene, e.g., unsubstituted, straight C₁₋₁₂ alkylene or unsubstituted, straight C₂₋₁₂ alkenylene. In some embodiments, each G^{1″} is independently unsubstituted C₆₋₁₂ alkylene or unsubstituted C₆₋₁₂ alkenylene, e.g., unsubstituted, straight C₆₋₁₂ alkylene or unsubstituted, straight C₆₋₁₂ alkenylene. In some embodiments, each G^{1″} is independently unsubstituted C₁₂ alkylene or unsubstituted C₈₋₁₂ alkenylene, e.g., unsubstituted, straight C₈₋₁₂ alkylene or unsubstituted, straight C₈₋₁₂ alkenylene. In some embodiments, each G^{1″} is independently unsubstituted C₆₋₁₀ alkylene or unsubstituted C₆₋₁₀ alkenylene, e.g., unsubstituted, straight C₆₋₁₀ alkylene or unsubstituted, straight C₆₋₁₀ alkenylene. In some embodiments, each G^{1″} is independently unsubstituted alkylene having 8, 9 or 10 carbon atoms, e.g., unsubstituted, straight alkylene having 8, 9 or 10 carbon atoms. In some embodiments, where R^{1″} and R^{2″} are both independently -G^{1″}-L^{1″}-R^{6″}, G^{1″} for R^{1″} may be different from G^{1″} for R^{2″}. In some of these embodiments, for example, G^{1″} for R^{1″} is unsubstituted, straight C₁₋₁₂ alkylene and G^{1″} for R^{2″} is unsubstituted, straight C₂₋₁₂ alkenylene; or G^{1″} for R^{1″} is an unsubstituted, straight C₁₋₁₂ alkylene group and G^{1″} for R^{2″} is a different unsubstituted, straight C₁₋₁₂ alkylene group. In some embodiments, where R^{1″} and R^{2″} are both independently -G^{1″}-L^{1″}-R^{6″}, G^{1″} for R^{1″} may be identical to G^{1″} for R^{2″} In some of these embodiments, for example, each G^{1″} is the same unsubstituted, straight C₁₂ alkylene, such as unsubstituted, straight C₁₀ alkylene, or each G^{1″} is the same unsubstituted, straight C₆₋₁₂ alkenylene.

In some of the foregoing embodiments of Formula (XI), each L^{1″} is independently selected from the group consisting of —O(C═O)—, —(C═O)O—, —C(═O)S—, —SC(═O)—, —NR^{a″}C(═O)—, and —C(═O)NR^{a″}—. In some embodiments, R^{a″} of L^{1″} is H or C₁₂ alkyl. In some embodiments, R^{a″} of L^{1″} is H or C₁₋₆ alkyl, e.g., H or C₁₋₃ alkyl. In some embodiments, R^{a″} of L^{1″} is H, methyl, or ethyl. In some embodiments, each L^{1″} is independently selected from the group consisting of —O(C═O)—, —(C═O)O—, —C(═O)S—, and —SC(═O)—. In some embodiments, each L^{1″} is independently —O(C═O)— or —(C═O)O—. In some embodiments, where R^{1″} and R^{2″} are both independently -G^{1″}-L^{1″}-R^{6″}, L^{1″} for R^{1″} may be different from L^{1″} for R^{2″}. In some of these embodiments, for example, L^{1″} for R^{1″} is one moiety selected from the group consisting of —O(C═O)—, —(C═O)O—, —C(═O)S—, —SC(═O)—, —NR^{a″}C(═O)—, and —C(═O)NR^{a″}— (e.g., L^{1″} for R^{1″} is —O(C═O)—), and L^{1″} for R^{2″} is a different moiety selected from the group consisting of —O(C═O)—, —(C═O)O—, —C(═O)S—, —SC(═O)—, —NR^{a″}C(═O)—, and —C(═O)NR^{a″}— (e.g., L^{1″} for R^{2″} is —(C═O)O—). In some embodiments, where R^{1″} and R^{2″} are both independently -G^{1″}-L^{1″}-R^{6″}, L^{1″} for R^{1″} may be identical to L^{1″} for R^{2″}. In some of these embodiments, for example, each L^{1″} is the same moiety selected from the group consisting of —O(C═O), —(C═O)O—, —C(═O)S—, —SC(═O)—, —NR^{a″}C(═O)—, and —C(═O)NR^{a″}—, e.g., each L^{1″} is —O(C═O)— or each L^{1″} is —(C═O)O—.

In some of the foregoing embodiments of Formula (XI), each R^{6″} is independently a non-cyclic hydrocarbyl group having at least 10 carbon atoms, e.g., a straight hydrocarbyl group having at least 10 carbon atoms. In some embodiments, each R^{6″} has independently at most 30 carbon atoms, such as at most 28, at most 26, at most 24, at most 22, or at most 20 carbon atoms. In some embodiments, each R^{6″} is independently a non-cyclic hydrocarbyl group having 10 to 30 carbon atoms (such as 10 to 28, 10 to 26, 10 to 24, 10 to 22, or 10 to 20 carbon atoms), e.g., a straight hydrocarbyl group having 10 to 30 carbon atoms (such as 10 to 28, 10 to 26, 10 to 24, 10 to 22, or 10 to 20 carbon atoms). In some embodiments, each R^{6″} is attached to L^{1″} via an internal carbon atom of R^{6″}. In some embodiments, each R^{6″} has independently at most 30 carbon atoms (such as at most 28, at most 26, at most 24, at most 22, or at most 20 carbon atoms), and each R^{6″} is attached to L^{1″} via an internal carbon atom of R^{6″}. In some embodiments, each R^{6″} is independently a non-cyclic hydrocarbyl group having at least 10 carbon atoms, e.g., a straight hydrocarbyl group having at least 10 carbon atoms, and each R^{6″} is attached to L^{1″} via an internal carbon atom of R^{6″}. In some embodiments, each R^{6″} is independently a non-cyclic hydrocarbyl group having 10 to 30 carbon atoms (such as 10 to 28, 10 to 26, 10 to 24, 10 to 22, or 10 to 20 carbon atoms), e.g., a straight hydrocarbyl group having 10 to 30 carbon atoms (such as 10 to 28, 10 to 26, 10 to 24, 10 to 22, or to 20 carbon atoms), and each R^{6″} is attached to L^{1″} via an internal carbon atom of R^{6″}. In some embodiments, the hydrocarbyl group of R^{6″} is an alkyl or alkenyl group, e.g., a C₁₀₋₃₀ alkyl or alkenyl group. Thus, in some embodiments, each R^{6″} is independently a non-cyclic alkyl group having at least 10 carbon atoms or a non-cyclic alkenyl group having at least 10 carbon atoms, e.g., a straight alkyl group having at least 10 carbon atoms or a straight alkenyl group having at least 10 carbon atoms. In some embodiments, each R^{6″} is independently a non-cyclic alkyl group having 10 to 30 carbon atoms (such as 10 to 28, 10 to 26, 10 to 24, 10 to 22, or 10 to 20 carbon atoms) or a non-cyclic alkenyl group having 10 to 30 carbon atoms (such as 10 to 28, 10 to 26, 10 to 24, 10 to 22, or 10 to 20 carbon atoms), e.g., a straight alkyl group having 10 to 30 carbon atoms (such as 10 to 28, 10 to 26, 10 to 24, 10 to 22, or 10 to 20 carbon atoms) or a straight alkenyl group having 10 to 30 carbon atoms (such as 10 to 28, 10 to 26, 10 to 24, 10 to 22, or 10 to 20 carbon atoms). In some embodiments, each R^{6″} is independently a non-cyclic alkyl group having 11 to 19 carbon atoms (such as 11, 13, 15, 17, or 17 carbon atoms), e.g., a straight alkyl group having 11 to 19 carbon atoms (such as 11, 13, 15, 17, or 17 carbon atoms). In some embodiments, each R^{6″} is independently a non-cyclic alkyl group having 10 to 30 carbon atoms (such as 10 to 28, 10 to 26, 10 to 24, 10 to 22, or 10 to 20 carbon atoms) or a non-cyclic alkenyl group having 10 to 30 carbon atoms (such as 10 to 28, 10 to 26, 10 to 24, 10 to 22, or 10 to 20 carbon atoms), e.g., a straight alkyl group having 10 to 30 carbon atoms (such as 10 to 28, 10 to 26, 10 to 24, to 22, or 10 to 20 carbon atoms) or a straight alkenyl group having 10 to 30 carbon atoms (such as 10 to 28, 10 to 26, 10 to 24, 10 to 22, or 10 to 20 carbon atoms), and each R^{6″} is attached to L^{1″} via an internal carbon atom of R^{6″}. In some embodiments, each R^{6″} is independently a non-cyclic alkyl group having 11 to 19 carbon atoms (such as 11, 13, 15, 17, or 17 carbon atoms), e.g., a straight alkyl group having 11 to 19 carbon atoms (such as 11, 13, 15, 17, or 17 carbon atoms), and each R^{6″} is attached to L^{1″} via an internal carbon atom of R^{6″}. The expression “internal carbon atom” means that the carbon atom of R^{6″} by which R^{6″} is attached to L^{1″} is directly bonded to at least 2 other carbon atoms of R^{6″}. For example, for the following C₁₁ alkyl group, each carbon atom at any one of positions 2, 3, 4, 5, and 7 qualifies as “internal carbon atom” according to the present disclosure, whereas the carbon atoms at positions 1, 6, 8, 9, 10, and 11 do not.

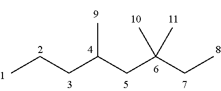

Consequently, R^{6″} being a C₁₁ alkyl group attached to L^{1″} via an internal carbon of R^{6″} includes the following groups:

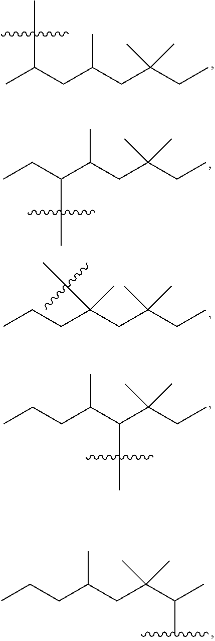

wherein [symbol] represents the bond by which R^{6″} is bound to L^{1″}. Furthermore, for a straight alkyl group, e.g., a straight C₁₁ alkyl group, each carbon atom except for the first and last carbon atoms of the straight alkyl group (i.e., except the carbon atoms at positions 1 and 11 of the straight C₁₁ alkyl group) qualifies as “internal carbon atom”. Thus, in some embodiments, R^{6″} being a straight alkyl group having p carbon atoms and being attached to L^{1″} via an internal carbon atom of R^{6″} means that R^{6″} is attached to L^{1″} via a carbon atom of R^{6″} at any one of positions 2 to (p−1) (thereby excluding the terminal C atoms at positions 1 and p). In some embodiments, where R^{6″} is a straight alkyl group having p′ carbon atoms (wherein p′ is an even number) and being attached to L^{1″} via an internal carbon atom of R^{6″}, R^{6″} is attached to L^{1″} via a carbon at any one of positions (p′/2−1), (p′/2), and (p′/2+1) of R^{6″} (e.g., if p′ is 10, R^{6″} is attached to L^{1″} via a carbon atom at any one of positions 4, 5, and 6 of R^{6″}). In some embodiments, where R^{6″} is a straight alkyl group having p″ carbon atoms (wherein p″ is an uneven number) and being attached to L^{1″} via an internal carbon atom of R^{6″}, R^{6″} is attached to L^{1″} via a carbon atom at any one of positions (p″−1)/2 and (p″+1)/2 of R^{6″} (e.g., if p″ is 11, R^{6″} is attached to L^{1″} via a carbon at any one of positions 5 and 6 of R^{6″}). Generally, it is to be understood that if both R^{1″} and R^{2″} are -G^{1″}-L^{1″}-R^{6″} and each R^{6″} is attached to L^{1″} via an internal carbon atom of R^{6″}, R^{6″} of R^{1″} is attached to L^{1″} of R^{1″} (and not to L^{1″} of R^{2″}) via an internal carbon atom of R^{6″} of R^{1″} and R^{6″} of R^{2″} is attached to L^{1″} of R^{2″} (and not to L^{1″} of R^{1″}) via an internal carbon atom of R^{6″} of R^{2″}. In some embodiments, each R^{6″} is independently selected from the group consisting of:

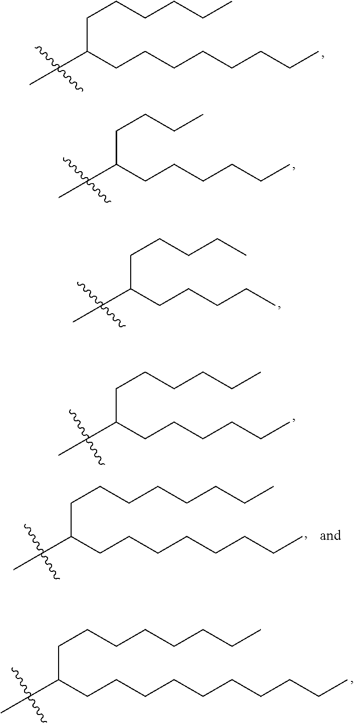

wherein [symbol] represents the bond by which R^{6″} is bound to L^{1″}. In some embodiments, where R^{1″} and R^{2′} are both independently -G^{1″}-L^{1″}-R^{6″}, R^{6″} for R^{1″} is different from R^{6″} for R^{2″}. In some of these embodiments, for example, R^{6″} for R^{1″} may be a non-cyclic, preferably straight, hydrocarbyl group having at least 10 carbon atoms (e.g., R^{6″} for R^{1″} is

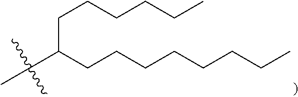 [SMILES: *C(CCCCCC)CCCCCCCC]

and R^{6″} for R^{2″} may be a different non-cyclic, preferably straight, hydrocarbyl group having at least 10 carbon atoms (e.g., R^{6″} for R^{2″} is

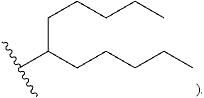 [SMILES: *C(CCCCC)CCCCC]

In some embodiments, where R^{1″} and R^{2″} are both independently -G^{1″}-L^{1″}-R^{6″}, R^{6″} for R^{1″} is identical to R^{6″} for R^{2″}. In some of these embodiments, for example, each R^{6″} is the same non-cyclic, preferably straight, hydrocarbyl group having at least 10 carbon atoms (e.g., each R^{6″} is

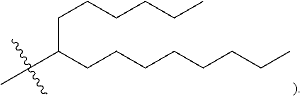 [SMILES: *C(CCCCCC)CCCCCCCC]

In some of the foregoing embodiments of Formula (XI), R^{5″} is a non-cyclic hydrocarbyl group having at least 10 carbon atoms, e.g., a straight hydrocarbyl group having at least carbon atoms. In some embodiments, R^{5″} is a non-cyclic hydrocarbyl group having at least 12 carbon atoms, such as at least 14, at least 16, or at least 18 carbon atoms, e.g., a straight hydrocarbyl group having at least 12, at least 14, at least 16, or at least 18 carbon atoms. In some embodiments, R^{5″} has at most 30 carbon atoms, such as at most 28, at most 26, at most 24, at most 22, or at most 20 carbon atoms. In some embodiments, R^{5″} is a non-cyclic hydrocarbyl group, e.g., a straight hydrocarbyl group, wherein each hydrocarbyl group has 10 to 30 carbon atoms (such as 10 to 28, 10 to 26, to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments, the hydrocarbyl group of R^{5″} is an alkyl or alkenyl group, e.g., a C₁₀₋₃₀ alkyl or alkenyl group. Thus, in some embodiments, R^{5″} is a non-cyclic alkyl group having at least 10 carbon atoms (such as at least 12, at least 14, at least 16, or at least 18 carbon atoms) or a non-cyclic alkenyl group having at least 10 carbon atoms (such as at least 12, at least 14, at least 16, or at least 18 carbon atoms), e.g., a straight alkyl group having at least 10 carbon atoms (such as at least 12, at least 14, at least 16, or at least 18 carbon atoms) or a straight alkenyl group having at least 10 carbon atoms (such as at least 12, at least 14, at least 16, or at least 18 carbon atoms). In some embodiments, R^{5″} is a non-cyclic alkyl group or a non-cyclic alkenyl group, e.g., a straight alkyl group or a straight alkenyl group, wherein each of the alkyl and alkenyl groups has independently to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments, the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments, R^{5″} is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments, R^{5″} is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments, R^{5″} has the following structure:

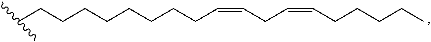

wherein [symbol] represents the bond by which R^{5″} is bound to the remainder of the compound.

In some of the foregoing embodiments of Formula (XI), L^{2″} is selected from the group consisting of —O(C═O)—, —(C═O)O—, —C(═O)—, —S—S—, —C(═O)S—, —SC(═O)—, —NR^{a″}C(═O)—, —C(═O)NR^{a″}—, —NR^{a″}C(═O)NR^{a″}, —OC(═O)NR^{a″}— and —NR^{a″}C(═O)O—. In some embodiments, L^{2″} is selected from the group consisting of —O(C═O)—, —(C═O)O—, —C(═O)—, —C(═O)S—, —SC(═O)—, —NR^{a″}C(═O)—, and —C(═O)NR^{a″}—. In some embodiments, R^{a″} of L^{2″} is H or C₁₋₁₂ alkyl. In some embodiments, R^{a″} of L^{2″} is H or C₁₋₆ alkyl, e.g., H or C₁₋₃ alkyl. In some embodiments, R^{a″} of L^{2″} is H, methyl, or ethyl. In some embodiments, L^{2″} is selected from the group consisting of —O(C═O)—, —(C═O)O—, —C(═O)S—, and —SC(═O). In some embodiments, L^{2″} is —O(C═O)— or —(C═O)O—.

In some of the foregoing embodiments of Formula (XI), G^{2″} is unsubstituted C₁₋₁₂ alkylene or unsubstituted C₂₋₁₂ alkenylene, e.g., unsubstituted, straight C₁₋₁₂ alkylene or unsubstituted, straight C₂₋₁₂ alkenylene. In some embodiments, G^{2″} is unsubstituted C₂₋₁₀ alkylene or unsubstituted C₂₋₁₀ alkenylene, e.g., unsubstituted, straight C₂₋₁₀ alkylene or unsubstituted, straight C₂₋₁₀ alkenylene. In some embodiments, G^{2″} is unsubstituted C₂₋₆ alkylene or unsubstituted C₂₋₆ alkenylene, e.g., unsubstituted, straight C₂₋₆ alkylene or unsubstituted, straight C₂₋₆ alkenylene. In some embodiments, G^{2″} is unsubstituted C₂₋₄ alkylene or unsubstituted C₂₋₄ alkenylene, e.g., unsubstituted, straight C₂₋₄ alkylene or unsubstituted, straight C₂₋₄ alkenylene. In some embodiments, G^{2″} is ethylene or trimethylene.

In some of the foregoing embodiments of Formula (XI), each of R^{3″} and R^{4″} is independently C₁₋₆ alkyl or C₂₋₆ alkenyl. In some embodiments, each of R^{3″} and R^{4″} is independently C₁₋₄ alkyl or C₂₋₄ alkenyl. In some embodiments, each of R^{3″} and R^{4″} is independently C₁₋₃ alkyl. In some embodiments, each of R^{3″} and R^{4″} is independently methyl or ethyl. In some embodiments, each of R^{3″} and R^{4″} is methyl.

In some of the foregoing embodiments of Formula (XI), m″ is 0, 1, 2 or 3. In some embodiments, m″ is 0 or 2. In some embodiments, m″ is 0. In some embodiments, m″ is 2.

In some of the foregoing embodiments of Formula (XI), the cationically ionizable lipid has the structure of Formula (XIIa) or (XIIb):

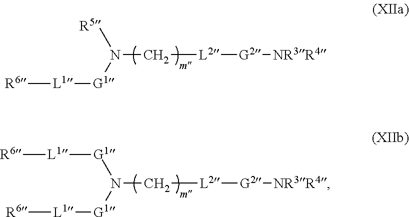

wherein
each of R^{3″} and R^{4″} is independently C₁-C₆ alkyl or C₂₋₆ alkenyl;
R^{5″} is a straight hydrocarbyl group having at least 14 carbon atoms (such as at least 16 carbon atoms), wherein the hydrocarbyl group preferably has at least 2 carbon-carbon double bonds;
each R^{6″} is independently a straight hydrocarbyl group (e.g., a straight alkyl group) having at least 10 carbon atoms and/or each R^{6″} is attached to L^{1″} via an internal carbon atom of R^{6″}, preferably each R^{6″} is independently a straight hydrocarbyl group (e.g., a straight alkyl group) having at least 10 carbon atoms and each R^{6″} is attached to L^{1″} via an internal carbon atom of R^{6″};
each G^{1″} is independently unsubstituted, straight C₄₋₁₂ alkylene or C₄₋₁₂ alkenylene, e.g., unsubstituted, straight C₆₋₁₂ alkylene or C₆₋₁₂ alkenylene, such as unsubstituted, straight C₁₂ alkylene or unsubstituted, straight C₁₂ alkenylene;
G^{2″} is unsubstituted C₂-C₁₀ alkylene or C₂₋₁₀ alkenylene, preferably unsubstituted C₂-C₆ alkylene or C₂₋₆ alkenylene;
each of L^{1″} and L^{2″} is independently —O(C═O)— or —(C═O)O—; and
m″ is 0, 1, 2 or 3, preferably 0 or 2.

In some of the foregoing embodiments of Formula (XIIa), R^{5″} has at most 30 carbon atoms, such as at most 28, at most 26, at most 24, at most 22, or at most 20 carbon atoms. In some embodiments of formulas (XIIa), R^{5″} is a straight hydrocarbyl group having 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIa), R^{5″} is a straight alkyl or alkenyl group having 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIa), the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments of formula (XIIa), R^{5″} is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments of formula (XIIa), R^{5″} is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments of formula (XIIa), R^{5″} is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has 2 or 3 carbon-carbon double bonds, wherein at least 1 carbon-carbon double bond, such as 1, 2, or 3 carbon-carbon double bonds, is in cis configuration. In some embodiments of formula (XIIa), R^{5″} has the following structure:

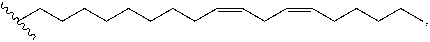

wherein [symbol] represents the bond by which R^{5″} is bound to the remainder of the compound. In some embodiments of formula (XIIa), R^{6″} has at most 30 carbon atoms, such as at most 28, at most 26, at most 24, at most 22, or at most 20 carbon atoms. In some embodiments of formula (XIIa), R^{6″} is a non-cyclic hydrocarbyl group (e.g., a non-cyclic alkyl group) having 10 to 30 carbon atoms (such as 10 to 28, 10 to 26, 10 to 24, 10 to 22, or 10 to 20 carbon atoms), e.g., a straight hydrocarbyl group (e.g., a straight alkyl group) having 10 to 30 carbon atoms (such as 10 to 28, 10 to 26, 10 to 24, 10 to 22, or 10 to 20 carbon atoms). In some embodiments of formula (XIIa), R^{6″} is a straight hydrocarbyl group (e.g., a straight alkyl group) having at least 10 carbon atoms and R^{6″} is attached to L^{1″} via an internal carbon atom of R^{6″}. In some embodiments of formula (XIIa), R^{6″} is a non-cyclic hydrocarbyl group (e.g., a non-cyclic alkyl group) having 10 to 30 carbon atoms (such as 10 to 28, 10 to 26, to 24, 10 to 22, or 10 to 20 carbon atoms), e.g., a straight hydrocarbyl group (e.g., a straight alkyl group) having 10 to 30 carbon atoms (such as 10 to 28, 10 to 26, 10 to 24, to 22, or 10 to 20 carbon atoms), and R^{6″} is attached to L^{1″} via an internal carbon atom of R^{6″}. In some embodiments of formula (XIIa), G^{1″} is independently unsubstituted, straight C₄₋₁₂ alkylene or C₄₋₁₂ alkenylene, e.g., unsubstituted, straight C₆₋₁₂ alkylene or C₆₋₁₂ alkenylene. In some embodiments of formula (XIIa), R^{5″} is a straight hydrocarbyl group, e.g., a straight alkenyl group, having at least 14 carbon atoms (such as 14 to 30 carbon atoms) and 2 or 3 carbon-carbon double bonds; R^{6″} is a straight hydrocarbyl group (e.g., a straight alkyl group) having at least 10 carbon atoms (e.g., having 10 to 30 carbon atoms) and R^{6″} is attached to L^{1″} via an internal carbon atom of R^{6″}; and G^{1″} is independently unsubstituted, straight C₄₋₁₂ alkylene or C₄₋₁₂ alkenylene, e.g., unsubstituted, straight C₆₋₁₂ alkylene or C₆₋₁₂ alkenylene.

In some of the foregoing embodiments of Formula (XIIb), each R^{6″} has independently at most 30 carbon atoms, such as at most 28, at most 26, at most 24, at most 22, or at most 20 carbon atoms. In some embodiments of formula (XIIb), each R^{6″} is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms). In some embodiments of formula (XIIb), each R^{6″} is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and each R^{6″} is attached to L^{1″} via an internal carbon atom of R^{6″}. In some embodiments of formula (XIIb), each R^{6″} is independently selected from the group consisting of:

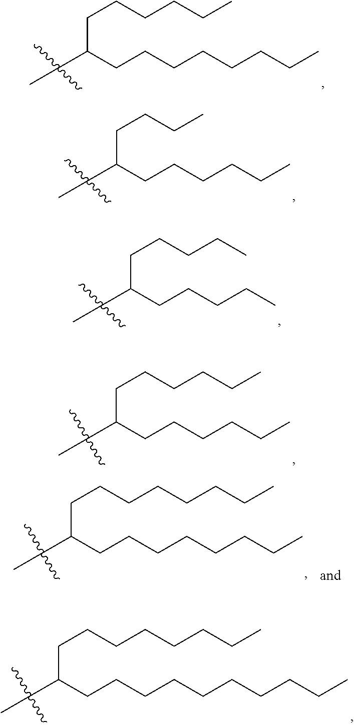

wherein [symbol] represents the bond by which R^{6″} is bound to L^{1″}. In some embodiments of formula (XIIb), each G^{1″} is independently unsubstituted, straight C₆₋₁₂ alkylene or C₆₋₁₂ alkenylene. In some embodiments of formula (XIIb), each G^{1″} is independently unsubstituted, straight C₈₋₁₂ alkylene or C₈₋₁₂ alkenylene. In some embodiments of formula (XIIb), each R^{6″} is independently a straight hydrocarbyl group (e.g., a straight alkyl group) having at least carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and is attached to L^{1″} via an internal carbon atom of R^{6″}; and each G^{1″} is independently unsubstituted, straight C₈₋₁₂ alkylene or C₈₋₁₂ alkenylene.

In some of the foregoing embodiments of Formula (XI), the cationically ionizable lipid has the structure of Formula (XIIIa) or (XIIIb):

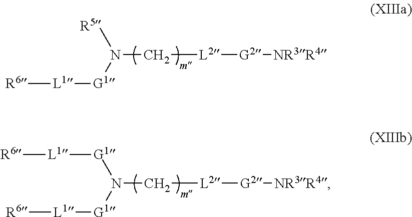

wherein
each of R^{3″} and R^{4″} is independently C₁₋₄ alkyl or C₂₋₄ alkenyl, more preferably C₁₋₃ alkyl, such as methyl or ethyl;
R^{5″} is a straight alkyl or alkenyl group having at least 16 carbon atoms, wherein the alkenyl group preferably has at least 2 carbon-carbon double bonds;
each R^{6″} is independently a straight hydrocarbyl group having at least 10 carbon atoms, wherein R^{6″} is attached to L^{1″} via an internal carbon atom of R^{6″};
each G^{1″} is independently unsubstituted, straight C₆₋₁₂ alkylene or unsubstituted, straight C₆₋₁₂ alkenylene, e.g., unsubstituted, straight C₈₋₁₂ alkylene or unsubstituted, straight C₈₋₁₂ alkenylene, such as unsubstituted, straight C₈₋₁₀ alkylene or unsubstituted, straight C₈ alkenylene, such as unsubstituted, straight C₈ alkylene;
G^{2″} is unsubstituted C₂₋₆ alkylene or C₂₋₆ alkenylene, preferably unsubstituted C₂₋₄ alkylene or C₂₋₄ alkenylene, such as ethylene or trimethylene;
each of L^{1″} and L^{2″} is independently —O(C═O)— or —(C═O)O—; and
M″ is 0, 1, 2 or 3, preferably 0 or 2.

In some of the foregoing embodiments of Formula (XIIIa), R^{5″} has at most 30 carbon atoms, such as at most 28, at most 26, at most 24, at most 22, or at most 20 carbon atoms. In some embodiments of formulas (XIIIa), R^{5″} is a straight alkyl or alkenyl group having 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIIa), the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments of formula (XIIIa), R^{5″} is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments of formula (XIIIa), R^{5″} is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments of formula (XIIIa), R^{5″} is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has 2 or 3 carbon-carbon double bonds, wherein at least 1 carbon-carbon double bond, such as 1, 2, or 3 carbon-carbon double bonds, is in cis configuration. In some embodiments of formula (XIIIa), R^{5″} has the following structure:

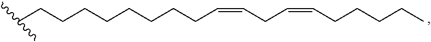 [SMILES: *CCCCCCCC/C=C\C/C=C\CCCCC]

[symbol]

represents the bond by which R^{5″} is bound to the remainder of the compound. In some embodiments of formula (XIIIa), R^{6″} has at most 30 carbon atoms, such as at most 28, at most 26, at most 24, at most 22, or at most 20 carbon atoms. In some embodiments of formula (XIIIa), R^{6″} is a straight hydrocarbyl group (e.g., a straight alkyl group) having 10 to 30 carbon atoms (such as 10 to 28, 10 to 26, 10 to 24, 10 to 22, or to 20 carbon atoms) and R^{6″} is attached to L^{1″} via an internal carbon atom of R^{6″}. In some embodiments of formula (XIIIa), R^{6″} is a straight alkyl group having 10 to 30 carbon atoms (such as 10 to 28, 10 to 26, 10 to 24, 10 to 22, or 10 to 20 carbon atoms) and R^{6″} is attached to L^{1″} via an internal carbon atom of R^{6″}. In some embodiments of formula (XIIIa), G^{1″} is independently unsubstituted, straight C₄₋₁₂ alkylene or C₄₋₁₂ alkenylene, e.g., unsubstituted, straight C₆₋₁₂ alkylene or C₆₋₁₂ alkenylene. In some embodiments of formula (XIIIa), R^{5″} is a straight hydrocarbyl group, e.g., a straight alkenyl group, having at least 16 carbon atoms (such as 16 to 30 carbon atoms) and 2 or 3 carbon-carbon double bonds; R^{6″} is a straight hydrocarbyl group (e.g., a straight alkyl group) having at least 10 carbon atoms (e.g., having 10 to 30 carbon atoms) and R^{6″} is attached to L^{1″} via an internal carbon atom of R^{6″}; and G^{1″} is independently unsubstituted, straight C₄₋₁₂ alkylene or C₄₋₁₂ alkenylene, e.g., unsubstituted, straight C₆₋₁₂ alkylene or C₆₋₁₂ alkenylene.

In some of the foregoing embodiments of Formula (XIIIb), each R^{6″} has independently at most 30 carbon atoms, such as at most 28, at most 26, at most 24, at most 22, or at most 20 carbon atoms. In some embodiments of formula (XIIIb), each R^{6″} is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and each R^{6″} is attached to L^{1″} via an internal carbon atom of R^{6″}. In some embodiments of formula (XIIIb), each R^{6″} is attached to L^{1″} via an internal carbon atom of R^{6″} and is independently selected from the group consisting of:

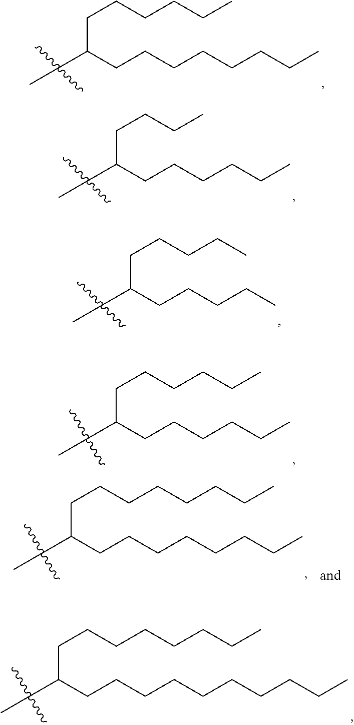

wherein [symbol] represents the bond by which R^{6″} is bound to L^{1″}. In some embodiments of formula (XIIIb), each G^{1″} is independently unsubstituted, straight C₁₂ alkylene or C₁₂ alkenylene, e.g., unsubstituted, straight C₈₋₁₀ alkylene or C₈₋₁₀ alkenylene. In some embodiments of formula (XIIIb), each R^{6″} is independently a straight hydrocarbyl group (e.g., a straight alkyl group) having at least 10 carbon atoms (such as 10 to 28, 10 to 26, to 24, 10 to 22, or 10 to 20 carbon atoms, or 11 to 19 carbon atoms, such as 11, 13, 15, 17, or 17 carbon atoms) and is attached to L^{1″} via an internal carbon atom of R^{6″}; and each G^{1″} is independently unsubstituted, straight C₁₂ alkylene or C₁₂ alkenylene, e.g., unsubstituted, straight C₈₋₁₀ alkylene or C₈₋₁₀ alkenylene.

In some of the foregoing embodiments of Formula (XI), the cationically ionizable lipid has one of the following formulas (XIV-1), (XIV-2), and (XIV-3):

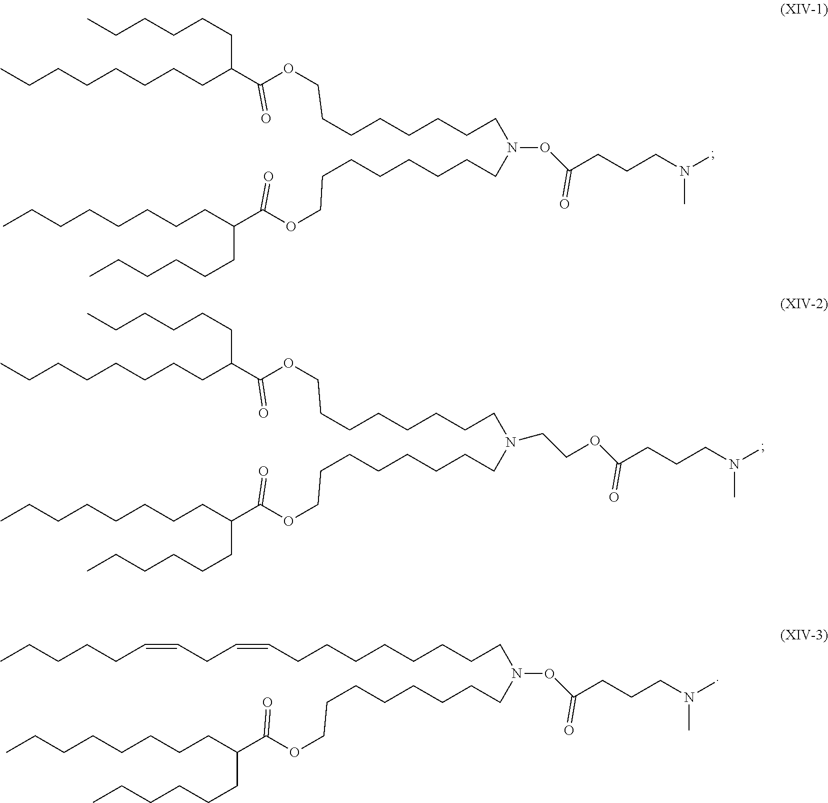

In some embodiments, the cationically ionizable lipid is (6Z,16Z)-12-((Z)-dec-4-en-1-yl)docosa-6,16-dien-11-yl 5-(dimethylamino)pentanoate (3D-P-DMA). The structure of 3D-P-DMA may be represented as follows:

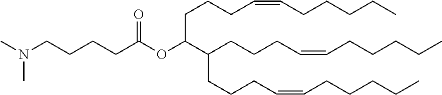 [SMILES: CCCCC/C=C\CCCC(CCC/C=C\CCCCC)C(CCC/C=C\CCCCC)OC(=O)CCCCN(C)C]

In various different embodiments, the cationically ionizable lipid is selected from the group consisting of N,N-dimethyl-2,3-dioleyloxypropylamine (DODMA), 1,2-dioleoyl-3-dimethylammonium-propane (DODAP), heptatriaconta-6,9,28,31-tetraen-19-yl-4-(dimethylamino)butanoate (DLin-MC3-DMA), and 4-((di((9Z,12Z)-octadeca-9,12-dien-1-yl)amino)oxy)-N,N-dimethyl-4-oxobutan-1-amine (DPL-14).

Further examples of cationically ionizable lipids include, but are not limited to, 3-(N-(N′,N′-dimethylaminoethane)-carbamoyl)cholesterol (DC-Chol), 1,2-dioleoyl-3-dimethylammonium-propane (DODAP); 1,2-diacyloxy-3-dimethylammonium propanes; 1,2-dialkyloxy-3-dimethylammonium propanes, 1,2-distearyloxy-N,N-dimethyl-3-aminopropane (DSDMA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), 2-({8-[(3p)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), di((Z)-non-2-en-1-yl)-9-((4-(dimethylaminobutanoyl)oxy)heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-aminol-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C₁₋₂-200).

In certain embodiments, the cationically ionizable lipid is or comprises X-3.

In certain embodiments, the cationically ionizable lipid is or comprises X-45.

In some embodiments, the cationic lipid for use herein is or comprises DPL-14. As used herein, “DPL-14” is a lipid comprising the following general formula:

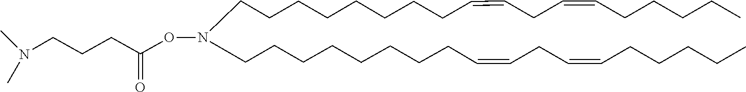

In some embodiments, the cationic lipid for use herein is or comprises EA-2. As used herein, “EA-2” is a lipid comprising the following general formula:

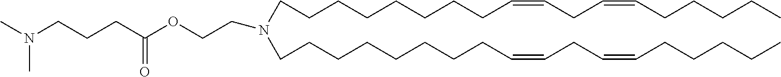

It is to be understood that any reference to a cationic or cationically ionizable lipid disclosed herein also includes the salts (in particular pharmaceutically acceptable salts), tautomers, stereoisomers, solvates (e.g., hydrates), and isotopically labeled forms thereof.

In some embodiments, the cationic/cationically ionizable lipid may comprise from about mol % to about 100 mol %, about 20 mol % to about 100 mol %, about 30 mol % to about 100 mol %, about 40 mol % to about 100 mol %, or about 50 mol % to about 100 mol % of the total lipid present in the composition/particle. In some embodiments, the cationic/cationically ionizable lipid comprises from about 40 mol % to about 75 mol %, preferably from about 40 mol % to about 70 mol %, more preferably from about 45 mol % to about 65 mol %, of the total lipid present in the composition/particles.

In some embodiments, wherein the nucleic acid compositions (in particular the DNA or RNA compositions) described herein comprise a cationic or cationically ionizable lipid and one or more additional lipids, the cationic or cationically ionizable lipid comprises from about 10 mol % to about 80 mol %, from about 20 mol % to about 75 mol %, from about 20 mol % to about 70 mol %, from about 20 mol % to about 60 mol %, from about mol % to about 55 mol %, from about 30 mol % to about 50 mol %, from about 35 mol % to about 45 mol %, or from about 40 mol % to about 55 mol % of the total lipid present in the composition.

In some embodiments of the nucleic acid (such as DNA or RNA) compositions (especially the mRNA compositions) described herein, where at least a portion of (i) the nucleic acid and (ii) the cationic or cationically ionizable lipid form particles (e.g., LNPs), the cationic or cationically ionizable lipid may comprise from about 10 mol % to about 80 mol %, from about 20 mol % to about 75 mol %, from about 20 mol % to about 70 mol %, from about 20 mol % to about 60 mol %, from about 25 mol % to about 55 mol %, from about 30 mol % to about 50 mol %, from about 35 mol % to about 45 mol %, or from about 40 mol % to about 55 mol % of the total lipid present in the particles.

### Helper Lipids

As described herein, LNPs of the present disclosure comprise a helper lipid. In some embodiments, a helper lipid is a phospholipid. In some embodiments, a helper lipid is or comprises 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC), phophatidyl ethanol amines such as 1,2-dioleoyl-sn-glycero-3-phosphoethanolamine (DOPE), sphingomyelins (SM), 1,2-diacylglyceryl-3-O-4′-(N,N,N-trimethyl)-homoserine (DGTS), ceramides, cholesterol, steroids, such as sterols and their derivatives.

In some embodiments, a helper lipid is or comprises phosphatidylcholines, phosphatidylethanolamines, phosphatidylglycerols, phosphatidic acids, phosphatidylserines or sphingomyelin. In some embodiments, a helper lipid is or comprises diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, including, for example diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM). In some embodiments, a helper lipid is selected from the group consisting of DSPC, DOPC, DMPC, DPPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DSPC.

Helper lipids may be synthetic or naturally derived. Other helper lipids suitable for use in a lipid nanoparticle are described in WO2021/026358, WO 2017/075531, and WO 2018/081480, the entire contents of each of which are incorporated herein by reference in their entirety.

In some embodiments, a lipid nanoparticle comprises about 5 to about 15 mol % of a phospholipid. In some embodiments, a lipid nanoparticle comprises about 8 to about 12 mol % of a phospholipid. In some embodiments, a lipid nanoparticle comprises about 10 mol % of a phospholipid. In some embodiments, a lipid nanoparticle comprises about 5 to about 15 mol % of DSPC. In some embodiments, a lipid nanoparticle comprises about 8 to about 12 mol % of DSPC. In some embodiments, a lipid nanoparticle comprises about 10 mol % of DSPC.

### Polymer-Conjugated Lipids

As described herein, LNPs of the present disclosure comprise a polymer-conjugated lipid. In some embodiments, a polymer conjugated lipid is a lipid conjugated to polyethylene glycol (PEG-lipid). In some embodiments, a PEG lipid is selected from pegylated diacylglycerol (PEG-DAG) such as l-(monomethoxy-polyethyleneglycol)-2,3-dimyristoylglycerol (PEG-DMG) (e.g., 1,2-dimyristoyl-rac-glycero-3-methoxypolyethylene glycol-2000 (PEG2000-DMG)), a pegylated phosphatidylethanoloamine (PEG-PE), a PEG succinate diacylglycerol (PEG-S-DAG) such as 4-O-(2′,3′-di(tetradecanoyloxy)propyl-1-O-(w-methoxy(polyethoxy)ethyl)butanedioate (PEG-S-DMG), 1,2-distearoyl-sn-glycero-3-phosphoethanolamine-N-[amino(polyethylene glycol)-2000](DSPE-PEG2000 amine), a pegylated ceramide (PEG-cer), or a PEG dialkoxypropylcarbamate such as w-m ethoxy (polyethoxy)ethyl-N-(2,3-di(tetradecanoxy)propyl)carbamate, and 2,3-di(tetradecanoxy)propy 1-N-(w methoxy(polyethoxy)ethyl)carbamate.

In some embodiments, a PEG-lipid is PEG2000-DMG:

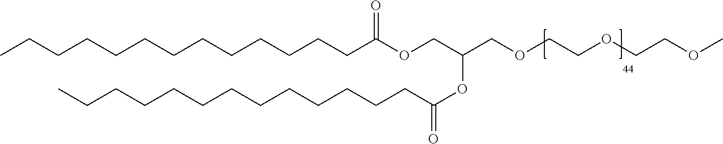

In some embodiments, a PEG-lipid is DMG-PEG.

In some embodiments, a PEG-lipid is provided in WO2021/026358, WO 2017/075531, or WO 2018/081480, each of which is incorporated by reference in its entirety.

In some embodiments, a PEG lipid is a compound of Formula II:

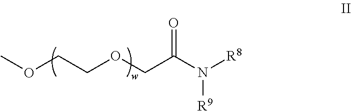

or a pharmaceutically acceptable salt thereof, wherein R⁸ and R⁹ are each independently C₁₀-C₃₀ aliphatic, optionally interrupted by one or more ester bonds, and w is an integer from 30 to 60.

As described herein, R⁸ and R⁹ are each independently C₁₀-C₃₀ aliphatic, optionally interrupted by one or more ester bonds. In some embodiments, R⁸ and R⁹ are each independently C₁₀-C₃₀ aliphatic. In some embodiments, R⁸ and R⁹ are each independently selected from C₁₋₂-C₁₆ aliphatic. In some embodiments, R⁸ and R⁹ are each independently selected from C₁₋₂-C₁₆ alkyl. In some embodiments, R⁸ and R⁹ are each independently selected form straight chain C₁₋₂-C₁₆ alkyl. In some embodiments, w is an integer from 40 to 50. In some embodiments, w is 45 to 47. In some embodiments, w is 45.

In some embodiments, a compound of Formula II is 2-[(polyethylene glycol)-2000]-N,N-ditetradecylacetamide (ALC-0159). In some embodiments, a compound of Formula II is:

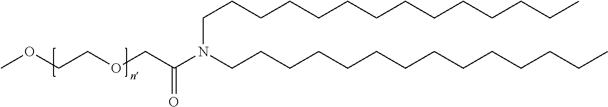

or a pharmaceutically acceptable salt thereof, where n′ is an integer from about 45 to about 50.

In some embodiments, the PEG-lipid has the following structure:

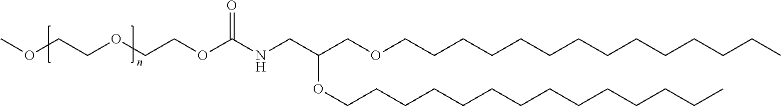

wherein n has a mean value ranging from 30 to 60, such as about 50. In one embodiment, the PEG-conjugated lipid (pegylated lipid) is PEG₂₀₀₀-C-DMA which preferably refers to 3-N-[(w-methoxy poly(ethylene glycol)2000)carbamoyl]-1,2-dimyristyloxy-propylamine (MPEG-(2 kDa)-C-DMA) or methoxy-polyethylene glycol-2,3-bis(tetradecyloxy)propylcarbamate (2000).

In some embodiments, a PEG-lipid is selected from PEG-DAG, PEG-PE, PEG-S-DAG, PEG2000-DMG, PEG-S-DMG, PEG-cer, a PEG dialkyoxypropylcarbamate (e.g., w-methoxy(polyethoxy)ethyl-N-(2,3-di(tetradecanoxy)propyl)carbamate or 2,3-di(tetradecanoxy)propyl-N-(o methoxy(polyethoxy)ethyl)carbamate), ALC-0159, and combinations thereof. In some embodiments, a PEG-lipid is ALC-0159 or PEG2000-DMG. In some embodiments, a PEG-lipid is ALC-0159. In some embodiments, a PEG-lipid is PEG2000-DMG. In some embodiments, a PEG-lipid is PEG-DAG. In some embodiments, a PEG-lipid is PEG-PE. In some embodiments, a PEG-lipid is PEG-S-DAG. In some embodiments, a PEG-lipid is PEG-cer. In some embodiments, a PEG-lipid is a PEG dialkyoxypropylcarbamate.

In some embodiments, a PEG group that is part of a PEG-lipid has, on average in a composition comprising one or more PEG-lipid molecules, a number average molecular weight (Mₙ) of about 2000 g/mol.

In some embodiments, a polymer-conjugated lipid is a polysarcosine-conjugated lipid, also referred to herein as sarcosinylated lipid or pSar-lipid. The term “sarcosinylated lipid” refers to a molecule comprising both a lipid portion and a polysarcosine (poly(N-methylglycine) portion.

In some embodiments, a polymer-conjugated lipid is a polyoxazoline (POX)-conjugated and/or polyoxazine (POZ)-conjugated lipid, also referred to herein as a conjugate of a POX and/or POZ polymer and one or more hydrophobic chains or as oxazolinylated and/or oxazinylated lipid or POX- and/or POZ-lipid. The term “oxazolinylated lipid” or “POX-lipid” refers to a molecule comprising both a lipid portion and a polyoxazoline portion. The term “oxazinylated lipid” or “POZ-lipid” refers to a molecule comprising both a lipid portion and a polyoxazine portion. The term “oxazolinylated/oxazinylated lipid” or “POX/POZ-lipid” or “POXZ-lipid” refers to a molecule comprising both a lipid portion and a portion of a copolymer of polyoxazoline and polyoxazine.

In some embodiments, an LNP described herein may comprise a sarcosinylated lipid. In some embodiments, the nucleic acid compositions (such as DNA or RNA compositions, especially mRNA compositions) described herein comprise a sarcosinylated lipid and are substantially free of a pegylated lipid (or do not contain a pegylated lipid).

In some embodiments, the nucleic acid compositions (such as DNA or RNA compositions, especially mRNA compositions) described herein comprise a cationic/cationically ionizable lipid as described herein and a sarcosinylated lipid (pSAR-conjugated lipid). In some embodiments, the nucleic acid compositions (such as DNA or RNA compositions, especially mRNA compositions) described herein may further comprise a neutral lipid (e.g., a phospholipid, cholesterol or a derivative thereof) or a combination of neutral lipids (e.g., a phospholipid, and cholesterol or a derivative thereof). In some embodiments, the nucleic acid compositions (such as DNA or RNA compositions, especially mRNA compositions) described herein comprise a cationic/cationically ionizable lipid as described herein, a sarcosinylated lipid, a neutral lipid (e.g., a phospholipid), and cholesterol or a derivative thereof. In some embodiments, the phospholipid is DSPC. In some embodiments, the cationic/cationically ionizable lipid is a cationically ionizable lipid of formula (X) (such as a cationically ionizable lipid of formula (X-3) or (X-45)). In some embodiments, the cationic/cationically ionizable lipid is a cationically ionizable lipid of formula (XI) (such as a cationically ionizable lipid of formula (XIV-1), (XIV-2), or (XIV-3)). In some embodiments, the cationic/cationically ionizable lipid is DPL14, EA-2, or 3D-P-DMA.

In some embodiments of the nucleic acid compositions (such as DNA or RNA compositions, especially mRNA compositions) described herein which comprise a sarcosinylated lipid, said compositions are substantially free of a pegylated lipid (or do not contain a pegylated lipid).

In some embodiments, the sarcosinylated lipid comprises between 2 and 200 sarcosine units, such as between 5 and 100 sarcosine units, between 10 and 50 sarcosine units, between 15 and 40 sarcosine units, e.g., about 23 sarcosine units.

In some embodiments, the sarcosinylated lipid comprises the structure of the following general formula (XVII):

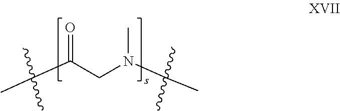

wherein s is the number of sarcosine units.

In some embodiments, the sarcosinylated lipid comprises the structure of the following general formula (XVIII):

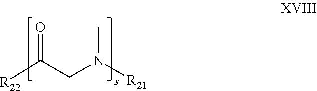

wherein one of R₂₁ and R₂₂ comprises a hydrophobic group and the other is H, a hydrophilic group or a functional group optionally comprising a targeting moiety; and x is the number of sarcosine units.

In some embodiments of formula (XVIII), R₂₁ is H, a hydrophilic group or a functional group optionally comprising a targeting moiety; and R₂₂ comprises one or two straight alkyl or alkenyl groups each having at least 12 carbon atoms, such as at least 14 carbon atoms. In some embodiments, each of the straight alkyl and alkenyl groups has at most carbon atoms, such as at most 28, at most 26, at most 24, at most 22, at most 20, or at most 18 carbon atoms. In some embodiments, R₂₂ comprises one or two straight alkyl or alkenyl groups each having 12 to 30 carbon atoms (such as 12 to 28 carbon atoms, 12 to 26 carbon atoms, 12 to 24 carbon atoms, 12 to 22 carbon atoms, 12 to 20 carbon atoms, or 12 to 18 carbon atoms).

In some embodiments, the sarcosinylated lipid has the structure of the following general formula (IXX):

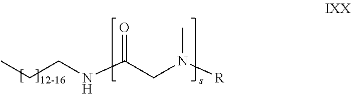

wherein R is H, a hydrophilic group or a functional group optionally comprising a targeting moiety; and s is the number of sarcosine units.

In some embodiments, the sarcosinylated lipid has the structure of the following formula (IXX-1):

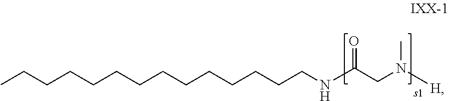

wherein si is 23. The sarcosinylated lipid of formula (IXX-1) is also referred to herein as “C14pSar23”.

In some embodiments, an LNP herein may comprise an oxazolinylated and/or/oxazinylated lipid. In some embodiments, the nucleic acid compositions (such as DNA or RNA compositions, especially mRNA compositions) described herein comprise an oxazolinylated and/or/oxazinylated lipid and are substantially free of a pegylated lipid (or do not contain a pegylated lipid).

In some embodiments, the nucleic acid compositions (such as DNA or RNA compositions, especially mRNA compositions) described herein comprise a cationic/cationically ionizable lipid as described herein and an oxazolinylated and/or oxazinylated lipid (POX and/or POZ-conjugated lipid). In some embodiments, the nucleic acid compositions (such as DNA or RNA compositions, especially mRNA compositions) described herein may further comprise a neutral lipid (e.g., a phospholipid, cholesterol or a derivative thereof) or a combination of neutral lipids (e.g., a phospholipid, and cholesterol or a derivative thereof). In some embodiments, the nucleic acid compositions (such as DNA or RNA compositions, especially mRNA compositions) described herein comprise a cationic/cationically ionizable lipid as described herein, an oxazolinylated and/or oxazinylated lipid, a neutral lipid (e.g., a phospholipid), and cholesterol or a derivative thereof. In some embodiments, the phospholipid is DSPC. In some embodiments, the cationic/cationically ionizable lipid is a cationically ionizable lipid of formula (X) (such as a cationically ionizable lipid of formula (X-3) or (X-45)). In some embodiments, the cationic/cationically ionizable lipid is a cationically ionizable lipid of formula (XI) (such as a cationically ionizable lipid of formula (XIV-1), (XIV-2), or (XIV-3)). In some embodiments, the cationic/cationically ionizable lipid is DPL14, EA-2, or 3D-P-DMA. In some embodiments of the nucleic acid compositions (such as DNA or RNA compositions, especially mRNA compositions) described herein which comprise an oxazolinylated and/or oxazinylated lipid, said compositions are substantially free of a pegylated lipid (or do not contain a pegylated lipid).

In some embodiments, in the oxazolinylated and/or oxazinylated lipid (i.e., the conjugate comprising (i) a POX and/or POZ polymer and (ii) one or more hydrophobic chains), components (i) and (ii) are linked to each other via a linker which comprises at least one functional moiety. In some embodiments, said linker comprises an alkylene moiety substituted with at least one monovalent functional moiety. In some embodiments, said linker comprises an alkylene group and a divalent functional moiety, wherein the divalent functional moiety links the alkylene group to the one or more hydrophobic chains, and the alkylene group is attached to the POX and/or POZ polymer. In some embodiments, said linker comprises an alkylene group and a divalent functional moiety, wherein the divalent functional moiety links the alkylene group to the one or more hydrophobic chains, the alkylene group is substituted with at least one monovalent functional moiety, and the alkylene group is attached to the POX and/or POZ polymer.

In some embodiments of the oxazolinylated and/or oxazinylated lipid, each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties.

In some embodiments of the oxazolinylated and/or oxazinylated lipid, each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties.

In some embodiments, the oxazolinylated and/or oxazinylated lipid comprises one of the following structures (in particular, if the one or more hydrophobic chains are attached to the N-end (i.e., the terminal N atom) of the POX and/or POZ polymer, as shown, for example in formula (XXI) herein):

(hydrophobic chain)₁₋₂-(alkylene moiety substituted with at least one monovalent functional moiety)-(POX and/or POZ polymer)

[(hydrophobic chain)-(divalent functional moiety)]₁₋₂-(alkylene moiety)-(POX and/or POZ polymer).

In some embodiments, the oxazolinylated and/or oxazinylated lipid has one of the following formulas (in particular, if the one or more hydrophobic chains are attached to the N-end (i.e., the terminal N atom) of the POX and/or POZ polymer, as shown, for example in formula (XXI) herein below):

(hydrophobic chain)₁₋₂-(alkylene moiety substituted with at least one monovalent functional moiety)-(POX and/or POZ polymer)-(end group)

[(hydrophobic chain)-(divalent functional moiety)]₁₋₂-(alkylene moiety)-(POX and/or POZ polymer)-(end group).

In some embodiments of the oxazolinylated and/or oxazinylated lipid, the alkylene moiety substituted with at least one monovalent functional moiety is substituted with one or more (such as 1 to the maximum number of hydrogen atoms bound to the alkylene moiety, e.g., 1, 2, 3, 4, 5, or 6, such as between 1 to 5, 1 to 4, or 1 to 3, or 1 or 2) independently selected monovalent functional moieties.

In some embodiments of the oxazolinylated and/or oxazinylated lipid, the alkylene moiety is C₁₋₆-alkylene, such as C₁₋₃-alkylene, e.g., methylene, ethylene, or trimethylene.

In some embodiments (in particular those, where the one or more hydrophobic chains are attached to the C-end (i.e., the terminal C atom) of the POX and/or POZ polymer, as shown, for example in formula (XXI′) herein), the linker comprises at least one difunctional moiety via which the one or more hydrophobic chains are attached to the POX and/or POZ polymer. In some embodiments, the linker may additionally comprise an alkylene moiety (such as a C₁₋₆ alkylene moiety, e.g., a C₁₋₃ alkylene moiety), a cycloalkylene moiety (preferably a C₃₋₈-cycloalkylene, such as C₃₋₆-cycloalkylene moiety), or a cycloalkenylene moiety (preferably a C₃₋₈-cycloalkenylene, such as C₃₋₆-cycloalkenylene moiety) each of which connects the difunctional moiety to the POX and/or POZ polymer (either directly to the end of the POX and/or POZ polymer or, preferably, via a further difunctional moiety). For example, one hydrophobic chain may be attached to the end of the POX and/or POZ polymer via one difunctional moiety (either directly or via an alkylene, cycloalkylene, or cycloalkenylene moiety or via an alkylene, cycloalkylene, or cycloalkenylene moiety which bears another difunctional moiety); two hydrophobic chains may be attached to the end of the POX and/or POZ polymer via two difunctional moieties (which in turn are preferably attached to an alkylene, cycloalkylene, or cycloalkenylene moiety or to an alkylene, cycloalkylene, or cycloalkenylene moiety bearing another difunctional moiety); or two hydrophobic chains may be attached to the end of the POX and/or POZ polymer via the same difunctional moiety (which is then a trifunctional moiety and which may be attached to the end of the POX and/or POZ polymer either directly or via an alkylene, cycloalkylene, or cycloalkenylene moiety or to an alkylene, cycloalkylene, or cycloalkenylene moiety bearing another difunctional moiety). In some embodiments, each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties.

In some embodiments of the oxazolinylated and/or oxazinylated lipid, the cycloalkylene moiety is C₃₋₈-cycloalkylene, such as C₃₋₆-cycloalkylene, e.g., cyclopropylene, cyclobutylene, cyclopentylene, cyclohexylene, wherein the cycloalkylene moiety is optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments of the oxazolinylated and/or oxazinylated lipid, the cycloalkenylene moiety is C₃₋₈-cycloalkenylene, such as C₃₋₆-cycloalkenylene, e.g., cyclopropenylene, cyclobutenylene, cyclopentenylene, cyclohexenylene, wherein the cycloalkenylene moiety is optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments of the oxazolinylated and/or oxazinylated lipid, the alkylene moiety is C₁₋₆-alkylene, such as C₁₋₃-alkylene, e.g., methylene, ethylene, or trimethylene, or C₂₋₃ alkylene.

In some embodiments (in particular those, where the one or more hydrophobic chains are attached to the C-end (i.e., the terminal C atom) of the POX and/or POZ polymer, as shown, for example in formula (XXI′) herein below), the oxazolinylated and/or oxazinylated lipid comprises one of the following structures (and may have the general formula (XXI′)):

(hydrophobic chain)-(divalent functional moiety)-(POX and/or POZ polymer)

[(hydrophobic chain)-(divalent functional moiety)]₁₋₂-(alkylene moiety)-(divalent functional moiety)-(POX and/or POZ polymer)

(hydrophobic chain)-(divalent functional moiety)-(cycloalkylene moiety)-(divalent functional moiety)-(POX and/or POZ polymer)

(hydrophobic chain)-(divalent functional moiety)-(cycloalkenylene moiety)-(divalent functional moiety)-(POX and/or POZ polymer)

(hydrophobic chain)-(divalent functional moiety)-(alkylene moiety)-(POX and/or POZ polymer)

[(hydrophobic chain)₂-(trivalent functional moiety)]-(alkylene moiety)-(divalent functional moiety)-(POX and/or POZ polymer)

In some embodiments (in particular those, where the one or more hydrophobic chains are attached to the C-end (i.e., the terminal C atom) of the POX and/or POZ polymer, as shown, for example in formula (XXI′) herein below), the oxazolinylated and/or oxazinylated lipid has one of the following formulas (and may fall within general formula (XXI′)):

(hydrophobic chain)-(divalent functional moiety)-(POX and/or POZ polymer)-(end group)

[(hydrophobic chain)-(divalent functional moiety)]₁₋₂-(alkylene moiety)-(divalent functional moiety)-(POX and/or POZ polymer)-(end group)

(hydrophobic chain)-(divalent functional moiety)-(cycloalkylene moiety)-(divalent functional moiety)-(POX and/or POZ polymer)-(end group)

(hydrophobic chain)-(divalent functional moiety)-(cycloalkenylene moiety)-(divalent functional moiety)-(POX and/or POZ polymer)-(end group)

(hydrophobic chain)-(divalent functional moiety)-(alkylene moiety)-(POX and/or POZ polymer)-(end group)

[(hydrophobic chain)₂-(trivalent functional moiety)]-(alkylene moiety)-(divalent functional moiety)-(POX and/or POZ polymer)-(end group)

The POX and/or POZ polymer may comprise a neutral end group (such as H, alkyl, alkoxy, ester, or amide end group) or a functionalized end group (e.g., hydroxy, thiol, cyano, azido, or amino end group). In the case of nucleic acid-lipid particles, the POX and/or POZ polymer is conjugated to, preferably covalently bound to one or more hydrophobic chains.

In certain embodiments of the oxazolinylated and/or oxazinylated lipid, the end groups of the POX and/or POZ polymer may be functionalized with one or more molecular moieties conferring certain properties, such as positive or negative charge, or a targeting agent that will direct the particle to a particular cell type, collection of cells, or tissue.

A variety of suitable targeting agents are known in the art. Non-limiting examples of targeting agents include a peptide, a protein, an enzyme, a nucleic acid, a fatty acid, a hormone, an antibody, a carbohydrate, mono-, oligo- or polysaccharides, a peptidoglycan, a glycopeptide, or the like. In some embodiments, targeting agents include targeting pairs, such as the following pairs: antigen-antibody specific for said antigen; avidin-streptavidin; folate-folate receptor; transferrin-transferrin receptor; aptamer-molecule for which the aptamer is specific (e.g., pegaptanib-VEGF receptor); arginine-glycine-aspartic acid (RGD) peptide-αᵥβ integrin; asparagine-glycine-arginine (NGR) peptide-aminopeptidase N; galactose-asialoglyco-protein receptor. For example, any of a number of different materials that bind to antigens on the surfaces of target cells can be employed. Antibodies to target cell surface antigens will generally exhibit the necessary specificity for the target. In addition to antibodies, suitable immunoreactive fragments can also be employed, such as the Fab, Fab′, F(ab′)₂ or scFv fragments or single-domain antibodies (e.g. camelids V_{H}H fragments). Many antibody fragments suitable for use in forming the targeting mechanism are already available in the art. Similarly, ligands for any receptors on the surface of the target cells can suitably be employed as targeting agent. These include any small molecule or biomolecule, natural or synthetic, which binds specifically to a cell surface receptor, protein or glycoprotein found at the surface of the desired target cell.

In certain embodiments of the oxazolinylated and/or oxazinylated lipid, the POX and/or POZ polymer comprises between 2 and 200, between 2 and 190, between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between and 200, between 10 and 190, between 10 and 180, between 10 and 170, between and 160, between 10 and 150, between 10 and 140, between 10 and 130, between and 120, between 10 and 110, between 10 and 100, between 10 and 90, between 10 and 80, or between 10 and 70 POX and/or POZ repeating units.

In some embodiments, the POX and/or POZ polymer in the oxazolinylated and/or oxazinylated lipid comprises the following general formula (XX):

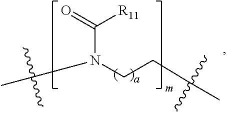

wherein a is an integer between 1 and 2; R₁₁ is alkyl, in particular C₁₋₃ alkyl, such as methyl, ethyl, iso-propyl, or n-propyl, and is independently selected for each repeating unit; and m refers to the number of POX and/or POZ repeating units.

In some embodiments of the oxazolinylated and/or oxazinylated lipid, the POX and/or POZ polymer is a polymer of POX and comprises repeating units of the following general formula (XXa):

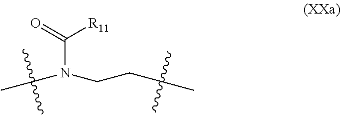

In some embodiments of the oxazolinylated and/or oxazinylated lipid, the POX and/or POZ polymer is a polymer of POZ and comprises repeating units of the following general formula (XXb):

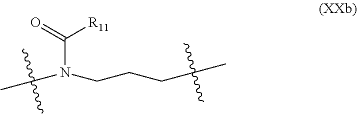

In any of the above embodiments of formulas (XX), (XXa), and (XXb), m (i.e., the number of repeating units of formula (XXa) or formula (XXb) in the polymer) preferably is between 2 and 190, such as between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70. In certain embodiments of any of the above embodiments of formulas (XX), (XXa), and (XXb), m is 2 to 180, such as 4 to 160, 6 to 140, 8 to 120 or 10 to 100, e.g., 20 to 80, 30 to 70, or to 50.

In some embodiments of the oxazolinylated and/or oxazinylated lipid, the POX and/or POZ polymer is a copolymer comprising repeating units of the following general formulas (XXa) and (XXb):

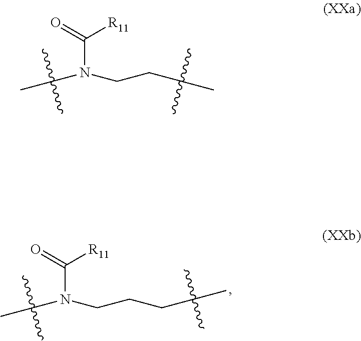

wherein the number of repeating units of formula (XXa) in the copolymer is 1 to 199; the number of repeating units of formula (XXb) in the copolymer is 1 to 199; and the sum of the number of repeating units of formula (XXa) and the number of repeating units of formula (XXb) in the copolymer is 2 to 200.

In some embodiments of the oxazolinylated and/or oxazinylated lipid, the number of repeating units of formula (XXa) in the copolymer is 1 to 179, such as 1 to 159, 1 to 139, 1 to 119 or 1 to 99; the number of repeating units of formula (XXb) in the copolymer is 1 to 179, such as 1 to 159, 1 to 139, 1 to 119 or 1 to 99; and the sum of the number of repeating units of formula (XXa) and the number of repeating units of formula (XXb) in the copolymer is 2 to 180, such as 4 to 160, 6 to 140, 8 to 120 or 10 to 100, e.g., 20 to 80, 30 to 70, or 40 to 50.

In some of the above embodiments of formulas (XX), (XXa), and (XXb), R₁₁ at each occurrence (i.e., in each repeating unit) may be the same alkyl group (e.g., R₁₁ may be methyl in each repeating unit). In some alternative embodiments of formulas (XX), (XXa), and (XXb), R₁₁ in at least one repeating unit differs from R₁₁ in another repeating unit (e.g., for at least one repeating unit R₁₁ is one specific alkyl (such as ethyl), and for at least one different repeating unit R₁₁ is a different specific alkyl (such as methyl)). For example, each R₁₁ may be selected from two different alkyl groups (such as methyl and ethyl) and not all R₁₁ are the same alkyl.

In any of the above embodiments of formulas (XX), (XXa), and (XXb), R₁₁ preferably is methyl or ethyl, more preferably methyl. Thus, in some embodiments of formulas (XX), (XXa), and (XXb), each R₁₁ is methyl or each R₁₁ is ethyl. In some alternative embodiments of formulas (XX), (XXa), and (XXb), R₁₁ is independently selected from methyl and ethyl for each repeating unit, wherein in at least one repeating unit R₁₁ is methyl, and in at least one repeating unit R₁₁ is ethyl.

In some embodiments, the oxazolinylated and/or oxazinylated lipid has the following general formula (XXI) or (XXI′):

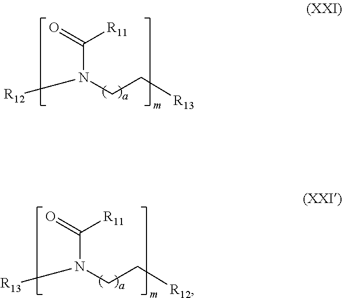

wherein:
a is an integer between 1 and 2;
R₁₁ is alkyl, in particular C₁₋₃ alkyl, such as methyl, ethyl, iso-propyl, or n-propyl, and is independently selected for each repeating unit;
m is 2 to 200;
R₁₂ is R₁₄ or -L₁₁(R₁₄)ₚ, wherein each R₁₄ is independently a hydrocarbyl group; L₁₁ is a linker; and p is 1 or 2; and
R₁₃ is selected from the group consisting of H, C₁₋₆ alkyl, C₂₋₆ alkynyl, —OR₂₀, —SR₂₀, halogen,
—CN, —N₃, —OC(O)R₂₁, —C(O)R₂₁, —NR₂₂R₂₃, —COOH, —C(O)NR₂₂R₂₃, —NR₂₂C(O)R₂₁, a sugar, an amino acid, a peptide, and a member of a targeting pair, wherein the C₁₋₆ alkyl group is optionally substituted with one or more substituents independently selected from the group consisting of —OH, SH, halogen, —CN, —N₃, C₂₋₆ alkynyl, —COOH, —NR₂₂R₂₃, —C(O)NR₂₂R₂₃, —NR₂₂C(O)R₂₁, a sugar, an amino acid, a peptide, and a member of a targeting pair; R₂₀ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR₂₂R₂₃, a sugar, an amino acid, a peptide, and a member of a targeting pair; R₂₁ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR₂₂R₂₃, a sugar, an amino acid, a peptide, and a member of a targeting pair; and each of R₂₂ and R₂₃ is independently selected from the group consisting of H, alkyl, alkenyl, alkynyl, cycloalkyl, aryl, heteroaryl, and heterocyclyl, or R₂₂ and R₂₃ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, a sugar, an amino acid, a peptide, and a member of a targeting pair. In formula (XXI) R₁₂ is attached to the N-end (i.e., the terminal N atom) of the POX and/or POZ polymer and R₁₃ is attached to the C-end (i.e., the terminal C atom) of the POX and/or POZ polymer, whereas in formula (XXI′) R₁₂ is attached to the C-end (i.e., the terminal C atom) of the POX and/or POZ polymer and R₁₃ is attached to the N-end (i.e., the terminal N atom) of the POX and/or POZ polymer.

In some embodiments (in particular of the oxazolinylated and/or oxazinylated lipid of formulas (XXI) and (XXI′)), the targeting pair is selected from the following pairs: antigen antibody specific for said antigen; avidin-streptavidin; folate-folate receptor; transferrin-transferrin receptor; aptamer-molecule for which the aptamer is specific; -arginine-glycine-aspartic acid (RGD) peptide-αᵥβ₃ integrin; asparagine-glycine-arginine (NGR) peptide-aminopeptidase N; galactose-asialoglyco-protein receptor. Thus, in some embodiments, a member of a targeting pair includes one of the following: an antigen, an antibody, avidin, streptavidin, folate, transferrin, an aptamer; an RGD peptide; an NGR peptide; and galactose.

In some embodiments of formula (XXI), a is 1, i.e., the oxazolinylated and/or oxazinylated lipid has the following general formula (XXIa) or (XXIa′):

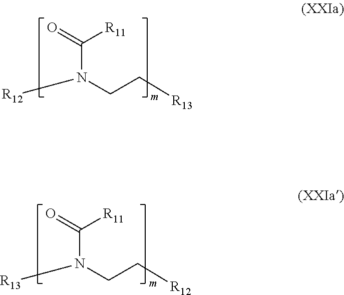

In some embodiments of formula (XXI), a is 2, i.e., the oxazolinylated and/or oxazinylated lipid has the following general formula (XXIb) or (XXIb′):

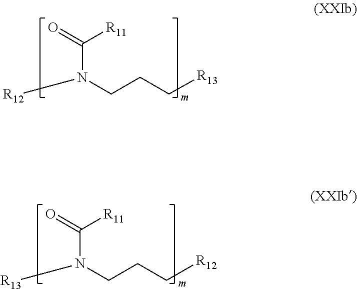

In any of the above embodiments of formulas (XXIa), (XXIa′), (XXIb), and (XXIb′), R₁₁, R₁₂, R₁₃, and m are as defined for formula (XXI)/(XXI′).

In some of the above embodiments of formulas (XXI), (XXI′), (XXIa), (XXIa′), (XXIb), and (XXIb′), R₁₁ at each occurrence (i.e., in each repeating unit) may be the same alkyl group (e.g., R₁₁ may be methyl in each repeating unit). In some alternative embodiments of formulas (XXI), (XXI′), (XXIa), (XXIa′), (XXIb), and (XXIb′), R₁₁ in at least one repeating unit differs from R₁₁ in another repeating unit (e.g., for at least one repeating unit R₁₁ is one specific alkyl (such as ethyl), and for at least one different repeating unit R₁₁ is a different specific alkyl (such as methyl)). For example, each R₁₁ may be selected from two different alkyl groups (such as methyl and ethyl) and not all R₁₁ are the same alkyl.

In any of the above embodiments of formulas (XXI), (XXI′), (XXIa), (XXIa′), (XXIb), and (XXIb′), R₁₁ preferably is methyl or ethyl, more preferably methyl. Thus, in some embodiments of formulas (XXI), (XXI′), (XXIa), (XXIa′), (XXIb), and (XXIb′), each R₁₁ is methyl or each R₁₁ is ethyl. In some alternative embodiments of formulas (XXI), (XXI′), (XXIa), (XXIa′), (XXIb), and (XXIb′), R₁₁ is independently selected from methyl and ethyl for each repeating unit, wherein in at least one repeating unit R₁₁ is methyl, and in at least one repeating unit R₁₁ is ethyl.

In any of the above embodiments of formulas (XXI), (XXI′), (XXIa), (XXIa′), (XXIb), and (XXIb′), m preferably is between 2 and 190, such as between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between and 190, between 10 and 180, between 10 and 170, between 10 and 160, between and 150, between 10 and 140, between 10 and 130, between 10 and 120, between and 110, between 10 and 100, between 10 and 90, between 10 and 80, or between and 70. In certain embodiments of formulas (XXI), (XXI′), (XXIa), (XXIa′), (XXIb), and (XXIb′), m is 2 to 180, such as 4 to 160, 6 to 140, 8 to 120 or 10 to 100, e.g., 20 to 80, to 70, or 40 to 50.

In some embodiments of formulas (XXI), (XXI′), (XXIIa), (XXIa′), (XXIb), and (XXIb′), L₁₁ comprises at least one functional moiety, such as an alkylene moiety substituted with at least one monovalent functional moiety and/or linked, at the end by which the alkylene group is attached to R₁₄, to a divalent functional moiety, wherein preferably each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide; and/or each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide.

In some embodiments of formulas (XXI), (XXI′), (XXIa), (XXIa′), (XXIb), and (XXIb′), L₁₁ comprises an alkylene moiety substituted with at least one monovalent functional moiety as specified above. Thus, in some embodiments, the oxazolinylated and/or oxazinylated lipid may comprise the following structure (in particular, if the one or more hydrophobic chains are attached to the N-end (i.e., the terminal N atom) of the POX and/or POZ polymer, as shown, for example in formula (XXI)):

(hydrophobic chain)₁₋₂-(alkylene moiety substituted with at least one monovalent functional moiety)-(POX and/or POZ polymer),
wherein “hydrophobic chain” represents R₁₄; “alkylene moiety substituted with at least one monovalent functional moiety” represents L₁₁; and “POX and/or POZ polymer” represents the polymer specified in formula (XX).

In some embodiments, the oxazolinylated and/or oxazinylated lipid has the following formula (XXIc) (in particular, if the one or more hydrophobic chains are attached to the N-end (i.e., the terminal N atom) of the POX and/or POZ polymer, as shown, for example in formula (XXI)):

(hydrophobic chain)₁₋₂-(alkylene moiety substituted with at least one monovalent functional moiety)-(POX and/or POZ polymer)-R₁₃

In some embodiments (in particular of the oxazolinylated and/or oxazinylated lipid of formula (XXIc)), the at least one monovalent functional moiety may be any one of the monovalent functional moieties specified herein, e.g., selected from the groups consisting of hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide.

In some embodiments (in particular of the oxazolinylated and/or oxazinylated lipid of formula (XXIc)), the alkylene moiety substituted with at least one monovalent functional moiety is C₁₋₆-alkylene, such as C₁₋₃-alkylene, e.g., methylene, ethylene, or trimethylene.

In some embodiments (in particular of the oxazolinylated and/or oxazinylated lipid of formula (XXIc)), the alkylene moiety substituted with at least one monovalent functional moiety is substituted with one or more (such as 1 to the maximum number of hydrogen atoms bound to the alkylene moiety, e.g., 1, 2, 3, 4, 5, or 6, such as between 1 to 5, 1 to 4, or 1 to 3, or 1 or 2) independently selected monovalent functional moieties.

In some embodiments (in particular of the oxazolinylated and/or oxazinylated lipid of formula (XXIc)), the alkylene moiety substituted with at least one monovalent functional moiety is C₁₋₆-alkylene, such as C₁₋₃-alkylene, e.g., methylene, ethylene, or trimethylene, and is substituted with one or more (such as 1 to the maximum number of hydrogen atoms bound to the alkylene moiety, e.g., 1, 2, 3, 4, 5, or 6, such as between 1 to 5, 1 to 4, or 1 to 3, or 1 or 2) independently selected monovalent functional moieties.

In some embodiments of formulas (XXI), (XXI′), (XXIa), (XXIa′), (XXIb), and (XXIb′), L₁₁ comprises an alkylene moiety linked, at the end by which the alkylene group is attached to R₁₄, to a divalent functional moiety as specified above. Thus, in some embodiments, the oxazolinylated and/or oxazinylated lipid may comprise the following structure (in particular, if the one or more hydrophobic chains are attached to the N-end (i.e., the terminal N atom) of the POX and/or POZ polymer, as shown, for example in formula (XXI)):
[(hydrophobic chain)-(divalent functional moiety)]₁₋₂-(alkylene moiety)-(POX and/or POZ polymer),
wherein “hydrophobic chain” represents R₁₄; “-(divalent functional moiety)]₁₋₂-(alkylene moiety)” represents L₁₁; and “POX and/or POZ polymer” represents the polymer specified in formula (XX).

In some embodiments, the oxazolinylated and/or oxazinylated lipid has the following formula (XXId) (in particular, if the one or more hydrophobic chains are attached to the N-end (i.e., the terminal N atom) of the POX and/or POZ polymer, as shown, for example in formula (XXI)):

[(hydrophobic chain)-(divalent functional moiety)]₁₋₂-(alkylene moiety)-(POX and/or POZ polymer)-R₁₃

In some embodiments (in particular of the oxazolinylated and/or oxazinylated lipid of formula (XXId)), the divalent functional moiety may be any one of the divalent functional moieties specified herein, e.g., selected from the groups consisting of ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide. In some embodiments, the linker comprises at least one divalent functional moiety selected from the group consisting of ester, sulfide, disulfide, sulfone, orthoester, acylhydrazone, hydrazine, oxime, acetal, ketal, amino, and amide moieties. In some preferred embodiments, the linker comprises at least one divalent functional moiety selected from the group consisting of ester, sulfide, sulfone, amino, and amide moieties.

In some embodiments (in particular of the oxazolinylated and/or oxazinylated lipid of formula (XXId)), the alkylene moiety is C₁₋₆-alkylene, such as C₁₋₃-alkylene, e.g., methylene, ethylene, or trimethylene.

In some embodiments of formulas (XXI), (XXI′), (XXIa), (XXIa′), (XXIb), (XXIb′), (XXIc), and (XXId) (in particular those, where the one or more hydrophobic chains are attached to the N-end (i.e., the terminal N atom) of the POX and/or POZ polymer, as shown, for example in formula (XXI)), L₁₁ comprises at least one ester, sulfide, disulfide, sulfone, orthoester, acylhydrazone, hydrazine, oxime, acetal, ketal, or amide moiety. In certain embodiments, L₁₁ is selected from the group consisting of [*—NHC(O)]ₚ(C₁₋₆-alkylene), [*—C(O)NH]ₚ(C₁₋₆-alkylene)-, [*—C(O)O]ₚ(C₁₋₆-alkylene)-, [*—OC(O)]ₚ—(C₁₋₆-alkylene)-, [*—S]ₚ(C₁₋₆-alkylene)-, [*—SS]ₚ(C₁₋₆-alkylene)-, [*—S(O)₂]ₚ(C₁₋₆-alkylene)-, [(*—O)ᵣC(OR₂₅)₃₋ᵣ](C₁₋₆alkylene)-, [*—C(OR₂₅)₂O]ₚ(C₁₋₆-alkylene)-, [*—C(R₂₅)(═N—N(R₂₆)C(O)—)]ₚ(C₁₋₆-alkylene)-, [*—C(O)(N(R₂₆)—N═)C(R₂₅)—]ₚ(C₁₋₆-alkylene)-, [*═C(═N—N(R₂₆)C(O)(R₂₅))]ₚ(C₁₋₆-alkylene)-, [*N(R₂₆)N(R₂₆)]ₚ(C₁₋₆alkylene)-, [*═C(═N(OH))]ₚ(C₁₋₆-alkylene)-, and [*—OC(R₂₅)(R₂₆)O]ₚ(C₁₋₆-alkylene)-, wherein * represents the attachment point to R₁₄; p is 1 or 2; C₁₋₆-alkylene is either bivalent (if p is 1) or trivalent (if p is 2); R₂₅ is selected from the group consisting of C₁₋₆ alkyl, aryl, and aryl-(C₁₋₆ alkyl); R₂₆ is selected from the group consisting of H, C₁₋₆ alkyl, aryl, and aryl(C₁₋₆ alkyl); and r is an integer between 1 and 2.

For example, L₁₁ may be selected from the group consisting of [*—NHC(O)]ₚ(C-3-alkylene)-, [*—C(O)NH]ₚ(C₁₋₃-alkylene)-, [*—C(O)O]ₚ(C₁₋₃-alkylene)-, [*—OC(O)]ₚ(C₁₋₃-alkylene)-, [*—S]ₚ(C₁₋₃-alkylene)-, [*—SS]ₚ(C₁₋₃-alkylene)-, [*—S(O)₂]ₚ(C₁₋₃-alkylene)-, [(*—O)ᵣC(OR₂₅)₃₋ᵣ](C₁₋₃-alkylene)-, [*—C(OR₂₅)₂O]ₚ(C₁₋₃-alkylene)-, [*—C(R₂₅)(═N—N(R₂₆)C(O)—)]ₚ(C₁₋₃-alkylene)-, [*—C(O)(N(R₂₆)—N═)C(R₂₅)—]ₚ(C₁₋₃-alkylene)-, [*═C(═N—N(R₂₆)C(O)(R₂₅))]ₚ(C₁₋₃-alkylene)-, [*N(R₂₆)N(R₂₆)]ₚ(C-3-alkylene)-, [*═C(═N(OH))]ₚ(C₁₋₃-alkylene)-, and [*—OC(R₂₅)(R₂₆)O]ₚ(C₁₋₃-alkylene)-, wherein * represents the attachment point to R₁₄; p is 1 or 2; C₁₋₃-alkylene is either bivalent (if p is 1) or trivalent (if p is 2); R₂₅ is selected from the group consisting of C₁₋₆ alkyl, aryl, and aryl(C₁₋₆ alkyl); R₂₆ is selected from the group consisting of H, C₁₋₆ alkyl, aryl, and aryl(C₁₋₆ alkyl); and r is an integer between 1 and 2.

In some embodiments, R₂₅ is selected from the group consisting of C₁₋₃ alkyl, phenyl, and phenyl(C₁₋₃ alkyl), such as from the group consisting of methyl, ethyl, phenyl, benzyl, and phenylethyl.

In some embodiments, R₂₆ is selected from the group consisting of H, C₁₋₃ alkyl, phenyl, and phenyl(C₁₋₃ alkyl), such as from the group consisting of H, methyl, ethyl, phenyl, benzyl, and phenylethyl.

In some embodiments of formulas (XXI), (XXI′), (XXIa), (XXIa′), (XXIb), (XXIb′), (XXIc), and (XXId) (in particular those, where the one or more hydrophobic chains are attached to the N-end (i.e., the terminal N atom) of the POX and/or POZ polymer, as shown, for example in formula (XXI)), ₁₁ is selected from the group consisting of [*—NHC(O)]ₚ(C₁₋₆-alkylene)-, [*—C(O)N H]ₚ(C₁₋₆-alkylene)-, [*—C(O)O]ₚ(C₁₋₆-alkylene)-, [*—OC(O)]ₚ(C₁-alkylene)-, [*—S]ₚ(C₁₋₆-alkylene)-, and [*—S(O)₂]ₚ(C₁₋₆-alkylene)-, preferably from the group consisting of [*—NHC(O)]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, and [*—S(O)₂]ₚ(C₁₋₆-alkylene)-, more preferably from the group consisting of [*—NHC(O)]ₚ(C₁₋₆alkylene)- and [*—C(O)O]ₚ(C₁₋₆-alkylene)-, wherein * represents the attachment point to R₁₄; p is 1 or 2; and C₁₋₆-alkylene is either bivalent (if p is 1) or trivalent (if p is 2).

In some embodiments of formulas (XXI), (XXI′), (XXIa), (XXIa′), (XXIb), (XXIb′), (XXIc), and (XXId) (in particular those, where the one or more hydrophobic chains are attached to the N-end (i.e., the terminal N atom) of the POX and/or POZ polymer, as shown, for example in formula (XXI)), ₁₁ is selected from the group consisting of [*—NHC(O)]ₚ(C₁₋₃-alkylene)-, [*—C(O)NH]ₚ(C₁₋₃-alkylene)-, [*—C(O)O]ₚ(C₁₋₃-alkylene)-, [*—OC(O)]ₚ—(C₁₋₃-alkylene)-, [*—S]ₚ(C₁₋₃-alkylene)-, and [*—S(O)₂]ₚ(C₁₋₃-alkylene)-, preferably from the group consisting of [*—NHC(O)]ₚ(C₁₋₃-alkylene)-, [*—C(O)O]ₚ(C₁₋₃-alkylene)-, [*—OC(O)]ₚ(C₁₋₃-alkylene)-, [*—S]ₚ(C₁₋₃-alkylene)-, and [*—S(O)₂]ₚ(C₁₋₃-alkylene)-, more preferably from the group consisting of [*—NHC(O)]ₚ(C₁₋₃-alkylene)- and [*—C(O)O]ₚ(C₁₋₃-alkylene)-, wherein * represents the attachment point to R₁₄; p is 1 or 2; and C₁₋₃-alkylene is either bivalent (if p is 1) or trivalent (if p is 2).

In some embodiments of formulas (XXI), (XXI′), (XXIa), (XXIa′), (XXIb), (XXIb′), (XXIc), and (XXId) (in particular those, where the one or more hydrophobic chains are attached to the N-end (i.e., the terminal N atom) of the POX and/or POZ polymer, as shown, for example in formula (XXI)), L₁₁ is selected from the group consisting of *—NHC(O)—(CH₂)—*—NHC(O)—(CH₂)₂—, *—C(O)NH—(CH₂)—, *—C(O)NH—(CH₂)₂—, —(CH₂)—CH(OC(O)—*)—(CH₂OC(O)—*), —(CH₂)—CH(S—*)₂, —(CH₂)—CH(S—*)—CH₂(S—*), *—S—(CH₂)₃—, *—S(O)₂—(CH₂)₃—, and *—OC(O)—(CH₂)—, wherein * represents the attachment point to R₁₄. Thus, R₁₂ may be selected from the group consisting of R₁₄, -L₁₁R₁₄, —(CH₂)—CH(OC(O)R₁₄)(CH₂OC(O)R₁₄), —(CH₂)—CH(SR₁₄)₂, and —(CH₂)—CH(SR₁₄)—CH₂(SR₁₄); and L₁₁ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)—, preferably L₁₁ is *—NHC(O)—(CH₂)— or *—NHC(O)—(CH₂)₂—, wherein * represents the attachment point to R₁₄.

In some embodiments of formulas (XXI), (XXIa), and (XXIb), R₁₂ is -L₁₁(R₁₄)ₚ, i.e., the POX and/or POZ polymer is conjugated to the one or more hydrophobic chains (i.e., R₁₄) via the linker L,,.

In some embodiments of formulas (XXI′), (XXIa′), and (XXIb′), the linker comprises at least one difunctional moiety via which the one or more hydrophobic chains (R₁₄) are attached to the C-end of the POX and/or POZ polymer. In some embodiments, the linker may additionally comprise an alkylene moiety (such as a C₁₋₆ alkylene moiety, e.g., a C₁₋₃ alkylene moiety), a cycloalkylene moiety (preferably a C₃₋₈-cycloalkylene, such as C₃₋₆-cycloalkylene moiety), or a cycloalkenylene moiety (preferably a C₃₋₈-cycloalkenylene, such as C₃₋₆-cycloalkenylene moiety) each of which connects the difunctional moiety to the C-end POX and/or POZ polymer (either directly to the C-end or, preferably, via a further difunctional moiety). For example, one hydrophobic chain (R₁₄) may be attached to the C-end of the POX and/or POZ polymer via one difunctional moiety (either directly or via an alkylene, cycloalkylene, or cycloalkenylene moiety or via an alkylene, cycloalkylene, or cycloalkenylene moiety which bears another difunctional moiety); two hydrophobic chains (R₁₄) may be attached to the C-end of the POX and/or POZ polymer via two difunctional moieties (which in turn are preferably attached to an alkylene, cycloalkylene, or cycloalkenylene moiety or to an alkylene, cycloalkylene, or cycloalkenylene moiety bearing another difunctional moiety); or two hydrophobic chains (R₁₄) may be attached to the C-end of the POX and/or POZ polymer via the same difunctional moiety (which is then a trifunctional moiety and which may be attached to the C-end of the POX and/or POZ polymer either directly or via an alkylene, cycloalkylene, or cycloalkenylene moiety or to an alkylene, cycloalkylene, or cycloalkenylene moiety bearing another difunctional moiety). In some embodiments, each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties. In some preferred embodiments of formulas (XXI′), (XXIa′), and (XXIb′), the linker comprises at least one divalent functional moiety selected from the group consisting of amide, sulfide, sulfone, and amino moieties.

In some embodiments of formulas (XXI′), (XXIa′), and (XXIb′), the cycloalkylene moiety is C₃₋₈-cycloalkylene, such as C₃₋₆-cycloalkylene, e.g., cyclopropylene, cyclobutylene, cyclopentylene, cyclohexylene, wherein the cycloalkylene moiety is optionally substituted, such as optionally substituted with one or more (e.g., 1, 2, 3, or 4) substituents selected from the group consisting of —OH, ═O, —SH, halogen, —CN, —N₃, and C₁₋₃-alkyl.

In some embodiments of formulas (XXI′), (XXIa′), and (XXIb′), the cycloalkenylene moiety is C₃₋₈-cycloalkenylene, such as C₃₋₆-cycloalkenylene, e.g., cyclopropenylene, cyclobutenylene, cyclopentenylene, cyclohexenylene, wherein the cycloalkenylene moiety is optionally substituted, such as optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments of formulas (XXI′), (XXIa′), and (XXIb′), the alkylene moiety is C₁₋₆-alkylene, such as C₁₋₃-alkylene, e.g., methylene, ethylene, or trimethylene, or C₂₋₃ alkylene.

In some embodiments of formulas (XXI′), (XXIa′), and (XXIb′), the oxazolinylated and/or oxazinylated lipid comprises one of the following structures:

(hydrophobic chain)-(divalent functional moiety)-(POX and/or POZ polymer)

[(hydrophobic chain)-(divalent functional moiety)]₁₋₂-(alkylene moiety)-(divalent functional moiety)-(POX and/or POZ polymer)

(hydrophobic chain)-(divalent functional moiety)-(cycloalkylene moiety)-(divalent functional moiety)-(POX and/or POZ polymer)

(hydrophobic chain)-(divalent functional moiety)-(cycloalkenylene moiety)-(divalent functional moiety)-(POX and/or POZ polymer)

(hydrophobic chain)-(divalent functional moiety)-(alkylene moiety)-(POX and/or POZ polymer)

[(hydrophobic chain)₂-(trivalent functional moiety)]-(alkylene moiety)-(divalent functional moiety)-(POX and/or POZ polymer)

In some embodiments of formulas (XXI′), (XXIa′), and (XXIb′), the oxazolinylated and/or oxazinylated lipid has one of the following formulas (XXIe′) to (XXIj′):

(hydrophobic chain)-(divalent functional moiety)-(POX and/or POZ polymer)-R₁₃ (XXIe′)

[(hydrophobic chain)-(divalent functional moiety)]₁₋₂-(alkylene moiety)-(divalent functional moiety)-(POX and/or POZ polymer)-R₁₃ (XXIf′)

(hydrophobic chain)-(divalent functional moiety)-(cycloalkylene moiety)-(divalent functional moiety)-(POX and/or POZ polymer)-R₁₃ (XXIg′)

(hydrophobic chain)-(divalent functional moiety)-(cycloalkenylene moiety)-(divalent functional moiety)-(POX and/or POZ polymer)-R₁₃ (XXIh′)

(hydrophobic chain)-(divalent functional moiety)-(alkylene moiety)-(POX and/or POZ polymer)-R₁₃ (XXIi′)

[(hydrophobic chain)₂-(trivalent functional moiety)]-(alkylene moiety)-(divalent functional moiety)-(POX and/or POZ polymer)-R₁₃ (XXIj′)

In some embodiments of formulas (XXI′), (XXIa′), (XXIb′), (XXIe′), (XXIf′), (XXIg′), (XXIh′), (XXIi′), and (XXIj′), L₁₁ is selected from the group consisting of [*—Z]ₚ(C₁₋₆-alkylene)-Z—, *—Z—(C₃₋₈-cycloalkylene)-Z—, *—Z—(C₃₋₈-cycloalkenylene)-Z—, (*═N)(C₁₋₆-alkylene)-Z—, *—Z—(C₁₋₆-alkylene)-, and *—Z—, wherein * represents the attachment point to R₁₄; p is 1 or 2; C₁₋₃-alkylene is either bivalent (if p is 1) or trivalent (if p is 2); each of the C₃₋₈-cycloalkylene and C₃₋₈-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(C₁₋₃-alkylene)NH—, —NH(C₁₋₃-alkylene)OP(O)₂O—, —C(O)NH—, —NHC(O)—, —OC(O)NH—, —NHC(O)O—, —O—, —C(O)O—, —OC(O)—, —S—, —S(O)₂—, and —NR₂₂—, wherein R₂₂ is selected from the group consisting of H, alkyl, alkenyl, alkynyl, cycloalkyl, aryl, heteroaryl, and heterocyclyl. For example, the linker can be selected from the group consisting of [*—C(O)O]ₚ(C₁₋₆alkylene)-Z—, (*—NH)(C₁₋₆alkylene)-Z—, (*═N)(C₁₋₆-alkylene)-Z—, (*—NH)C(O)(C₁₋₆-alkylene)-Z—, (*—C(O)NH(C₁₋₆-alkylene)-Z—, (*—NH)C(O)(C₁₋₆-alkylene)-, (*—C(O)NH(C₁₋₆alkylene)-, (*—NH)C(O)—, *—C(O)NH—, *—Z—(C₃₋₈-cycloalkenylene)-Z—, —S—, and —S(O)₂—, wherein * represents the attachment point to R₁₄; p is 1 or 2; C₁₋₆-alkylene is either bivalent (if p is 1) or trivalent (if p is 2); the C₃₋₈-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(C₁₋₃-alkylene)NH—, —NH(C₁₋₃-alkylene)OP(O)₂O—, —OC(O)NH—, —NHC(O)O—, —O—, —S—, and —NH—

In some embodiments of formulas (XXI′), (XXIa′), (XXIb′), (XXIe′), (XXIf′), (XXIg′), (XXIh′), (XXIi′), and (XXIj′), L₁₁ is selected from the group consisting of [*—Z]ₚ(C₁₋₃-alkylene)-Z—, *—Z—(C₃₋₆-cycloalkylene)-Z—, *—Z—(C₃₋₆-cycloalkenylene)-Z—, (*═N)(C₁₋₃-alkylene)-Z—, *—Z(C₁₋₃-alkylene)-, and *—Z—, wherein * represents the attachment point to R₁₄; p is 1 or 2; C₁₋₃-alkylene is either bivalent (if p is 1) or trivalent (if p is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—. For example, the linker can be selected from the group consisting of [*—C(O)O]ₚ(C₁₋₃-alkylene)-Z—, (*—NH)(C₁₋₃-alkylene)-Z—, (*═N)(C₁₋₃-alkylene)-Z—, (*—NH)C(O)(C₁₋₃-alkylene)-Z—, (*—C(O)NH(C₁₋₃-alkylene)-Z—, (*—NH)C(O)(C₁₋₃-alkylene)-, (*—C(O)NH(C₁₋₃-alkylene)-, (*—NH)C(O)—, *—C(O)NH—, *—Z—(C₃₋₆-cycloalkenylene)-Z—. —S—, and —S(O)₂—, wherein * represents the attachment point to R₁₄; p is 1 or 2; C₁₋₃-alkylene is either bivalent (if p is 1) or trivalent (if p is 2); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(C₁₋₂-alkylene)NH—, —NH(C₁₋₂-alkylene)OP(O)₂O—, —OC(O)NH—, —NHC(O)O—, —O—, —S—, and —NH—.

In some embodiments of formulas (XXI′), (XXIa′), (XXIb′), (XXIe′), (XXIf′), (XXIg′), (XXIh′), (XXIi′), and (XXIj′), L₁₁ is selected from the group consisting of [*—Z]ₚ(C₁₋₃-alkylene)-Z—, *—Z—(C₃₋₆-cycloalkylene)-Z—, *—Z—(C₃₋₆-cycloalkenylene)-Z—, (*═N)(C₁₋₃-alkylene)-Z—, *—Z(C₁₋₃-alkylene)-, and *—Z—, wherein * represents the attachment to R₁₄; p is 1 or 2; C₁₋₃-alkylene is either bivalent (if p is 1) or trivalent (if p is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—. For example, the linker can be selected from the group consisting of [*—C(O)O]ₚ(C₁₋₃-alkylene)-Z—, (*—NH)(C₁₋₃-alkylene)-Z—, (*═N)(C₁₋₃-alkylene)-Z—, (*—NH)C(O)(C₁₋₃-alkylene)-Z—, (*—C(O)NH(C₁₋₃-alkylene)-Z—, (*—NH)C(O)(C₁₋₃-alkylene)-, (*—C(O)NH(C₁₋₃-alkylene)-, (*—NH)C(O)—, *—C(O)NH—, *—Z—(C₃₋₆-cycloalkenylene)-Z—, —S—, and —S(O)₂—, wherein * represents the attachment point to R₁₄; p is 1 or 2; C₁₋₃-alkylene is either bivalent (if p is 1) or trivalent (if p is 2); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(CH₂)₂NH—, —NH(CH₂)₂OP(O)₂O—, —OC(O)NH—, —NHC(O)O—, —O—, —S—, and —NH—.

In some embodiments of formulas (XXI′), (XXIa′), (XXIb′), (XXIe′), (XXIf′), (XXIg′), (XXIh′), (XXIi′), and (XXIj′), L₁₁ is selected from the group consisting of (*—C(O)O)(CH(OC(O)—*))(CH₂)—Z—, (*═N)(C₁₋₃-alkylene)-NHC(O)—, (*—Z)(C₁₋₃-alkylene)-Z—, *—Z—(C₃₋₆-cycloalkenylene)-Z—, and *—Z—, wherein * represents the attachment point to R₁₄; the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—. For example, the linker can be selected from the group consisting of (*—C(O)O)(CH(OC(O)—*))(CH₂)—Z—, (*═N)(C₁₋₃-alkylene)-NHC(O)—, (*—NH)(C₁₋₃-alkylene)-NHC(O)—, *—C(O)NH—, (*—NH)C(O)—, *—Z—(C₃₋₆-cycloalkenylene)-Z—, —S—, and —S(O)₂—, wherein * represents the attachment point to R₁₄; the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(CH₂)₂NH—, —NH(CH₂)₂OP(O)₂O—, —OC(O)NH—, —NHC(O)O—, —O—, —S—, and —NH.

In some embodiments of formulas (XXI′), (XXIa′), (XXIb′), (XXIe′), (XXIf′), (XXIg′), (XXIh′), (XXIi′), and (XXIj′), R₁₂ is selected from the group consisting of (R₁₄C(O)O)(CH(OC(O)R₁₄))(CH₂)—Z—, (R₁₄)₂N(C₁₋₃-alkylene)-NHC(O)—, R₁₄Z(C₁₋₃-alkylene)-Z—, R₁₄Z—(C₃₋₆-cycloalkenylene)-Z—, and R₁₄Z—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—. For example, the linker can be selected from the group consisting of (R₁₄C(O)O)(CH(OC(O)R₁₄))(CH₂)—Z—, (R₁₄)₂N(C₁₋₃-alkylene)-NHC(O)—, R₁₄NH(C₁₋₃-alkylene)-NHC(O)—, R₁₄C(O)NH—, (R₁₄NH)C(O)—, R₁₄Z—(C₃₋₆-cycloalkenylene)-Z—, R₁₄S—, and R₁₄S(O)₂—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(CH₂)₂NH—, —NH(CH₂)₂OP(O)₂O—, —OC(O)NH, —NHC(O)O—, —O—, —S—, —S(O)₂—, and —NH—.

In some embodiments of formulas (XXI′), (XXIa′), (XXIb′), (XXIe′), (XXIf′), (XXIg′), (XXIh′), (XXIi′), and (XXIj′), R₁₂ is -L₁₁(R₁₄)ₚ, i.e., the POX and/or POZ polymer is conjugated to the one or more hydrophobic chains (i.e., R₁₄) via the linker L₁₁.

In any of the above embodiments of formulas (XXI), (XXI′), (XXIa), (XXIa′), (XXIb), (XXIb′), (XXIc), (XXId), (XXIe′), (XXIf′), (XXIg′), (XXIh′), (XXIi′), and (XXIj′), each R₁₄ preferably is independently a non-cyclic, more preferably straight hydrocarbyl group. For example, each R₁₄ is independently a hydrocarbyl group having at least 8 carbon atoms, such as at least 10 carbon atoms, preferably up to 30 carbon atoms, such as up to 28, 26, 24, 22, or 20 carbon atoms, or up to 16 carbon atoms, such as up to 15, 14, 13, 12, 11, or 10 carbon atoms. In some embodiments, each R₁₄ is a hydrocarbyl group having to 16 carbon atoms, such as 10 to 15 or 10 to 14 carbon atoms. In some embodiments, each R₁₄ is a straight hydrocarbyl group having 10 to 16 carbon atoms, such as 10 to 15 or 10 to 14 carbon atoms.

In any of the above embodiments of formulas (XXI′), (XXIa′), (XXIb′), (XXIe′), (XXIf′), (XXIg′), (XXIh′), (XXIi′), and (XXIj′), each R₁₄ may preferably be a hydrocarbyl group having 10 to 18 carbon atoms, such as a straight alkyl group having 10 to 18 carbon atoms or a straight alkenyl group having 10 to 18 carbon atoms. For example, a straight alkyl group may have 10, 11, 12, 13, 14, 15, 16, 17, or 18 carbon atoms; and/or a straight alkenyl group may have 10, 11, 12, 13, 14, 15, 16, 17, or 18 carbon atoms and 1, 2, or 3 carbon-carbon double bonds.

In any of the above embodiments of formulas (XXI), (XXI′), (XXIa), (XXIa′), (XXIb), (XXIb′), (XXIc), (XXId), (XXIe′), (XXIf′), (XXIg′), (XXIh′), (XXIi′), and (XXIj′), R₁₃ is preferably selected from the group consisting of H, C₁₋₃ alkyl, —OR₂₀, —N₃, C₂₋₆ alkynyl, —OC(O)R₂₁, —C(O)R₂₁, —NR₂₂R₂₃, —COOH, —C(O)NR₂₂R₂₃, —NR₂₂C(O)R₂₁, and a member of a targeting pair, wherein the C₁₋₃ alkyl group is optionally substituted with one or more substituents independently selected from the group consisting of —OH, SH, halogen, —CN, —N₃, C₂₋₆ alkynyl, —COOH, —NR₂₂R₂₃, —C(O)NR₂₂R₂₃, —NR₂₂C(O)R₂₁, and a member of a targeting pair; R₂₀ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR₂₂R₂₃, and a member of a targeting pair; R₂₁ is selected from the group consisting of C₁₋₃ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR₂₂R₂₃, and a member of a targeting pair; and each of R₂₂ and R₂₃ is independently selected from the group consisting of H, C₁₋₆ alkyl, C₂₋₆ alkenyl, and C₂₋₆ alkynyl, or R₂₂ and R₂₃ may join together with the nitrogen atom to which they are attached to form a heterocyclyl group, wherein each of the C₁₋₆ alkyl, C₂₋₆ alkenyl, and C₂₋₆ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair.

In any of the above embodiments of formulas (XXI), (XXI′), (XXIa), (XXIa′), (XXIb), (XXIb′), (XXIc), (XXId), (XXIe′), (XXIf′), (XXIg′), (XXIh′), (XXIi′), and (XXIj′), R₁₃ is preferably selected from the group consisting of H, C₁₋₃ alkyl, —OR₂O, —N₃, C₂₋₆ alkynyl, —OC(O)R₂₁, —C(O)R₂₁, —NR₂₂R₂₃, —COOH, —C(O)NR₂₂R₂₃, —NR₂₂C(O)R₂₁, and a member of a targeting pair, wherein the C₁₋₃ alkyl group is optionally substituted with one or more substituents independently selected from the group consisting of —OH, —N₃, C₂₋₆ alkynyl, —COOH, —NH₂, —NHCH₃, —N(CH₃)₂, —C(O)NR₂₂R₂₃, —NR₂₂C(O)R₂₁, and a member of a targeting pair; R₂₀ is selected from the group consisting of H and C₁₋₃ alkyl; R₂₁ is C₁₋₃ alkyl optionally substituted with one or more substituents independently selected from the group consisting of —OH, SH, halogen, —CN, —N₃, C₂₋₆ alkynyl, —COOH, —NR₂₂R₂₃, and a member of a targeting pair; and each of R₂₂ and R₂₃ is independently selected from the group consisting of H, C₁₋₃ alkyl, C₂₋₃ alkenyl, and C₂₋₃ alkynyl, wherein each of the C₁₋₃ alkyl, C₂₋₃ alkenyl, and C₂₋₃ alkynyl groups is optionally substituted with one or more (such as 1, 2, 3, or 4) substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair, or R₂₂ and R₂₃ may join together with the nitrogen atom to which they are attached to form a 5- or 6-membered heterocyclyl group.

In any of the above embodiments of formulas (XXI), (XXI′), (XXIa), (XXIa′), (XXIb), (XXIb′), (XXIc), (XXId), (XXIe′), (XXIf′), (XXIg′), (XXIh′), (XXIi′), and (XXIj′), R₁₃ is preferably selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O)CH₃, —C(O)NH₂, —C(O)NHCH₃, —OC(O)(CH₂)₂COOH, and a member of a targeting pair, wherein the C₁₋₃ alkyl group is optionally substituted with one or more (such as 1 or 2) substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair.

In some embodiments of formulas (XXI), (XXIa), (XXIb), (XXIc), and (XXId), R₁₃ is selected from the group consisting of H, —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O)CH₃, —NH(CH₂CH₃), —C(O)NH₂, —C(O)NHCH₃, —OC(O)(CH₂)₂COOH, and a member of a targeting pair. In some embodiments of formulas (XXI), (XXIa), (XXIb), (XXIc), and (XXId), R₁₃ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O)CH₃, —NH(CH₂CH₃), —C(O)NH₂, —C(O)NHCH₃, —OC(O)(CH₂)₂COOH, and a member of a targeting pair. In some embodiments of formulas (XXI), (XXIa), (XXIb), (XXIc), and (XXId), R₁₃ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O)CH₃, —NH(CH₂CH₃), —OC(O)(CH₂)₂COOH, and a member of a targeting pair. In some embodiments of formulas (XXI), (XXIa), (XXIb), (XXIc), and (XXId), R₁₃ is selected from the group consisting of —OH, —N₃, —NH₂, —NHC(O)(CH₂)₂COOH, —N(CH₂CH₃)C(O)(CH₂)₂COOH, —N(CH₂CH₃)C(O)CH₃, —NH(CH₂CH₃), and —OC(O)(CH₂)₂COOH.

In some embodiments of formulas (XXI′), (XXIa′), (XXIb′), (XXIe′), (XXIf′), (XXIg′), (XXIh′), (XXIi′), and (XXIj′), R₁₃ is C₁₋₃ alkyl optionally substituted with one or two substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair. In some embodiments of formulas (XXI′), (XXIa′), (XXIb′), (XXIe′), (XXIf′), (XXIg′), (XXIh′), (XXIi′), and (XXIj′), R₁₃ is C₁₋₃ alkyl optionally substituted with one substituent selected from the group consisting of —COOH and —C(O)NH(CH₂)₂NH₂.

In certain embodiments, the oxazolinylated and/or oxazinylated lipid has the following general formula (XXII) or (XXII′):

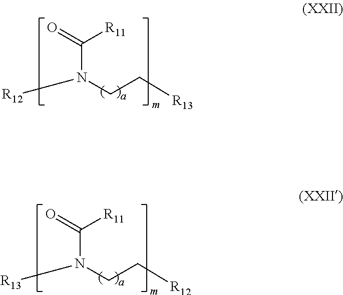

wherein:
a is an integer between 1 and 2;
R₁₁ is methyl or ethyl, and is independently selected for each repeating unit;
m is 10 to 100 (preferably 20 to 80, 30 to 70, or 40 to 50);
R₁₂, for formula (XXII), is selected from the group consisting of -L₁₁R₁₄, —(CH₂)—CH(OC(O)R₁₄)(CH₂OC(O)R₁₄), —(CH₂)—CH(SR₁₄)₂, and —(CH₂)—CH(SR₁₄)—CH₂(SR₁₄), wherein each R₁₄ is independently a straight hydrocarbyl group having at least 10 carbon atoms (preferably having 10 to 16 carbon atoms); and L₁₁ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L₁₁ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R₁₄; or R₁₂, for formula (XXII′), is selected from the group consisting of (R₁₄C(O)O)(CH(OC(O)R₁₄))(CH₂)—Z—, (R₁₄)₂N(C₁₋₃-alkylene)-Z—, R₁₄Z(C₁₋₃-alkylene)-Z—, R₁₄Z—(C₃₋₆-cycloalkenylene)-Z—, and R₁₄Z—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—; and
R₁₃ is selected from the group consisting of H, C₁₋₃ alkyl, —OH, —N₃, C₂₋₆ alkynyl, —COOH, —NH₂,
—NHCH₃, —N(CH₃)₂, —NH(CH₂CH₃), —NHC(O)(CH₂)₂COOH, —N(CH₂CH₃)C(O)(CH₂)₂COOH,
—N(CH₂CH₃)C(O)CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair.

In some embodiments of formula (XXII) or (XXII′), a is 1, i.e., the oxazolinylated and/or oxazinylated lipid has the following general formula (XXIIa) or (XXIIa′):

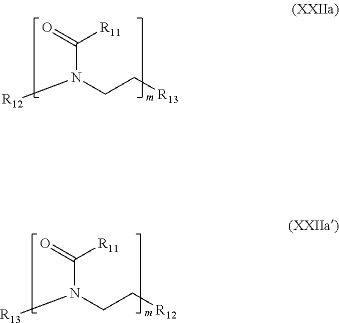

In some embodiments of formula (XXII) or (XXII′), a is 2, i.e., the oxazolinylated and/or oxazinylated lipid has the following general formula (XXIIb) or (XXIIb′):

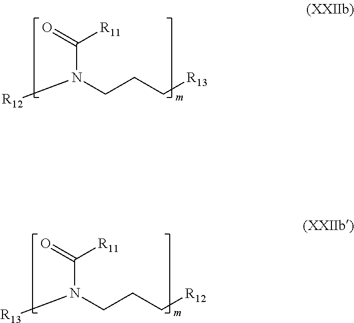

In any of the above embodiments of formulas (XXIIa), (XXIIa′), (XXIIb), and (XXIIb′), R₁₁, R₁₂, R₁₃, and m are as defined for formula (XXII)/(XXII′).

In some embodiments of formulas (XXII), (XXII′), (XXIIa), (XXIIa′), (XXIIb), and (XXIIb′), each R₁₁ is methyl or each R₁₁ is ethyl. In some alternative embodiments of formulas (XXII), (XXII′), (XXIIa), (XXIIa′), (XXIIb), and (XXIIb′), R₁₁ is independently selected from methyl and ethyl for each repeating unit, wherein in at least one repeating unit R₁₁ is methyl, and in at least one repeating unit R₁₁ is ethyl.

In some embodiments of formulas (XXII), (XXIIa), and (XXIIb), R₁₂ is selected from the group consisting of R₁₄—NHC(O)—(CH₂)—, R₁₄—NHC(O)—(CH₂)₂—, —(CH₂)—CH(OC(O)R₁₄)(CH₂OC(O)R₁₄), —(CH₂)—CH(SR₁₄)—CH₂(SR₁₄), R₁₄S—(CH₂)₃—, R₁₄S(O)₂—(CH₂)₃—, and R₁₄—OC(O)—(CH₂)—; and/or R₁₃ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O)CH₃, —NH(CH₂CH₃), —OC(O)(CH₂)₂COOH, and a member of a targeting pair (e.g., R₁₃ is selected from the group consisting of —OH, —N₃, —NH₂, —NHC(O)(CH₂)₂COOH, —N(CH₂CH₃)C(O)(CH₂)₂COOH, —N(CH₂CH₃)C(O)CH₃, —NH(CH₂CH₃), and —OC(O)(CH₂)₂COOH).

In some embodiments of formulas (XXII′), (XXIIa′), and (XXIIb′), R₁₂ is selected from the group consisting of R₁₄C(O)NH—, R₁₄S—, R₁₄S(O)₂—, and R₁₄NH—(3,4-dioxocyclobut-1-en-1,2-diyl)-NH—; and/or R₁₃ is C₁₋₃ alkyl optionally substituted with one or two substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair (e.g., R₁₃ is C₁₋₃ alkyl optionally substituted with one substituent selected from the group consisting of —COOH and —C(O)NH(CH₂)₂NH₂).

In some embodiments, the oxazolinylated and/or oxazinylated lipid has the following general formula (XXIII):

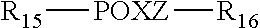

wherein:
R₁₅ is R₁₇ or -L₁₂(R₁₇)_{q}, wherein each R₁₇ is independently a hydrocarbyl group; L₁₂ is a linker; and q is 1 or 2;
POXZ is a copolymer containing repeating units of the following general formulas (XXa) and (XXb):

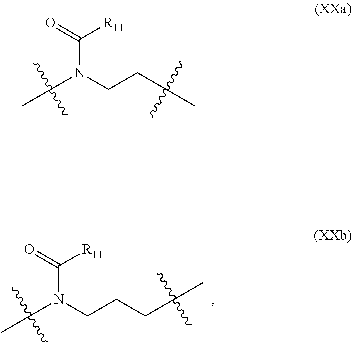

wherein each of R₁₁ is independently alkyl, in particular C₁₋₃ alkyl, such as methyl, ethyl, iso-propyl, or n-propyl, and is independently selected for each repeating unit; the number of repeating units of formula (XXa) in the copolymer is 1 to 199; the number of repeating units of formula (XXb) in the copolymer is 1 to 199; the sum of the number of repeating units of formula (XXa) and the number of repeating units of formula (XXb) in the copolymer is 2 to 200; and the repeating units of formulas (XXa) and (XXb) are arranged in a random, periodic, alternating or block wise manner; and
R₁₆ is selected from the group consisting of H, C₁₋₆ alkyl, C₂₋₆ alkynyl, —OR₂₀, —SR₂₀, halogen, —CN, —N₃, —OC(O)R₂₁, —C(O)R₂₁, —NR₂₂R₂₃, —COOH, —C(O)NR₂₂R₂₃, —NR₂₂C(O)R₂₁, a sugar, an amino acid, a peptide, and a member of a targeting pair, wherein the C₁₋₆ alkyl group is optionally substituted with one or more substituents independently selected from the group consisting of —OH, SH, halogen, —CN, —N₃, C₂₋₆ alkynyl, —COOH, —NR₂₂R₂₃, —C(O)NR₂₂R₂₃, —NR₂₂C(O)R₂₁, a sugar, an amino acid, a peptide, and a member of a targeting pair; R₂₀ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR₂₂R₂₃, a sugar, an amino acid, a peptide, and a member of a targeting pair; R₂₁ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR₂₂R₂₃, a sugar, an amino acid, a peptide, and a member of a targeting pair; and each of R₂₂ and R₂₃ is independently selected from the group consisting of H, alkyl, alkenyl, alkynyl, cycloalkyl, aryl, heteroaryl, and heterocyclyl, or R₂₂ and R₂₃ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂′, a sugar, an amino acid, a peptide, and a member of a targeting pair.

In some embodiments of formula (XXIII), the targeting pair is selected from the following pairs: antigen-antibody specific for said antigen; avidin-streptavidin; folate-folate receptor; transferrin-transferrin receptor; aptamer-molecule for which the aptamer is specific; arginine-glycine-aspartic acid (RGD) peptide-αᵥβ₃ integrin; asparagine-glycine-arginine (NGR) peptide-aminopeptidase N; galactose-asialoglyco-protein receptor. Thus, in some embodiments of formula (XXIII), a member of a targeting pair includes one of the following: an antigen, an antibody, avidin, streptavidin, folate, transferrin, an aptamer; an RGD peptide; an NGR peptide; and galactose.

In some of the above embodiments of formula (XXIII), R₁₁ at each occurrence (i.e., in each repeating unit) may be the same alkyl group (e.g., R₁₁ may be methyl in each repeating unit). In some alternative embodiments of formula (XXIII), R₁₁ in at least one repeating unit differs from R₁₁ in another repeating unit (e.g., for at least one repeating unit R₁₁ is one specific alkyl (such as ethyl), and for at least one different repeating unit R₁₁ is a different specific alkyl (such as methyl)). For example, each R₁₁ may be selected from two different alkyl groups (such as methyl and ethyl) and not all R¹ are the same alkyl.

In some embodiments of formula (XXIII), each of R₁₁ is independently methyl or ethyl, preferably methyl. Thus, in some embodiments of formula (XXIII), each R₁₁ is methyl or each R₁₁ is ethyl. In some alternative embodiments of formula (XXIII), R₁₁ is independently selected from methyl and ethyl for each repeating unit, wherein in at least one repeating unit R₁₁ is methyl, and in at least one repeating unit R₁₁ is ethyl.

In some embodiments of formula (XXIII), the sum of the number of repeating units of formula (XXa) and the number of repeating units of formula (XXb) in the copolymer preferably is between 2 and 190, such as between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 4 and 200, between 4 and 190, between 4 and 180, between 4 and 170, between 4 and 160, between 4 and 150, between 4 and 140, between 4 and 130, between 4 and 120, between 4 and 110, between 4 and 100, between 4 and 90, between 4 and 80, between 4 and 70, between 10 and 200, between and 190, between 10 and 180, between 10 and 170, between 10 and 160, between and 150, between 10 and 140, between 10 and 130, between 10 and 120, between and 110, between 10 and 100, between 10 and 90, between 10 and 80, or between and 70. In certain embodiments of formula (XXIII), the sum of the number of repeating units of formula (XXa) and the number of repeating units of formula (XXb) in the copolymer is 2 to 180, such as 4 to 160, 6 to 140, 8 to 120 or 10 to 100, e.g., 20 to 80, to 70, or 40 to 50.

Accordingly, in some embodiments of formula (XXIII), the number of repeating units of formula (XXa) in the copolymer is 1 to 179, such as 1 to 159, 1 to 139, 1 to 119 or 1 to 99; the number of repeating units of formula (XXb) in the copolymer is 1 to 179, such as 1 to 159, 1 to 139, 1 to 119 or 1 to 99; and the sum of the number of repeating units of formula (XXa) and the number of repeating units of formula (XXb) in the copolymer is 2 to 180, such as 4 to 160, 6 to 140, 8 to 120 or 10 to 100.

In some embodiments of formula (XXIII), L₁₂ comprises at least one functional moiety, such as an alkylene moiety substituted with at least one monovalent functional moiety and/or linked, at the end by which the alkylene group is attached to R₁₇, to a divalent functional moiety, wherein preferably each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide; and/or each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide.

In some embodiments of formula (XXIII), R₁₅ is attached to the N-end (i.e., the terminal N atom) of the POXZ copolymer and R₁₆ is attached to the C-end (i.e., the terminal C atom) of the POXZ copolymer. In some alternative embodiments of formula (XXIII), R₁₅ is attached to the C-end (i.e., the terminal C atom) of the POXZ copolymer and R₁₆ is attached to the N-end (i.e., the terminal N atom) of the POXZ copolymer. The latter alternative embodiments of formula (XXIII) (i.e., where R₁₅ is attached to the C-end (i.e., the terminal C atom) of the POXZ copolymer and R₁₆ is attached to the N-end (i.e., the terminal N atom) of the POXZ copolymer) are designated as formula (XXIII′) herein.

In some embodiments of formula (XXIII), L₁₂ comprises an alkylene moiety substituted with at least one monovalent functional moiety as specified above. Thus, in some embodiments, the oxazolinylated and/or oxazinylated lipid may comprise the following structure (optionally R₁₆ is attached to the terminal C atom of the POXZ copolymer):

(hydrophobic chain)₁₋₂-(alkylene moiety substituted with at least one monovalent functional moiety)-(POXZ copolymer),
wherein “hydrophobic chain” represents R₁₇; “alkylene moiety substituted with at least one monovalent functional moiety” represents L₁₂; and “POXZ copolymer” represents the copolymer specified in formula (XXIII).

In some embodiments, the oxazolinylated and/or oxazinylated lipid has the following formula (XXIIIa) (optionally R₁₆ is attached to the terminal C atom of the POXZ copolymer):

(hydrophobic chain)₁₋₂-(alkylene moiety substituted with at least one monovalent functional moiety)-(POXZ copolymer)-R₁₆

In some embodiments of formulas (XXIII) and (XXIIIa), the at least one monovalent functional moiety may be any one of the monovalent functional moieties specified herein, e.g., selected from the groups consisting of hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide.

In some embodiments of formulas (XXIII) and (XXIIIa), the alkylene moiety substituted with at least one monovalent functional moiety is C₁₋₆-alkylene, such as C₁₋₃-alkylene, e.g., methylene, ethylene, or trimethylene.

In some embodiments of formulas (XXIII) and (XXIIIa), the alkylene moiety substituted with at least one monovalent functional moiety is substituted with one or more (such as 1 to the maximum number of hydrogen atoms bound to the alkylene moiety, e.g., 1, 2, 3, 4, 5, or 6, such as between 1 to 5, 1 to 4, or 1 to 3, or 1 or 2) independently selected monovalent functional moieties.

In some embodiments, the alkylene moiety substituted with at least one monovalent functional moiety is C₁₋₆-alkylene, such as C₁₋₃-alkylene, e.g., methylene, ethylene, or trimethylene, and is substituted with one or more (such as 1 to the maximum number of hydrogen atoms bound to the alkylene moiety, e.g., 1, 2, 3, 4, 5, or 6, such as between 1 to 5, 1 to 4, or 1 to 3, or 1 or 2) independently selected monovalent functional moieties.

In some embodiments of formula (XXIII) (in particular those, where R₁₆ is attached to the terminal C atom of the POXZ copolymer), L₁₂ comprises an alkylene moiety linked, at the end by which the alkylene group is attached to R₁₇, to a divalent functional moiety as specified above. Thus, in some embodiments (in particular those, where R₁₆ is attached to the terminal C atom of the POXZ copolymer), the oxazolinylated and/or oxazinylated lipid may comprise the following structure:

[(hydrophobic chain)-(divalent functional moiety)]₁₋₂-(alkylene moiety)-(POXZ copolymer),
wherein “hydrophobic chain” represents R₁₇; “-(divalent functional moiety)]₁₋₂-(alkylene moiety)” represents L₁₂; and “POXZ copolymer” represents the copolymer specified in formula (XXIII).

In some embodiments (in particular those, where R₁₆ is attached to the terminal C atom of the POXZ copolymer), the oxazolinylated and/or oxazinylated lipid has the following formula (XXIIIb):

[(hydrophobic chain)-(divalent functional moiety)]₁₋₂-(alkylene moiety)-(POXZ copolymer)-R₁₆

In some embodiments of formulas (XXIII) and (XXIIIb), the divalent functional moiety may be any one of the divalent functional moieties specified herein, e.g., selected from the groups consisting of ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide.

In some embodiments of formulas (XXIII) and (XXIIIb), the alkylene moiety is C₁₋₆-alkylene, such as C₁₋₃-alkylene, e.g., methylene, ethylene, or trimethylene.

In some embodiments of formula (XXIII), where R₁₆ is attached to the terminal N atom of the POXZ copolymer (i.e., in the embodiments of formula (XXIII′)), L₁₂ comprises at least one difunctional moiety via which the one or more hydrophobic chains (R₁₇) are attached to the POXZ copolymer. In some embodiments, L₁₂ may additionally comprise an alkylene moiety (such as a C₁₋₆ alkylene moiety, e.g., a C₁₋₃ alkylene moiety), a cycloalkylene moiety (preferably a C₃₋₈-cycloalkylene, such as C₃₋₆-cycloalkylene moiety), or a cycloalkenylene moiety (preferably a C₃₋₈-cycloalkenylene, such as C₃₋₆-cycloalkenylene moiety) each of which connects the difunctional moiety to the POXZ copolymer (either directly to the end of the POXZ copolymer or, preferably, via a further difunctional moiety). For example, one hydrophobic chain may be attached to the end of the POXZ copolymer via one difunctional moiety (either directly or via an alkylene, cycloalkylene, or cycloalkenylene moiety or via an alkylene, cycloalkylene, or cycloalkenylene moiety which bears another difunctional moiety); two hydrophobic chains may be attached to the end of the POXZ copolymer via two difunctional moieties (which in turn are preferably attached to an alkylene, cycloalkylene, or cycloalkenylene moiety or to an alkylene, cycloalkylene, or cycloalkenylene moiety bearing another difunctional moiety); or two hydrophobic chains may be attached to the end of the POXZ copolymer via the same difunctional moiety (which is then a trifunctional moiety and which may be attached to the end of the POXZ copolymer either directly or via an alkylene, cycloalkylene, or cycloalkenylene moiety or to an alkylene, cycloalkylene, or cycloalkenylene moiety bearing another difunctional moiety). In some embodiments, each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties.

In some embodiments of formula (XXIII′), the cycloalkylene moiety is C₃₋₈-cycloalkylene, such as C₃₋₆-cycloalkylene, e.g., cyclopropylene, cyclobutylene, cyclopentylene, cyclohexylene, wherein the cycloalkylene moiety is optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments of formula (XXIII′), the cycloalkenylene moiety is C₃₋₈-cycloalkenylene, such as C₃₋₆-cycloalkenylene, e.g., cyclopropenylene, cyclobutenylene, cyclopentenylene, cyclohexenylene, wherein the cycloalkenylene moiety is optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments of formula (XXIII′), the alkylene moiety is C₁₋₆-alkylene, such as C₁₋₃-alkylene, e.g., methylene, ethylene, or trimethylene, or C₂₋₃ alkylene.

In some embodiments of formula (XXIII′), the oxazolinylated and/or oxazinylated lipid comprises one of the following structures:

(hydrophobic chain)-(divalent functional moiety)-(POX and/or POZ polymer)

[(hydrophobic chain)-(divalent functional moiety)]₁₋₂-(alkylene moiety)-(divalent functional moiety)-(POX and/or POZ polymer)

(hydrophobic chain)-(divalent functional moiety)-(cycloalkylene moiety)-(divalent functional moiety)-(POX and/or POZ polymer)

(hydrophobic chain)-(divalent functional moiety)-(cycloalkenylene moiety)-(divalent functional moiety)-(POX and/or POZ polymer)

(hydrophobic chain)-(divalent functional moiety)-(alkylene moiety)-(POX and/or POZ polymer)

[(hydrophobic chain)₂-(trivalent functional moiety)]-(alkylene moiety)-(divalent functional moiety)-(POX and/or POZ polymer)

In some embodiments formula (XXIII′), the oxazolinylated and/or oxazinylated lipid has one of the following formulas (XXIIIc′) to (XXIIIh′):

(hydrophobic chain)-(divalent functional moiety)-(POXZ copolymer)-R₁₆ (XXIIIc′)

[(hydrophobic chain)-(divalent functional moiety)]₁₋₂-(alkylene moiety)-(divalent functional moiety)-(POXZ copolymer)-R₁₆ (XXIIId′)

(hydrophobic chain)-(divalent functional moiety)-(cycloalkylene moiety)-(divalent functional moiety)-(POXZ copolymer)-R₁₆ (XXIIIe′)

(hydrophobic chain)-(divalent functional moiety)-(cycloalkenylene moiety)-(divalent functional moiety)-(POXZ copolymer)-R₁₆ (XXIIIf′)

(hydrophobic chain)-(divalent functional moiety)-(alkylene moiety)-(POXZ copolymer)-R₁₆ (XXIIIg′)

[(hydrophobic chain)₂-(trivalent functional moiety)]-(alkylene moiety)-(divalent functional moiety)-(POXZ copolymer)-R₁₆ (XXIIIh′)

In some embodiments of formulas (XXIII), (XXIIIa), and (XXIIIb), L₁₂ comprises at least one ester, sulfide, disulfide, sulfone, orthoester, acylhydrazone, hydrazine, oxime, acetal, ketal, or amide moiety. In some embodiments, L₁₂ is selected from the group consisting of [*—NHC(O)]_{q}(C₁₋₆-alkylene)-, [*—C(O)NH]_{q}(C₁₋₆-alkylene)-, [*—C(O)O]_{q}(C₁₋₆-alkylene)-, [*—OC(O)]_{q}(C₁₋₆-alkylene)-, [*—S]_{q}(C₁₋₆-alkylene)-, [*—SS]_{q}(C₁₋₆-alkylene)-, [*—S(O)₂]ₚ(C₁₋₆-alkylene)-, [(*—O)ₛC(OR₂₅)₃₋₈](C₁-s-alkylene)-, [*—C(OR₂₅)₂O]_{q}(C₁₋₆-alkylene)-, [*—C(R₂₅)(═N—N(R₂₆)C(O)—)]q(C₁₋₃-alkylene)-, [*—C(O)(N(R₂₆)—N═)C(R₂₅)—]q(C₁₋₃-alkylene)-, [*═C(═N—N(R₂₆)C(O)(R₂₅))]_{q}(C₁₋₃-alkylene)-, [*N(R₂₆)N(R₂₆)]_{q}—(C₁₋₆-alkylene)-, [*═C(═N(OH))]_{q}(C₁₋₆-alkylene)-, and [*—OC(R₂₅)(R₂₆)O]_{q}(C₁₋₆-alkylene)-, wherein * represents the attachment point to R₁₇; q is 1 or 2; C₁₋₆-alkylene is either bivalent (if q is 1) or trivalent (if q is 2); R₂₅ is selected from the group consisting of C₁₋₆ alkyl, aryl, and aryl(C₁₋₆ alkyl); R₂₆ is selected from the group consisting of H, C₁₋₆ alkyl, aryl, and aryl(C₁₋₆ alkyl); and s is an integer between 1 and 2. For example, L₁₂ may be selected from the group consisting of [*—NHC(O)]_{q}(C₁₋₃-alkylene)-, [*—C(O)NH]_{q}(C₁₋₃-alkylene)-, [*—C(O)O]_{q}(C₁₋₃-alkylene)-, [*—OC(O)]_{q}(C₁₋₃-alkylene)-, [*—S]_{q}(C₁₋₃-alkylene)-, [*—SS]_{q}(C₁₋₃-alkylene)-, [*—S(O)₂]ₚ(C₁₋₃-alkylene)-, [(*—O)ₛC(OR₂₅)₃₋₈](C₁₋₃-alkylene)-, [*—C(OR₂₅)₂O]_{q}(C₁₋₃-alkylene)-, [*—C(R₂₅)(═N—N(R₂)C(O)—)]q(C₁₋₃-alkylene)-, [*—C(O)(N(R₂₆)—N═)C(R₂₅)—]_{q}(C₁₋₃-alkylene)-, [*═C(═N—N(R₂₆)C(O)(R₂₅))]_{q}(C₁₋₃-alkylene)-, [*N(R₂₆)N(R₂₆)]_{q}(C₁₋₃-alkylene)-, [*═C(═N(OH))]_{q}(C₁₋₃-alkylene)-, and [*—OC(R₂₅)(R₂₆)O]_{q}(C₁₋₃-alkylene)-, wherein * represents the attachment point to R₁₇; q is 1 or 2; C₁₋₃-alkylene is either bivalent (if q is 1) or trivalent (if q is 2); R₂₅ is selected from the group consisting of C₁₋₆ alkyl, aryl, and aryl(C₁₋₈ alkyl); R₂₆ is selected from the group consisting of H, C₁₋₆ alkyl, aryl, and aryl(C₁₋₈ alkyl); and s is an integer between 1 and 2.

In some embodiments of formulas (XXIII), (XXIIIa), and (XXIIIb), R₂₅ is selected from the group consisting of C₁₋₃ alkyl, phenyl, and phenyl(C₁₋₃ alkyl), such as from the group consisting of methyl, ethyl, phenyl, benzyl, and phenylethyl.

In some embodiments of formulas (XXIII), (XXIIIa), and (XXIIIb), R₂₆ is selected from the group consisting of H, C₁₋₃ alkyl, phenyl, and phenyl(C₁₋₃ alkyl), such as from the group consisting of H, methyl, ethyl, phenyl, benzyl, and phenylethyl.

In some embodiments of formulas (XXIII), (XXIIIa), and (XXIIIb), L₁₂ is selected from the group consisting of [*—NHC(O)]_{q}(C₁₋₆alkylene)-, [*—C(O)NH]_{q}(C₁₋₆alkylene)-, [*—C(O)O]_{q}(C₁₋₆-alkylene)-, [*—OC(O)]_{q}(C₁₋₆-alkylene)-, [*—S]_{q}(C₁₋₆-alkylene)-, and [*—S(O)₂]ₚ(C₁₋₆-alkylene)-, preferably from the group consisting of [*—NHC(O)]_{q}C₁₋₆-alkylene)-, [*—C(O)O]_{q}(C₁₋₆-alkylene)-, [*—OC(O)]_{q}(C₁₋₆-alkylene)-, [*—S]_{q}(C₁₋₆-alkylene)-, and [*—S(O)₂]ₚ(C₁₋₆alkylene)-, more preferably from the group consisting of [*—NHC(O)]_{q}(C₁₋₆-alkylene)- and [*—C(O)O]_{q}(C₁₋₆alkylene)-, wherein * represents the attachment point to R₁₇; q is 1 or 2; and C₁₋₆-alkylene is either bivalent (if q is 1) or trivalent (if q is 2).

In some embodiments of formulas (XXIII), (XXIII a), and (XXIII b), L₁₂ is selected from the group consisting of [*—NHC(O)]_{q}(C₁₋₃-alkylene)-, [*—C(O)NH]_{q}(C₁₋₃-alkylene)-, [*—C(O)O]_{q}(C₁₋₃-alkylene)-, [*—OC(O)]_{q}(C₁₋₃-alkylene)-, [*—S]_{q}(C₁₋₃-alkylene)-, and [*—S(O)₂]ₚ(C₁₋₃-alkylene)-, preferably from the group consisting of [*—NHC(O)]_{q}(C₁₋₃-alkylene)-, [*—C(O)O]_{q}C₁₋₃-alkylene)-, [*—OC(O)]_{q}(C₁₋₃-alkylene)-, [*—S]_{q}(C₁₋₃-alkylene)-, and [*—S(O)₂]ₚ(C₁₋₃-alkylene)-, more preferably from the group consisting of [*—NHC(O)]_{q}(C₁₋₃-alkylene)- and [*—C(O)O]_{q}(C₁₋₃-alkylene)-, wherein * represents the attachment point to R₁₇; q is 1 or 2; and C₁₋₃-alkylene is either bivalent (if q is 1) or trivalent (if q is 2).

In some embodiments of formulas (XXIII), (XXIIIa), and (XXIIIb), L₁₂ is selected from the group consisting of *—NHC(O)—(CH₂)—, *—NHC(O)—(CH₂)₂—, *—C(O)NH—(CH₂)—, *—C(O)NH—(CH₂)₂—, —(CH₂)—CH(OC(O)—*)(CH₂OC(O)—*), —(CH₂)—CH(S—*)₂, —(CH₂)—CH(S—*)—CH₂(S—*), *—S—(CH₂)₃—, *—S(O)₂—(CH₂)₃—, and *—OC(O)—(CH₂)—, wherein * represents the attachment point to R₁₇. Thus, R₁₅ may be selected from the group consisting of R₁₇, -L₁₂R₁₇, —(CH₂)—CH(OC(O)R₁₇)(CH₂OC(O)R₁₇), —(CH₂)—CH(SR₁₇)₂, and —(CH₂)—CH(SR₁₇)—CH₂(SR₁₇); and L₁₂ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)—, preferably L₁₂ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—, wherein * represents the attachment point to R₁₇.

In some embodiments of formulas (XXIII), (XXIIIa), and (XXIIIb), R₁₅ is -L₁₂(R₁₅)_{q}, i.e., the POXZ copolymer is conjugated to the one or more hydrophobic chains (i.e., R₁₇) via the linker L₁₂.

In some embodiments of formulas (XXIII′), (XXIIIc′), (XXIIId′), (XXIIIe′), (XXIIIf′), (XXIIIg′) and (XXIIIh′), L₁₂ is selected from the group consisting of [*—Z₁]q(C₁-alkylene)-Z₁—, *—Z₁—(C₃₋₈-cycloalkylene)-Z₁—, *—Z₁—(C₃₋₈-cycloalkenylene)-Z₁—, (*═N)(C₁₋₆-alkylene)-Z₁—, *—Z₁(C₁₋₆-alkylene)-, and *—Z₁—, wherein * represents the attachment point to R₁₇; q is 1 or 2; C₁₋₆-alkylene is either bivalent (if q is 1) or trivalent (if q is 2); each of the C₃₋₈-cycloalkylene and C₃₋₈-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z₁ is independently selected from the group consisting of —OP(O)₂O(C₁₋₃-alkylene)NH—, —NH(C₁₋₃-alkylene)OP(O)₂O—, —C(O)NH—, —NHC(O)—, —OC(O)NH—, —NHC(O)O—, —O—, —C(O)O—, —OC(O)—, —S—, —S(O)₂—, and —NR₂₂. For example, L₁₂ can be selected from the group consisting of [*—C(O)O]_{q}(C₁₋₆-alkylene)-Z₁—, (*—NH)(C₁₋₆alkylene)-Z₁—, (*═N)(C₁-alkylene)-Z₁—, (*—NH)C(O)(C₁₋₆-alkylene)-Z₁—, (*—C(O)NH(C₁₋₆-alkylene)-Z₁—, (*—NH)C(O)(C₁₋₆alkylene)-, (*—C(O)NH(C₁₋₆-alkylene)-, *—Z₁—(C₃₋₈-cycloalkenylene)-Z₁—, *—S—, *—S(O)₂—, (*—NH)C(O)—, and *—C(O)NH—, wherein * represents the attachment point to R₁₇; q is 1 or 2; C₁₋₆-alkylene is either bivalent (if q is 1) or trivalent (if q is 2); and Z₁ is selected from the group consisting of —OP(O)₂O(C₁₋₃-alkylene)NH—, —NH(C₁₋₃-alkylene)OP(O)₂O—, —OC(O)NH—, —NHC(O)O—, —O—, —S—, and —NH—

In some embodiments of formulas (XXIII′), (XXIIIc′), (XXIIId′), (XXIIIe′), (XXIIIf′), (XXIIIg′) and (XXIIIh′), L₁₂ is selected from the group consisting of [*—Z₁]_{q}(C₁₋₃-alkylene)-Z₁—, *—Z₁—(C₃₋₆-cycloalkylene)-Z₁—, *—Z₁—(C₃₋₆-cycloalkenylene)-Z₁—, (*═N)(C₁₋₃-alkylene)-Z₁—, *—Z₁(C₁₋₃-alkylene)-, and *—Z₁—, wherein * represents the attachment point to R₁₇; q is 1 or 2; C₁₋₃-alkylene is either bivalent (if q is 1) or trivalent (if q is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z₁ is independently selected from the group consisting of —OP(O)₂O(C₁₋₂-alkylene)NH—, —NH(C₁₋₂-alkylene)OP(O)₂O—, —C(O)NH—, —NHC(O)—, —OC(O)NH—, —NHC(O)O—, —O—, —C(O)O—, —OC(O)—, —S—, —S(O)₂—, and —NR₂₂. For example, L₁₂ can be selected from the group consisting of [*—C(O)O]_{q}(C₁₋₃-alkylene)-Z₁—, (*—NH)(C₁₋₃-alkylene)-Z₁—, (*═N)(C₁₋₃-alkylene)-Z₁—, (*—NH)C(O)(C₁₋₃-alkylene)-Z₁—, (*—C(O)NH(C₁₋₃-alkylene)-Z₁—, (*—NH)C(O)(C₁₋₃-alkylene)-, (*—C(O)NH(C₁₋₃-alkylene)-, *—Z₁—(C₃₋₆-cycloalkenylene)-Z₁—, *—S—, *—S(O)₂—, (*—NH)C(O)—, and *—C(O)NH—, wherein * represents the attachment point to R₁₇; q is 1 or 2; C₁₋₃-alkylene is either bivalent (if q is 1) or trivalent (if q is 2); and Z₁ is selected from the group consisting of —OP(O)₂O(C₁₋₂-alkylene)NH—, —NH(C₁₋₂-alkylene)OP(O)₂O—, —OC(O)NH—, —NHC(O)O—, —O—, —S—, and —NH-

In some embodiments of formulas (XXIII′), (XXIIIc′), (XXIIId′), (XXIIIe′), (XXIIIf′), (XXIIIg′) and (XXIIIh′), L₁₂ is selected from the group consisting of [*—Z₁]q(C₁₋₃-alkylene)-Z₁—, *—Z₁—(C₃₋₆-cycloalkylene)-Z₁—, *—Z₁—(C₃₋₆-cycloalkenylene)-Z₁—, (*═N)(C₁₋₃-alkylene)-Z₁—, *—Z₁(C₁₋₃-alkylene)-, and *—Z₁—, wherein * represents the attachment point to R₁₇; p is 1 or 2; C₁₋₃-alkylene is either bivalent (if q is 1) or trivalent (if q is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z₁ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—. For example, L₁₂ can be selected from the group consisting of [*—C(O)O]_{q}(C₁₋₃-alkylene)-Z₁—, (*—NH)(C₁₋₃-alkylene)-Z₁—, (*═N)(C₁₋₃-alkylene)-Z₁—, (*—NH)C(O)(C₁₋₃-alkylene)-Z₁—, (*—C(O)NH(C₁₋₃-alkylene)-Z₁—, (*—NH)C(O)(C₁₋₃-alkylene)-, (*—C(O)NH(C₁₋₃-alkylene)-, *—Z₁—(C₃₋₆-cycloalkenylene)-Z₁—, *—S—, *—S(O)₂—, (*—NH)C(O)—, and *—C(O)NH—, wherein * represents the attachment point to R₁₇; p is 1 or 2; C₁₋₃-alkylene is either bivalent (if q is 1) or trivalent (if q is 2); and Z₁ is selected from the group consisting of —OP(O)₂O(CH₂)₂NH—, —NH(CH₂)₂OP(O)₂O—, —OC(O)NH—, —NHC(O)O—, —O—, —S—, and —NH—.

In some embodiments of formulas (XXIII′), (XXIIIc′), (XXIIId′), (XXIIIe′), (XXIIIf′), (XXIIIg′) and (XXIIIh′), L₁₂ is selected from the group consisting of (*—C(O)O)(CH(OC(O)—*))(CH₂)—Z₁—, (*═N)(C₁₋₃-alkylene)-NHC(O)—, (*—Z₁(C₁₋₃-alkylene)-Z₁—, *—Z₁—(C₃₋₆cycloalkenylene)-Z₁—, and *—Z₁—, wherein * represents the attachment point to R₁₇; the C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z₁ is independently selected from the group consisting of —OP(O)₂O(CH₂)₂—NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—. For example, L₁₂ can be selected from the group consisting of (*—C(O)O)(CH(OC(O)—*))(CH₂)—Z₁—, (*═N)(C₁₋₃-alkylene)-NHC(O)—, (*—NH)(C₁₋₃-alkylene)-NHC(O)—, *—Z₁—(C₃₋₆-cycloalkenylene)-Z₁—, *—S—, *—S(O)₂—, and *—C(O)NH—, wherein * represents the attachment point to R₁₇; and Z₁ is selected from the group consisting of —OP(O)₂O(CH₂)₂NH—, —NH(CH₂)₂OP(O)₂—O—, —OC(O)NH—, —NHC(O)O—, —O—, —S—, and —NH—. Thus, in some embodiments of formulas (XXIII′), (XXIIIc′), (XXIIId′), (XXIIIe′), (XXIIIf′), (XXIIIg′) and (XXIIIh′), R₁₅ is selected from the group consisting of (R₁₇C(O)O)(CH(OC(O)R₁₇))(CH₂)—Z₁—, (R₁₇)₂N(C₁₋₃-alkylene)-Z₁—, R₁₇Z₁(C₁₋₃-alkylene)-Z₁—, R₁₇Z₁—(C₃₋₆-cycloalkenylene)-Z₁—, and R₁₇Z₁—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z₁ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—. For example, R₁₅ can be selected from the group consisting of (R₁₇C(O)O)(CH(OC(O)R₁₇))(CH₂)—Z₁—, (R₁₇)₂N(C₁₋₃-alkylene)-NHC(O)—, R₁₇NH(C₁₋₃-alkylene)NHC(O)—, R₁₇Z₁—(C₃₋₆-cycloalkenylene)-Z₁—, R₁₇S—, R₁₇S(O)₂—, and R₁₇C(O)NH—, wherein Z₁ is selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—.

In some embodiments of formulas (XXIII′), (XXIIIc′), (XXIIId′), (XXIIIe′), (XXIIIf′), (XXIIIg′) and (XXIIIh′), R₁₅ is -L₁₂(R₁₅)_{q}, i.e., the POXZ copolymer is conjugated to the one or more hydrophobic chains (i.e., R₁₇) via the linker L₁₂.

In any of the above embodiments of formulas (XXIII), (XXIIIa), (XXIIIb), (XXIII′), (XXIIIc′), (XXIIId′), (XXIIIe′), (XXIIIf′), (XXIIIg′) and (XXIIIh′), R₁₇ preferably is independently a non-cyclic, preferably straight hydrocarbyl group. For example, each R₁₇ preferably is independently a hydrocarbyl group having at least 8 carbon atoms, such as at least 10 carbon atoms, preferably up to 30 carbon atoms, such as up to 28, 26, 24, 22, or 20 carbon atoms, or up to 16 carbon atoms, such as up to 15, 14, 13, 12, 11, or 10 carbon atoms. In some embodiments, each R₁₇ is a hydrocarbyl group having 10 to 16 carbon atoms, such as 10 to 15 or 10 to 14 carbon atoms. In some embodiments, each R₁₇ is a straight hydrocarbyl group having 10 to 16 carbon atoms, such as 10 to 15 or 10 to 14 carbon atoms.

In any of the above embodiments of formulas (XXIII′), (XXIIIc′), (XXIIId′), (XXIIIe′), (XXIIIf′), (XXIIIg′) and (XXIIIh′), each R₁₇ may preferably be a hydrocarbyl group having to 18 carbon atoms, such as a straight alkyl group having 10 to 18 carbon atoms or a straight alkenyl group having 10 to 18 carbon atoms. For example, straight alkenyl group may have 10, 11, 12, 13, 14, 15, 16, 17, or 18 carbon atoms and 1, 2, or 3 carbon-carbon double bonds.

In any of the above embodiments of formulas (XXIII), (XXIIIa), (XXIIIb), (XXIII′), (XXIIIc′), (XXIIId′), (XXIIIe′), (XXIIIf′), (XXIIIg′) and (XXIIIh′), R₁₆ is preferably selected from the group consisting of H, C₁₋₃ alkyl, —OR₂₀, —N₃, C₂₋₆ alkynyl, —OC(O)R₂₁, —C(O)R₂₁, —NR₂₂R₂₃, —COOH, —C(O)NR₂₂R₂₃, —NR₂₂C(O)R₂₁, and a member of a targeting pair, wherein the C₁₋₃ alkyl group is optionally substituted with one or more substituents independently selected from the group consisting of —OH, SH, halogen, —CN, —N₃, C₂₋₆ alkynyl, —COOH, —NR₂₂R₂₃, —C(O)NR₂₂R₂₃, —NR₂₂C(O)R₂₁, and a member of a targeting pair; R₂₀ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, —COOH, —NR₂₂R₂₃, and a member of a targeting pair; R₂₁ is selected from the group consisting of C₁₋₃ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR₂₂R₂₃, and a member of a targeting pair; and each of R₂₂ and R₂₃ is independently selected from the group consisting of H, C₁₋₆ alkyl, C₂₋₆ alkenyl, and C₂₋₆ alkynyl, or R₂₂ and R₂₃ may join together with the nitrogen atom to which they are attached to form a heterocyclyl group, wherein each of the C₁₋₆ alkyl, C₂₋₆ alkenyl, and C₂₋₆ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair.

In any of the above embodiments of formulas (XXIII), (XXIIIa), (XXIIIb), (XXIII′), (XXIIIc′), (XXIIId′), (XXIIIe′), (XXIIIf′), (XXIIIg′) and (XXIIIh′), R₁₆ is preferably selected from the group consisting of H, C₁₋₃ alkyl, —OR₂₀, —N₃, C₂₋₆ alkynyl, —OC(O)R₂₁, —C(O)R₂₁, —NR₂₂R₂₃, —COOH, —C(O)NR₂₂R₂₃, —NR₂₂C(O)R₂₁, and a member of a targeting pair, wherein the C₁₋₃ alkyl group is optionally substituted with one or more substituents independently selected from the group consisting of —OH, —N₃, C₂₋₆ alkynyl, —COOH, —NH₂, —NHCH₃, —N(CH₃)₂, —C(O)NR₂₂R₂₃, —NR₂₂C(O)R₂₁, and a member of a targeting pair; R₂₀ is selected from the group consisting of H and C₁₋₃ alkyl; R₂₁ is C₁₋₃ alkyl optionally substituted with one or more substituents independently selected from the group consisting of —OH, SH, halogen, —CN, —N₃, C₂₋₆ alkynyl, —COOH, —NR₂₂R₂₃, and a member of a targeting pair; and each of R₂₂ and R₂₃ is independently selected from the group consisting of H, C₁₋₃ alkyl, C₂₋₃ alkenyl, and C₂₋₃ alkynyl, wherein each of the C₁₋₃ alkyl, C₂₋₃ alkenyl, and C₂₋₃ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair, or R₂₂ and R₂₃ may join together with the nitrogen atom to which they are attached to form a 5- or 6-membered heterocyclyl group.

In any of the above embodiments of formulas (XXIII), (XXIIIa), (XXIIIb), (XXIII′), (XXIIIc′), (XXIIId′), (XXIIIe′), (XXIIIf′), (XXIIIg′) and (XXIIIh′), R₁₆ is preferably selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O)CH₃, —C(O)NH₂, —C(O)NHCH₃, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair.

In some embodiments of formulas (XXIII), (XXIIIa), and (XXIIIb), R₁₆ is selected from the group consisting of H, —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O)CH₃, —NH(CH₂CH₃), —C(O)NH₂, —C(O)NHCH₃, —OC(O)(CH₂)₂COOH, and a member of a targeting pair. In some embodiments of formulas (XXIII), (XXIIIa), and (XXIIIb), R₁₆ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O)CH₃, —NH(CH₂CH₃), —C(O)NH₂, —C(O)NHCH₃, —OC(O)(CH₂)₂COOH, and a member of a targeting pair. In some embodiments of formulas (XXIII), (XXIIIa), and (XXIIIb), R₁₆ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O)CH₃, —NH(CH₂CH₃), —OC(O)(CH₂)₂COOH, and a member of a targeting pair. In some embodiments of formulas (XXIII), (XXIIIa), and (XXIIIb), R₁₆ is selected from the group consisting of —OH, —N₃, —NH₂, —NHC(O)(CH₂)₂COOH, —N(CH₂CH₃)C(O)(CH₂)₂COOH, —N(CH₂CH₃)C(O)CH₃, —NH(CH₂CH₃), and —OC(O)(CH₂)₂COOH.

In some embodiments of formulas (XXIII′), (XXIIIc′), (XXIIId′), (XXIIIe′), (XXIIIf′), (XXIIIg′) and (XXIIIh′), R₁₆ is C₁₋₃ alkyl optionally substituted with one or two substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair. In some embodiments of formulas (XXIII′), (XXIIIc′), (XXIIId′), (XXIIIe′), (XXIIIf′), (XXIIIg′) and (XXIIIh′), R₁₆ is C₁₋₃ alkyl optionally substituted with one substituent selected from the group consisting of —COOH and —C(O)NH(CH₂)₂NH₂.

In certain embodiments, the oxazolinylated and/or oxazinylated lipid has the following general formula (XXIV):

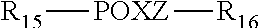

wherein:
R₁₅, when attached to the N-end of the POXZ copolymer, is selected from the group consisting of -L₁₂R₁₇, —(CH₂)—CH(OC(O)R₁₇)(CH₂OC(O)R₁₇), —(CH₂)—CH(SR₁₇)₂, and —(CH₂)—CH(SR₁₇)—CH₂(SR₁₇), wherein each R₁₇ is independently a straight hydrocarbyl group having at least 10 carbon atoms (preferably having 10 to 15 carbon atoms); and L₁₂ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L₁₂ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R₁₇, or R₁S, when attached to the C-end of the POXZ copolymer, is selected from the group consisting of (R₁₇C(O)O)(CH(OC(O)R₁₇))(CH₂)—Z₁—, (R₁₇)₂N(C₁₋₃-alkylene)-Z₁—, R₁₇Z₁(C₁₋₃-alkylene)-Z₁—, R₁₇Z₁—(C₃₋₆-cycloalkenylene)-Z₁—, and R₁₇Z₁—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z₁ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—; POXZ is a copolymer containing the repeating units of the following general formulas (XXa) and (XXb):

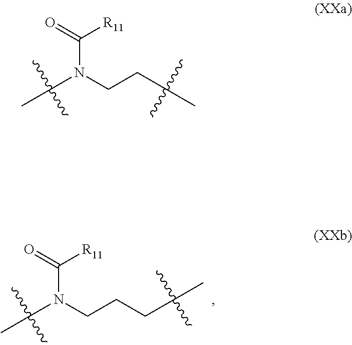

wherein each of R₁₁ is independently methyl or ethyl, and is independently selected for each repeating unit; the number of repeating units of formula (XXa) in the copolymer is 1 to 99 (preferably 1 to 79, 1 to 69, or 1 to 49); the number of repeating units of formula (XXb) in the copolymer is 1 to 99 (preferably 1 to 79, 1 to 69, or 1 to 49); the sum of the number of repeating units of formula (XXa) and the number of repeating units of formula (XXb) in the copolymer is 10 to 100 (preferably 20 to 80, 30 to 70, or 40 to 50); and the repeating units of formulas (XXa) and (XXb) are arranged in a random, periodic, alternating or block wise manner; and
R₁₆ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O)CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair.

Those embodiments of formula (XXIV), where R₁₅ is attached to the C-end (i.e., the terminal C atom) of the POXZ copolymer and R₁₆ is attached to the N-end (i.e., the terminal N atom) of the POXZ copolymer), are designated as formula (XXIV′) herein.

In some embodiments of formula (XXIV) or (XXIV′), each R₁₁ is methyl or each R₁₁ is ethyl. In some alternative embodiments of formula (XXIV) or (XXIV′), R₁₁ is independently selected from methyl and ethyl for each repeating unit, wherein in at least one repeating unit R₁₁ is methyl, and in at least one repeating unit R₁₁ is ethyl.

In some embodiments of formula (XXIV), where R₁₅ is attached to the N-end of the POXZ copolymer, R₁₅ is selected from the group consisting of R₁₇—NHC(O)—(CH₂)—, R₁₇—NHC(O)—(CH₂)₂—, —(CH₂)—CH(OC(O)R₁₇)(CH₂OC(O)R₁₇), —(CH₂)—CH(SR₁₇)—CH₂(SR₁₇), R₁₇S—(CH₂)₃—, R₁₇S(O)₂—(CH₂)₃—, and R₁₇—OC(O)—(CH₂)—; and/or R₁₆ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O)CH₃, —NH(CH₂CH₃), —OC(O)(CH₂)₂COOH, and a member of a targeting pair (e.g., R₁₆ is selected from the group consisting of —OH, —N₃, —NH₂, —NHC(O)(CH₂)₂COOH, —N(CH₂CH₃)C(O)(CH₂)₂COOH, —N(CH₂CH₃)C(O)CH₃, —NH(CH₂CH₃), and —OC(O)(CH₂)₂COOH).

In some embodiments of formula (XXIV′) (i.e., R₁₅ is attached to the C-end of the POXZ copolymer), R₁₅ is selected from the group consisting of R₁₇C(O)NH—, R₁₇S—, R₁₇S(O)₂—, and R₁₇NH—(3,4-dioxocyclobut-1-en-1,2-diyl)-NH—; and/or R₁₆ is C₁₋₃ alkyl optionally substituted with one or two substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair (e.g., R₁₆ is C₁₋₃ alkyl optionally substituted with one substituent selected from the group consisting of —COOH and —C(O)NH(CH₂)₂NH₂).

In some embodiments, the oxazolinylated and/or oxazinylated lipid has one of the following formulas (XXV), (XXV′), (XXVI), or (XXVI′):

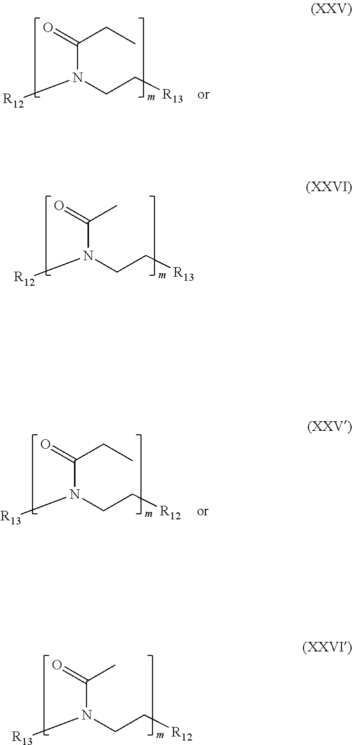

wherein R₁₂, R₁₃, and m are as specified above, in particular with respect to any of the formulas (XXI), (XXI′), (XXIa), (XXIa′), (XXIc), (XXId), (XXIe′), (XXIf′), (XXIg′), (XXIh′), (XXIi′), (XXIj′), (XXII), (XXII′), (XXIIa), and (XXIIa′).

In any of the above embodiments of formulas (XXV), (XXV′), (XXVI), and (XXVI′), m preferably is 10 to 100, such as 20 to 80, 30 to 70, or 40 to 50, e.g., 20 to 25 or 45 to 50.

In some embodiments, the oxazolinylated and/or oxazinylated lipid has one of the following formulas (XXVa), (XXVa′), (XXVIa), or (XXVIa′):

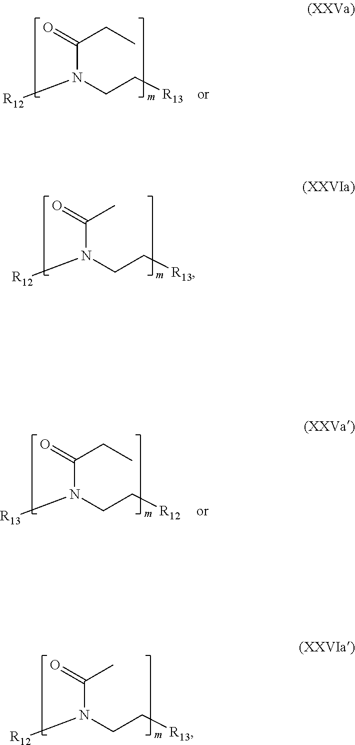

wherein m is either 20 to 25 or 45 to 50; and R₁₂ and R₁₃ are as specified above, in particular with respect to any of the formulas (XXI), (XXI′), (XXIa), (XXIa′), (XXIc), (XXId), (XXIe′), (XXIf′), (XXIg′), (XXIh′), (XXIi′), (XXIj′), (XXII), (XXII′), (XXIIa), and (XXIIa′).

In any of the above embodiments of formulas (XXV), (XXVa), (XXVI), and (XXVIa), R₁₂ is preferably selected from the group consisting of -L₁₁R₁₄, —(CH₂)—CH(OC(O)R₁₄)(CH₂OC(O)R₁₄), —(CH₂)—CH(SR₁₄)₂, and —(CH₂)—CH(SR₁₄)—CH₂(SR₁₄), wherein each R₁₄ is independently a straight hydrocarbyl group having at least 10 carbon atoms (and preferably up to 30 carbon atoms, such as up to 24 carbon atoms, e.g., having 10 to 16 carbon atoms such as 10 to 14 carbon atoms); and L₁₁ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L₁₁ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R₁₄. Thus, in any of the above embodiments of formulas (XXV), (XXVa), (XXVI), and (XXVIa), R₁₂ may be selected from the group consisting of R₁₄—NHC(O)—(CH₂)—, R₁₄—NHC(O)—(CH₂)₂—, —(CH₂)—CH(OC(O)R₁₄)(CH₂OC(O)R₁₄), —(CH₂)—CH(SR₁₄)—CH₂(SR₁₄), R₁₄S—(CH₂)₃—, R₁₄S(O)₂—(CH₂)₃—, and R₁₄—OC(O)—(CH₂)—.

In any of the above embodiments of formulas (XXV′), (XXVa′), (XXVI′), and (XXVIa′), R₁₂ is preferably selected from the group consisting of (R₁₄C(O)O)(CH(OC(O)R₁₄))(CH₂)—Z—, (R₁₄)₂N(C₁₋₃-alkylene)-Z—, R₁₄Z(C₁₋₃-alkylene)-Z—, R₁₄Z—(C₃₋₆-cycloalkenylene)-Z—, and R₁₄Z—, wherein each R₁₄ is independently a straight hydrocarbyl group having at least 10 carbon atoms (and preferably up to 30 carbon atoms, such as up to 24 carbon atoms, e.g., having 10 to 16 carbon atoms such as 10 to 14 carbon atoms); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—. Thus, in any of the above embodiments of formulas (XXV′), (XXVa′), (XXVI′), and (XXVIa′), R₁₂ may be selected from the group consisting of R₁₄C(O)NH—, R₁₄S—, R₁₄S(O)₂—, and R₁₄NH—(3,4-dioxocyclobut-1-en-1,2-diyl)-NH—.

In any of the above embodiments of formulas (XXV), (XXV′), (XXVa), (XXVa′), (XXVI), (XXVI′), (XXVIa), and (XXVIa′), R₁₃ is preferably selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O)CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair.

In any of the above embodiments of formulas (XXV), (XXVa), (XXVI), and (XXVIa), R₁₃ is preferably selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O)CH₃, —NH(CH₂CH₃), —OC(O)(CH₂)₂COOH, and a member of a targeting pair, such as from the group consisting of —OH, —N₃, —NH₂, —NHC(O)(CH₂)₂COOH, —N(CH₂CH₃)C(O)(CH₂)₂COOH, —N(CH₂CH₃)C(O)CH₃, —NH(CH₂CH₃), and —OC(O)(CH₂)₂COOH.

In any of the above embodiments of formulas (XXV′), (XXVa′), (XXVI′), and (XXVIa′), R₁₃ is preferably C₁₋₃ alkyl optionally substituted with one or two substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair, more preferably R₁₃ is C₁₋₃ alkyl optionally substituted with one substituent selected from the group consisting of —COOH and —C(O)NH(CH₂)₂NH₂.

In any of the above embodiments of formulas (XXV), (XXVa), (XXVI), and (XXVIa), it is preferred that:
R₁₂ is selected from the group consisting of -L₁₁R₁₄, —(CH₂)—CH(OC(O)R₁₄)(CH₂OC(O)R₁₄), —(CH₂)—CH(SR₁₄)₂, and —(CH₂)—CH(SR₁₄)—CH₂(SR₁₄), wherein each R₁₄ is independently a straight hydrocarbyl group having at least 10 carbon atoms (and preferably up to 30 carbon atoms, such as up to 24 carbon atoms, or up to 16 carbon atoms, such as up to 15, 14, 13, 12, 11, or 10 carbon atoms, e.g., having 10 to 16 carbon atoms, such as 10 to 15 or 10 to 14 carbon atoms); and L₁₁ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L₁₁ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R₁₄; and
R₁₃ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O)CH₃, —NH(CH₂CH₃), —OC(O)(CH₂)₂COOH, and a member of a targeting pair, such as from the group consisting of —OH, —N₃, —NH₂, —NHC(O)(CH₂)₂COOH, —N(CH₂CH₃)C(O)(CH₂)₂COOH, —N(CH₂CH₃)C(O)CH₃, —NH(CH₂CH₃), and —OC(O)(CH₂)₂COOH.

In any of the above embodiments of formulas (XXV′), (XXVa′), (XXVI′), and (XXVIa′), it is preferred that:
R₁₂ is selected from the group consisting of (R₁₄C(O)O)(CH(OC(O)R₁₄))(CH₂)—Z—, (R₁₄)₂N(C₁₋₃-alkylene)-Z—, R₁₄Z(C₁₋₃-alkylene)-Z—, R₁₄Z—(C₃₋₆-cycloalkenylene)-Z—, and R₁₄Z—, wherein each R₁₄ is independently a straight hydrocarbyl group having at least 10 carbon atoms (and preferably up to 30 carbon atoms, such as up to 24 carbon atoms, e.g., having 10 to 16 carbon atoms such as 10 to 14 carbon atoms); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—; and
R₁₃ is C₁₋₃ alkyl optionally substituted with one or two substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair, preferably R₁₃ is C₁₋₃ alkyl optionally substituted with one substituent selected from the group consisting of —COOH and —C(O)NH(CH₂)₂NH₂.

In some embodiments, the oxazolinylated and/or oxazinylated lipid has one of the following formulas (XXVII), (XXVII′), (XXVIII), or (XXVIII′):

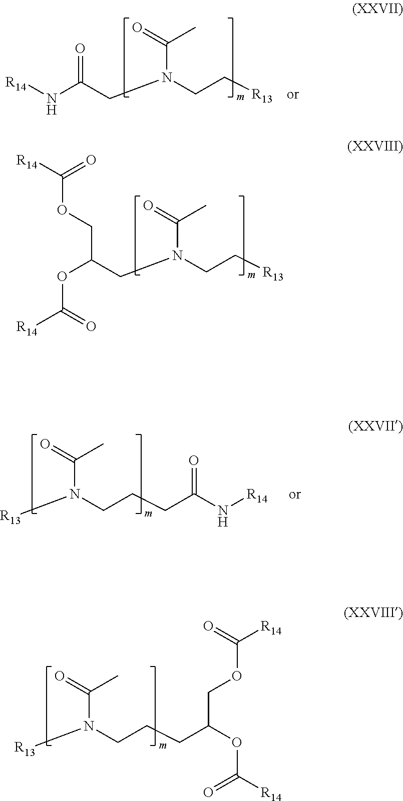

wherein R₁₃, R₁₄, and m are as specified above, in particular with respect to any of the formulas (XXI), (XXI′), (XXIa), (XXIa′), (XXII), (XXII′), (XXIIa), and (XXIIa′). In some embodiments of formula (XXVII) or (XXVII′), R₁₄ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms. In some embodiments of formula (XXVIII) or (XXVIII′), R₁₄ is independently a straight hydrocarbyl group having at least 10 carbon and up to 30 carbon atoms, such as up to 24, 22, or 20 carbon atoms, e.g., having 10 to 16 carbon atoms, such as 10 to or 10 to 14 carbon atoms.

In any of the above embodiments of formulas (XXVII), (XXVII′), (XXVIII), or (XXVIII′), m preferably is 10 to 100, such as 20 to 80, 30 to 70, or 40 to 50, e.g., 20 to 25 or 45 to 50.

In some embodiments, the oxazolinylated and/or oxazinylated lipid has one of the following formulas (XXVIIa) or (XXVIIIa):

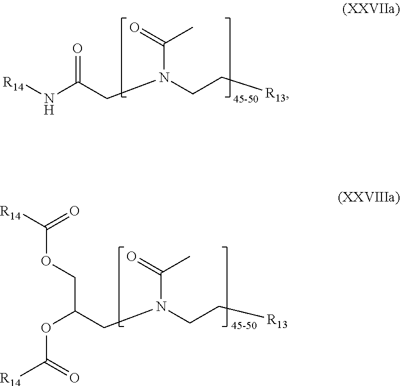

wherein R₁₃, R₁₄, and m are as specified above, in particular with respect to any of the formulas (XXI), (XXI′), (XXIa), (XXIa′), (XXII), (XXII′), (XXIIa), and (XXIIa′).

In any of the above embodiments of formulas (XXVII), (XXVII′), (XXVIIa), (XXVIII), (XXVIII′), and (XXVIIIa), each R₁₄ is preferably independently a straight hydrocarbyl group having at least 10 carbon atoms (and preferably up to 30 carbon atoms, such as up to 24 carbon atoms, e.g., having 10 to 16 carbon atoms). In some embodiments of formulas (XXVII), (XXVII′), and (XXVIIa), R₁₄ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms.

In some embodiments of formulas (XXVIII), (XXVIII′), and (XXVIIIa), R₁₄ is independently a straight hydrocarbyl group having at least 10 carbon and up to 30 carbon atoms, such as up to 24, 22, or 20 carbon atoms, e.g., having 10 to 16 carbon atoms, such as 10 to or 10 to 14 carbon atoms.

In any of the above embodiments of formulas (XXVII), (XXVII′), (XXVIIa), (XXVIII), (XXVIII′), and (XXVIIIa), R₁₃ is preferably selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O)CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair.

In any of the above embodiments of formulas (XXVII), (XXVII′), (XXVIIa), (XXVIII), (XXVIII′), and (XXVIIIa), it is preferred that: each R₁₄ is independently a straight hydrocarbyl group having at least 10 carbon atoms (and preferably up to 30 carbon atoms, such as up to 24 carbon atoms, e.g., having 10 to 16 carbon atoms, such as 10 to 14 carbon atoms); and
R₁₃ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O)CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair.

In this respect, for formulas (XXVII′) and (XXVIII′), it is preferred that R₁₃ is C₁₋₃ alkyl optionally substituted with one or two substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair, more preferably R₁₃ is C₁₋₃ alkyl optionally substituted with one substituent selected from the group consisting of —COOH and —C(O)NH(CH₂)₂NH₂.

In any of the above embodiments of formulas (XXVII), (XXVII′), and (XXVIIa), it is preferred that:
each R₁₄ is independently a straight hydrocarbyl group having at least 10 carbon atoms (and preferably up to 16 carbon atoms, such as up to 15, 14, 13, 12, 11, or 10 carbon atoms, e.g., having 10 to 16 carbon atoms, such as 10 to 15 or 10 to 14 carbon atoms); and
R₁₃ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O)CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair.

In this respect, for formula (XXVII′), it is preferred that R₁₃ is C₁₋₃ alkyl optionally substituted with one or two substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair, more preferably R₁₃ is C₁₋₃ alkyl optionally substituted with one substituent selected from the group consisting of —COOH and —C(O)NH(CH₂)₂NH₂.

In any of the above embodiments of formulas (XXVIII), (XXVIII′), and (XXVIIIa), it is preferred that:
each R₁₄ is independently a straight hydrocarbyl group having at least 10 carbon atoms (and preferably up to 30 carbon atoms, such as up to 24 carbon atoms, e.g., having 10 to 15 carbon atoms); and
R₁₃ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O)CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair.

In this respect, for formula (XXVIII′), it is preferred that R₁₃ is C₁₋₃ alkyl optionally substituted with one or two substituents independently selected from the group consisting of —OH, —N₃, C₂₋₆ alkynyl, —COOH, —NH₂, —NHCH₃, —N(CH₃)₂, —C(O)NH₂, —C(O)NHCH₃, —C(O)NH(CH₂)₂NH₂, and a member of a targeting pair, more preferably R₁₃ is C₁₋₃ alkyl optionally substituted with one substituent selected from the group consisting of —COOH and —C(O)NH(CH₂)₂NH₂.

Particular examples of the oxazolinylated and/or oxazinylated lipid are the following compounds (XXI-1) to (XXI-34) and (XXI′-35) to (XXI′-48):

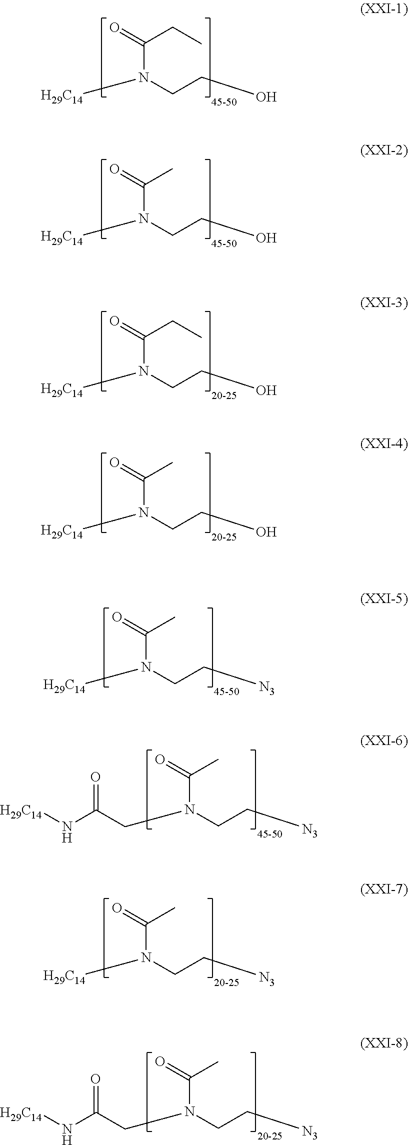

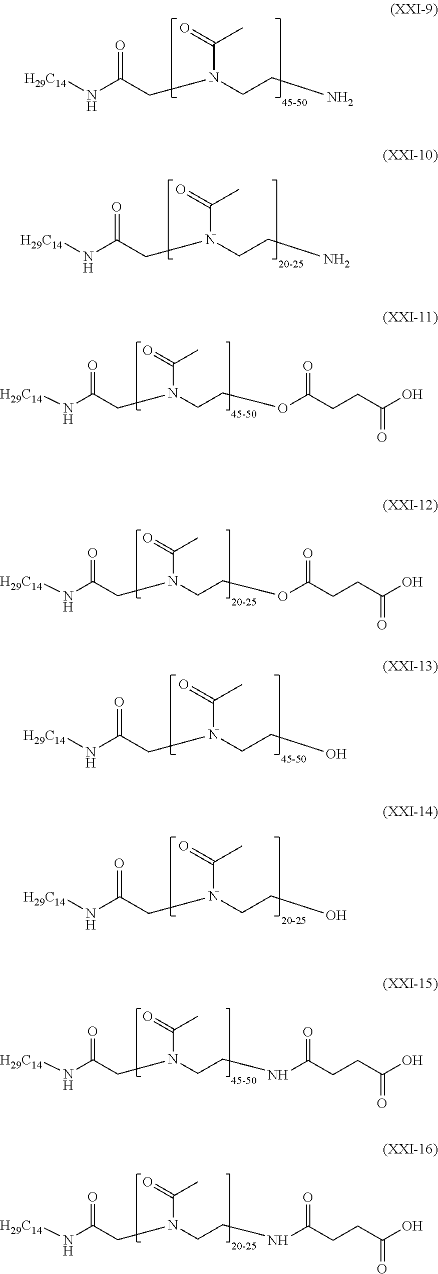

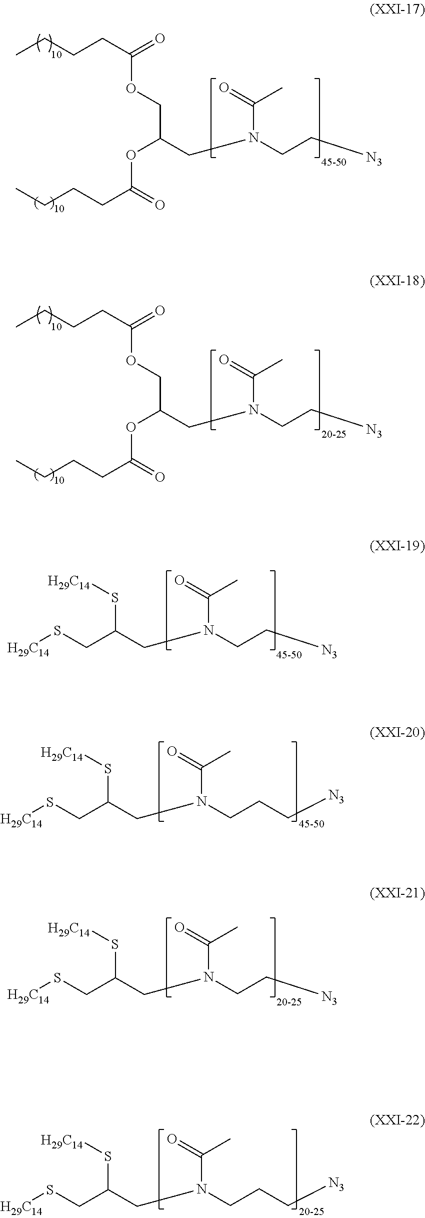

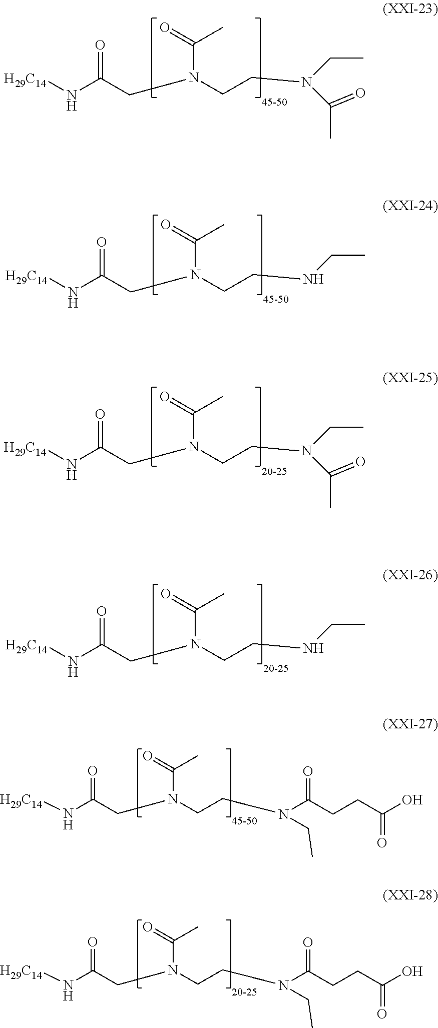

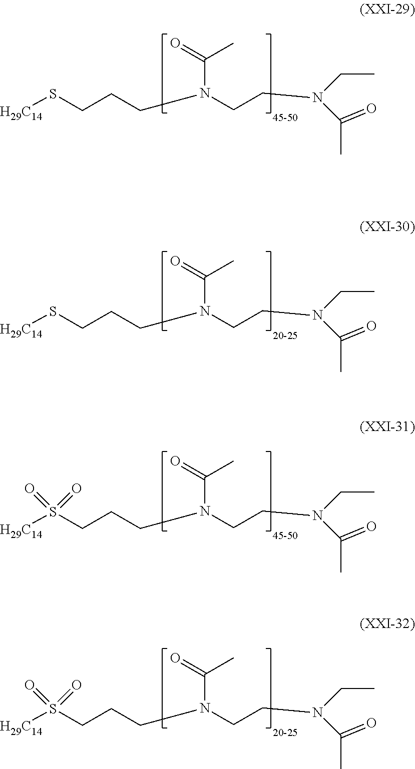

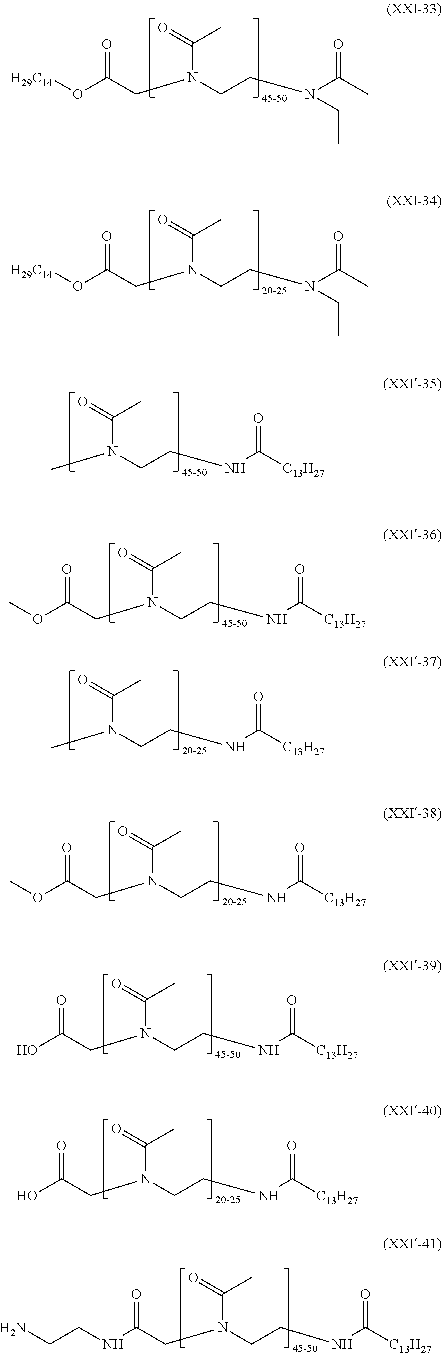

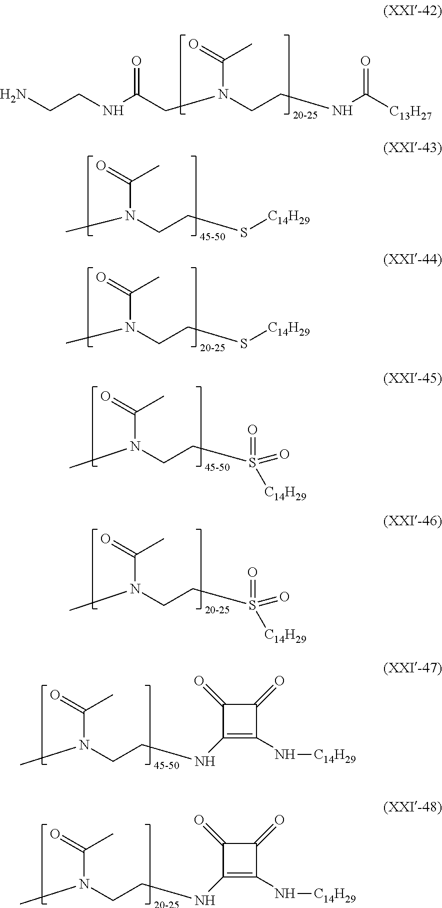

wherein in each case C₁₄H₂₉ refers to the moiety —(CH₂)₁₃CH₃, and in each case C₁₃H₂₇ refers to the moiety —(CH₂)₁₂CH₃.

In some embodiments, the oxazolinylated and/or oxazinylated lipid is selected from any one of the formulas (XXIa), (XXIa′), (XXIb), (XXIb′), (XXIc), (XXId), (XXIe′), (XXIf′), (XXIg′), (XXIh′), (XXIi′), (XXIj′), (XXII), (XXII′), (XXIIa), (XXIIa′), (XXIIb), (XXIIb′), (XXIII), (XXIII′), (XXIIIa), (XXIIIb), (XXIIIc′), (XXIIId′), (XXIIIe′), (XXIIIf′), (XXIIIg′), (XXIIIh′), (XXIV), (XXIV′), (XXV), (XXV′), (XXVa), (XXVa′), (XXVI), (XXVI′), (XXVIa), (XXVIa′), (XXVII), (XXVII′), (XXVIIa), (XXVIII), (XXVIII′), (XXVIIIa), (XXI-1), (XXI-2), (XXI-3), (XXI-4), (XXI-5), (XXI-6), (XXI-7), (XXI-8), (XXI-9), (XXI-10), (XXI-11), (XXI-12), (XXI-13), (XXI-14), (XXI-15), (XXI-16), (XXI-17), (XXI-18), (XXI-19), (XXI-20), (XXI-21), (XXI-22), (XXI-23), (XXI-24), (XXI-25), (XXI-26), (XXI-27), (XXI-28), (XXI-29), (XXI-30), (XXI-31), (XXI-32), (XXI-33), (XXI-34), (XXI′-35), (XXI′-36), (XXI′-37), (XXI′-38), (XXI′-39), (XXI′-40), (XXI′-41), (XXI′-42), (XXI′-43), (XXI′-44), (XXI′-45), (XXI′-46), (XXI′-47), and (XXI′-48).

In some embodiments, the nucleic acid compositions (such as DNA or RNA compositions, especially mRNA compositions) described herein may comprise a cationic/cationically ionizable lipid as described herein (e.g., a cationically ionizable lipid of formula (I), (X), or (XI)), an oxazolinylated and/or oxazinylated lipid as described herein, a phospholipid as described herein, and cholesterol, wherein the oxazolinylated and/or oxazinylated lipid preferably is or comprises any one of the formulas (XXIa), (XXIa′), (XXIb), (XXIb′), (XXIc), (XXId), (XXIe′), (XXIf′), (XXIg′), (XXIh′), (XXIi′), (XXIj′), (XXII), (XXII′), (XXIIa), (XXIIa′), (XXIIb), (XXIIb′), (XXIII), (XXIII′), (XXIIIa), (XXIIIb), (XXIIIc′), (XXIIId′), (XXIIIe′), (XXIIIf′), (XXIIIg′), (XXIIIh′), (XXIV), (XXIV′), (XXV), (XXV′), (XXVa), (XXVa′), (XXVI), (XXVI′), (XXVIa), (XXVIa′), (XXVII), (XXVII′), (XXVIIa), (XXVIII), (XXVIII′), (XXVIIIa), (XXI-1), (XXI-2), (XXI-3), (XXI-4), (XXI-5), (XXI-6), (XXI-7), (XXI-8), (XXI-9), (XXI-10), (XXI-11), (XXI-12), (XXI-13), (XXI-14), (XXI-15), (XXI-16), (XXI-17), (XXI-18), (XXI-19), (XXI-20), (XXI-21), (XXI-22), (XXI-23), (XXI-24), (XXI-25), (XXI-26), (XXI-27), (XXI-28), (XXI-29), (XXI-30), (XXI-31), (XXI-32), (XXI-33), (XXI-34), (XXI′-35), (XXI′-36), (XXI′-37), (XXI′-38), (XXI′-39), (XXI′-40), (XXI′-41), (XXI′-42), (XXI′-43), (XXI′-44), (XXI′-45), (XXI′-46), (XXI′-47), and (XXI′-48).

In some embodiments, the oxazolinylated and/or oxazinylated lipid comprises from about 0.1 mol % to about 10 mol %, such as from about 0.2 mol % to about 9 mol %, from about 0.5 mol % to about 8 mol %, from about 1 mol % to about 7.5 mol %, from about 1.5 mol % to about 7 mol %, from about 2 mol % to about 6.5 mol %, from about 2.5 mol % to about 6 mol %, or from about 3 mol % to about 5 mol %, of the total lipid present in the nucleic acid compositions (such as DNA or RNA compositions, especially mRNA compositions) described herein.

In some embodiments, where at least a portion of (i) the nucleic acid (such as DNA or RNA, especially mRNA), (ii), the cationic/cationically ionizable lipid, and (iii) the oxazolinylated and/or oxazinylated lipid form particles (e.g., nanoparticles, such as LNPs), the oxazolinylated and/or oxazinylated lipid may comprise from about 0.1 mol % to about 10 mol %, such as from about 0.2 mol % to about 9 mol %, from about 0.5 mol % to about 8 mol %, from about 1 mol % to about 7.5 mol %, from about 1.5 mol % to about 7 mol %, from about 2 mol % to about 6.5 mol %, from about 2.5 mol % to about 6 mol %, or from about 3 mol % to about 5 mol %, of the total lipid present in the particles.

In some embodiments, the nucleic acid compositions (such as DNA or RNA compositions, especially mRNA compositions) described herein may comprise a cationic/cationically ionizable lipid as described herein (e.g., a cationically ionizable lipid of formula (X) or (XI)), an oxazolinylated and/or oxazinylated lipid as described herein, a phospholipid, and cholesterol, wherein the cationic/cationically ionizable lipid comprises from about 40 mol % to about 50 mol % of the total lipid present in the composition, the oxazolinylated and/or oxazinylated lipid comprises from about 0.5 mol % to about 10 mol % (such as from about 2 mol to about 5 mol %) of the total lipid present in the composition, the phospholipid comprises from about 5 mol % to about 15 mol % of the total lipid present in the composition, and the cholesterol comprises from about 30 mol % to about 50 mol % of the total lipid present in the composition. In some embodiments, the oxazolinylated and/or oxazinylated lipid has any one of the formulas (XXIa), (XXIa′), (XXIb), (XXIb′), (XXIc), (XXId), (XXIe′), (XXIf′), (XXIg′), (XXIh′), (XXIi′), (XXIj′), (XXII), (XXII′), (XXIIa), (XXIIa′), (XXIIb), (XXIIb′), (XXIII), (XXIII′), (XXIIIa), (XXIIIb), (XXIIIc′), (XXIIId′), (XXIIIe′), (XXIIIf′), (XXIIIg′), (XXIIIh′), (XXIV), (XXIV′), (XXV), (XXV′), (XXVa), (XXVa′), (XXVI), (XXVI′), (XXVIa), (XXVIa′), (XXVII), (XXVII′), (XXVIIa), (XXVIII), (XXVIII′), (XXVIIIa), (XXI-1), (XXI-2), (XXI-3), (XXI-4), (XXI-5), (XXI-6), (XXI-7), (XXI-8), (XXI-9), (XXI-10), (XXI-11), (XXI-12), (XXI-13), (XXI-14), (XXI-15), (XXI-16), (XXI-17), (XXI-18), (XXI-19), (XXI-20), (XXI-21), (XXI-22), (XXI-23), (XXI-24), (XXI-25), (XXI-26), (XXI-27), (XXI-28), (XXI-29), (XXI-30), (XXI-31), (XXI-32), (XXI-33), (XXI-34), (XXI′-35), (XXI′-36), (XXI′-37), (XXI′-38), (XXI′-39), (XXI′-40), (XXI′-41), (XXI′-42), (XXI′-43), (XXI′-44), (XXI′-45), (XXI′-46), (XXI′-47), and (XXI′-48).

In some embodiments, a polymer-conjugated lipid is about 0.5 to about 5 mol % relative to total lipids in the LNP. In some embodiments, an LNP comprises about 1.0 to about 2.5 mol % of a polymer-conjugated lipid. In some embodiments, an LNP comprises about 1.5 to about 2.0 mol % of a polymer-conjugated lipid. In some embodiments, an LNP comprises about 1.5 to about 1.8 mol % of a polymer-conjugated lipid.

In some embodiments, a molar ratio of total cationic lipid to total polymer-conjugated lipid (e.g., PEG-conjugated lipid) is from about 100:1 to about 20:1. In some embodiments, a molar ratio of total cationic lipid to total polymer-conjugated lipid (e.g., PEG-conjugated lipid) is from about 50:1 to about 20:1. In some embodiments, a molar ratio of total cationic lipid to total polymer-conjugated lipid (e.g., PEG-conjugated lipid) is from about 40:1 to about 20:1. In some embodiments, a molar ratio of total cationic lipid to total polymer-conjugated lipid (e.g., PEG-conjugated lipid) is from about 35:1 to about 25:1.

### Immunomodulators

As described herein, a lipid nanoparticle comprises an immunomodulator. In some embodiments, an immunomodulator is an immunosuppressant. In some embodiments, an immunomodulator is an immunostimulant. In some embodiments, an immunomodulator is an agent (e.g., a small molecule) that is an agonist or antagonist of a toll-like receptor (e.g., TLR1, TLR2, TLR3, TLR4, TLR5, TLR6, TLR7, TLR8, TLR9, TLR10) or a pattern recognition receptor (PRR). In some embodiments, an immunomodulator is an sp²-iminosugar glycolipid. In some embodiments, an immunomodulator is a small molecule downstream inhibitor of NF-κβ. In some embodiments, an immunomodulator is an sp²-iminosugar glycolipid. In some embodiments, an immunomodulator is an inflammasome inhibitor.

In some embodiments, a lipid nanoparticle described herein does not comprise dexamethasone or a dexamethasone prodrug (e.g., dexamethasone conjugated to a biodegradeable linker).

In some embodiments, an immunomodulator is a TLR inhibitor. In some embodiments, a TLR inhibitor is an inhibitor of TLR2, TLR4, and/or TLR6. In some embodiments, a TLR inhibitor is an inhibitor of TLR2. In some embodiments, an immunomodulator is an inhibitor of TLR4. In some embodiments, an immunomodulator is an inhibitor of TLR6.

In some embodiments, an inhibitor of TLR4 is TAK-242:

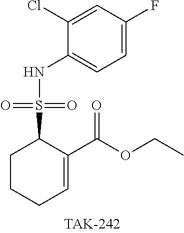 [SMILES: CCOC(=O)C1=CCCC[C@H]1S(=O)(=O)Nc1ccc(F)cc1Cl]

or a pharmaceutically acceptable salt thereof.

In some embodiments, an immunomodulator is a terpenoid. In some embodiments, a terpenoid is a triterpene. In some embodiments, an immunomodulator is a triterpenoid.

In some embodiments, a triterpenoid is a synthetic or natural derivative of amyrin, betulinic acid, oleanolic acid, sterol, squalene, or ursolic acid.

In some embodiments, an immunomodulator is a corticosteroid. In some embodiments, a corticosteroid is a glucocorticoid. In some embodiments, a glucocorticoid is selected from: dexamethasone, prednisolone, fluticasone propionate, budesonide or a pharmaceutically acceptable salt thereof. In some embodiments, a glucocorticoid is dexamethasone, or a pharmaceutically acceptable salt thereof. In some embodiments, a glucocorticoid is prednisolone, or a pharmaceutically acceptable salt thereof. In some embodiments, a glucocorticoid is fluticasone, or a pharmaceutically acceptable salt thereof. In some embodiments, a glucocorticoid is propionate, or a pharmaceutically acceptable salt thereof. In some embodiments, a glucocorticoid is budesonide, or a pharmaceutically acceptable salt thereof.

In some embodiments, an immunomodulator is an inflammasome inhibitor. In some embodiments, an inflammasome inhibitor is a NLRP3 inflammasome inhibitor, a AIM2 inflammasome inhibitor, a caspase-1 inhibitor, or a pan-casase inhibitor. In some embodiments, an inflammasome inhibitor is selected from glyburide (i.e., glibenclamide), 5-chloro-2-methoxy-N-[2-(4-sulfamoylphenyl)-ethyl]-benzamide (i.e., 16673-34-0), JC124, FC11A-2, parthenolide, VX-740, VX-765, BAY 11-7082, BHB, MCC950, MNS, CY-09, Tranilast, OLT1177, and oridonin. In some embodiments, an inflammasome inhibitor is glyburide (i.e., glibenclamide).

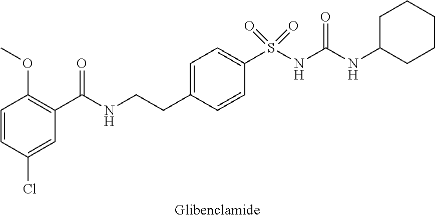 [SMILES: COc1ccc(Cl)cc1C(=O)NCCc1ccc(S(=O)(=O)NC(=O)NC2CCCCC2)cc1]

In some embodiments, an inflammasome inhibitor is 5-chloro-2-methoxy-N-[2-(4-sulfamoylphenyl)-ethyl]-benzamide (i.e., 16673-34-0).

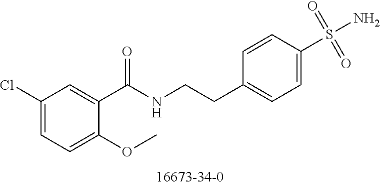 [SMILES: COc1ccc(Cl)cc1C(=O)NCCc1ccc(S(N)(=O)=O)cc1]

In some embodiments, an inflammasome inhibitor is JC124.

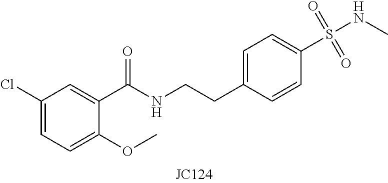 [SMILES: CNS(=O)(=O)c1ccc(CCNC(=O)c2cc(Cl)ccc2OC)cc1]

In some embodiments, an inflammasome inhibitor is FC11A-2.

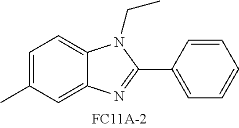 [SMILES: CCn1c(-c2ccccc2)nc2cc(C)ccc21]

In some embodiments, an inflammasome inhibitor is parthenolide.

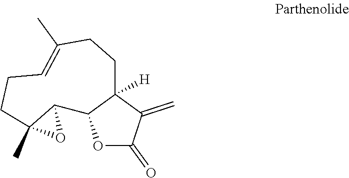 [SMILES: C=C1C(=O)O[C@@H]2[C@H]3O[C@]3(C)CC/C=C(\C)CC[C@@H]12]

In some embodiments, an inflammasome inhibitor is VX-740.

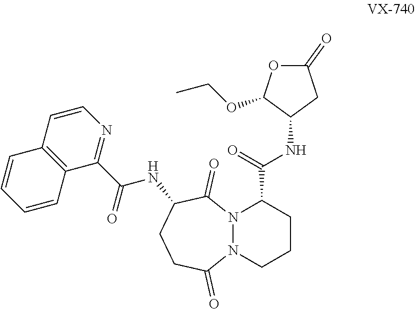 [SMILES: CCO[C@@H]1OC(=O)C[C@@H]1NC(=O)[C@@H]1CCCN2C(=O)CC[C@H](NC(=O)c3nccc4ccccc34)C(=O)N12]

In some embodiments, an inflammasome inhibitor is VX-765.

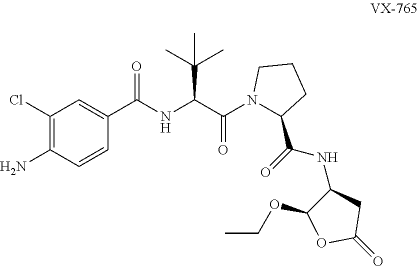 [SMILES: CCO[C@@H]1OC(=O)C[C@@H]1NC(=O)[C@@H]1CCCN1C(=O)[C@@H](NC(=O)c1ccc(N)c(Cl)c1)C(C)(C)C]

In some embodiments, an inflammasome inhibitor is BAY 11-7082.

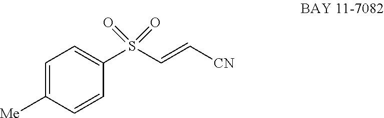 [SMILES: Cc1ccc(S(=O)(=O)/C=C/C#N)cc1]

In some embodiments, an inflammasome inhibitor is beta hydroxybutyrate (BHB).

In some embodiments, an inflammasome inhibitor is MCC950.

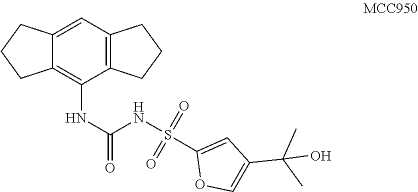 [SMILES: CC(C)(O)c1coc(S(=O)(=O)NC(=O)Nc2c3c(cc4c2CCC4)CCC3)c1]

In some embodiments, an inflammasome inhibitor is 3,4-methylenedioxy-p-nitrostyrene (MNS).

In some embodiments, an inflammasome inhibitor is CY-09.

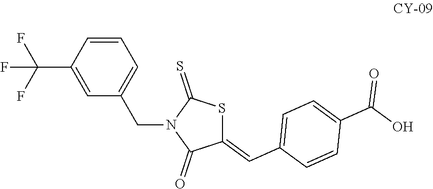 [SMILES: O=C(O)c1ccc(/C=C2\SC(=S)N(Cc3cccc(C(F)(F)F)c3)C2=O)cc1]

In some embodiments, an inflammasome inhibitor is Tranilast.

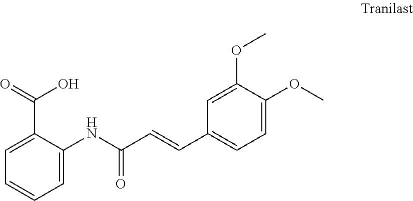 [SMILES: COc1ccc(/C=C/C(=O)Nc2ccccc2C(=O)O)cc1OC]

In some embodiments, an inflammasome inhibitor is OLT1177.

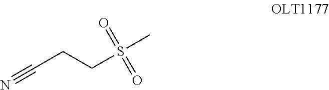

In some embodiments, an inflammasome inhibitor is oridonin.

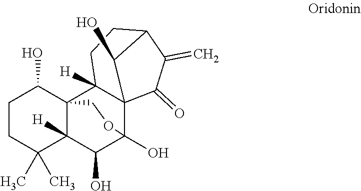

In some embodiments, a lipid nanoparticle described herein comprises more than one immunomodulator. In some embodiments, a lipid nanoparticle comprises an immunomodulator and one or more additional immunomodulators. In some embodiments, a lipid nanoparticle comprises a terpenoid (e.g., a corticosteroid, a glucocorticoid such as dexamethasone, prednisolone, fluticasone propionate, or budesonide, or an inflammasome inhibitor), and one or more additional immumomodulators (e.g., another of a corticosteroid, or a glucocorticoid such as dexamethasone, prednisolone, fluticasone propionate, or budesonide, or a TLR or PRR agonist or antagonist, or an inflammaosome inhibitor, as described herein). In some embodiments, a lipid nanoparticle comprises an immunomodulator that is a terpenoid (including any subclasses described herein) and a small molecular agonist or antagonist of TLR or PRR (including any subclasses described herein, such as, e.g., TAK-242).

In some embodiments, a lipid nanoparticle described herein comprises a cationic lipid and an immunomodulator, wherein the immunomodulator is a TLR inhibitor. In some embodiments, a lipid nanoparticle comprises a cationic lipid and an immunomodulator, wherein the immunomodulator is an inflammasome inhibitor. In some embodiments, a lipid nanoparticle comprises a cationic lipid, RNA, and an immunomodulator selected from TAK-242, MCC950, and BAY 11-7082. In some embodiments, a lipid nanoparticle comprises a cationic lipid, RNA, and TAK-242. In some embodiments, a lipid nanoparticle comprises a cationic lipid, RNA, and MCC950. In some embodiments, a lipid nanoparticle comprises a cationic lipid, RNA, and BAY 11-7082.

In some embodiments, a lipid nanoparticle comprises about 0.1 to about 50 mol % of an immunomodulator (relative to the total amount of lipids and immunomodulators). In some embodiments, a lipid nanoparticle comprises about 1 to about 50 mol % of an immunomodulator (relative to the total amount of lipids and immunomodulators). In some embodiments, a lipid nanoparticle comprises about 30 to about 50 mol % of an immunomodulator (relative to the total amount of lipids and immunomodulators). In some embodiments, a lipid nanoparticle comprises about 30 to about 50 mol % of an immunomodulator (relative to the total amount of lipids and immunomodulators). In some embodiments, a lipid nanoparticle comprises about 30 to about 35 mol % of an immunomodulator (relative to the total amount of lipids and immunomodulators). In some embodiments, a lipid nanoparticle comprises about 35 to about 45 mol % of an immunomodulator (relative to the total amount of lipids and immunomodulators). In some embodiments, a lipid nanoparticle comprises about 40 to about 50 mol % of an immunomodulator (relative to the total amount of lipids and immunomodulators). In some embodiments, a lipid nanoparticle comprises about 30, 31, 32, 33, 34, 35, 36, 37, 38, 39, 40, 41, 42, 43,44, 45, 46, 47, 48, 49, or 50 mol % of an immunomodulator (relative to the total amount of lipids and immunomodulators).

In some embodiments, a nucleic acid particle comprises i) about 30 to about 50 mol % of the cationic lipid; ii) about 1 to about 5 mol % of a polymer-conjugated lipid; iii) about 5 to about 15 mol % of a helper lipid, and iv) an immunomodulator. In some embodiments, a nucleic acid particle comprises: i) about 30 to about 50 mol % of ALC-0315; ii) about 1 to about 5 mol % of ALC-0159; iii) about 5 to about 15 mol % of DSPC, and iv) an immunomodulator. In some embodiments, a nucleic acid particle comprises: i) about to about 50 mol % of ALC366; ii) about 1 to about 5 mol % of ALC-0159; iii) about 5 to about 15 mol % of DSPC, and iv) an immunomodulator.

In some embodiments, a nucleic acid particle comprises: i) about 47.5 mol % of ALC-0315; ii) about 1.8 mol % of ALC-0159; and iii) about 10 mol % of DSPC. In some embodiments, a nucleic acid particle comprises: i) about 47.5 mol % of ALC366; ii) about 1.8 mol % of ALC-0159; and iii) about 10 mol % of DSPC.

In some embodiments, nucleic acid particle comprises: i) about 30 to about 50 mol % of SM-102; ii) about 1 to about 5 mol % of a PEG2000-DMG; iii) about 5 to about 15 mol % of DSPC; and iv) an immunomodulator. In some embodiments, a nucleic acid particle comprises i) about 50 mol % of SM-102; ii) about 1.5 mol % of PEG2000-DMG; iii) about mol % of DSPC; and iv) an immunomodulator. In some embodiments, an immunomodulator is TAK-242, MCC950, or BAY 11-7082. In some embodiments, a nucleic acid particle comprises i) about 50 mol % of SM-102; ii) about 1.5 mol % of PEG2000-DMG; iii) about 10 mol % of DSPC; and iv) TAK-242. In some embodiments, a nucleic acid particle comprises i) about 50 mol % of SM-102; ii) about 1.5 mol % of PEG2000-DMG; iii) about 10 mol % of DSPC; and iv) MCC950. In some embodiments, a nucleic acid particle comprises i) about 50 mol % of SM-102; ii) about 1.5 mol % of PEG2000-DMG; iii) about 10 mol % of DSPC; and iv) BAY 11-7082.

### Terpenoids

As described herein, in some embodiments, a nucleic acid particle further comprises a terpenoid. In some embodiments, a terpenoid is a triterpene. In some embodiments, a terpenoid is a steroid. In some embodiments, a steroid is a sterol. In some embodiments, a sterol is β-sitosterol, stigmasterol, cholesterol, cholecalciferol, ergocalciferol, calcipotriol, botulin, lupeol, ursolic acid, oleanolic acid, cycloartenol, lanosterol, or α-tocopherol. In some embodiments, a sterol is β-sitosterol. In some embodiments, a sterol is stigmasterol. In some embodiments, a sterol is cholesterol. In some embodiments, a sterol is cholecalciferol. In some embodiments, a sterol is ergocalciferol. In some embodiments, a sterol is calcipotriol. In some embodiments, a sterol is botulin. In some embodiments, a sterol is lupeol. In some embodiments, a sterol is ursolic acid. In some embodiments, a sterol is oleanolic acid. In some embodiments, a sterol is cycloartenol. In some embodiments, a sterol is lanosterol. In some embodiments, a sterol is α-tocopherol.

In some embodiments, a lipid nanoparticle comprises about 30 to about 50 mol % of a steroid. In some embodiments, a lipid nanoparticle comprises about 35 to about 45 mol % of a steroid. In some embodiments, a lipid nanoparticle comprises about 38 to about 40 mol % of a steroid. In some embodiments, a lipid nanoparticle comprises about 38.5 mol % of a steroid. In some embodiments, a lipid nanoparticle comprises about 40 mol % of a steroid.

In some embodiments, a lipid nanoparticle comprises about 30 to about 50 mol % of cholesterol. In some embodiments, a lipid nanoparticle comprises about 35 to about 45 mol % of cholesterol. In some embodiments, a lipid nanoparticle comprises about 38 to about 41 mol % of cholesterol. In some embodiments, a lipid nanoparticle comprises about 38.5 mol % of cholesterol. In some embodiments, a lipid nanoparticle comprises about 40.7 mol % of cholesterol.

### Methods of Making Lipid Nanoparticles

Lipids and lipid nanoparticles comprising nucleic acids and their method of preparation are known in the art, including, e.g., as described in U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2018/081480, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548, the full disclosures of which are herein incorporated by reference in their entirety for the purposes described herein.

For example, in some embodiments, cationic lipids, helper lipids, and immunomodulators are solubilized in ethanol at a pre-determined weight or molar ratios/percentages (e.g., ones described herein). In some embodiments, lipid nanoparticles (LNP) are prepared at a total lipid to RNA weight ratio of approximately 10:1 to 30:1. In some embodiments, such RNA can be diluted to 0.2 mg/mL in acetate buffer.

In some embodiments, using an ethanol injection technique, a colloidal lipid dispersion comprising RNAs can be formed as follows: an ethanol solution comprising lipids, such as cationic lipids, helper lipids, immunomodulators, and polymer-conjugated lipids, is injected into an aqueous solution comprising RNAs (e.g., ones described herein).

In some embodiments, lipid and RNA solutions can be mixed at room temperature by pumping each solution (e.g., a lipid solution comprising a cationic lipid, a helper lipid, immunomodulators, and any other additives) at controlled flow rates into a mixing unit, for example, using piston pumps. In some embodiments, the flow rates of a lipid solution and a RNA solution into a mixing unit are maintained at a ratio of 1:3. Upon mixing, nucleic acid-lipid particles are formed as the ethanolic lipid solution is diluted with aqueous RNAs. The lipid solubility is decreased, while cationic lipids bearing a positive charge interact with the negatively charged RNA.

In some embodiments, a solution comprising RNA-encapsulated lipid nanoparticles can be processed by one or more of concentration adjustment, buffer exchange, formulation, and/or filtration.

### Polymer-Based Particles

In some embodiments, a nucleic acid particle described herein comprises a cationic polymer, RNA, and an immunomodulator. Examples of nucleic acid particles that are suitable for RNA delivery are described in PCT App. Pub. No. WO 2021/001417, which is incorporated herein by reference in its entirety. As described generally above, a complex described herein comprises a cationic polymer. In some embodiments, a cationic polymer is a polycationic polymer, e.g., a polymer having one or more cationic or ionizable groups. As described herein, a “cationic” group is a group having a net positive charge. As used herein, an “ionizable” group is a group that may have a neutral charge at a certain pH, but may become charged (e.g., cationic) at a different pH. For example, in some embodiments, an ionizable group becomes cationic (i.e., positively charged) at physiological pH (e.g., a pH of about 7.4). Accordingly, it is understood that reference to a cationic polymer refers to both the neutral form and the charged (i.e., ionic) form. A person of skill in the art will also appreciate that cationic groups described herein can also exist as a salt (e.g., a pharmaceutically acceptable salt) that comprises a cationic group and one or more suitable counterions. For example, in some embodiments, a cationic group described herein comprises ammonium chloride. Suitable counterions include halogens (e.g., Br⁻, Cl⁻, I⁻, F⁻), acetates (e.g., C(O)O—), and the like. For additional examples, see the definition for pharmaceutically acceptable salts described herein.

In some embodiments, one or more cationic or ionizable groups comprise a nitrogen atom. Cationic polymers useful for preparing complexes described herein can be homopolymers heteropolymers, or block-co-polymers.

In some embodiments, a cationic polymer is poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

In some embodiments, a cationic polymer is a homopolymer. In some embodiments, a cationic polymer is a homopolymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

It is understood that a cationic polymer described herein can be linear or branched. In some embodiments, a cationic polymer is linear. In some embodiments, a cationic polymer is a linear polymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a cationic polymer is a branched polymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a cationic polymer is linear poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

In some embodiments, a cationic polymer is poly(ethylenimine). In some embodiments, a cationic polymer is poly(propylenimine). In some embodiments, a cationic polymer is polybrene. In some embodiments, a cationic polymer is polyallylamine. In some embodiments, a cationic polymer is polyvinylamine. In some embodiments, a cationic polymer is polyamidoamine. In some embodiments, a cationic polymer is poly-L-lysine.

In some embodiments, a cationic polymer is poly-L-arginine. In some embodiments, a cationic polymer is poly-L-histidine. In some embodiments, a cationic polymer is poly(2-aminoethyl methacrylate).

In some embodiments, a cationic polymer is a heteropolymer comprising copolymers of one or more of poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a cationic polymer is a heteropolymer comprising poly(ethylenimine) and poly(propylenimine). In some embodiments, a cationic polymer is a linear heteropolymer comprising copolymers of one or more of poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a cationic polymer is a heteropolymer comprising poly(ethylenimine) and poly(propylenimine).

In some embodiments, a cationic polymer is a block-co-polymer comprising blocks of polymers selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a cationic polymer is a block-co-polymer of poly(ethylenimine) and poly(propylenimine) (i.e., poly(ethylenimine)-block-poly(propylenimine)). In some embodiments, a cationic polymer is a linear block-co-polymer comprising blocks of polymers selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate). In some embodiments, a cationic polymer is a linear block-co-polymer of poly(ethylenimine) and poly(propylenimine) (i.e., poly(ethylenimine)-block-poly(propylenimine)).

In some embodiments, a cationic polymer has between 250 and 2000 repeating monomer units. In some embodiments, a cationic polymer has between 500 and 2000 repeating monomer units. In some embodiments, a cationic polymer has between 1000 and 2000 repeating monomer units. In some embodiments, a cationic polymer has between 1500 and 2000 repeating monomer units. In some embodiments, a cationic polymer comprises about 250, about 300, about 350, about 400, about 500, about 600, about 700, about 800, about 900, about 1000, about 1100, about 1200, about 1300, about 1400, about 1500, about 1600, about 1700, about 1800, about 1900, or about 2000 monomer units.

In some embodiments, a cationic polymer is a polymer or copolymer comprising blocks of formula I and/or II:

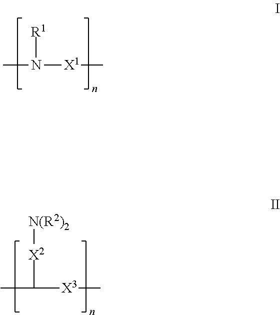

or a pharmaceutically acceptable salt thereof, wherein:
R¹ is H, —C(O)-optionally substituted C₁-C₆ aliphatic, optionally substituted C₁-C₆ aliphatic or G;
each R² is independently H, optionally substituted C₁-C₆ aliphatic, or G;
X¹ and X³ are each independently C₁-C₆ aliphatic;
X² is a bond or optionally substituted C₁-C₆ aliphatic;
m is an integer between 2 and 2000;
n is an integer between 2 and 2000;
each G is independently:

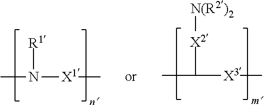

R^{1′} is H, —C(O)-optionally substituted C₁-C₆ aliphatic, optionally substituted C₁-C₆ aliphatic, or G′;
each R^{2′} is independently H, optionally substituted C₁-C₆ aliphatic, or G′;
X^{1′} and X^{3′} are each independently optionally substituted C₁-C₆ aliphatic;
X^{2′} is a bond or optionally substituted C₁-C₆ aliphatic;
n′ is an integer between 2 and 2000;
m′ is an integer between 2 and 2000;
each G′ is independently:

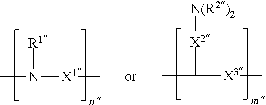

R^{1″} is H, —C(O)-optionally substituted C₁-C₆ aliphatic, or optionally substituted C₁-C₆ aliphatic;
each R^{2″} is independently H or C₁-C₆ aliphatic;
X^{1″} and X^{3″} are each independently optionally substituted C₁-C₆ aliphatic;
X^{2″} is a bond or optionally substituted C₁-C₆ aliphatic;
n″ is an integer between 2 and 2000; and
m″ is an integer between 2 and 2000.

In some embodiments, a cationic polymer is a polymer described herein, having a number average molecular weight (Mₙ) of about 600 Daltons (Da) to about 400,000 Da. In some embodiments, a cationic polymer has a Mₙ of about 1,000 Da to about 300,000 Da. In some embodiments, a cationic polymer has a Mₙ of about 10,000 to about 250,000 Da. In some embodiments, a cationic polymer has a Mₙ of about 10,000 to about 120,000 Da. In some embodiments, a cationic polymer has a Mₙ of about 20,000 to about 120,000 Da.

In some embodiments, a nucleic acid particle described herein comprises a cationic polymer, RNA, an immunomodulator, and optionally a secondary polymer. In some embodiments, a nucleic acid particle comprises a cationic polymer, RNA, a secondary polymer, and an immunomodulator. In some embodiments, a secondary polymer is polysarcosine.

As described herein, a nucleic acid particle described herein comprises a cationic polymer, RNA, optionally a secondary polymer, and an immunomodulator. In some embodiments, an immunomodulator is an immunosuppressant. In some embodiments, an immunomodulator is an immunostimulant. In some embodiments, an immunomodulator is an agent (e.g., a small molecule) that is an agonist or antagonist of a toll-like receptor (e.g., TLR1, TLR2, TLR3, TLR4, TLR5, TLR6, TLR7, TLR8, TLR9, TLR10) or a pattern recognition receptor (PRR). In some embodiments, an immunomodulator is an sp²-iminosugar glycolipid. In some embodiments, an immunomodulator is a small molecule downstream inhibitor of NF-κβ. In some embodiments, an immunomodulator is an sp²-iminosugar glycolipid. In some embodiments, an immunomodulator is an inflammasome inhibitor.

In some embodiments, a nucleic acid particle described herein does not comprise dexamethasone.

In some embodiments, an immunomodulator is a TLR inhibitor. In some embodiments, a TLR inhibitor is an inhibitor of TLR2, TLR4, and/or TLR6. In some embodiments, a TLR inhibitor is an inhibitor of TLR2. In some embodiments, an immunomodulator is an inhibitor of TLR4. In some embodiments, an immunomodulator is an inhibitor of TLR6.

In some embodiments, an inhibitor of TLR4 is TAK-242:

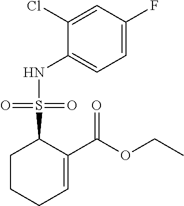 [SMILES: CCOC(=O)C1=CCCC[C@H]1S(=O)(=O)Nc1ccc(F)cc1Cl]

or a pharmaceutically acceptable salt thereof.

In some embodiments, an immunomodulator is a terpenoid. In some embodiments, a terpenoid is a triterpene. In some embodiments, an immunomodulator is a triterpenoid. In some embodiments, a triterpenoid is a synthetic or natural derivative of amyrin, betulinic acid, oleanolic acid, sterol, squalene, or ursolic acid.

In some embodiments, an immunomodulator is a corticosteroid. In some embodiments, a corticosteroid is a glucocorticoid. In some embodiments, a glucocorticoid is selected from: dexamethasone, prednisolone, fluticasone propionate, budesonide or a pharmaceutically acceptable salt thereof. In some embodiments, a glucocorticoid is dexamethasone, or a pharmaceutically acceptable salt thereof. In some embodiments, a glucocorticoid is prednisolone, or a pharmaceutically acceptable salt thereof. In some embodiments, a glucocorticoid is fluticasone, or a pharmaceutically acceptable salt thereof. In some embodiments, a glucocorticoid is propionate, or a pharmaceutically acceptable salt thereof. In some embodiments, a glucocorticoid is budesonide, or a pharmaceutically acceptable salt thereof.

In some embodiments, an immunomodulator is an inflammasome inhibitor. In some embodiments, an inflammasome inhibitor is a NLRP3 inflammasome inhibitor, a AIM2 inflammasome inhibitor, a caspase-1 inhibitor, or a pan-casase inhibitor. In some embodiments, an inflammasome inhibitor is selected from glyburide (i.e., glibenclamide), 5-chloro-2-methoxy-N-[2-(4-sulfamoylphenyl)-ethyl]-benzamide (i.e., 16673-34-0), JC124, FC11A-2, parthenolide, VX-740, VX-765, Bay 11-7082, BHB, MCC950, MNS, CY-09, Tranilast, OLT1177, and oridonin. In some embodiments, an inflammasome inhibitor is glyburide (i.e., glibenclamide).

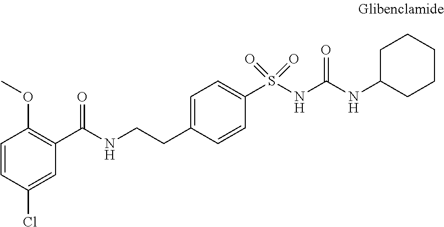 [SMILES: COc1ccc(Cl)cc1C(=O)NCCc1ccc(S(=O)(=O)NC(=O)NC2CCCCC2)cc1]

In some embodiments, an inflammasome inhibitor is 5-chloro-2-methoxy-N-[2-(4-sulfamoylphenyl)-ethyl]-benzamide (i.e., 16673-34-0).

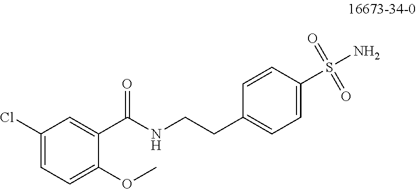 [SMILES: COc1ccc(Cl)cc1C(=O)NCCc1ccc(S(N)(=O)=O)cc1]

In some embodiments, an inflammasome inhibitor is JC124.

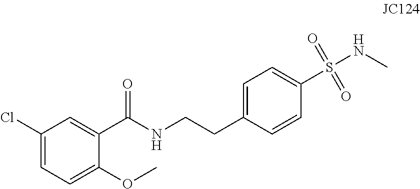 [SMILES: CNS(=O)(=O)c1ccc(CCNC(=O)c2cc(Cl)ccc2OC)cc1]

In some embodiments, an inflammasome inhibitor is FC11A-2.

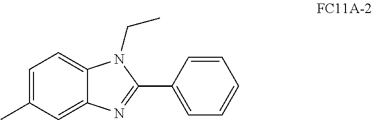 [SMILES: CCn1c(-c2ccccc2)nc2cc(C)ccc21]

In some embodiments, an inflammasome inhibitor is parthenolide.

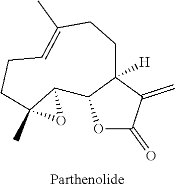 [SMILES: C=C1C(=O)O[C@@H]2[C@H]3O[C@]3(C)CC/C=C(\C)CC[C@@H]12]

In some embodiments, an inflammasome inhibitor is VX-740.

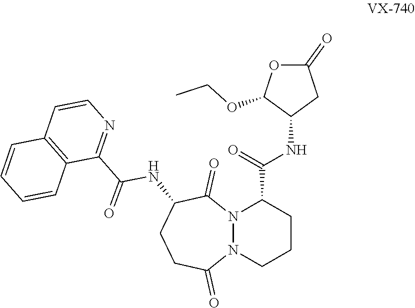 [SMILES: CCO[C@@H]1OC(=O)C[C@@H]1NC(=O)[C@@H]1CCCN2C(=O)CC[C@H](NC(=O)c3nccc4ccccc34)C(=O)N12]

In some embodiments, an inflammasome inhibitor is VX-765.

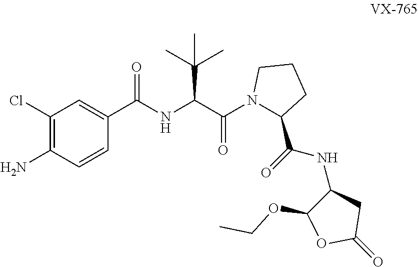 [SMILES: CCO[C@@H]1OC(=O)C[C@@H]1NC(=O)[C@@H]1CCCN1C(=O)[C@@H](NC(=O)c1ccc(N)c(Cl)c1)C(C)(C)C]

In some embodiments, an inflammasome inhibitor is BAY 11-7082.

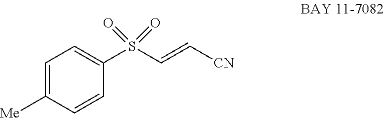 [SMILES: Cc1ccc(S(=O)(=O)/C=C/C#N)cc1]

In some embodiments, an inflammasome inhibitor is beta hydroxybutyrate (BHB).

In some embodiments, an inflammasome inhibitor is MCC950.

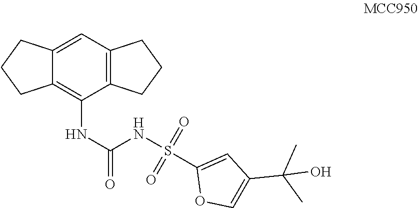 [SMILES: CC(C)(O)c1coc(S(=O)(=O)NC(=O)Nc2c3c(cc4c2CCC4)CCC3)c1]

In some embodiments, an inflammasome inhibitor is 3,4-methylenedioxy-p-nitrostyrene (MNS).

In some embodiments, an inflammasome inhibitor is CY-09.

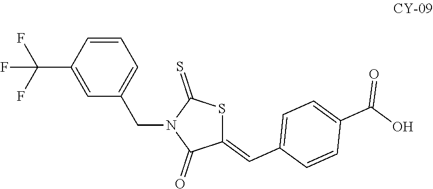 [SMILES: O=C(O)c1ccc(/C=C2\SC(=S)N(Cc3cccc(C(F)(F)F)c3)C2=O)cc1]

In some embodiments, an inflammasome inhibitor is Tranilast.

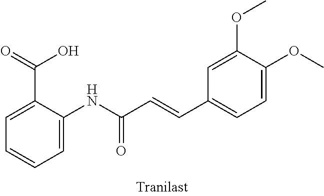 [SMILES: COc1ccc(/C=C/C(=O)Nc2ccccc2C(=O)O)cc1OC]

In some embodiments, an inflammasome inhibitor is OLT1177.

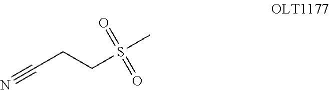

In some embodiments, an inflammasome inhibitor is oridonin.

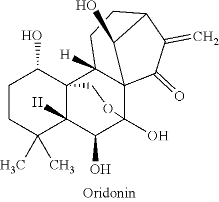

In some embodiments, a nucleic acid particle comprises a cationic polymer, RNA, and an immunomodulator, wherein the immunomodulator is a TLR inhibitor. In some embodiments, a nucleic acid particle comprises a cationic polymer, RNA, and an immunomodulator, wherein the immunomodulator is an inflammasome inhibitor.

In some embodiments, a nucleic acid particle comprising a cationic polymer, RNA, a secondary polymer, an immunomodulator, and optionally a secondary polymer, described herein, comprises more than one immunomodulator. In some embodiments, a nucleic acid particle comprises a cationic polymer, RNA, an immunomodulator, optionally a secondary polymer, and one or more additional immunomodulators. In some embodiments, a nucleic acid particle comprises a cationic polymer, RNA, a terpenoid (e.g., a corticosteroid, a glucocorticoid such as dexamethasone, prednisolone, fluticasone propionate, or budesonide, or an inflammasome inhibitor), and one or more additional immumomodulators (e.g., another of a corticosteroid, or a glucocorticoid such as dexamethasone, prednisolone, fluticasone propionate, or budesonide, or a TLR or PRR agonist or antagonist, or an inflammaosome inhibitor, as described herein), and optionally a secondary polymer. In some embodiments, a nucleic acid particle comprises a cationic polymer, RNA, and an immunomodulator that is a terpenoid (including any subclasses described herein) and a small molecular agonist or antagonist of TLR or PRR (including any subclasses described herein, such as, e.g., TAK-242), and optionally a secondary polymer. In some embodiments, a nucleic acid particle comprises a cationic polymer, RNA, and an immunomodulator that is an NLRP3 inflammasome inhibitor, and optionally a secondary polymer. In some embodiments, a nucleic acid particle comprises a cationic polymer, RNA, and an immunomodulator that is MCC850 or BAY 11-7082, and optionally a secondary polymer. In some embodiments, a nucleic acid particle comprises a cationic polymer, RNA, and an immunomodulator that is an NLRP3 inflammasome inhibitor, and optionally a secondary polymer. In some embodiments, a nucleic acid particle comprises a cationic polymer, RNA, and an immunomodulator that is MCC850, and optionally a secondary polymer. In some embodiments, a nucleic acid particle comprises a cationic polymer, RNA, and an immunomodulator that is an NLRP3 inflammasome inhibitor, and optionally a secondary polymer. In some embodiments, a nucleic acid particle comprises a cationic polymer, RNA, and an immunomodulator that is BAY 11-7082, and optionally a secondary polymer.

In some embodiments, a secondary polymer is a polysarcosine polymer. In some embodiments, a polysarcosine comprises between 2 and 200 sarcosine units, such as between 5 and 100 sarcosine units, between 10 and 50 sarcosine units, between 15 and sarcosine units, e.g., about 23 sarcosine units. In some embodiments, a secondary polymer is a polyglutamate/polysarcosine polymer. In some embodiments, a secondary polymer is a polyglutamate/polysarcosine polymer comprising a molar ratio of glutamate units:sarcosine units that is from about 1:99 to about 99:1. In some embodiments, a secondary polymer is a polyglutamate/polysarcosine polymer comprising a molar ratio of glutamate units:sarcosine units that is from about 25:75 to about 75:25. In some embodiments, a secondary polymer is a polyglutamate/polysarcosine polymer comprising a molar ratio of glutamate units:sarcosine units that is about 50:50.

In some embodiments, a polysarcosine polymer comprises the structure of the following general formula (XVII):

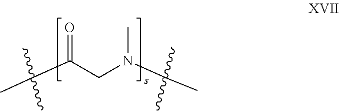

wherein s is the number of sarcosine units.

In some embodiments, a polysarcosine polymer comprises the structure of the following general formula (XVIII):

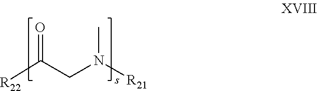

wherein one of R₂₁ and R₂₂ comprises a hydrophobic group and the other is H, a hydrophilic group or a functional group optionally comprising a targeting moiety; and x is the number of sarcosine units.

In some embodiments of formula (XVIII), R₂₁ is H, a hydrophilic group or a functional group optionally comprising a targeting moiety; and R₂₂ comprises one or two straight alkyl or alkenyl groups each having at least 12 carbon atoms, such as at least 14 carbon atoms. In some embodiments, each of the straight alkyl and alkenyl groups has at most carbon atoms, such as at most 28, at most 26, at most 24, at most 22, at most 20, or at most 18 carbon atoms. In some embodiments, R₂₂ comprises one or two straight alkyl or alkenyl groups each having 12 to 30 carbon atoms (such as 12 to 28 carbon atoms, 12 to 26 carbon atoms, 12 to 24 carbon atoms, 12 to 22 carbon atoms, 12 to 20 carbon atoms, or 12 to 18 carbon atoms).

In some embodiments, a polysarcosine polymer has the structure of the following general formula (IXX):

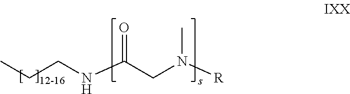

wherein R is H, a hydrophilic group or a functional group optionally comprising a targeting moiety; and s is the number of sarcosine units.

In some embodiments, a polysarcosine polymer has the structure of the following formula (IXX-1):

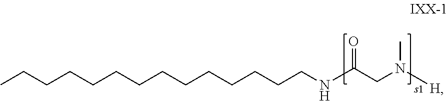

wherein si is 23. The polyarcosine polymer of formula (IXX-1) is also referred to herein as “C14pSar23”.

As described herein, in some embodiments, provided compounds are provided and/or utilized in a salt form (e.g., a pharmaceutically acceptable salt form). Reference to a compound provided herein is understood to include reference to salts thereof, unless otherwise indicated.

### Cargo

In some embodiments, a complex described herein further comprises a cargo. In some embodiments, a cargo is a therapeutic agent, e.g., a small molecule, a peptide, a nucleic acid, or a combination thereof. In some embodiments, a cargo is a small molecule. In some embodiments, a cargo is a peptide. In some embodiments, a cargo is a nucleic acid.

### Nucleic Acids

Nucleic acid particles described herein comprise one or more nucleic acids. In some embodiments, a nucleic acid is RNA.

In some embodiments, an RNA amenable to technologies described herein is a single-stranded RNA. In some embodiments, an RNA as disclosed herein is a linear RNA. In some embodiments, a single-stranded RNA is a non-coding RNA in that its nucleotide sequence does not include an open reading frame (or complement thereof). In some embodiments, a single-stranded RNA has a nucleotide sequence that encodes (or is the complement of a sequence that encodes) a polypeptide or a plurality of polypeptides (e.g., epitopes) of the present disclosure.

In some embodiments, an RNA is or comprises an siRNA, an miRNA, or other non-coding RNA.

In many embodiments, a relevant RNA includes at least one open reading frame (ORF) (e.g., is an mRNA); in some embodiments, a relevant RNA includes a single ORF; in some embodiments, a relevant RNA includes more than one ORF.

In some embodiments, an RNA comprises an ORF, e.g., encoding a polypeptide of interest or encoding a plurality of polypeptides of interest. In some embodiments, an RNA produced in accordance with technologies provided herein comprises a plurality of ORFs (e.g., encoding a plurality of polypeptides). In some embodiments, an RNA produced in accordance with technologies herein comprises a single ORF that encodes a plurality of polypeptides. In some such embodiments, polypeptides are or comprise antigens or epitopes thereof (e.g., relevant antigens).

In some embodiments, an ORF for use in accordance with the present disclosure encodes a polypeptide that includes a signal sequence, e.g., that is functional in mammalian cells, such as an intrinsic signal sequence or a heterologous signal sequence. In some embodiments, a signal sequence directs secretion of an encoded polypeptide, in some embodiments, a signal sequence directs transport of an encoded polypeptide into a defined cellular compartment, preferably the cell surface, the endoplasmic reticulum (ER) or the endosomal-lysosomal compartment.

In some embodiments, an ORF encodes a polypeptide that includes a multimerization element (e.g., an intrinsic or heterologous multimerization element). In some embodiments, an ORF that encodes a surface polypeptide (e.g., that includes a signal sequence directing surface localization) includes a multimerization element.

In some embodiments, an ORF encodes a polypeptide that includes a transmembrane element or domain.

In some embodiments, an ORF is codon-optimized for expression in a cells of a particular host, e.g., a mammalian host, e.g., a human.

In some embodiments, an RNA includes unmodified uridine residues; an RNA that includes only unmodified uridine residues may be referred to as a “uRNA”. In some embodiments, an RNA includes one or more modified uridine residues; in some embodiments, such an RNA (e.g., an RNA including entirely modified uridine residues) is referred to as a “modRNA”. In some embodiments, an RNA may be a self-amplifying RNA (saRNA). In some embodiments, an RNA may be a trans-amplifying RNA (taRNA) (see, for example, WO2017/162461). In some embodiments, an RNA may be a covalently closed RNA molecule that forms a covalent closed continuous loop (circRNA).

In some embodiments, a relevant RNA includes a polypeptide-encoding portion or a plurality of polypeptide-encoding portions. In some particular embodiments, such a portion or portions may encode a polypeptide or polypeptides that is or comprises a biologically active polypeptide or portion thereof (e.g., an enzyme or cytokine or therapeutic protein such as a replacement protein or antibody or portion thereof). In some particular embodiments, such a portion or portions may encode a polypeptide or polypeptides that is or comprises an antigen (or an epitope thereof), a cytokine, an enzyme, etc. In some embodiments, an encoded polypeptide or polypeptides may be or include one or more neoantigens or neoepitopes associated with a tumor. In some embodiments, an encoded polypeptide or polypeptides may be or include one or more antigens (or epitopes thereof) of an infectious agent (e.g., a bacterium, fungus, virus, etc.). In certain embodiments, an encoded polypeptide may be a variant of a wild type polypeptide.

In some embodiments, a single-stranded RNA (e.g., mRNA) may comprise a secretion signal-encoding region (e.g., a secretion signal-encoding region that allows an encoded target entity or entities to be secreted upon translation by cells). In some embodiments, such a secretion signal-encoding region may be or comprise a non-human secretion signal. In some embodiments, such a secretion signal-encoding region may be or comprise a human secretion signal.

In some embodiments, a single-stranded RNA (e.g., mRNA) may comprise at least one non-coding element (e.g., to enhance RNA stability and/or translation efficiency). Examples of non-coding elements include but are not limited to a 3′ untranslated region (UTR), a 5′ UTR, a cap structure (e.g., in some embodiments, an enzymatically-added cap; in some embodiments, a co-transcriptional cap), a poly adenine (polyA) tail (e.g., that, in some embodiments, may be or comprise 100 A residues or more, and/or in some embodiments may include one or more “interrupting” [i.e., non-A]sequence elements), and any combinations thereof. Exemplary embodiments of such non-coding elements may be found, for example, in WO2011015347, WO2017053297, U.S. Ser. No. 10/519,189, U.S. Ser. No. 10/494,399, WO2007024708, WO2007036366, WO2017060314, WO2016005324, WO2005038030, WO2017036889, WO2017162266, and WO2017162461, each of which is incorporated herein by referenced in its entirety.

At least four formats useful for RNA pharmaceutical compositions (e.g., immunogenic compositions or vaccines) have been developed, namely non-modified uridine containing mRNA (uRNA), nucleoside modified mRNA (modRNA), self-amplifying mRNA (saRNA), and trans-amplifying RNAs.

Features of a non-modified uridine platform may include, for example, one or more of intrinsic adjuvant effect, good tolerability and safety, and strong antibody and T cell responses.

Features of modified uridine (e.g., pseudouridine) platform may include reduced adjuvant effect, blunted immune innate immune sensor activating capacity and thus augmented antigen expression, good tolerability and safety, and strong antibody and CD4-T cell responses. As noted herein, the present disclosure provides an insight that such strong antibody and CD4 T cell responses may be particularly useful for vaccination.

Features of self-amplifying platform may include, for example, long duration of polypeptide (e.g., protein) expression, good tolerability and safety, higher likelihood for efficacy with very low vaccine dose.

In some embodiments, a self-amplifying platform (e.g., RNA) comprises two nucleic acid molecules, wherein one nucleic acid molecule encodes a replicase (e.g., a viral replicase) and the other nucleic acid molecule is capable of being replicated (e.g., a replicon) by said replicase in trans (trans-replication system). In some embodiments, a self-amplifying platform (e.g., RNA) comprises a plurality of nucleic acid molecules, wherein said nucleic acids encode a plurality of replicases and/or replicons.

In some embodiments, a trans-replication system comprises the presence of both nucleic acid molecules in a single host cell.

In some such embodiments, a nucleic acid encoding a replicase (e.g., a viral replicase) is not capable of self-replication in a target cell and/or target organism. In some such embodiments, a nucleic acid encoding a replicase (e.g., a viral replicase) lacks at least one conserved sequence element important for (−) strand synthesis based on a (+) strand template and/or for (+) strand synthesis based on a (−) strand template.

In some embodiments, a self-amplifying RNA comprises a 5′-cap; in some trans-replication systems, at least an RNA encoding a replicase is capped. Without wishing to be bound by any one theory, it has been found that a 5′-cap can be important for high level expression of a gene of interest in trans.

In some embodiments, a self-amplifying platform does not require propagation of virus particles (e.g., is not associated with undesired virus-particle formation). In some embodiments, a self-amplifying platform is not capable of forming virus particles.

In some embodiments, an RNA may comprise an Internal Ribosomal Entry Site (IRES) element. In some embodiments, an RNA does not comprise an IRES site; in particular, in some embodiments, an saRNA does not comprise an IRES site. In some such embodiments, translation of a gene of interest and/or replicase is not driven by an IRES element. In some embodiments, an IRES element is substituted by a 5′-cap. In some such embodiments, substitution by a 5′-cap does not affect the sequence of a polypeptide encoded by an RNA.

In some embodiments, a nucleic acid particle described herein comprises modRNA, circRNA, saRNA, taRNA, or uRNA. In some embodiments, a nucleic acid particle comprises modRNA. In some embodiments, a nucleic acid particle comprises saRNA.

In some embodiments, a nucleic acid particle comprises taRNA. In some embodiments, a nucleic acid particle comprises uRNA.

In some embodiments, a nucleic acid particle comprises two or more RNA selected from modRNA, circRNA, saRNA, taRNA, and uRNA.

### Methods of Use

Nucleic acid particles (and compositions comprising said nucleic acid particles) described herein are useful in the treatment and prophylaxis in a subject of diseases, disorders, and conditions described herein. In some embodiments, the present disclosure provides a method of treating a disease, disorder, or condition in a subject comprising administering to the subject a composition comprising nucleic acid particles described herein. In some embodiments, a disease, disorder, or condition is an infectious disease, cancer, an autoimmune disease, or a rare disease.

In some embodiments, the present disclosure provides a nucleic acid particle as described herein for use as a medicament.

In some embodiments, the present disclosure provides a nucleic acid particle as described herein, for use in the treatment and/or prevention of a disease or disorder, wherein the disease or disorder is an infectious disease, cancer, a genetic disorder, an autoimmune disease, or a rare disease.

In some embodiments, an infectious disease is caused by or associated with a viral pathogen. In some embodiments, a viral pathogen is of a family selected from poxviridae, rhabdoviridae, filoviridae, paramyxoviridae, hepadnaviridae, coronaviridae, caliciviridae, picornaviridae, reoviridae, retroviridae, and orthomyxoviridae. In some embodiments, an infectious disease is caused by or associated with a virus selected from SARS-CoV-2, influenza, Crimean-Congo Hemorhhagic Fever (CCHF), Ebola virus, Lassa virus, Marburg virus, HIV, Nipah virus, and MERS-CoV.

In some embodiments, an infectious disease is caused by or associated with a bacterial pathogen. In some embodiments, a bacterial pathogen is of a species selected from Actinomyces israelii, bacillus antracis, Bacteroides fragilis, Bordetella pertussis, Borrelia burgdorferi, Borrelia garinii, Borrelia afzelii, Borrelia recurrentis, Brucella abortus, Brucella canis, Brucella melitensis, Brucella suis, Campolobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium idphteriae, Ehrlichia canis, Ehrlichia chaffeensis, Enterococcus faecalis, Enterococcus faecium, Escherichia coli, Francisella tularensis, Haemophilus influenzae, Helicobacter pylori, Klebsiella pneumoniae, Legionella pneumophila, Leptospira, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Nocardia asteroids, Rickettsia ricektssii, Salmonella typhi, Salmonella typhimurium, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Vibrio cholerae, and Yersinia pestis. In some embodiments, an infectious disease is caused by or associated with a parasite.

In some embodiments, a parasite is of a family selected from Plasmodium, Leishmania, Cryptosporidium, Entamoeba, Trypanosomas, Schistosomes, Ascaris, Echinococcus and Taeniidae.

In some embodiments, a disease, disorder, or condition is a cancer. In some embodiments, a cancer is selected from bladder cancer, breast cancer, colorectal cancer, kidney cancer, lung cancer, lymphoma, melanoma, oral/oropharyngeal cancer, pancreatic cancer, prostate cancer, thyroid cancer, and uterine cancer.

In some embodiments, a disease, disorder, or condition is a genetic disorder. In some embodiments, a genetic disorder is associated with a gain-of-function mutation or a loss-of-function mutation.

In some embodiments, a disease, disorder, or condition is an autoimmune disease. In some embodiments, an autoimmune disease is selected from addison disease, celiac disease, rheumatoid arthritis, lupus, inflammatory bowel disease, dermatomyositis, multiple sclerosis, diabetes, guillain-barre syndrome, chronic inflammatory demyelinating polyneuropathy, psoriasis, pernicious anemia, Graves' disease, Hashimoto's thyroiditis, myasthenia gravis, and vasculitis sjörgen syndrome.

In some embodiments, a disease, disorder, or condition is a rare disease. As described herein, a rare disease refers to a life-threatening or chronically debilitating diseases which are of such low prevalence (e.g., fewer than 1/2000 people) that special combined efforts are needed to address them.

In some embodiments, a rare disease is selected from Hereditary Angioedema, Hemophilia B, Hereditary Transthyretin Amyloidosis, Transthyretin-related hereditary amyloidosis, Familial amyloid polyneuropathy, Ornithine transcarbamylase deficiency, Phenylketonuria, Primary hyperoxaluria type 1, Wilson's disease, Galactosemia, Methylmalonic acidemia, Glycogen storage disease, Duchenne muscular dystrophy, Spinal muscular atrophy, Cystic fibrosis, Hemophilia A, Myotonic dystrophy type 1, Alpha-1 antitripsyn deficiency, Huntington's disease, Spinocerebellar ataxia, Mitochondrial neurogastrointestinal encephalomyopathy, Charcot-Marie-Tooth Disease, Stargardt Disease, Leber congenital amaurosis, Usher Syndrome, Heterozygous or homozygous familial hypercholesterolemia, Gaucher disease, Fabry disease, Propionic acidemia, Argininosuccinic aciduria, Maple Syrup Urine Disease, and Citrullinemia type 1.

In some embodiments, the present disclosure provides complexes that can selectively target particular systems within a body. As used herein, reference to “targeting” a particular system refers to causing increased expression of RNA derived from cargo in the complex in the desired system. For example, in some embodiments, complexes described herein can selectively target the lungs, liver, spleen, heart, brain, lymph nodes, bladder, kidneys, and pancreas. As described herein, a complex “selectively targets” an organ when a single target expresses mRNA in an amount that is 65% or greater than expression in other organs post administration (e.g., 65% or more of mRNA throughout the body is expressed from a single organ, with the remaining 35% distributed between one or more different organs). In some embodiments, a complex described herein selectively targets the lungs. In some embodiments, a complex described herein selectively targets the liver. In some embodiments, a complex described herein selectively targets the spleen. In some embodiments, a complex described herein selectively targets the heart.

In some embodiments, the present disclosure provides a method of increasing or causing increased expression of RNA in a target in a subject comprising administering to the subject a complex described herein. In some embodiments, a target is selected from the lungs, liver, spleen, heart, brain, lymph nodes, bladder, kidneys, and pancreas.

### Methods of Delivery

The present disclosure provides, among other things, a nucleic acid particle (e.g., in a pharmaceutical composition or a pharmaceutical formulation, as referred to herein) to be administered to a subject. For example, in some embodiments, a composition comprising nucleic acid particles described herein is administered as a monotherapy. In some embodiments, a composition comprising nucleic acid particles described herein is administered as part of a combination therapy. In some embodiments, a concentration of total RNA (e.g., a total concentration of all of the one or more RNA molecules) in a pharmaceutical composition described herein is of about 0.01 mg/mL to about 0.5 mg/mL, or about 0.05 mg/mL to about 0.1 mg/mL.

Pharmaceutical compositions may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety). In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference in its entirety).

Pharmaceutical complexes and compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In some embodiments, pharmaceutical compositions described herein are formulated for intravenous administration. In some embodiments, pharmaceutically acceptable carriers that may be useful for intravenous administration include sterile aqueous solutions or dispersions and sterile powders for preparation of sterile injectable solutions or dispersions.

In some embodiments, a pharmaceutical compositon comprising nucleic acid particles described herein are administered intramuscularly, subcutaneously, intradermally, or intravenously. In some embodiments, a pharmaceutical compositon comprising nucleic acid particles described herein are formulated for subcutaneous (s.c) administration. In some embodiments, a pharmaceutical compositon comprising nucleic acid particles described herein are formulated for intramuscular (i.m) administration. In some embodiments, a pharmaceutical composition comprising nucleic acid particles described herein are formulated for intravenous (i.v.) administration.

Therapeutic compositions typically must be sterile and stable under the conditions of manufacture and storage. The composition can be formulated as a solution, dispersion, powder (e.g., lyophilized powder), microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration.

In some embodiments, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Examples of suitable aqueous and nonaqueous carriers which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

In some embodiments, an active agent that may be included in a pharmaceutical composition described herein is or comprises a therapeutic agent administered in a combination therapy described herein. Pharmaceutical compositions described herein can be administered in combination therapy, i.e., combined with other agents. In some embodiments, such therapeutic agents may include agents leading to depletion or functional inactivation of regulatory T cells. For example, in some embodiments, a combination therapy can include a provided pharmaceutical composition with at least one immune checkpoint inhibitor.

In some embodiments, pharmaceutical composition described herein may be administered in conjunction with radiotherapy and/or autologous peripheral stem cell or bone marrow transplantation.

In some embodiments, a pharmaceutical composition described herein can be frozen to allow long-term storage.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

### EXEMPLARY EMBODIMENTS

The present disclosure provides the following non-limiting numbered embodiments.

Embodiment 1. A nucleic acid particle comprising RNA, an immunomodulator, and a cationic lipid or a cationic polymer.

Embodiment 2. The nucleic acid particle of Embodiment 1, wherein the immunomodulator is not dexamethasone or a dexamethasone prodrug.

Embodiment 3. The nucleic acid particle of Embodiments 1 or 2, wherein the immunomodulator is about 0.001 to about 50 mol % of the total amount of lipids or polymers and immunomodulators in the nucleic acid particle.

Embodiment 4. The nucleic acid particle of any one of Embodiments 1-3, wherein the immunomodulator is about 0.01 to about 0.9 mol % of the total amount of lipids or polymers and immunomodulators in the nucleic acid particle.

Embodiment 5. The nucleic acid particle of any one of Embodiments 1-4, wherein the immunomodulator is a small molecule agonist or antagonist of TLR or PRR receptors.

Embodiment 6. The nucleic acid particle of any one of Embodiments 1-4, wherein the immunomodulator is a small molecule downstream inhibitor of NF-κβ.

Embodiment 7. The nucleic acid particle of any one of Embodiments 1-4, wherein the immunomodulator is an sp²-iminosugar glycolipid.

Embodiment 8. The nucleic acid particle of any one of Embodiment 1-4, wherein the immunomodulator is a TLR inhibitor.

Embodiment 9. The nucleic acid particle of Embodiment 8, wherein the TLR inhibitor is an inhibitor of TLR2, TLR4, and/or TLR6.

Embodiment 10. The nucleic acid particle of Embodiment 9, wherein the TLR inhibitor is an inhibitor of TLR4.

Embodiment 11. The nucleic acid particle of Embodiment 10, wherein the inhibitor of TLR4 is TAK-242.

Embodiment 12. The nucleic acid particle of any one of Embodiments 1-4, wherein the immunomodulator is a terpenoid.

Embodiment 13. The nucleic acid particle of Embodiment 12, wherein the terpenoid is a triterpene.

Embodiment 14. The nucleic acid particle of Embodiment 13, wherein the triterpene is synthetic or natural derivative of amyrin, betulinic acid, oleanolic acid, sterols, squalene or ursolic acid.

Embodiment 15. The nucleic acid particle of Embodiment 14, wherein the immunomodulator is a sterol.

Embodiment 16. The nucleic acid particle of Embodiment 15, wherein the sterol is a cortiocosteroid.

Embodiment 17. The nucleic acid particle of Embodiment 16, wherein the corticosteroid is a glucocorticoid.

Embodiment 18. The nucleic acid particle of Embodiment 17, wherein the glucocorticoid is prednisolone, fluticasone propionate, budesonide or a pharmaceutically acceptable salt thereof.

Embodiment 19. The nucleic acid particle of any one of Embodiments 1-4, wherein the immunomodulator is an inflammasome inhibitor.

Embodiment 20. The nucleic acid particle of Embodiment 19, wherein the inflammasome inhibitor is a NLRP3 inflammasome inhibitor, an AIM2 inflammasome inhibitor, a caspase-1 inhibitor, or a pan-casase inhibitor.

Embodiment 21. The nucleic acid particle of Embodiment 20, wherein the inflammasome inhibitor is selected from glyburide (e.g., glibenclamide), 5-chloro-2-methoxy-N-[2-(4-sulfamoylphenyl)-ethyl]-benzamide (e.g., 16673-34-0), JC124, FC11A-2, parthenolide, VX-740, VX-765, BAY 11-7082, BHB, MCC950, MNS, CY-09, Tranilast, OLT1177, and oridonin.

Embodiment 22. The nucleic acid particle of Embodiments 20 or 21, wherein the inflammasome inhibitor is MCC950 or BAY 11-7082.

Embodiment 23. The nucleic acid particle of any one of Embodiments 1-22, wherein the lipid nanoparticle further comprises one or more additional immunomodulators.

Embodiment 24. The nucleic acid particle of Embodiment 23, where the one or more additional immunomodulators are or comprise a small molecule agonist or antagonist of TLR or PRR receptors.

Embodiment 25. The nucleic acid particle of Embodiments 23, where the one or more additional immunomodulators are or comprise an inflammasome inhibitor.

Embodiment 26. The nucleic acid particle of any one of Embodiments 1-25, wherein the nucleic acid particle comprises the immunomodulator, a cationic lipid, and RNA.

Embodiment 27. The nucleic acid particle of Embodiment 26, wherein the nucleic acid particle is in the form of a lipid nanoparticle.

Embodiment 28. The nucleic acid particle of any one of Embodiments 1-27, wherein the cationic lipid is a lipid comprising one or more nitrogen atoms that are cationic or ionizable at physiological pH (e.g., about 7.4).

Embodiment 29. The nucleic acid particle of any one of Embodiments 1-28, wherein the cationic lipid is selected from Table 1 and/or Table 2.

Embodiment 30. The nucleic acid particle of Embodiment 29, wherein the cationic lipid is ALC-0315, SM-102, ALC366, DODMA, or HY-501.

Embodiment 31. The nucleic acid particle of any one of Embodiments 27-30, wherein the cationic lipid is about 30 to about 50 mol % of the total amount of lipids and immunomodulators in the lipid nanoparticle.

Embodiment 32. The nucleic acid particle of any one of Embodiments 27-31, further comprising a helper lipid.

Embodiment 33. The nucleic acid particle of Embodiment 32, wherein the helper lipid is a phospholipid.

Embodiment 34. The nucleic acid particle of Embodiment 33, wherein the helper lipid is selected from: phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₋₆ Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and combinations thereof.

Embodiment 35. The nucleic acid particle of Embodiment 34, wherein the helper lipid is DSPC.

Embodiment 36. The nucleic acid particle of any one of Embodiments 32-34, wherein the lipid nanoparticle comprises about 5 to about 15 mol % of the helper lipid relative to the total amount of lipids and immunomodulators.

Embodiment 37. The nucleic acid particle of any one of Embodiments 27-36, further comprising a polymer-conjugated lipid.

Embodiment 38. The nucleic acid particle of Embodiment 37, wherein the polymer-conjugated lipid is a PEG-lipid selected from PEG-DAG, PEG-PE, PEG-S-DAG, PEG2000-DMG, PEG-cer, a PEG dialkyoxypropylcarbamate, ALC-0159, and combinations thereof.

Embodiment 39. The nucleic acid particle of Embodiment 38, wherein the PEG-lipid is ALC-0159 or PEG2000-DMG.

Embodiment 40. The nucleic acid particle of Embodiment 39, wherein the PEG-lipid is ALC-0159.

Embodiment 41. The nucleic acid particle of Embodiment 39, wherein the PEG-lipid is PEG2000-DMG.

Embodiment 42. The nucleic acid particle of any one of Embodiments 38-41, wherein the lipid nanoparticle comprises about 1 to about 5 mol % of the PEG-lipid relative to the total amount of lipids and immunomodulators.

Embodiment 43. The nucleic acid particle of Embodiment 27, wherein the cationic lipid is ALC-0315, and the lipid nanoparticle further comprises a helper lipid that is DSPC, and a PEG-lipid that is ALC-0159.

Embodiment 44. The nucleic acid particle of Embodiment 27, wherein the cationic lipid is SM-102, and the lipid nanoparticle further comprises a helper lipid that is DSPC, and a PEG-lipid that is PEG2000-DMG.

Embodiment 45. The nucleic acid particle of Embodiment 27, wherein the cationic lipid is HY-501, and the lipid nanoparticle further comprises a helper lipid that is DSPC.

Embodiment 46. The nucleic acid particle of any one of Embodiments 27-44 further comprising an additional terpenoid.

Embodiment 47. The nucleic acid particle of Embodiment 46, wherein the additional terpenoid is a triterpene.

Embodiment 48. The nucleic acid particle of Embodiment 47, wherein the triterpene is a sterol.

Embodiment 49. The nucleic acid particle of Embodiment 48, wherein the sterol is selected from β-sitosterol, stigmasterol, cholesterol, cholecalciferol, ergocalciferol, calcipotriol, botulin, lupeol, ursolic acid, oleanolic acid, cycloartenol, lanosterol, or a-tocopherol.

Embodiment 50. The nucleic acid particle of any one of Embodiments 27-49, wherein the lipid nanoparticle further comprises a surfactant that is or comprises a polysorbate, a poloxamer, and/or a compound comprising an amphiphilic moiety selected from polyalkylene glycols (e.g., polyethylene glycol), poly(2-oxazoline), poly(2-oxazine), polysarcosine, polyvinylpyrrolidone, and poly[N-(2-hydroxypropyl)methacrylamide, wherein the amphiphilic moiety is bonded to one or more C₁₋₂-C₂₀ aliphatic groups.

Embodiment 51. The nucleic acid particle of Embodiment 50, wherein the surfactant is or comprises a polysorbate selected from polysorbate 20, polysorbate 40, polysorbate 60, polysorbate 80, and combinations thereof.

Embodiment 52. The nucleic acid particle of Embodiment 27, wherein the lipid nanoparticle comprises:
i) about 30 to about 50 mol % of the cationic lipid;
ii) about 1 to about 5 mol % of a PEG-lipid; and
iii) about 5 to about 15 mol % of a helper lipid.

Embodiment 53. The nucleic acid particle of Embodiment 52, wherein the lipid nanoparticle comprises:
i) about 30 to about 50 mol % of ALC-0315;
ii) about 1 to about 5% mol % of ALC-0159; and
iii) about 5 to about 15% mol % of DSPC.

Embodiment 54. The nucleic acid particle of Embodiment 53, wherein the lipid nanoparticle comprises:
i) about 47.5 mol % of ALC-0315;
ii) about 1.8 mol % of ALC-0159; and
iii) about 10 mol % of DSPC.

Embodiment 55. The nucleic acid particle of Embodiment 27, wherein the lipid nanoparticle comprises:
i) about 30 to about 50 mol % of SM-102;
ii) about 1 to about 5 mol % of a PEG2000-DMG; and
iii) about 5 to about 15 mol % of DSPC.

Embodiment 56. The nucleic acid particle of Embodiment 55, wherein the lipid nanoparticle comprises:
i) about 50 mol % of SM-102;
ii) about 1.5 mol % of PEG2000-DMG; and
iii) about 10 mol % of DSPC.

Embodiment 57. The nucleic acid particle of any one of Embodiments 27-56, wherein the lipid nanoparticle is characterized by an N/P ratio that is from about 4:1 to about 12:1.

Embodiment 58. The nucleic acid particle of Embodiment 57, wherein the lipid nanoparticle is characterized by an N/P ratio that is about 6:1.

Embodiment 59. The nucleic acid particle of any one of Embodiments 27-58, wherein the lipid nanoparticle has a diameter of about 50 nm to about 150 nm.

Embodiment 60. The nucleic acid particle of Embodiments 1-25, wherein the nucleic acid particle comprises the immunomodulator, the cationic polymer, and RNA.

Embodiment 61. The nucleic acid particle of Embodiment 60, wherein the cationic polymer is poly(ethyleneimine) or poly(propyleneimine).

Embodiment 62. The nucleic acid particle of Embodiments 60 or 61, wherein the nucleic acid particle further comprises a secondary polymer.

Embodiment 63. The nucleic acid particle of Embodiment 62, wherein the secondary polymer is polysarcosine.

Embodiment 64. The nucleic acid particle of any one of Embodiments 1-63, wherein the RNA is mRNA Embodiment 65. The nucleic acid particle of Embodiment 64, wherein the RNA is modRNA, circRNA, saRNA, taRNA, or uRNA.

Embodiment 66. A method of increasing or causing increased expression of RNA in a target in a subject, the method comprising administering to the subject the nucleic acid particle of any one of Embodiments 1-65.

Embodiment 67. The method of Embodiment 66, wherein the target is selected from the lungs, liver, spleen, heart, brain, lymph nodes, bladder, kidneys, and pancreas.

Embodiment 68. A method of treating a disease, disorder, or condition in a subject comprising administering to the subject the nucleic acid particle of any one of Embodiments 1-65.

Embodiment 69. The method of Embodiment 68, wherein the disease, disorder, or condition is an infectious disease, cancer, a genetic disorder, an autoimmune disease, or a rare disease.

Embodiment 70. The method of any one of Embodiments 66-69, wherein the nucleic acid particle is administered parenterally or intranasally.

Embodiment 71. The method of Embodiment 70, wherein the nucleic acid particle is administered intramuscularly, subcutaneously, intradermally, or intravenously.

Embodiment 72. A nucleic acid particle of any one of Embodiments 1-65 for use as a medicament.

Embodiment 73. A nucleic acid particle of any one of 1-65 for use in the treatment and/or prevention of a disease or disorder, wherein the disease or disorder is an infectious disease, cancer, a genetic disorder, an autoimmune disease, or a rare disease.

### EXAMPLES

As described in the Examples below, in certain exemplary embodiments, compositions are prepared according to the following general procedures. It will be appreciated that, although the general methods depict the synthesis of certain compositions of the present disclosure, the following general methods and other methods known to one of ordinary skill in the art can be applied to all compositions and subclasses and species of each of these compositions, as described herein.

Table of Abbreviations

| | |
|---|---|
| DMSO | Dimethylsulfoxide |
| DSPC | 1,2-distearoyl-sn-glycero-3-phosphocholine |
| HBS | HEPES buffered saline |
| LNP | Lipid nanoparticle |
| PCL | Polymer-conjugated lipid |
| PEG-DMG or | 1,2-Dimyristoyl-sn-glycero-3-methoxypolyethylene |
| PEG-DMG 2000 | glycol-2000 |
| PEI | Poly(ethyleneimine) |
| PMBC or | Peripheral blood mononuclear cells or human |
| hPMBC | peripheral blood mononuclear cells |
| pSar | polysarcosine |
| PGA | polyglutamate |

### Analytical Methods

### Size Determination

Particle sizes were determined by dynamic light scattering (DLS) using DynaPro Plate Reader II (Wyatt, Dernbach, Germany). Measurements were conducted in 96-well plate with LNP samples diluted to 0.005 mg/mL (RNA concentration) in 1×PBS (pH 7.4) and measured in at least duplicates at 23° C. A refractive index of 1.33, dispersant viscosity of 1.019 and a run of 10 acquisitions were used as settings for the measurement. Average size and size distribution expressed as polydispersity index (PDI) were obtained using Dynamics software 7.8.1.3 (Wyatt Technology, Santa Barbara, CA, USA).

### Zeta Potential Determination

Surface charges of the particles were assessed by photon correlation spectroscopy using Nicomp© 380 DLS/ZLS System (Anasysta e. k., Mulheim, Germany). LNPs were diluted to 0.002 mg/mL (RNA concentration) using 0.1×PBS (pH 7.4) in a single use plastic cuvette. Measurements were conducted with an electric field strength of 4 V/cm during 180 sec at 23° C.

### Encapsulation Efficiency and RNA Content Determination

The encapsulation efficiency of mRNA in LNPs was quantified using Invitrogen™ Quant-iT RiboGreen assay (Fisher Scientific GmbH, Schwerte, Germany). Samples of 0.05 mg/mL (RNA concentration) were diluted 100-fold in 1×Tris-HCl-EDTA buffer (TE Buffer 20×, Thermo Fisher Scientific, Waltham, MA, USA) to determine RNA accessibility and/or free RNA and diluted in 1×TE buffer containing 0.5% Triton X-100 (Merck Chemicals GmbH/Millipore, Darmstadt, Germany) to determine the total RNA in LNPs. RNA standards (0.1 to 1.5 μg/mL) were also prepared in 1×TE buffer and 0.5% Triton X-100 to account for any variation in fluorescence. Samples were loaded in black 96-well plate and RiboGreen reagent was added to all samples as well as standards. Fluorescence signal is measured after 5 min incubation in dark using microplate reader (Tecan Infinite F200PRO, Maennedorf, Switzerland) at an excitation of 485 nm and emission at 528 nm.

### RNA Integrity

Integrity of the encapsulated mRNA was measured through a Fragment Analyzer device (FA), which is a parallel capillary electrophoresis instrument (Agilent, California, USA). Samples of 10 ul LNPs (0.05 mg/mL RNA concentration) were mixed with 20 ul of 20% Triton in 30% EtOH. Mixed samples were incubated 20 min at 30° C. at 600 rpm. Next, 2 ul of the samples were mixed with 18 ul of the High Sensitivity RNA Kit (Agilent, California, USA) and placed in a 96-well plate. Samples were denatured by incubation for 2 min at 70° C. Plate is cool-down 5 min on ice and then introduced on the FA device. Naked mRNA is measured as a control. RNA integrity is reported in values (%) normalized to naked mRNA.

### Example 1—LNPs Comprising TLR/PRR Inhibitors

In the present example, the impact of different TLR and/or PRR inhibitors is tested for benchmarking. Said benchmark LNP comprises a cationic lipid (e.g., a cationic lipid from Table 1 or Table 2), cholesterol, DSPC and PEG-DMG at molar ratios of 47.5:40.5:10:2. Each of the TLR/PRR inhibitors might inhibit one or more of: TLR, RLR, CDS & STING, CLR, and AhR. The different TLR/PRR inhibitors are tested individually with the benchmark LNPs by inclusion of the respective inhibitor in the lipid mixture in ethanol required for the manufacturing of the LNP. LNPs are prepared according to methods known to those of skill in the art, as referenced in the present disclosure.

The concentration of the TLR/PRR inhibitor is calculated as a molar ratio to the cationic lipid. Suitable molar ratios of cationic lipid to inhibitor are between 1:1 and 1:0.01. The LNPs of the present example are produced in hepes buffered sucrose (HBS, hepes 10 mM, 10% w/v sucrose) following a standard LNP production method known to those of skill in the art and referenced herein. Briefly, the LNP is produced at a mixing ratio of lipid mixture (optionally including a TLR/PRR inhibitor) in ethanol mixed at a 3:1 ratio with modRNA in a citrate buffer (pH 4.5) and at a speed of 12 mL/min. The LNP is then dialyzed for 3 hours followed by a final exchange to HBS.

IL-6, TNF-α, IL1-β, IFN-γ, MIP1-, IP-10 and MCP-1 secretion is measured after 24 hrs of transfection of 0.5e⁵ human PMBCs with doses from 0.01 to 3 μg LNP-mRNA per well. Said cells will be evaluated in supernatant using MSD V-Plex Kit Standard Protocol. Primary human hepatocytes are transfected with a dose of modRNA that can vary between 0.01 and 1 μg LNP-mRNA per well (in total volume of 70 μl) and luciferase expression is evaluated over the course of up to 7 days. LNP hydrodynamic diameter was evaluated with dynamic light scattering and RNA concentrations was measured through UV/Vis absorption of RNA.

In vivo studies are performed in mice using intravenous (i.v.) single and repetitive four weekly injections of LNP-mRNA with/without inhibitors. Cytokine/chemokine expression (IL-6, TNFa, MCP-1 and MIP1b) is evaluated in blood serum 6 hours after single or 6 hours after each repetitive injection. OTC mRNA expression in liver using quantitative Western Blot (Jess) and OTC enzyme activity (colorimetric assay) is evaluated at day 1, day 3 and day 7 after the single injection and on day 7 after the fourth repetitive injection. Applied dose range for both single and repetitive injections include dose of: 5 μg, 10 μg, 20 μg and 60 μg of LNP-mRNA per mouse.

### Example 2—LNPs Comprising TLR/PRR Inhibitors or Terpenoids and Cholesterol

In the present example, the impact of different TLR/PRR inhibitors or terpenoids as immunomodulators are tested by gradual substitution of the cholesterol fraction in example LNPs. Example LNPs comprise a cationic lipid (e.g., a cationic lipid of Table 1 or Table 2), cholesterol, DSPC and PEG-DMG at a molar ratio 47.5:40.5:10:2. Each of the immunomodulator might modulate (activate or inhibit) one or more of TLR, RLR, CDS & STING, CLR, AhR, NLRP3/AIM2 sensors, JAK1/JAK2, TBK1/IKKε, PI3K, IκB-α, MAPK, or MEK1/MEK2. Different immunomodulators are tested individually with the example LNP by substitution of cholesterol with the respective immunomodulator in the lipid mixture in ethanol when manufacturing the LNP. A concentration of immunomodulator is calculated from the cholesterol fraction, said concentration being expressed as a percent of the molar fraction of the total. The concentration can be any within the range of 47.5:40.4:10:2:0.1 to 47.5:0:10:2:40.5 (cationic lipid: cholesterol: DSPC:PEG-DMG: immunomodulator).

The LNP formulation is produced in hepes buffered sucrose (HBS, hepes 10 mM, 10% w/v sucrose) following standard LNP production method widely described in the literature. Briefly, the LNP is produced at a mixing ratio of lipid mixture (optionally including an immunomodulator) in ethanol mixed at a 3:1 ratio with modRNA in a citrate buffer (pH 4.5) and at a speed of 12 mL/min. The LNP was then dialyzed for 3 hours followed by a final exchange to HBS.

IL-6, TNF-α, IL1-β, IFN-γ, MIP1-, IP-10 and MCP-1 secretion is measured after 24 hrs of transfection of 0.5e⁵ human PMBCs. Said cells will be evaluated in supernatant using MSD Kit Standard Protocol. Primary human hepatocytes are transfected with a dose of modRNA that can vary between 0.01 and 1 μg per well (in total volume of 70 μl ) and luciferase expression is evaluated over the course of up to 7 days. LNP hydrodynamic diameter was evaluated with dynamic light scattering and RNA concentrations were measured through UV/Vis absorption of RNA.

In vivo studies are performed in mice using i.v. single and repetitive four weekly injections of LNP-mRNA with/without inhibitors. Cytokine/chemokine expression (IL-6, TNFa, MCP-1 and MIP1b) is evaluated in blood serum 6 hours after single or 6 hours after each repetitive injection. OTC mRNA expression in liver using quantitative Western Blot (Jess) and OTC enzyme activity (colorimetric assay) is evaluated at day 1, day 3 and day 7 after the single injection and on day 7 after the fourth repetitive injection. Applied dose range for both single and repetitive injections include dose of: 5 μg, 10 μg, 20 μg and 60 μg of LNP-mRNA per mouse.

### Example 3—Methods of Preparing Particles

The present example describes a method of preparing lipid nanoparticles (LNPs) and polyplex particles with and without an immunomodulator. Purified RNA was formulated into lipid nanoparticles or polyplex particles using an ethanolic lipid mixture of ionizable cationic lipid or ionizable cationic polymer and transferred into an aqueous buffer system via diafiltration to yield a lipid nanoparticle composition or a polyplex particle composition. To prepare a particle comprising an immunomodulator, the immunomodulator was dissolved in DMSO to 50 mM and then mixed under the sterile conditions with particle described above to 0.5 mol % particle (i.e., particle without an immunomodulator) or the concentration of the particle comprising the immunomodulator was diluted to final RNA concentration of 0.15-0.20 mg/ml, using MES buffered glucose buffer. The final immunomodulator concentration was about 0.99 (pmols/ml of particle). The RNA integrity ranged from about 100 to about 110%

The formulations in the table below comprise modified Luciferase RNA incorporated into the particles described in Table 3:

TABLE 3

| | | | | | | | RNA | RNA | Immunomodulator |
|---|---|---|---|---|---|---|---|---|---|
| | | | | Z-Av | | Zeta | Integrity | Conc. | Conc. (μmols/ml |
| No. | Components | Mol % | Immunomodulator | (nm) | PDI | (mV) | (%) | (mg/mL) | of particle) |
| 1 | SM-102; | 47.5 | N/A | 69.4 | 0.16 | −2.63 | 105.5 | 0.2 | N/A |
| | PEG-DMG; | 2 | | | | | | | |
| | DSPC; | 10 | | | | | | | |
| | Cholesterol | 40.5 | | | | | | | |
| 2 | SM-102; | 47.5 | TAK-242 | 76.8 | 0.13 | −3.3 | 103.2 | 0.2 | 0.99 |
| | PEG-DMG; | 2 | | | | | | | |
| | DSPC; | 10 | | | | | | | |
| | Cholesterol | 40.5 | | | | | | | |
| 3 | SM-102; | 47.5 | MCC950 | 72.4 | 0.11 | −0.69 | 105.0 | 0.2 | 0.99 |
| | PEG-DMG; | 2 | | | | | | | |
| | DSPC; | 10 | | | | | | | |
| | Cholesterol | 40.5 | | | | | | | |
| 4 | SM-102; | 47.5 | BAY 11-7082 | 71.8 | 0.13 | −2.13 | 105.0 | 0.2 | 0.99 |
| | PEG-DMG; | 2 | | | | | | | |
| | DSPC; | 10 | | | | | | | |
| | Cholesterol | 40.5 | | | | | | | |
| 5 | HY-501; | 47.5 | N/A | 70.3 | 0.14 | −2.76 | 100.2 | 0.2 | N/A |
| | PCL | 2 | | | | | | | |
| | DSPC; | 10 | | | | | | | |
| | Cholesterol | 40.5 | | | | | | | |
| 6 | HY-501; | 47.5 | TAK-242 | 82.0 | 0.2 | +2.52 | 100.7 | 0.2 | 0.99 |
| | PCL; | 2 | | | | | | | |
| | DSPC; | 10 | | | | | | | |
| | Cholestero | 40.5 | | | | | | | |
| 7 | HY-501; | 47.5 | MCC950 | 80.6 | 0.1 | −3.53 | 102.2 | 0.2 | 0.99 |
| | PCL; | 2 | | | | | | | |
| | DSPC; | 10 | | | | | | | |
| | Cholesterol | 40.5 | | | | | | | |
| 8 | HY-501; | 47.5 | BAY 11-7082 | 79.5 | 0.1 | −3.75 | 102.3 | 0.2 | 0.99 |
| | PCL; | 2 | | | | | | | |
| | DSPC; | 10 | | | | | | | |
| | Cholesterol | 40.5 | | | | | | | |
| 9 | PEI | 50 | N/A | 50.5 | 0.19 | +5.9 | 83.73 | 0.15 | N/A |
| | PGA-pSar | 50 | | | | | | | |
| 10 | PEI | 50 | TAK-242 | 53.2 | 0.19 | +4.8 | 83.73 | 0.15 | 0.99 |
| | PGA-pSar | 50 | | | | | | | |
| 11 | PEI | 50 | MCC950 | 50.9 | 0.15 | +3.41 | 83.73 | 0.15 | 0.99 |
| | PGA-pSar | 50 | | | | | | | |
| 12 | PEI | 50 | BAY 11-7082 | 54.5 | 0.27 | +3.8 | 83.73 | 0.15 | 0.99 |
| | PGA-pSar | 50 | | | | | | | |
| 13 | PEI | 100 | N/A | 33.9 | 0.22 | 25 | | 0.15 | N/A |
| 14 | PEI | 100 | TAK-242 | 52.8 | Multimodal | +36.3 | | 0.15 | 0.99 |
| 15 | PEI | 100 | MCC950 | 35.7 | 0.24 | 35 | | 0.15 | 0.99 |
| 16 | PEI | 100 | BAY 11-7082 | 36.2 | 0.24 | +28.2 | | 0.15 | 0.99 |
| 17 | DODMA | 47.5 | N/A | 80.9 | 0.318 | 6.44 | 99 | 0.2 | 0.99 |
| | PEG-DMG | 2 | | | | | | | |
| | DSPC | 10 | | | | | | | |
| | Cholesterol | 40.5 | | | | | | | |
| 18 | DODMA | 47.5 | TAK-242 | 87.85 | 0.313 | N/A | 99 | 0.2 | 0.99 |
| | PEG-DMG | 2 | | | | | | | |
| | DSPC | 10 | | | | | | | |
| | Cholesterol | 40.5 | | | | | | | |
| 19 | DODMA | 47.5 | MCC950 | 182.1 | 0.3055 | N/A | 99 | 0.2 | 0.99 |
| | PEG-DMG | 2 | | | | | | | |
| | DSPC | 10 | | | | | | | |
| | Cholesterol | 40.5 | | | | | | | |
| 20 | DODMA | 47.5 | BAY 11-7082 | 64.85 | 0.2965 | N/A | 99 | 0.2 | 0.99 |
| | PEG-DMG | 2 | | | | | | | |
| | DSPC | 10 | | | | | | | |
| | Cholesterol | 40.5 | | | | | | | |

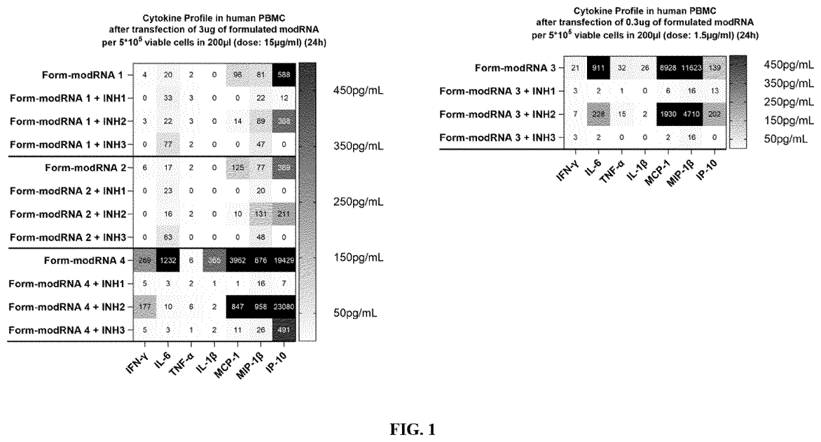

Table 4 is a leqend that corresponds to FIGS. 1-11

| No. | Name |
|---|---|
| 1 | Form-modRNA 1 |
| 2 | Form-modRNA 1 + INH1 |
| 3 | Form-modRNA 1 + INH2 |
| 4 | Form-modRNA 1 + INH3 |
| 5 | Form-modRNA 2 |
| 6 | Form-modRNA 2 + INH1 |
| 7 | Form-modRNA 2 + INH2 |
| 8 | Form-modRNA 2 + INH3 |
| 9 | Form-modRNA 4 |
| 10 | Form-modRNA 4 + INH1 |
| 11 | Form-modRNA 4 + INH2 |
| 12 | Form-modRNA 4 + INH3 |
| 13 | Form-modRNA 3 |
| 14 | Form-modRNA 3 + INH1 |
| 15 | Form-modRNA 3 + INH2 |
| 16 | Form-modRNA 3 + INH3 |
| 17 | Form-modRNA 5 |
| 18 | Form-modRNA 5 + INH1 |
| 19 | Form-modRNA 5 + INH2 |
| 20 | Form-modRNA 5 + INH3 |

### Example 4—Cytokine and Chemokine Profile in hPBMCs

Particles used in the present example were prepared according to Example 3. Human buffy coats from healthy individuals were obtained from the Faculty of Medicine of Johannes Gutenberg University, Mainz and used to isolate peripheral blood mononuclear cells (PBMCs) by Ficoll-Paque™ PLUS (Cytiva, Marlborough, MA, USA) density gradient. For mRNA transfection, cryopreserved PBMCs were thawed and seeded into 96-well plates at a density of 5×10⁵ cells per well in 180 μL RPMI supplemented with 1% non-essential amino acids (NEAA), 1% sodium pyruvate and 10% Fetal Bovine Serum (Merck, Germany). Cells were maintained at 37° C. with 5% CO₂ until transfection with 0.01; 0.04; 0.1; 0.3; 1 and 3 μg/well of the particles reported in Tables 3 and 4 to provide a final volume of 20 μl of transfection mixture added to cells resulting in a total volume of 200 μL per well. The complexed RNA was added to each well in triplicates and supernatants were collected for measurement of cytokine and chemokine secretion profile at 24 h after transfection.

To determine the production of selected cytokines and chemokines, supernatants from human PBMCs transfected with LNP-RNA were subjected to cytokine/chemokine profile analysis using the Meso Scale Discovery V-PLEX Custom Human Biomarkers Proinflammatory and Chemokine Panel (Meso Scale Diagnostics—MSD, Rockville, MD, USA) according to the manufacturer's instructions. A sample dilution of 1:5=supernatant:MSD diluent was used in each experiment. The levels of TNF-α (Tumor necrosis factor alpha), IFN-γ (Interferon gamma), IL-6 (Interleukin 6), IL-1p (Interleukin 1 beta), MIP-1β (Macrophage inflammatory protein 1 beta), MCP-1 (Monocyte chemoattractant protein-1) and IP-10 (Interferon gamma-induced protein 10) were quantified 24 h after the mRNA transfection. To determine viability of the cells, Cell Proliferation Kit II (XTT), Roche was used according to manufacturer's recommendation. In short, after collecting supernatants for cytokine quantification, 50 μl of media and 50 μl of the XTT labeling mixture was added to the cells and incubated for 24 h at at 37° C. and 5% CO₂. The colorimetric assay was measured on 550-670 nm using a Tecan or CLARIOstar readers.

FIG. 1 is a heatmap showing cytokine profile concentration in human PBMC (hPBMC) for Formulations 1-16 described herein after transfection of 3 μg or 0.3 μg of formulated modRNA per 5×105 viable cells.

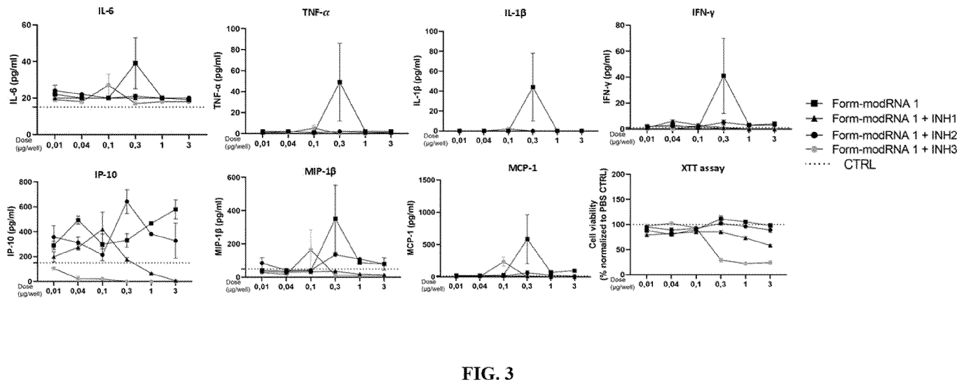

FIG. 3 is a series of plots providing the cytokine profile across a variety of concentrations (0.01 to 3 μg/well) for Formulations 1-4, and the results from an XTT assay of Formulations 1-4.

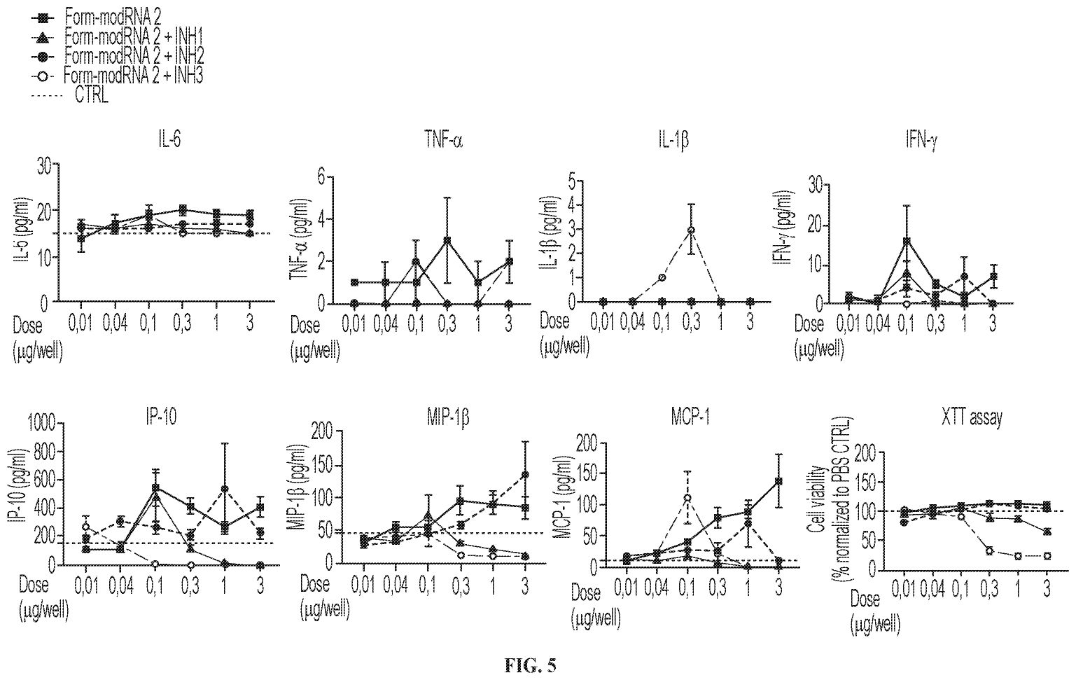

FIG. 5 is a series of plots providing the cytokine profile across a variety of concentrations (0.01 to 3 μg/well) for Formulations 5-8, and the results from an XTT assay of Formulations 5-8.

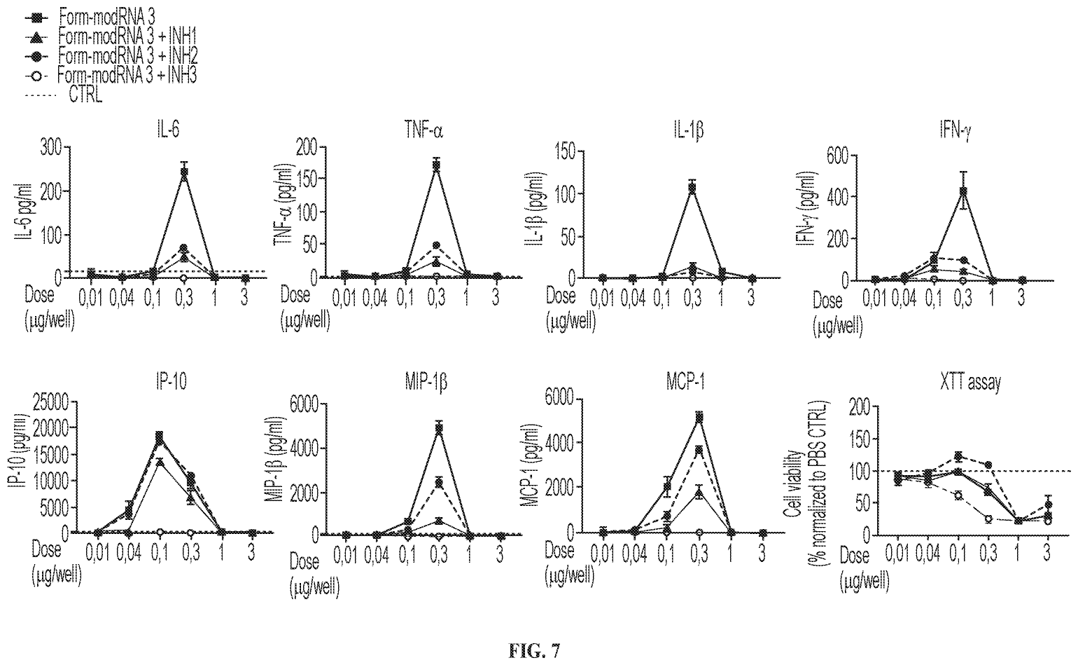

FIG. 7 is a series of plots providing the cytokine profile across a variety of concentrations (0.01 to 3 μg/well) for Formulations 13-16, and the results from an XTT assay of Formulations 13-16.

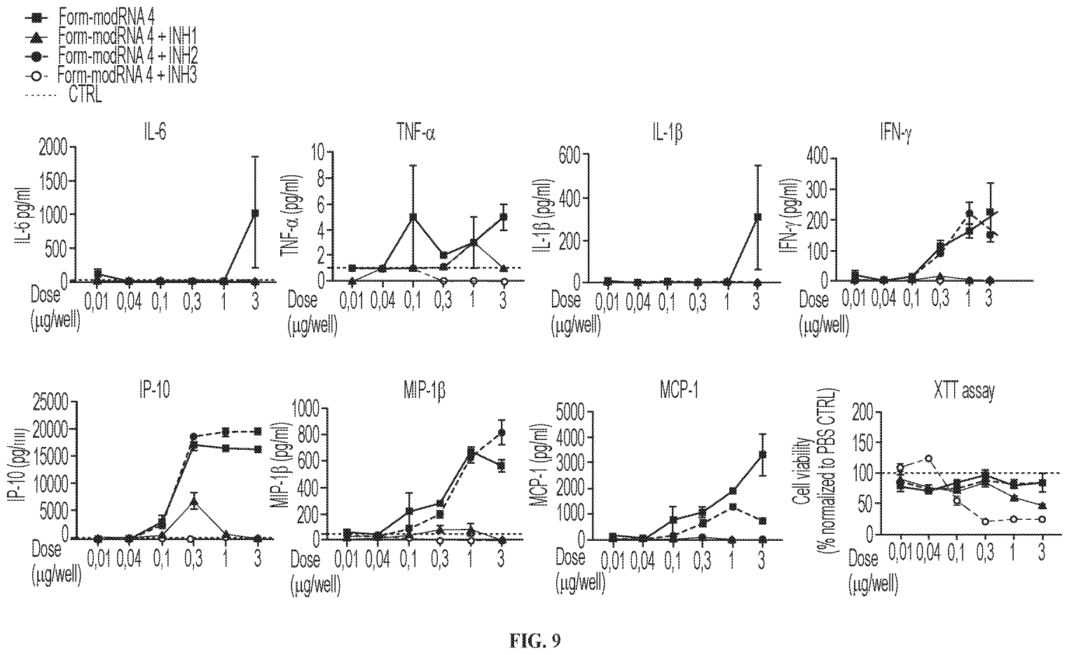

FIG. 9 is a series of plots providing the cytokine profile across a variety of concentrations (0.01 to 3 μg/well) for Formulations 9-12, and the results from an XTT assay of Formulations 9-12.

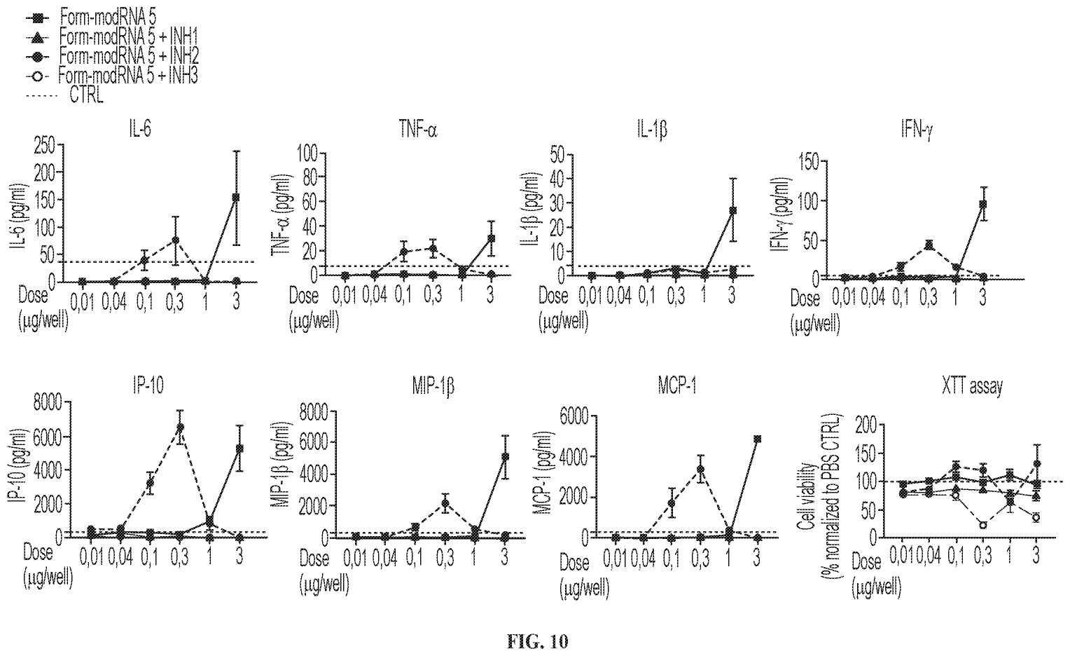

FIG. 10 is a series of plots providing the cytokine profile across a variety of concentrations (0.01 to 3 μg/well) for Formulations 17-20, and the results from an XTT assay of Formulations 17-20.

### Example 5—Primary Human Hepatocytes Culture and Transfection

The present example evaluates the translation of particles reported in Table 3, in-vitro transfection experiments using primary human hepatocytes were performed. Male human cryoplateable hepatocytes were cultured according to the manufacturer's instructions. Cells were stored in liquid nitrogen and after thawing, transferred to 5 mL of InVitroGRO™ CP medium containing Torpedo™ antibiotic mix. The total cell count was determined by using a CASY cell counter. 25,000 cells per well were plated on 96-well collagen I multiwell microplates, and after 2-4 h growth at 37° C. and 5% CO₂, the medium was disposed and replaced with fresh InVitroGRO™ CP medium with Torpedo™ antibiotic mix.

A dose range: 0.01; 0.04; 0.1; 0.3 and 1 μg/well of the particles of Table 3 was prepared by diluting the particles in phosphate-buffered saline (PBS) and resuspending in InVitroGRO HI medium with Torpedo™ antibiotic mix in a volume of 70 μL per well. Primary human hepatocytes were cultured for 24 h, the medium was disposed, and cells were transfected in triplicates using prepared mixture of the particles with a medium. In dose-response experiments, a time course including day 1, day 2 or 3 and day 7 was followed. In experiments longer than 4 days, cells were fed with 20 μL of fresh InVitroGRO HI medium with Torpedo™ antibiotic mix on the fourth day. At selected time points, the medium was disposed and cells were lysed in 100 μL/well of Luciferase Assay System-Cat E1501, Promega lysis buffer. Samples were than diluted 1:2 in lysis buffer and prepared with LUC substrate according to the manufacturer's instructions. For detection of the firefly luciferase activity, photon luminescence emission was measured using a Tecan Infinite 200 Pro (Tecan Trading AG, Mannedorf, Switzerland) or using a CLARIOstar reader.

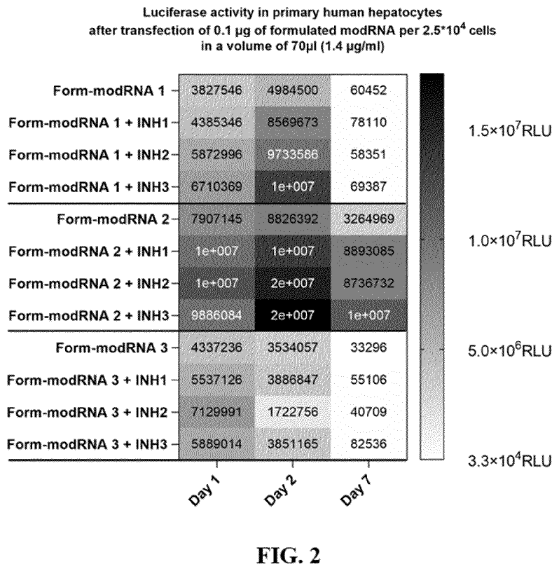

FIG. 2 is a heatmap illustrating luciferase activity in hepatocytes after transfection of 0.1 μg of formulated modRNA per 2.5×10⁴ cells.

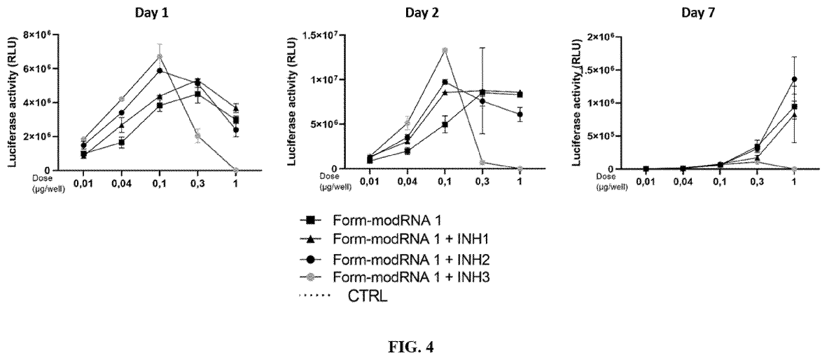

FIG. 4 is a series of plots illustrating luciferase activity (measured as RLUs) on Days 1, 2, and 7 for provided Formulations 1-4.

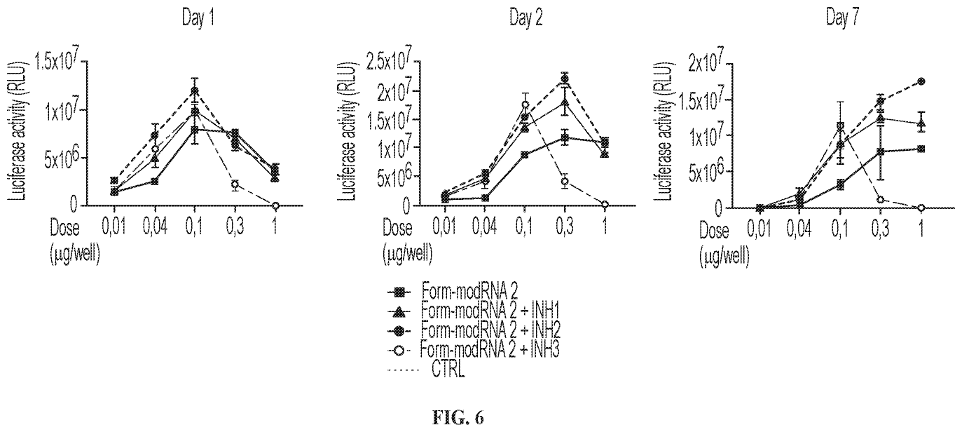

FIG. 6 is a series of plots illustrating luciferase activity (measured as RLUs) on Days 1, 2, and 7 for provided Formulations 5-8.

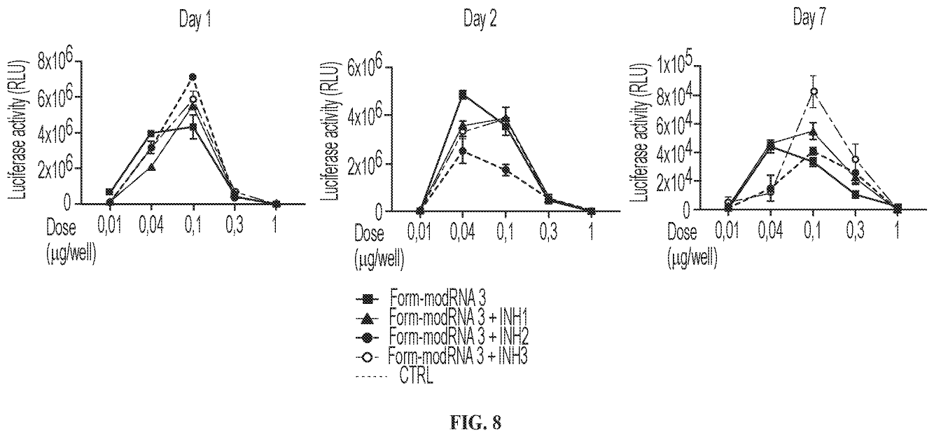

FIG. 8 is a series of plots illustrating luciferase activity (measured as RLUs) on Days 1, 2, and 7 for provided Formulations 13-16.

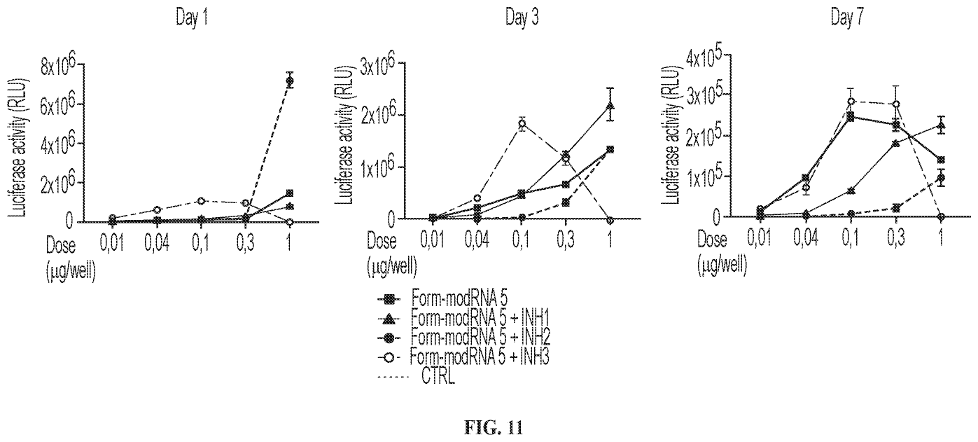

FIG. 11 is a series of plots illustrating luciferase activity (measured as RLUs) on Days 1, 3, and 7 for provided Formulations 17-20.

The embodiments of the disclosure described above are intended to be merely exemplary, numerous variations and modifications will be apparent to those skilled in the art. All such variations and modifications are intended to be within the scope of the present invention as defined in any appended claims.

1. A nucleic acid particle comprising RNA, an immunomodulator, and a cationic lipid or a cationic polymer.

2. The nucleic acid particle of claim 1, wherein the immunomodulator is not dexamethasone or a dexamethasone prodrug.

3. The nucleic acid particle of claim 1 or 2, wherein the immunomodulator is about 0.001 to about 50 mol % of the total amount of lipids or polymers and immunomodulators in the nucleic acid particle.

4. The nucleic acid particle of any one of claims 1-3, wherein the immunomodulator is about 0.01 to about 0.9 mol % of the total amount of lipids or polymers and immunomodulators in the nucleic acid particle.

5. The nucleic acid particle of any one of claims 1-4, wherein the immunomodulator is a small molecule agonist or antagonist of TLR or PRR receptors.

6. The nucleic acid particle of any one of claims 1-4, wherein the immunomodulator is a small molecule downstream inhibitor of NF-κβ.

7. The nucleic acid particle of any one of claims 1-4, wherein the immunomodulator is an sp²-iminosugar glycolipid.

8. The nucleic acid particle of any one of claim 1-4, wherein the immunomodulator is a TLR inhibitor.

9. The nucleic acid particle of claim 8, wherein the TLR inhibitor is an inhibitor of TLR2, TLR4, and/or TLR6.

10. The nucleic acid particle of claim 9, wherein the TLR inhibitor is an inhibitor of TLR4.

11. The nucleic acid particle of claim 10, wherein the inhibitor of TLR4 is TAK-242.

12. The nucleic acid particle of any one of claims 1-4, wherein the immunomodulator is a terpenoid.

13. The nucleic acid particle of claim 12, wherein the terpenoid is a triterpene.

14. The nucleic acid particle of claim 13, wherein the triterpene is synthetic or natural derivative of amyrin, betulinic acid, oleanolic acid, sterols, squalene or ursolic acid.

15. The nucleic acid particle of claim 14, wherein the immunomodulator is a sterol.

16. The nucleic acid particle of claim 15, wherein the sterol is a cortiocosteroid.

17. The nucleic acid particle of claim 16, wherein the corticosteroid is a glucocorticoid.

18. The nucleic acid particle of claim 17, wherein the glucocorticoid is prednisolone, fluticasone propionate, budesonide or a pharmaceutically acceptable salt thereof.

19. The nucleic acid particle of any one of claims 1-4, wherein the immunomodulator is an inflammasome inhibitor.

20. The nucleic acid particle of claim 19, wherein the inflammasome inhibitor is a NLRP3 inflammasome inhibitor, an AIM2 inflammasome inhibitor, a caspase-1 inhibitor, or a pan-casase inhibitor.

21. The nucleic acid particle of claim 20, wherein the inflammasome inhibitor is selected from glyburide (e.g., glibenclamide), 5-chloro-2-methoxy-N-[2-(4-sulfamoylphenyl)-ethyl]-benzamide (e.g., 16673-34-0), JC124, FC11A-2, parthenolide, VX-740, VX-765, BAY 11-7082, BHB, MCC950, MNS, CY-09, Tranilast, OLT1177, and oridonin.

22. The nucleic acid particle of claim 20 or 21, wherein the inflammasome inhibitor is MCC950 or BAY 11-7082.

23. The nucleic acid particle of any one of claims 1-22, wherein the lipid nanoparticle further comprises one or more additional immunomodulators.

24. The nucleic acid particle of claim 23, where the one or more additional immunomodulators are or comprise a small molecule agonist or antagonist of TLR or PRR receptors.

25. The nucleic acid particle of claim 23, where the one or more additional immunomodulators are or comprise an inflammasome inhibitor.

26. The nucleic acid particle of any one of claims 1-25, wherein the nucleic acid particle comprises the immunomodulator, a cationic lipid, and RNA.

27. The nucleic acid particle of claim 26, wherein the nucleic acid particle is in the form of a lipid nanoparticle.

28. The nucleic acid particle of any one of claims 1-27, wherein the cationic lipid is a lipid comprising one or more nitrogen atoms that are cationic or ionizable at physiological pH (e.g., about 7.4).

29. The nucleic acid particle of any one of claims 1-28, wherein the cationic lipid is selected from Table 1 and/or Table 2.

30. The nucleic acid particle of claim 29, wherein the cationic lipid is ALC-0315, SM-102, ALC366, DODMA, or HY-501.

31. The nucleic acid particle of any one of claims 27-30, wherein the cationic lipid is about 30 to about 50 mol % of the total amount of lipids and immunomodulators in the lipid nanoparticle.

32. The nucleic acid particle of any one of claims 27-31, further comprising a helper lipid.

33. The nucleic acid particle of claim 32, wherein the helper lipid is a phospholipid.

34. The nucleic acid particle of claim 33, wherein the helper lipid is selected from: phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₋₆ Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and combinations thereof.

35. The nucleic acid particle of claim 34, wherein the helper lipid is DSPC.

36. The nucleic acid particle of any one of claims 32-34, wherein the lipid nanoparticle comprises about 5 to about 15 mol % of the helper lipid relative to the total amount of lipids and immunomodulators.

37. The nucleic acid particle of any one of claims 27-36, further comprising a polymer-conjugated lipid.

38. The nucleic acid particle of claim 37, wherein the polymer-conjugated lipid is a PEG-lipid selected from PEG-DAG, PEG-PE, PEG-S-DAG, PEG2000-DMG, PEG-cer, a PEG dialkyoxypropylcarbamate, ALC-0159, and combinations thereof.

39. The nucleic acid particle of claim 38, wherein the PEG-lipid is ALC-0159 or PEG2000-DMG.

40. The nucleic acid particle of claim 39, wherein the PEG-lipid is ALC-0159.

41. The nucleic acid particle of claim 39, wherein the PEG-lipid is PEG2000-DMG.

42. The nucleic acid particle of any one of claims 38-41, wherein the lipid nanoparticle comprises about 1 to about 5 mol % of the PEG-lipid relative to the total amount of lipids and immunomodulators.

43. The nucleic acid particle of claim 27, wherein the cationic lipid is ALC-0315, and the lipid nanoparticle further comprises a helper lipid that is DSPC, and a PEG-lipid that is ALC-0159.

44. The nucleic acid particle of claim 27, wherein the cationic lipid is SM-102, and the lipid nanoparticle further comprises a helper lipid that is DSPC, and a PEG-lipid that is PEG2000-DMG.

45. The nucleic acid particle of claim 27, wherein the cationic lipid is HY-501, and the lipid nanoparticle further comprises a helper lipid that is DSPC.

46. The nucleic acid particle of any one of claims 27-45 further comprising an additional terpenoid.

47. The nucleic acid particle of claim 46, wherein the additional terpenoid is a triterpene.

48. The nucleic acid particle of claim 47, wherein the triterpene is a sterol.

49. The nucleic acid particle of claim 48, wherein the sterol is selected from p-sitosterol, stigmasterol, cholesterol, cholecalciferol, ergocalciferol, calcipotriol, botulin, lupeol, ursolic acid, oleanolic acid, cycloartenol, lanosterol, or α-tocopherol.

50. The nucleic acid particle of any one of claims 27-49, wherein the lipid nanoparticle further comprises a surfactant that is or comprises a polysorbate, a poloxamer, and/or a compound comprising an amphiphilic moiety selected from polyalkylene glycols (e.g., polyethylene glycol), poly(2-oxazoline), poly(2-oxazine), polysarcosine, polyvinylpyrrolidone, and poly[N-(2-hydroxypropyl)methacrylamide, wherein the amphiphilic moiety is bonded to one or more C₁₋₂-C₂₀ aliphatic groups.

51. The nucleic acid particle of claim 50, wherein the surfactant is or comprises a polysorbate selected from polysorbate 20, polysorbate 40, polysorbate 60, polysorbate 80, and combinations thereof.

52. The nucleic acid particle of claim 27, wherein the lipid nanoparticle comprises:
i) about 30 to about 50 mol % of the cationic lipid;
ii) about 1 to about 5 mol % of a PEG-lipid; and
iii) about 5 to about 15 mol % of a helper lipid.

53. The nucleic acid particle of claim 52, wherein the lipid nanoparticle comprises:
i) about 30 to about 50 mol % of ALC-0315;
ii) about 1 to about 5% mol % of ALC-0159; and
iii) about 5 to about 15% mol % of DSPC.

54. The nucleic acid particle of claim 53, wherein the lipid nanoparticle comprises:
i) about 47.5 mol % of ALC-0315;
ii) about 1.8 mol % of ALC-0159; and
iii) about 10 mol % of DSPC.

55. The nucleic acid particle of claim 27, wherein the lipid nanoparticle comprises:
i) about 30 to about 50 mol % of SM-102;
ii) about 1 to about 5 mol % of a PEG2000-DMG; and
iii) about 5 to about 15 mol % of DSPC.

56. The nucleic acid particle of claim 55, wherein the lipid nanoparticle comprises:
i) about 50 mol % of SM-102;
ii) about 1.5 mol % of PEG2000-DMG; and
iii) about 10 mol % of DSPC.

57. The nucleic acid particle of any one of claims 27-56, wherein the lipid nanoparticle is characterized by an N/P ratio that is from about 4:1 to about 12:1.

58. The nucleic acid particle of claim 57, wherein the lipid nanoparticle is characterized by an N/P ratio that is about 6:1.

59. The nucleic acid particle of any one of claims 27-58, wherein the lipid nanoparticle has a diameter of about 50 nm to about 150 nm.

60. The nucleic acid particle of claims 1-25, wherein the nucleic acid particle comprises the immunomodulator, the cationic polymer, and RNA.

61. The nucleic acid particle of claim 60, wherein the cationic polymer is poly(ethyleneimine) or poly(propyleneimine).

62. The nucleic acid particle of claim 60 or 61, wherein the nucleic acid particle further comprises a secondary polymer.

63. The nucleic acid particle of claim 62, wherein the secondary polymer is polysarcosine.

64. The nucleic acid particle of any one of claims 1-63, wherein the RNA is mRNA

65. The nucleic acid particle of claim 64, wherein the RNA is modRNA, circRNA, saRNA, taRNA, or uRNA.

66. A method of increasing or causing increased expression of RNA in a target in a subject, the method comprising administering to the subject the nucleic acid particle of any one of claims 1-65.

67. The method of claim 66, wherein the target is selected from the lungs, liver, spleen, heart, brain, lymph nodes, bladder, kidneys, and pancreas.

68. A method of treating a disease, disorder, or condition in a subject comprising administering to the subject the nucleic acid particle of any one of claims 1-65.

69. The method of claim 68, wherein the disease, disorder, or condition is an infectious disease, cancer, a genetic disorder, an autoimmune disease, or a rare disease.

70. The method of any one of claims 66-69, wherein the nucleic acid particle is administered parenterally or intranasally.

71. The method of claim 70, wherein the nucleic acid particle is administered intramuscularly, subcutaneously, intradermally, or intravenously.

72. A nucleic acid particle of any one of claims 1-65 for use as a medicament.

73. A nucleic acid particle of any one of claims 1-65 for use in the treatment and/or prevention of a disease or disorder, wherein the disease or disorder is an infectious disease, cancer, a genetic disorder, an autoimmune disease, or a rare disease.
